SEQUENCE LISTING (3 sequences)

SEQ ID NO: 1 - RNA, 29 nt, Artificial Sequence
  Synthesized nucleic acid substrate
ccucucaaaaacaggagcagaaagacaag

SEQ ID NO: 2 - DNA, 12 bp, Artificial Sequence
  Synthesized nucleic acid substrate
gtctttctgctc

SEQ ID NO: 3 - DNA, 12 bp, Artificial Sequence
  Synthesized nucleic acid probe
gagcagaaagac

(19) United States
(12) Patent Application Publication
(10) Pub. No.: US 2010/0056516 A1
(43) Pub. Date: Mar. 4, 2010

(54) 1-HYDROXY NAPHTHYRIDINE COMPOUNDS AS ANTI-HIV AGENTS

(76) Inventors: Peter D. Williams, Harleysville, PA (US); Shankar Venkatraman, Lansdale, PA (US); H. Marie Langford, Phoenixville, PA (US); Boyoung Kim, Lansdale, PA (US); Theresa M. Booth, Warminster, PA (US); Jay A. Grobler, Gwynedd, PA (US); Donnette Staas, Harleysville, PA (US); Rowena D. Ruzek, North Wales, PA (US); Mark W. Embrey, Harleysville, PA (US); Catherine M. Wiscount, Allentown, PA (US); Terry A. Lyle, Port Townsend, WA (US)

Correspondence Address: MERCK AND CO., INC, P O BOX 2000, RAHWAY, NJ, 07065-0907, US

(21) Appl. No.: 12/373,907

(22) PCT Filed: Jul. 13, 2007
(86) PCT No.: PCT/US07/16052

§ 371 Date: Jan. 15, 2009

Related U.S. Application Data
(60) Provisional application No. 60/831,415, filed on Jul. 17, 2006.

(51) Int. Cl. A61K 31/4375; C07D 471/04; C07D 413/14; A61K 31/5377; A61P 31/18

(52) U.S. Cl. 514/234.5; 546/123; 544/127; 514/300

(57) ABSTRACT

1-Hydroxy naphthyridine compounds (e.g., 1-hydroxy naphthyridin-2(1H)-one compounds of Formula I are inhibitors of HIV integrase and/or HIV RNase H and inhibitors of HIV replication: (I) wherein X and R1-R6 are as defined herein. The compounds are useful in the prophylaxis and treatment of infection by HIV and in the prophylaxis, delay in the onset, and treatment of AIDS. The compounds are employed against HIV infection and AIDS as compounds per se or in the form of pharmaceutically acceptable salts. The compounds and their salts can be employed as ingredients in pharmaceutical compositions, optionally in combination with other anti-HIV agents such as HIV antivirals, immunomodulators, antibiotics and vaccines.

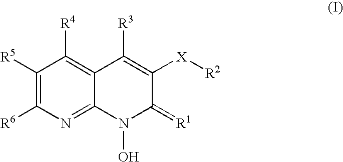

This application claims the benefit of U.S. Provisional Application No. 60/831,415, filed Jul. 17, 2006, the disclosure of which is hereby incorporated by reference in its entirety.

### FIELD OF THE INVENTION

The present invention is directed to 1-hydroxy naphthyridine derivatives and pharmaceutically acceptable salts thereof, their synthesis, and their use as inhibitors against HIV integrase and/or RNase H. The compounds and pharmaceutically acceptable salts thereof of the present invention are useful for preventing or treating infection by HIV and for preventing or treating or delaying the onset of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277 (1985)]. Amino acid sequence homology provides evidence that the pol sequence encodes reverse transcriptase (RT), integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

Reverse transcriptase has three known enzymatic functions. The enzyme acts as an RNA-dependent DNA polymerase, as a ribonuclease H, and as a DNA-dependent DNA polymerase. In its role as an RNA-dependent DNA polymerase; RT uses viral RNA as a template to produce an RNA-DNA hybrid. The ribonuclease H activity of RT has two functions: it makes specific cleavages in the RNA of the RNA-DNA hybrid to create defined RNA primers; and it makes non-specific cleavages in the RNA of the RNA-DNA hybrid resulting in dissociation of the RNA and creating single-stranded DNA. As a DNA-dependent DNA polymerase, RT makes a second, complementary DNA strand using the first DNA strand as a template. The two strands form proviral double-stranded DNA, which is integrated into the host cell's genome by the viral enzyme, integrase.

It is known that compounds that inhibit the enzymatic functions of HIV RT or HIV integrase will inhibit HIV replication in infected cells. These compounds are useful in the prophylaxis or treatment of HIV infection in humans. Among the compounds approved for use in treating HIV infection and AIDS are the RT polymerase inhibitors 3′-azido-3′-deoxythymidine (AZT), 2′,3′-dideoxyinosine (ddI), 2′,3′-dideoxycytidine (ddC), d4T, 3TC, nevirapine, delavirdine, efavirenz and abacavir. These drugs work by inhibiting the polymerase activity of RT.

While each of the foregoing drugs is effective in treating HIV infection and AIDS, there remains a need to develop additional HIV antiviral drugs, including additional RT inhibitors, because of the growing problem of resistance. The continued use of antiviral drugs to prevent HIV infection results in the emergence of mutant strains of HIV which are resistant to the drugs. Mutant HIV strains that are resistant to the approved RT inhibitor drugs named above have already been observed in infected patients. These mutant strains of HIV most commonly contain amino acid mutations near the polymerase active site of RT, the site where these drugs bind to RT. The RNase H active site of RT is remote from the polymerase active site of RT and thus it is expected that compounds which inhibit RT function by binding in or near to the RNase active site will be efficacious at inhibiting RT function in the mutant strains.

The following references are of interest as background:

E. M. Hawes et al., J. Chem. Soc. (C) 1966, pp. 315-321 disclose the preparation of ethyl 1,2-dihydro-1-hydroxy-2-oxo-1,8-naphthyridine-3-carboxylate and 1,2-dihydro-1-hydroxy-2-oxo-1,8-naphthyridine-3-carboxylic acid.

US2004/167123 A1 and US2004/162285 A1 relate to certain 1,1-dioxido-4H-1,2,4-benzothiadiazines as hepatitis C polymerase inhibitors and anti-infective agents.

US2004/162285 A1 relates to certain 1,8-naphthyridines as anti-infective agents.

WO2006/026619 A2 relates to certain substituted thienes as inhibitors of RNase H.

US 2005/0203176 A1 relates to certain dithiocarbamates as inhibitors of the RNase H activity of RT.

US 2005/0203156 A1 relates to certain hydantoin derivatives as inhibitors of the RNase H activity of RT.

US 2005/0203129 A1 relates to certain dihydroquinoline derivatives as inhibitors of the RNase H activity of RT.

US 2004/0138166 A1 relates to oligonucleotide agents that inhibit the RNase H activity of HIV RT.

U.S. Pat. No. 5,527,819 relates to certain compounds related to the natural product, mappicine, as inhibitors of the RNase H activity of RT.

WO 2006026619 A2 relates to certain thiophene derivatives as inhibitors of the RNase H activity of RT.

US 2005203176 A1 relates to certain carbamate derivatives as inhibitors of the RNase H activity of RT.

US 2005203156 A1 relates to certain hydantoins as inhibitors of the RNase H activity of RT.

US 2005203129 A1 relates to certain 1,2-dihydroquinoline derivatives as inhibitors of the RNase H activity of RT.

Dat, et al., Journal of Natural Products (2007), vol. 70, pp. 839-841 describes a natural product lactone with inhibitory activity for HIV Ribonuclease H.

Didieijean, et al., Antimicrobial Agents and Chemotherapy (2005), vol. 49, pp. 4884-4894 56 discuss hydroxylated tropolones with HIV RNase H inhibitory activity.

S. R. Budihas et al., Nucleic Acids Res. (2005) vol. 33, pp. 1249-56 discuss hydroxylated tropolones with HIV RNase H inhibitory activity.

A. Somasunderam et al., Biochemistry (2005) vol. 44, pp. 10388-95 discuss DNA thioaptamers as inhibitors of HIV RNase H activity.

C. A. Shaw-Reid et al., Biochemistry (2005) vol. 44, pp. 1595-1606 and C. A. Shaw-Reid et al., J. Biol. Chem. (2003) vol. 278, pp. 2777-80 discuss a diketoacid HIV RNase H inhibitor.

R. N. Hannoush et al., Nucleic Acids Res. (2004) vol. 32, pp. 6164-6175 discuss oligonucleotide hairpins as inhibitors of HIV RNase H activity.

K. Klumpp et al., Nucleic Acids Res. (2003) vol. 31, No. 23, pp. 6852-59 and J. Qi Hang et al., Biochem. Biophy. Res. Comm. (2004) vol. 317, No. 23, pp. 321-29 discuss 2-hydroxyisoquinoline-1,3(2H,4H)-dione inhibitors of HIV RT RNase H activity.

G. Borko et al., Biochemistry (1997), vol. 36, pp. 3179-3185 discuss acylhydrazone inhibitors HIV RT RNase H activity.

I. W. Althaus et al., Experimentia 52 (1996), Birkhauser-Verlag, pp. 329-335 discuss natural product novenamines as inhibitors HIV RT RNase H activity.

P. Mohan et al., J. Med. Chem. (1994), vol. 37, pp. 2513-2519 discuss naphthalenesulfonic acid derivatives as inhibitors HIV RT RNase H and RT DNA polymerase activities.

P. Hafkemer et al., Nucleic Acids Res. (1991) vol. 19, pp. 4059-65 discuss HIV RNase H inhibitory activity of a cephalosporin degradation product.

S. Loya et al., Antimicrobial Agents and Chemother. (1990) vol. 34, pp. 2009-12 discuss a quinone natural product inhibitor of HIV RNase H activity.

U.S. Pat. No. 6,380,249, U.S. Pat. No. 6,306,891, and U.S. Pat. No. 6,262,055 relate to certain 2,4-dioxobutyric acids and acid esters useful as HIV integrase inhibitors.

WO 01/00578 relates to certain 1-(aromatic- or heteroaromatic-substituted)-3-(heteroaromatic substituted)-1,3-propanediones useful as HIV integrase inhibitors.

US 2003/0055071 (corresponding to WO 02/30930), WO 02/30426, and WO 02/55079 each relate to certain 8-hydroxy-1,6-naphthyridine-7-carboxamides as HIV integrase inhibitors.

WO 02/036734 relates to certain aza- and polyaza-naphthalenyl ketones to be HIV integrase inhibitors.

WO 03/016275 relates to certain compounds having integrase inhibitory activity.

WO 03/35076 relates to certain 5,6-dihydroxypyrimidine-4-carboxamides as HIV integrase inhibitors, and WO 03/35077 relates to certain N-substituted 5-hydroxy-6-oxo-1,6-dihydropyrimidine-4-carboxamides as HIV integrase inhibitors.

WO 03/062204 relates to certain hydroxynaphthyridinone carboxamides that are useful as HIV integrase inhibitors.

WO 04/004657 relates to certain hydroxypyrrole derivatives that are HIV integrase inhibitors.

### SUMMARY OF THE INVENTION

The present invention is directed to 1-hydroxy-1,8-naphthyridine compounds (e.g., 1-hydroxy-1,8-naphthyridin-2(1H)-one compounds). These compounds are useful in the inhibition of HIV RNase H and/or HIV integrase; i.e., certain of the compounds inhibit RNase H, certain of the compounds inhibit integrase, and certain of the compounds inhibit both RNase H and integrase. These compounds are useful for the prophylaxis of infection by HIV, the treatment of infection by HIV and in the prophylaxis, treatment, and delay in the onset of AIDS and/or ARC, either as compounds or their pharmaceutically acceptable salts and/or hydrates (when appropriate), or as pharmaceutical composition ingredients, whether or not in combination with other HIV antiviral agents, anti-infectives, immunomodulators, antibiotics or vaccines. More particularly, one embodiment of the present invention (referred to herein as “Embodiment D0”) includes compounds of Formula I, and pharmaceutically acceptable salts and/or hydrates thereof:

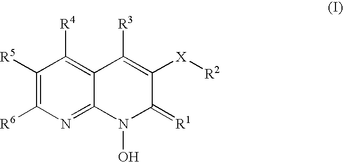

wherein:

### R¹ is O, S, or N—R^{A};

X is a bond, C(O), SO₂, C₁-C₆ alkylene, O, N(R^{A}), or S;
R² is H, halo, CN, C₁-C₁₂ alkyl, C₃-C₈ cycloalkyl, aryl, heteroaryl, N(R⁷)R⁸, or OR⁹; wherein:
the alkyl is optionally substituted with from 1 to 3 substituents each of which is independently selected from the group consisting of halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, and C(O)N(R^{A})—C₁-C₆ alkylene-AryB;
   wherein AryB is phenyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₈ cycloalkyl, CN, SO₂(C₁-C₆ alkyl), S(O)(C₁-C₆ alkyl), N(R^{A})R^{B}, NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-N(R^{A})R^{B}, C₁-C₆ alkylene-CO₂R^{A}, C₁-C₆ alkylene-C(O)R^{A}, or C₁-C₆ alkylene-C(O)N(R^{A})R^{B}
the cycloalkyl, aryl, or heteroaryl is optionally substituted with from 1 to 3 substituents each of which is independently selected from the group consisting of halo, OR^{A}, SR^{A}, C₁-C₆ alkyl, C₁-C₆ haloalkyl, N(R^{A})R^{B}, C₁-C₆ alkylene-N(R^{A})R^{B}, CO₂R^{A}, C₁-C₆ alkylene-CO₂R^{A}: NR^{A}SO₂R^{B}, C₁-C₆ alkylene-NR^{A}SO₂R^{B}, C(O)N(R^{A})R^{B}, C₁-C₆ alkylene-C(O)N(R^{A})R^{B}, C₁-C₆ alkylene-OR^{A}, C₁-C₆ alkylene-SR^{A}, SO₂N(R^{A})R^{B}, SO₂(C₁-C₆ alkyl), S(O)(C₁-C₆ alkyl), C(O)R^{A}, C₁-C₆ alkyene-C(O)R^{A}, NR^{A}CO₂R^{B}, NR^{A}C(O)R^{B}, NR^{A}C(O)N(R^{A})R^{B}, CN, R^{C}, and NO₂;
the alkyl or cycloalkyl is optionally also substituted with an oxo group; and
any two adjacent substituents of the cycloalkyl are optionally taken together with the ring atoms to which they are attached to form a ring fused to the cycloalkyl which is (i) a 5- to 7-membered unsaturated but non-aromatic carbocyclic ring, (ii) a benzene ring, (iii) a 5- or 6-membered heteroaromatic ring containing from 1 to 3 heteroatoms independently selected from N, O and S, or (iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂; and wherein the ring fused to the cycloalkyl is optionally substituted with from 1 to 3 substituents each of which is independently selected from the group consisting of halo, OR^{A}, SR^{A}, N(R^{A})R^{B}, R^{C}, C₁-C₆ alkyl, C₁-C₆ haloalkyl, O—C₁-C₆ haloallyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
   and with the proviso (A) that XR² is not C(O)-halo, C(O)—CN, SO₂-halo, SO₂—CN, O-halo, O—CN, O—OR⁹, N(R^{A})-halo, N(R^{A})—CN, N(R^{A})—OR⁹, N(R^{A})—N(R¹)R⁸, S-halo, S—CN, S—OR⁹, S—N(R⁷)R⁸, N(R^{A})-heteroaryl when the heteroaryl is attached to the N via a ring heteroatom, or S-heteroaryl when the heteroaryl is attached to the S via a ring heteroatom;
   R³ is H, OH, halo, SO₂N(R⁷)R⁸, C₁-C₁₂ alkyl, OR⁹, N(R⁷)R⁸, NR^{A}C(O)R⁸, aryl, heteroaryl other than HetZ, HetZ, or C(O)-heteroaryl; wherein
the alkyl is optionally substituted with from 1 to 3 substituents each of which is independently selected from the group consisting of halo, OR^{A}, OR^{E}, SR^{A}, SR^{E}, N(R^{A})R^{B}, R^{D}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}C₂R^{B}, NR^{A}C(O)R^{B}, NR^{A}C(O)N(R^{A})R^{B}, CO₂R^{A}, C(O)R^{A}, and C(O)N(R^{A})R^{B};
the aryl or heteroaryl is optionally substituted with 1 to 3 substituents each of which is independently selected from the group consisting of halo, OR^{A}, OR^{E}, SR^{A}, SR^{E}, N(R^{A})R^{B}, R^{P}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(A)R^{B}, NR^{A}CO₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ allene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂B, C₁-C₆ alkylene-NR^{A}(O)R^{B}, C₁-C₆ alkylene-NR^{A}(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, N(R^{A})—C₁-C₆ alkylene-C(O)N(R^{A})R^{B}, C(O)N(R^{A})R^{D}, C(O)—HetX, N(R^{A})—C₁-C₆ alkylene-HetX, and C₁-C₆ alkylene-HetX; and wherein HetX independently has the same definition as HetY; and
the HetZ is a fused bicyclic heteroaryl selected from the group consisting of:

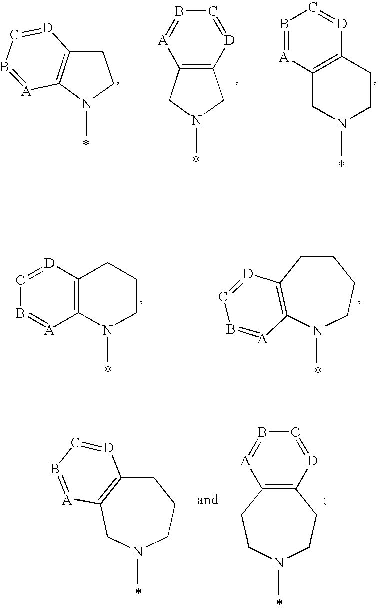

wherein A, B, C and D are each independently N or C-T, with the proviso that no more than two of A, B, C and D is N; and wherein each T is independently H, halo, CN, CO₂R^{B}, OR^{A}, SR^{A}, N(R^{A})R^{B}, N(R^{A})SO₂R^{B}, N(R^{A})CO₂R^{B}, N(R^{A})C(O)R^{B}, N(R^{A})C(O)N(A)R^{B}, NO₂, CN, SO₂(C₁-C₆ alkyl), S(O)(C₁-C₆ alkyl), SO₂N(R^{A})(R^{B}), NR^{A}SO₂R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkyl, C₁-C₆ haloalkyl, C₁-C₆ alkylene-OR^{A}, C₁-C₆ alkylene-SR^{A}, C₁-C₆ alkylene-N(R^{A})R^{B}, C₁-C₆ alkylene-N(e)SO₂R^{B}, C₁-C₆ alkylene-N(R^{A})CO₂R^{A}, C₁-C₆ alkylene-N(R^{A})C(O)R^{B}, C₁-C₆ alkylene-N(R^{A})C(O)N(R^{A}) R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-SO₂N(R^{A})(R^{B}), C₁-C₆ alkylene-NR^{A}SO₂R^{B}, C₁-C₆ alkylene-NR^{A}CO₂R^{B}, C₁-C₆ alkylene-NR^{A}C(O)R^{B}, C₁-C₆ alkylene-NR^{A}C(O)N(A)R^{B}, C₁-C₆ alkylene-C(O)R^{A}, C₁-C₆ alkylene-C(O)R^{A}, C₁-C₆ alkylene-C(O)N(R^{A})R^{B}, C₃-C₉ cycloalkyl, O—C₃-C₈ cycloalkyl, O—C₁-C₆ alkylene-C₃-C₉ cycloalkyl, S—C₃-C₈ cycloalkyl, S—C₁-C₆ alkylene-C₃-C₉ cycloalkyl, aryl, O-aryl, O—C₁-C₆ alkylene-aryl, S-aryl, S—C₁-C₆ alkylene-aryl, N(R^{A})—C₁-C₆ alkylene-aryl, C(O)N(R^{A})—C₁-C₆ alkylene-aryl, heteroaryl, O-heteroaryl, O—C₁-C₆ alkylene-heteroaryl, S-heteroaryl, S—C₁-C₆ alkylene-heteroaryl, N(R^{A})—C₁-C₆ alkylene-heteroaryl, or C(O)N(R^{A})—C₁-C₆ alkylene-heteroaryl, wherein
wherein in each T which is or contains C₃-C₉ cycloalkyl, the C₃-C₈ cycloalkyl is optionally and independently substituted with 1 to 3 substituents each of which is independently halogen, C₁-C₆ alkyl, C₁-C₆ haloalkyl, C₁-C₆ hydroxyalkyl, OR^{A}, N(R^{A})R^{B}, N(R^{A})R^{C}, N(R^{A})R^{E}, N(R^{A})SO₂R^{B}, N(R^{A})CO₂R^{B}, N(R^{A})C(O)R^{B}, N(R^{A})C(O)N(R^{A})R^{B}; NO₂, CN, SO₂(C₁-C₆ alkyl), S(O)(C₁-C₆ alkyl), SO₂N(R^{A})(R^{B}), NR^{A}SO₂R^{B}, NR^{A}CO₂R^{B}, NR^{A}C(O)R^{B}, NR^{A}C(O)N(R^{A})R³, CO₂R^{A}, C(O)R^{A}, or C(O)N(R^{A})R^{B};
wherein in each T which is or contains aryl or heteroaryl, the aryl or heteroaryl is optionally substituted with 1 to 3 substituents each of which is independently selected from the group consisting of halo, OR^{A}, OR^{E}, SR^{A}, SR^{E}, N(A)R^{B}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}-C₁-C₆alkylene-C(O)N(R^{A})R^{B}, CO₂R^{A}, C(O)R^{A}, C(O)N(R^{A})R^{B}, C₁-C₆ alkylene-O, C₁-C₆ alkylene-SO, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-O—C(O)N(O)R^{B}, C₁-C₆ alkylene-CO₂R^{A}, C₁-C₆ alkylene-C(O)R^{A}, C₁-C₆ alkylene-C(O)N(R^{A})R^{B}, C(O)—HetY, and C₁-C₆ alkylene-HetY;
and wherein each HetY is independently a 4- to 7-membered saturated heterocyclyl containing a total of 1 or 2 heteroatoms selected from 1 or 2 N, zero or 10, and zero or 1S, wherein the heterocyclyl is optionally substituted with from 1 to 3 substituents each of which is independently halo, OH, O—C₁-C₆ alkyl, C₁-C₆ alkyl, O—C₁-C₆ haloalkyl, C₁-C₆ haloalkyl, C(O)R^{A}, CO₂R^{A}, or oxo;
   alternatively, XR² and R³ are taken together with the carbon atoms to which each is attached to form:
(i) a 5- to 7-membered unsaturated but non-aromatic carbocyclic ring,
(ii) a benzene ring,
(iii) a 5- or 6-membered heteroaromatic ring containing from 1 to 3 heteroatoms independently selected from N, O and S, wherein each N is optionally oxidized,
(iv) a 5- to 7-membered unsaturated but non-aromatic heterocyclic ring containing from 1 to 3 heteroatoms independently selected from N, O and S, wherein each N is optionally oxidized and each S is optionally in the form of S(O) or S(O)₂, or
(v) a 5- to 7-membered unsaturated but non-aromatic heterocyclic ring having a 5- to 7-membered carbocyclic ring fused thereto via two adjacent carbon atoms in the heterocyclic ring, wherein the heterocyclic ring contains from 1 to 3 heteroatoms independently selected from N, O and S, wherein each N is optionally oxidized and each S is optionally in the form of S(O) or S(O)₂;
   wherein:
the carbocyclic ring of (i), the benzene ring of (ii), the heteroaromatic ring of (iii), the heterocyclic ring of (iv) is fused to the naphthyridine ring to provide a fused tricyclic ring system, or the heterocylic ring of (v) is fused to the naphthyridine ring to provide a fused tetracyclic ring system;
the carbocyclic ring of (i), the benzene ring of (ii), the heteroaromatic ring of (iii), or the heterocyclic ring of (iv) is optionally substituted with from 1 to 4 substituents each of which is independently halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}C₂R^{B}, NR^{A}C(O)R^{B}, NR^{A}C(O)N(A)R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene, —C(O)N(O)R^{B} or phenyl,
   wherein each phenyl is independently and optionally substituted with 1 to 3 substituents each of which is independently halo, C₁-C₆ alkyl, C₁-C₆ haloalkyl, CN, CO₂R^{A}, OR^{A}, SR^{A}, N(R^{A})R^{B}, N(A)SO₂R^{B}, N(R^{A})CO₂R^{B}, N(R^{A})C(O)R^{B}, N(R^{A})C(O)N(R^{A})R^{B}, NO₂, SO₂(C₁₋₆ alkyl), S(O)(C₁-C₆ alkyl), SO₂N(R^{A})(R^{B}), NR^{A}SO₂R^{B}, NR^{A}CO₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-N(e)SO₂R^{B}, C₁-C₆ alkylene-N(R^{A})CO₂R^{B}, C₁-C₆ alkylene-OR^{A}), C₁-C₆ alkylene-N(R^{A})C(O)N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-SO₂N(R^{A})(R^{B}), C₁-C₆ alkylene-NR^{A}SO₂R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, C₃-C₈ cycloalkyl, AryC, O-AryC, O—C₁-C₆ alkylene-AryC, heteroaryl, HetW, C₁-C₆ alkylene-HetW; wherein:
   each AryC independently has the same definition as AryA;
   each HetW independently has the same definition as HetY; and
   each heteroaryl is a 5- or 6-membered heteroaromatic ring containing from 1 to 4 heteroatoms selected from N, O and S, wherein the heteroaromatic ring is optionally substituted with 1 to 3 substituents each of which is independently halo, C₁-C₆ alkyl, C₁-C₆ haloalkyl, CO₂R^{A}, OR^{A}, SR^{A}, N(R^{A})R^{B}, CO₂R^{A}, C(O)R^{A}, C(O)N(R^{A})R^{B}, C₁-C₆ alkylene-OR^{A}, C₁-C₆ alkylene-N(R^{A})R^{B}, C₁-C₆ alkylene-CO₂R^{A}, C₁-C₆ alkylene-C(O)R^{A}, or C₁-C₆ alkylene-C(O)N(R^{A})R^{B};
the carbocyclic ring of (i), the heterocyclic ring of (iv), or the heterocyclic ring of (v) is optionally also substituted with 1 or 2 oxo groups; and
the carbocyclic ring fused to the heterocyclic ring of (v) is optionally substituted with 1 to 3 substituents each of which is independently halogen, OH, C₁-C₆ alkyl, O—C₁-C₆ alkyl, C₁-C₆ haloalkyl, O—C₁-C₆ haloalkyl, N(R^{A})R^{B}, or C₁-C₆ alkylene-N(R^{A})R^{B}, and wherein the heterocyclic ring of (v), in addition to being fused to the carbocyclic ring, is optionally substituted with 1 to 3 substituents each of which is independently OR^{A}, N(R^{A})R^{B}, C₁-C₆ alkyl, C₁-C₆ haloalkyl, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-N(R^{A}), C₁-C₆ alkylene-N(O)C₁-C₆ alkylene-CO₂R^{A}, C₁-C₆ alkylene-C(O)R^{A}, C₁-C₆ alkylene-C(O)N(R^{A})R^{B}, or oxo;
   R⁴, R⁵, and R⁶ are each independently H, OH, halo, C₁-C₁₂ alkyl, C₂-C₁₂ alkenyl, aryl, heteroaryl, C(O)N(R⁷)R⁸, N(R⁷)R⁸, C(O)N(R⁷)R⁸, SO₂N(R⁷)R⁸, C₃-C₉ cycloalkyl, heterocyclyl, OR⁹, CO₂R⁹, or C(O)R¹⁰; wherein:
the alkyl, alkenyl, cycloalkyl, or heterocyclyl is optionally substituted with 1 to 3 substituents each of which is independently selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(A)R^{B}, CO₂R^{A}, C(O)R^{A}, C(O)N(O)R^{B}, C(O)N(R^{A})R^{D}, and C₁-C₆ alkylene-N(R^{A})R^{B};
the alkyl, cycloalkyl, or heterocyclyl is optionally also substituted with an oxo group; and
the aryl or heteroaryl is optionally substituted with 1 to 3 substituents each of which is independently selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C(O)N(R^{A})R^{D}, C₁-C₆ alkylene-N(R^{A})R^{B}, C₁-C₆ alkylene-OR^{A}, C₁-C₆ alkylene-SR^{A}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, and C(O)—HetS; wherein each HetS independently has the same definition as HetY;
   alternatively, R⁴ and R⁵ taken together with the carbons to which each is attached form:
(i) a 5- to 7-membered unsaturated but non-aromatic carbocyclic ring,
(ii) a benzene ring,
(iii) a 5- or 6-membered heteroaromatic ring containing from 1 to 3 heteroatoms independently selected from N, O and S, or
(iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂,
   wherein the carbocyclic ring of (i), the benzene ring of (ii), the heteroaromatic ring of (iii), or the heterocyclic ring of (iv) is fused to the naphthyridine ring to provide a fused tricyclic ring system,
   wherein the carbocyclic ring of (i), the benzene ring of (ii), the heteroaromatic ring of (iii), or the heterocyclic ring of (iv) is optionally substituted with from 1 to 4 substituents each of which is independently C₁-C₆ alkyl, C₃-C₇ cycloalkyl, aryl, or heteroaryl, wherein the alkyl, cycloallyl, aryl or heteroaryl is optionally substituted with from 1 to 3 substituents each of which is independently halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, or C(O)N(R^{A})R^{B}, and
   wherein the carbocyclic ring of (i) or the heterocyclic ring of (iv) is optionally also substituted with 1 or 2 oxo groups;
      each R⁷ is independently H or C₁-C₁₂ alkyl, wherein the alkyl is optionally substituted with 1 to 3 substituents each of which is independently selected from the group consisting of oxo, halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
      each R⁸ is independently H, C₁-C₁₂ alkyl, C₃-C₈ cycloalkyl, C₁-C₆ alkylene-C₃-C₈ cycloalkyl, aryl, C₁-C₆ alkylene-aryl, heteroaryl, C₁-C₆ alkylene-heteroaryl, heterocyclyl, or C₁-C₆ alkylene-heterocyclyl; wherein:
the alkyl, cycloalkyl, aryl, heteroaryl, or heterocyclyl which is or is a part of R⁸ is optionally substituted with 1 to 3 substituents each of which is independently halo, OR^{A}, OR^{E}, SR^{A}, SR^{E}, N(R^{A})R^{B}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, C₁-C₆ alkylene-NR^{A}SO₂R^{B}, SO₂N(R^{A})R^{B}, NR^{A}CO₂R^{B}, NR^{A}C(O)R^{B}, NR^{A}-C₁-C₆ alkylene-C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-0e, C₁-C₆ alkylene-SR^{A}, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂₁e, C₁-C₆ alkylene-CO₂R^{A}, C₁-C₆ alkylene-C(O)R^{A}, C₁-C₆ alkylene-C(O)N(R^{A})R^{B}, O-AryC, or O—C₁-C₆ alkylene-AryC, wherein AryC is aryl which is optionally substituted with from 1 to 3 substituents each of which is independently halo, OH, C₁-C₆ alkyl, C₁-C₆ haloalkyl, O—C₁-C₆ alkyl, O—C₁-C₆ haloalkyl, N(R^{A})R^{B}, CO₂R^{A}, or C(O)N(R^{A})R^{B}; and
the alkyl, cycloalkyl or heterocyclyl is optionally also substituted with an oxo group;
   or R⁷ and R⁸ are optionally taken together with the N atom to which they are attached to form a 5- to 7-membered saturated heterocyclic ring, an unsaturated non-aromatic heterocyclic ring, or an aromatic heterocyclic ring, wherein the heterocyclic ring has from zero to 2 heteroatoms independently selected from N, O and S in addition to the N atom to which the R⁷ and R⁸ are attached; wherein each S atom in the saturated or unsaturated non-aromatic ring is optionally in the form S(O) or S(O)₂; and wherein the ring is optionally substituted with from 1 to 4 substituents each of which is independently halo, OR^{A}, SR^{A}, N(R^{A})R^{B}, C₁-C₆ alkyl, C₁-C₆ haloalkyl, NO₂, CN, SO₂(C₁-C₆ alkyl), S(O)(C₁-C₆ alkyl), CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(e)R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-CO₂R^{A}, C₁-C₆ alkylene-C(O)R^{A}, C₁-C₆ alkylene-C(O)N(R^{A})R^{B}, oxo, aryl, C₁-C₆ alkylene-aryl, HetV, C₁-C₆ alkylene-HetV, with the proviso that no more than one substituent on the ring is aryl, C₁-C₆ alkylene-aryl, HetV, or C₁-C₆ alkylene-HetV; wherein:
HetV independently has the same definition as HetY; and
in any substituent of the heterocyclic ring formed from R⁷ and R⁸ taken together which is or contains aryl, the aryl is optionally substituted with from 1 to 3 substituents each of which is independently halo, OH, SH, S—C₁-C₆ alkyl, N(R^{A})R^{B}, C₁-C₆ alkyl, O—C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, C₁-C₆ alkylene-NR^{A}C(O)R^{B}, NR^{A}—C(O)N(R^{A})R^{B}, NR^{B}, C₁-C₆ alkylene-C(O)N(R^{A})R^{B}, CO₂R^{A}, C(O)R^{A}, C(O)N(R^{A})R^{B}, C₁-C₆ alkylene-OH, C₁-C₆ alkylene-O—C₁-C₆ alkyl, C₁-C₆ alkylene-SH, C₁-C₆ alkylene-S—C₁-C₆ alkyl, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-CO₂R^{A}, C₁-C₆ alkylene-C(O)R^{A}, or C₁-C₆ alkylene-C(O)N(R^{A})R^{B};
   each R⁹ is independently C₁-C₁₂ alkyl or aryl, wherein the aryl is optionally substituted with 1 to 3 substituents each of which is independently selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(A)R^{B}, NR^{A}-C₁-C₆ alkylene-C(O)(R^{A})R^{B}, CO₂R^{A}, C(O)R^{A}, C(O)N(R^{A})R^{B}, C(O)N(R^{A})R^{D}, C₁-C₆ alkylene-N(R^{A})R^{B}, C₁-C₆ alkylene-OR^{A}, C₁-C₆ alkylene-SR^{A}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-CO₂R^{A}, C₁-C₆ alkylene-C(O)R^{A}, or C₁-C₆ alkylene-C(O)N(R^{A})R^{B};
   R¹⁰ is H or C₁-C₆ alkyl;
   R^{A} is H, C₁-C₆ alkyl, C₁-C₆ haloalkyl, or C₃-C₈ cycloalkyl;
   R^{B} is H, C₁-C₆ alkyl, C₁-C₆ haloalkyl, or C₃-C₈ cycloalkyl;
   R^{C} is aryl or C₁-C₆ alkyl substituted with aryl;
   R^{D} is aryl, C₁-C₆ alkyl substituted with aryl, heterocyclyl, C₁-C₆ alkyl substituted with heterocyclyl, heteroaryl, C₁-C₆ alkyl substituted with heteroaryl, C₃-C₇ cycloalkyl, or C₁-C₆ alkyl substituted with C₃-C₇ cycloalkyl, wherein:
in any substituted alkyl set forth in R^{D}, the alkyl is optionally substituted with 1 to 3 substituents each of which is independently selected from the group consisting of halo, OR^{A}, SR^{A}, N(R^{A})R^{B}, R^{C}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B}; and
in any R^{D} which is or contains cycloalkyl or heterocyclyl, the cycloalkyl or heterocyclyl is optionally substituted with 1 to 3 substituents each of which is independently selected from the group consisting of halo, OR^{A}, SR^{A}, N(R^{A})R^{B}, R^{C}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-NR^{A}SO₂R^{B}, C₁-C₆ alene-SO₂N(R^{A})R^{B}, C₁-C₆ alkylene-NR^{A}CO₂R^{B}, C₁-C₆ alkylene-NC(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, AryA, C₁-C₆ alkylene-AryA, C₁-C₆ alkylene-HetU, C(O)—HetU, C₁-C₆ alkylene-C(O)—HetU, C₁-C₆ alkylene-(AryA)₁₋₂, and oxo;
in any R^{D} which is or contains aryl or heteroaryl, the aryl or heteroaryl is optionally substituted with 1 to 3 substituents each of which is independently selected from the group consisting of halo, OR^{A}, SR^{A}, N(R^{A})R^{B}, R^{C}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, OR^{A}—C₁-C₆ alkylene-C(O)N(R^{A})R^{b}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})O, C₁-C₆ alkylene-NR^{A}SO₂R^{B}, C₁-C₆ alkylene-SO₂N(NA)R^{B}, C₁-C₆ alkylene-NR^{A}CO₂R^{B}, C₁-C₆ alkylene-NR^{A}C(O)R^{B}, C₁-C₆ alkylene-NR^{A}C(O)(R^{A})R^{B}, C₁-C₆ alkylene-CO₂R^{A}, C₁-C₆ alkylene-C(O)R^{B}, C₁-C₆ alkylene-C(O)N(R^{A})R^{B}, CycA, AryA, C₁-C₆ alkylene-AryA, HetU, C(O)—HetU, C₁-C₆ alkylene-HetU, C₁-C₆ alkylene-C(O)—HetU, C₁-C₆ alkylene-CO₂O, C₁-C₆ alkylene-C(O)R^{A}, C₁-C₆ alkylene-C(O)N(R^{A})R^{B}, C₁-C₆ alkylene-AxyA and C₁-C₆ alkylene-R^{F};
wherein:
   each AryA is independently phenyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₈ cycloalkyl, CN, SO₂(C₁-C₆ alkyl), S(O)(C₁-C₆ alkyl), N(R^{A})R^{B}, NR^{A}SO₂R^{B}, SO₂N(R^{A})R^{B}, NR^{A}C₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁₋₆ alkylene-OH, C₁-C₆ alkylene-N(A)R^{B}, C₁-C₆ alkylene-NR^{A}SO₂R^{B}, C₁₋₆ alkylene-N(R^{A})R^{B}SO₂N(R^{A})R^{B}, C₁-C₆ alkylene-N(R^{A})R^{B}NR^{A}CO₂R^{B}, C₁-C₆ alkylene-NR^{A}C(O)R^{B}, C₁-C₆ alkylene-NR^{A}C(O)N(R^{A})R^{B}, C₁₋₆ alkylene-CO₂R^{B}, C₁-C₆ alkylene-C(O)R^{A}, or C₁-C₆ alkylene-C(O)N(R^{A})R^{B};
   CycA is C₃-C₈ cycloalkyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, N(R^{A})R^{B}, or C₁-C₆ alkylene-N(R^{A})R^{B};
   RF is C(O)-aryl, N(R^{A})-aryl, N(R^{A})—C₁-C₆ alkylene-aryl, C(O)N(10)-aryl, S-aryl, SO₂-aryl, C(O)-heteroaryl, N(R^{A})-heteroaryl, C(O)N(R^{A})-heteroaryl, S-heteroaryl, or SO₂-heteroaryl, wherein the aryl or heteroaryl is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₉ cycloalkyl, CN, SO₂(C₁-C₆ alkyl), S(O)(C₁-C₆ alkyl), N(R^{A})R^{B}, NR^{A}SO₂R^{B}, SO₂N(R^{A})R^{A}, NR^{A}CO₂R^{B}, NR^{A}C(O)R^{B}, NR^{A}C(O)N(R^{A})B, CO₂R^{A}, C(O)R^{A}, C(O)N(R^{A})R^{B}, or C₁-C₆ alkylene-OH, C₁-C₆ alkylene-N(R^{A})R^{B}, C₁-C₆ alene-N(R^{A})R^{B}NR^{A}SO₂R^{B}, C₁-C₆ alkylene-N(R^{A})R^{B}SO₂N(R^{A})R^{B}, C₁-C₆ alkylene-N(R^{A})R^{B}NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{B}, or C₁-C₆ alkylene-C(O)N(R^{A})R^{B};
   each HetU independently has the same definition as HetY; and
      R^{E} is heteroaryl or C₁-C₆ alkyl substituted with heteroaryl;
      and with the provisos that:

(B) when R¹ is O, R³ is H, and R⁴═R⁵═R⁶═H, then XR² is not C(O)OCH₂CH₃;

(C) when R¹ is O, XR² is C(O)N(R⁷)R⁸, R⁴═R⁵═R⁶═H, then R⁸ is not (pyridin-2-ylmethoxy)phenyl; and

(D) when R¹ is O, XR² is C(O)OR⁹, R⁴═R⁶═H, and R⁹ is ethyl, then R⁵ is not 3-cyanophenyl.

Another embodiment of the present invention (referred to herein as “Embodiment E0”) includes compounds of Formula I, and pharmaceutically acceptable salts and/or hydrates thereof, wherein:

R² is H, halo, CN, C₁-C₁₂ alkyl, C₃-C₈ cycloalkyl, aryl, heteroaryl, N(R⁷)R⁸, or OR⁹; wherein the alkyl, cycloalkyl, aryl, or heteroaryl is optionally substituted with from 1 to 3 substituents selected from the group consisting of halo, OR^{A}, SR^{A}, N(R^{A})R^{B}, R^{C}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B}; the alkyl or cycloalkyl is optionally also substituted with an oxo group; and any two adjacent substituents of the cycloalkyl are optionally taken together with the ring atoms to which they are attached to form a ring fused to the cycloalkyl which is (i) a 5- to 7-membered unsaturated but non-aromatic carbocyclic ring, (ii) a benzene ring, (iii) a 5- or 6-membered heteroaromatic ring containing from 1 to 3 heteroatoms independently selected from N, O and S, or (iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂; and wherein the ring fused to the cycloalkyl is optionally substituted with from 1 to 3 substituents selected from the group consisting of halo, OR^{A}, SR^{A}, N(R^{A})R^{B}, R^{C}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
and with the proviso (A) that XR² is not C(O)-halo, C(O)—CN, SO₂-halo, SO₂—CN, O-halo, O—CN, O—OR⁹, N(R^{A})-halo, N(R^{A})—CN, N(R^{A})—OR⁹, N(R^{A})—N(R⁷)R⁸, S-halo, S—CN, S—OR⁹, or S—N(R⁷)R⁸;
R³ is H, OH, NH₂, halo, SO₂N(R⁷)R⁸, C₁-C₁₂ alkyl, OR⁹, N(R⁷)R⁸, NR^{A}C(O)R⁸, or aryl, wherein the aryl is optionally substituted with 1 to 3 substituents selected from the group consisting of halo, OR^{A}, OR^{E}, SR^{A}, SR^{E}, N(R^{A})R^{B}, R^{D}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
alternatively, R³ and XR² are taken together with the carbon atoms to which each is attached to form:
(i) a 5- to 7-membered unsaturated but non-aromatic carbocyclic ring,
(ii) a benzene ring,
(iii) a 5- or 6-membered heteroaromatic ring containing from 1 to 3 heteroatoms independently selected from N, O and S, wherein each N is optionally oxidized, or
(iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂;
   wherein the carbocyclic ring of (i), the benzene ring of (ii), the heteroaromatic ring of (iii), or the heterocyclic ring of (iv) is fused to the naphthyridine ring to provide a fused tricyclic ring system,
   wherein the carbocyclic ring of (i), the benzene ring of (ii), the heteroaromatic ring of (iii), or the heterocyclic ring of (iv) is optionally substituted with from 1 to 4 substituents each of which is independently halo, OR^{A}, SR^{A}, N(R^{A})R^{B}, R^{C}, C₁-C₆ alkyl, C₁-C₆ haloalkyl, O—C₁-C₆ haloalkcyl, NO₂, CN, SO₂(C₁-C₆ alkyl), S(O)(C₁-C₆ alkyl), NR^{A}SO₂R^{B}, SO₂N(A)R^{B}, NR^{A}CO₂R^{B}, NR^{A}OC(O)R^{B}, NR^{A}C(O)N(R^{A})R^{B}, CO₂R^{A}, C(O)R^{A}, or C(O)N(R^{A})R^{B}, and
   wherein the carbocyclic ring of (i) or the heterocyclic ring of (iv) is optionally also substituted with 1 or 2 oxo groups;
      R⁴, R⁵, and R⁶ are each independently H, OH, halo, NH₂, N(R⁷)R⁸, SO₂N(R⁷)R⁸, C₁-C₁₂ alkyl, C₂-C₁₂ alkenyl, aryl, heteroaryl, OR⁹, CO₂R⁹, C(O)N(R⁷)R⁸, N(R⁷)R⁸, C₃-C₉ cycloalkyl, or heterocyclyl; wherein the alkyl, alkenyl, cycloalkyl, aryl, heteroaryl, or heterocyclyl is optionally substituted with 1 to 3 substituents selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}C₂R^{B}, NR^{A}OC(O)R^{B}, NR^{A}C(O)N(R^{A})R^{B}, CO₂R^{A}, C(O)R^{A}, C(O)N(R^{A})R^{B}, C(O)N(A)R^{D}, and C₁₋₆ alkylene-N(R^{A})R^{B}; and the alkyl, cycloalkyl, or heterocyclyl is optionally also substituted with an oxo group;
      alternatively, R⁴ and R⁵ taken together with the carbons to which each is attached form any of rings (i) to (iv) as defined in Embodiment D0;
      each R⁸ is independently H, C₁-C₁₂ alkyl, C₃-C₈ cycloalkyl, C₁-C₆ alkylene-C₃-C₈ cycloalkyl, aryl, C₁-C₆ alkylene-aryl, heteroaryl, C₁-C₆ alkylene-heteroaryl, heterocyclyl, or C₁-C₆ alkylene-heterocyclyl; wherein the alkyl, cycloalkyl, aryl, heteroaryl, or heterocyclyl is optionally substituted with 1 to 3 substituents selected from the group consisting of halo, OR^{A}, OR^{E}, SR^{A}, SR^{E}, N(R^{A})R^{B}, R^{D}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B}; and the alkyl, cycloalkyl or heterocyclyl is optionally also substituted with an oxo group;
      or R⁷ and R⁸ are optionally taken together with the N atom to which they are attached to form a 5- to 7-membered saturated, unsaturated non-aromatic, or aromatic heterocyclic ring having from zero to 2 heteroatoms independently selected from N, O and S in addition to the N atom to which the R⁷ and R⁸ are attached; wherein each S atom in the saturated or unsaturated non-aromatic ring is optionally in the form S(O) or S(O)₂; and wherein the ring is optionally substituted with from 1 to 4 substituents each of which is independently halo, OR^{A}, SR^{A}, N(R^{A})R^{B}, C₁₋₆ alkyl, C₁-C₆ haloalkyl, O—C₁-C₆ haloalkyl, NO₂, CN, SO₂(C₁-C₆ alkyl), S(O)(C₁-C₆ alkyl), CO₂R^{A}, C(O)R^{A}, or C(O)N(R^{A})R^{B};
      each R⁹ is independently C₁-C₁₂ alkyl or aryl, wherein the aryl is optionally substituted with 1 to 3 substituents selected from the group consisting of halo, OR^{A}, SR^{A}, N(R^{A})R^{B}, R^{D}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
      R^{D} is aryl, C₁-C₆ alkyl substituted with aryl, heterocyclyl, C₁-C₆ alkyl substituted with heterocyclyl, heteroaryl, C₁-C₆ alkyl substituted with heteroaryl, C₃-C₇ cycloalkyl, or C₁-C₆ alkyl substituted with C₃-C₇ cycloalkyl, wherein the alkyl, aryl, cycloalkyl, heterocyclyl, or heteroaryl is optionally substituted with 1 to 3 substituents selected from the group consisting of halo, OR^{A}, SR^{A}, N(R^{A})R^{B}, R^{C}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B}; and
      and with the proviso (B) that when R¹ is O, R³ is H, and R⁴═R⁵═R⁶═H, then XR² is not C(O)OCH₂CH₃;
      and all other variables are as defined in Embodiment D0.

The present invention also includes pharmaceutical compositions containing a compound of the present invention and methods of preparing such pharmaceutical compositions. The present invention further includes methods for the treatment of AIDS, the delay in the onset of AIDS, prophylaxis of AIDS, treatment of infection by HIV, and prophylaxis of infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula I as described above, and pharmaceutically acceptable salts thereof. These compounds and their pharmaceutically acceptable salts are HIV RT inhibitors (e.g., HIV-1 RNase H inhibitors) and/or HIV integrase inhibitors (e.g., HIV-1 integrase inhibitors).

An embodiment of the present invention (alternatively referred to herein as “Embodiment D1”) is a compound of Formula I (alternatively and more simply referred to as “Compound I”), or a pharmaceutically acceptable salt thereof, wherein D1 is identical to Embodiment D0 except that each occurrence in Embodiment D0 of the term “C₁-C₁₂ alkyl” is replaced with “C₁-C₆ alkyl” and each occurrence in Embodiment D0 of the term “C₂-C₁₂ alkenyl” is replaced with “C₂-C₆ alkenyl”.

Embodiment D2 of the present invention is Compound I, or a pharmaceutically acceptable salt thereof, wherein R¹ is O; and all other variables are as originally defined in Embodiment D0 set forth in the Summary of the Invention or as defined in Embodiment D1.

Embodiment D3 of the present invention is a compound of Formula I, or a pharmaceutically acceptable salt thereof, wherein at least one of R⁴ and R⁵ is H; R⁶ is H, OH, or NH₂; and all other variables are as defined in any one of Embodiments D0, D1, or D2. In an aspect of Embodiment D3, each R^{A} is independently H or C₁-C₆ alkyl; each R^{B} is independently H or C₁-C₆ alkyl; and all other variables are as originally defined in D3. In another aspect of D3, each R^{A} is independently H or C₁-C₄ alkyl, and each R^{B} is independently H or C₁-C₄ alkyl; and all other variables are as originally defined in D3.

Embodiment D4 of the present invention is a compound of Formula I, or a pharmaceutically acceptable salt thereof, wherein XR² is H, Cl, Br, F, C₁-C₄ alkyl, C(O)O—C₁-C₄ alkyl, C(O)—C₁-C₄ alkyl, cyclopentyl, cyclohexyl, phenyl, CH₂-phenyl, pyridyl, pyrimidinyl, C(O)N(R^{7A})R^{8A}, or O—C₁-C₄ alkyl; wherein:
the C₁-C₄ alkyl is optionally substituted with C(O)O—C₁-C₄ alkyl or C(O)N(H)CH₂-phenyl, wherein the phenyl is optionally substituted with 1 or 2 substituents each of which is independently Cl, Br, F, OH, CH₃, OCH₃, CF₃, OCF₃, N(R^{A})R^{B}, or (CH₂)₁₋₂—N(R^{A})R^{B};
the phenyl or the phenyl which is part of CH₂-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{A}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
R^{7A} is the R⁷ associated with R² and is H or methyl;
R^{8A} is the R⁸ associated with R² and is H, C₁-C₄ alkyl, CH₂CF₃, CH₂CH₂CF₃, cyclopropyl, phenyl, CH₂-phenyl, CH(CH₃)-phenyl, heteroaryl, heterocyclyl, or CH₂-heterocyclyl, wherein:
   the phenyl or the phenyl in CH₂-phenyl or CH(CH₃)-phenyl is optionally substituted with 1 or 2 substituents each of which is independently Cl, Br, F, OH, methyl, CN, OCH₃, CF₃, OCF₃, C(O)CH₃, N(H)C(O)CH₃, CO₂CH₃, C(O)NH₂, C(O)N(H)CH₃, or C(O)N(CH₃)₂;
   the heteroaryl is pyridyl, pyrimidinyl, pyrrolyl, thienyl, furanyl, pyrazolyl, imidazolyl, oxazolyl, or thiazolyl, wherein the heteroaryl is optionally substituted with O-phenyl or OCH₂-phenyl, and is optionally also substituted with 1 or 2 substituents each of which is independently Cl, Br, F, OH, methyl, OCH₃, CF₃, OCF₃, C(O)CH₃, CO₂CH₃, C(O)NH₂, C(O)N(H)CH₃, or C(O)N(CH₃)₂, wherein the total number of substituents ranges from zero to 2;
   the heterocyclyl or the heterocyclyl in CH₂-heterocyclyl is pyrrolidinyl, piperidinyl, piperazinyl, morpholinyl, or thiomorpholinyl, wherein the beterocyclyl is optionally substituted with oxo and is optionally also substituted with C₁-C₄ alkyl, C(O)O—C₁-C₄ alkyl or CH₂-phenyl;
alternatively the R^{7A} and R^{8A} are optionally taken together with the N atom to which they are bonded to form a saturated heterocyclic ring selected from the group consisting of piperidinyl, piperazinyl, pyrrolidinyl, morpholinyl, and thiomorphinyl, wherein the heterocyclic ring is optionally substituted with 1 to 3 substituents each of which is independently halo, OH, methyl, OCH₃, CF₃, OCF₃, C(O)R^{A}, CO₂R^{A}, C(O)N(R^{A})R^{B}, and oxo;
   and all other variables are as defined in any one of Embodiments D0 to D3. In an aspect of Embodiment D4, each R^{A} is independently H or C₁-C₆ alkyl; each R^{B} is independently H or C₁-C₆ alkyl; and all other variables are as originally defined in D4. In another aspect of D4, each R^{A} is independently H or C₁-C₄ alkyl, and each R^{B} is independently H or C₁-C₄ alkyl; and all other variables are as originally defined in D4.

Embodiment D5 of the present invention is a compound of Formula I, or a pharmaceutically acceptable salt thereof, wherein R³ is OH, NH₂, methyl, phenyl, naphthyl, 3,4-dihydronaphthyl, heteroaryl other than HetZ, HetZ, C(O)—HetZ, NR^{A}C(O)R^{8C}, or N(R^{7C})R^{8C}, wherein:
the methyl is substituted with phenyl or (CH₂)₁₋₂-phenyl, wherein either phenyl is further substituted by (i) another phenyl or (ii) another (CH₂)₁₋₂-phenyl, wherein the phenyl in (i) or (ii) is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(A)R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(EA)R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) CH₂-phenyl, (32) CH(CH₃)-phenyl, (33) CH₂CH₂-phenyl, (34) heteroaryl, (35) CH₂-heteroaryl, (36) CH₂CH₂-heteroaryl, (37) CH(CH₃)-heteroaryl, (38) heterocyclyl, (39) CH₂-heterocyclyl, (40) CH(CH₃)-heterocyclyl, or (41) C(O)-heterocyclyl;
   wherein the phenyl in (30), (31), (32), or (33) is optionally substituted with 1 or 2 substituents each of which is independently (a) Cl, (b) Br, (c) F. (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(A)R^{B}, (cc) CN, (dd) cyclopropyl optionally substituted with N(R^{A})R^{B} (such as

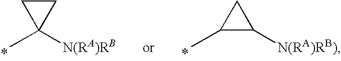

(ee) CH₂—N(R^{A})CH₂-phenyl, (ff) heterocyclyl (gg) C(O)-heterocyclyl, (hh) CH₂-heterocyclyl, or (ii) CH(CH₃)-heterocyclyl; wherein the heterocyclyl in (ff), (gg), (hh) or (ii) is piperidinyl, piperazinyl (optionally substituted with C₁-C₄ alkyl), morpholinyl, pyrrolidinyl, or thiomorpholinyl;
wherein the heteroaryl in (34), (35), (36), or (37) is pyridyl, pyrimidinyl, pyrrolyl, thienyl, furanyl, pyrazolyl, imidazolyl, oxazolyl, or thiazolyl, and the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, or (p) CH₂CH₂—CO₂R^{A};
wherein the heterocyclyl in (38), (39), (40), or (41) is piperidinyl, piperazinyl, morpholinyl, pyrrolidinyl, or thiomorpholinyl, wherein the heterocyclyl is optionally substituted with oxo, and is also optionally substituted with (a) CO₂R^{A}, (b) CH₂—CO₂R^{A} (c) C(O)(R^{A}), (d) N(R^{A})R^{B}, (e) (CH₂)₁₋₃—N(R^{A})R^{B}, (f) C(O)N(R^{A})R^{B}, (g) (CH₂)₁₋₃—C(O)N(R^{A})R^{B}, (h) CH₂C(O)-heterocyclyl, (i) phenyl, (j) CH₂-phenyl, (k) CH(CH₃)-phenyl, (l) CH(phenyl)₂, wherein the heterocyclyl in (h) is piperidinyl, piperazinyl (optionally substituted with C₁-C₄ alkyl), morpholinyl, pyrrolidinyl, or thiomorpholinyl, and wherein the phenyl in (i), (j), (k), or (l) is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(A)R^{B}, CH₂CH₂—N(R^{A})R^{B}, CO₂R^{A}, CH₂—CO₂R^{A}, or CH₂CH₂—CO₂R^{A};
the heteroaryl is
(A) pyridyl, pyrimidinyl, pyrrolyl, thienyl, furanyl, pyrazolyl, imidazolyl, oxazolyl, or thiazolyl, any of which is optionally substituted with 1 or 2 substitutents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) C(O)R^{A}, (18) CH₂—C(O)R^{A}, (19) SO₂(C₁-C₄ alkyl), (20) SO₂N(R^{A})R^{B}, (21) NHSO₂CH₃, (22) CH₂NHSO₂CH₃, (23) C(O)N(R^{A})R^{B}, (24) CH₂C(O)N(R^{A})R^{B}, (25) CH₂OH, (26) CH₂CH₂OH, (27) CN, (28) phenyl, (29) CH₂-phenyl, (30) CH(CH₃)-phenyl, (31) CH₂CH₂-phenyl, or (32) N(e)(CH₂)₁₋₂-heterocyclyl;
   wherein the phenyl in (28), (29), (30) or (31) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(A)R^{B}, (u) NHSO₂CH₃, (v) CH₂NHSO₂CH₃, (w) C(O)N(R^{A})R^{B}, (x) CH₂C(O)N(A)R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN; and
   wherein the heterocyclyl in (32) is piperidinyl, piperazinyl (optionally substituted with C₁-C₄ alkyl), morpholinyl, pyrrolidinyl, or thiomorpholinyl; or

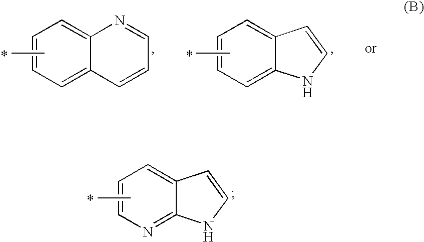

### the HetZ is:

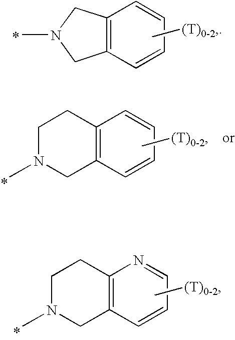 [0128] wherein each T is independently (1) H, (2) Cl, (3) Br, (4) F, (5) OH, (6) CH₃, (7) OCH₃, (8) CH₂F, (9) CF₃, (10) OCH₂F, (11) OCF₃, (12) N(R^{A})R^{B}, (13) CH₂—N(R^{A})R^{B}, (14) CH₂CH₂—N(R^{A})R^{B}, (15) CO₂R^{A}, (16) CH₂—CO₂R^{A}, (17) CH₂CH₂—CO₂R^{A}, (18) CN, (19) pyridyl, (20) pyrimidinyl, (21) phenyl, or (22) C(O)NH(CH₂)₁₋₂-phenyl;
   wherein the phenyl in (21) or (22) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
      R^{7C} is the R⁷ associated with R³ and is H or C₁-C₄ alkyl;
      R^{8C} is the R⁸ associated with R³ and is C₁-C₄ alkyl, phenyl, CH₂-phenyl, CH₂CH₂-phenyl, CH(CH₃)-phenyl, indenyl, dihydroindenyl, 1,2,3,4-tetrahydronaphthyl, heteroaryl, CH₂-heteroaryl, CH(CH₃)-heteroaryl, CH₂CH₂-heteroaryl, heterocyclyl, CH₂-heterocyclyl, CH₂CH₂-heterocyclyl, or CH(CH₃)-heterocyclyl; wherein:
the C₁-C₄ alkyl is optionally substituted with 2 substituents one of which is phenyl and the other of which is OH, (CH₂)₁₋₂—N(R^{A})R^{B}, piperidinyl, piperazinyl (optionally substituted with C₁-C₄ alkyl), morpholinyl, pyrrolidinyl, or thiomorpholinyl;
the phenyl which is or is part of the R^{8C} is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) heteroaryl, (32) heterocyclyl, or (33) CH₂-heterocyclyl;
   wherein the phenyl in (30) is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
   wherein the heteroaryl in (31) is which is pyridyl, pyrimidinyl, pyrrolyl, thienyl, furanyl, pyrazolyl, imidazolyl, oxazolyl, thiazolyl, or triazolyl, and wherein the heteroaryl is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
   wherein the heterocyclyl in (32) or (33) is piperidinyl, piperazinyl, morpholinyl, pyrrolidinyl, or thiomorpholinyl and is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A};
the heteroaryl which is or is part of R^{8C} is pyridyl, pyrimidinyl, pyrrolyl, thienyl, furanyl, pyrazolyl, imidazolyl, oxazolyl, or thiazolyl, and is optionally substituted with phenyl, CH₂-phenyl, heterocyclyl, or CH₂-heterocyclyl in which the heterocyclyl is piperidinyl, piperazinyl (optionally substituted with C₁-C₄ alkyl), morpholinyl, pyrrolidinyl, or thiomorpholinyl;
the heterocyclyl which is or is part of the R^{8C} is piperidinyl, piperazinyl, morpholinyl, pyrrolidinyl, or thiomorpholinyl, wherein the heterocyclyl is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, CO₂R^{A}, phenyl, or CH₂-phenyl;
   alternatively the R^{7C} and R^{8C} together with the N to which both are bonded form a heterocycyl which is piperidinyl, piperazinyl, morpholinyl, pyrrolidinyl, or thiomorpholinyl, wherein the heterocyclyl is optionally substituted with oxo and is also optionally substituted with from 1 to 3 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) C(O)R^{A}, (12) CO₂R^{A}, (13) CH₂C(O)R^{A}, (14) CH₂CO₂R^{A}, (15) phenyl, (16) CH₂-phenyl, (17) CH(CH₁₃)-phenyl, (18) heterocyclyl, (19) CH₂-heterocyclyl, or (20) CH(CH₃)-heterocyclyl;
wherein the phenyl in (15), (16), or (17) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j)OCF₃, (k) N(R^{A})R^{B}, (l) CH₂—N(A)R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN; and
wherein the heterocyclyl in (18), (19) or (20) is piperidinyl, piperazinyl, morpholinyl, pyrrolidinyl, or thiomorpholinyl, wherein the heterocyclyl is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A};
   and all other variables are as defined in any one of Embodiments D0 to D4. In an aspect of Embodiment D5, each R^{A} is independently H or C₁-C₆ alkyl; each R^{B} is independently H or C₁-C₆ alkyl; and all other variables are as originally defined in D5. In another aspect of D5, each R^{A} is independently H or C₁-C₄ alkyl, and each R^{B} is independently H or C₁-C₄ alkyl; and all other variables are as originally defined in D5.

Embodiment D6 of the present invention is a compound of Formula I, or a pharmaceutically acceptable salt thereof, wherein alternatively XR² and R³ are taken together with the carbon atoms to which each is attached to provide:

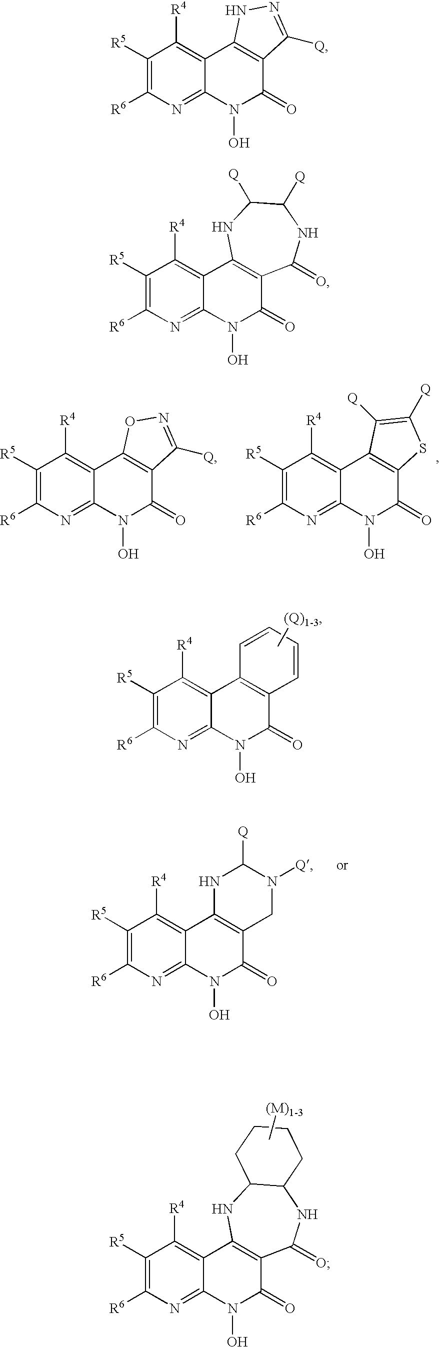

wherein:
each M is independently H, OH, Cl, Br, F, C₁-C₄ alkyl, N(R^{A})R^{B}, or (CH₂)₁₋₂—N(R^{A})R^{B},
each Q is independently H, Cl, Br, F, C₁-C₄ alkyl, C(O)N(R^{A})R^{B}, (CH₂)₁₋₂—C(O)N(R^{A})R^{B}, N(R^{A})R^{B}, (CH₂)₁₋₂—N(R^{A})R^{B}, or phenyl, wherein:
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) O-phenyl, (32) (CH₂)₁₋₂-phenyl, (33) O—(CH₂)₁₋₂-phenyl, (34) heteroaryl, (35) heterocyclyl, or (36) (CH₂)₁₋₂-heterocyclyl,
   wherein the phenyl in (30), (31), (32), or (33) is optionally substituted with 1 or 2 substituents each of which is independently Cl, Br, F, OH, CH₃, OCH₃, CH₂F, CF₃, OCH₂F, OCF₃, N(R^{A})ₖO, CH₂—N(R^{A})R^{B}, CH₂CH₂—N(R^{A})R^{B}, CO₂R^{A}, CH₂—CO₂R^{A}, or CH₂CH₂—CO₂R^{A};
   wherein the heteroaryl in (34) is pyridyl, pyrimidinyl, pyrrolyl, thienyl, furanyl, pyrazolyl, imidazolyl, oxazolyl, thiazolyl, or triazolyl, and wherein the heteroaryl is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
   wherein the heterocyclyl in (35) or (36) is piperidinyl, piperazinyl, morpholinyl, pyrrolidinyl, or thiomorpholinyl and is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A};
      Q′ is H or C₁-C₄ alkyl;
      and all other variables are as defined in any one of Embodiments D0 to D5. In an aspect of Embodiment D6, each R^{A} is independently H or C₁-C₆ alkyl; each R^{B} is independently H or C₁-C₆ alkyl; and all other variables are as originally defined in D6. In another aspect of D6, each R^{A} is independently H or C₁-C₄ alkyl, and each R^{B} is independently H or C₁-C₄ alkyl; and all other variables are as originally defined in D6.

Embodiment D7 of the present invention is a compound of Formula I, or a pharmaceutically acceptable salt thereof, wherein:

R⁴ is H, phenyl, CH₂-phenyl, or C(O)O—C₁-C₄ alkyl wherein:
the phenyl or the phenyl in CH₂-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN; (30) phenyl, (31) CH₂-phenyl, (32) CH(CH₃)-phenyl, (33) CH₂CH₂-phenyl, or (34) heteroaryl;
   wherein the phenyl in (30), (31), (32), or (33) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
   wherein the heteroaryl in (34) is pyridyl, pyrimidinyl, pyrrolyl, thienyl, furanyl, pyrazolyl, imidazolyl, oxazolyl, or thiazolyl, and wherein the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o) CH₂—CO₂R^{B}, or (p) CH₂CH₂—CO₂R^{A};
      R⁵ is H, Cl, Br, F, C₁-C₄ alkyl, C₂-C₄ alkenyl, phenyl, O-phenyl, naphthyl, heteroaryl, NH₂, C(O)N(R^{7B})R^{8B}, SO₂N(R^{7B})R^{8B}, C(O)O—C₁-C₄ alkyl, C(O)H, or C(O)—C₁-C₄ alkyl, wherein:
the C₁-C₄ alkyl is optionally substituted with 1 or 2 substituents each of which is independently (1) Cl, (2) Br, (3) F, (4) OH, (5) OCH₃, (6) CH₂F, (7) CF₃, (8) OCH₂F, (9) OCF₃, (10) N(R^{A})R^{B}, (11) phenyl, or (12) N(R^{A})CH₂-phenyl;
   wherein the phenyl in (11) or (12) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o) CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
the C₂-C₄ alkenyl is optionally substituted with (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, or (12) phenyl;
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) CH₂-phenyl, (32) CH(CH₃)-phenyl, (33) CH₂CH₂-phenyl, (34) heteroaryl, (35) CH₂-heteroaryl, (36) CH₂CH₂-heteroaryl, (37) CH(CH₃)-heteroaryl, (38) heterocyclyl, (39) CH₂-heterocyclyl, (40) CH(CH₃)-heterocyclyl, or (41) C(O)-heterocyclyl;
   wherein the phenyl in (30), (31), (32), or (33) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
   wherein the heteroaryl in (34), (35), (36), or (37) is pyridyl, pyrimidinyl, pyrrolyl, thienyl, furanyl, pyrazolyl, imidazolyl, oxazolyl, or thiazolyl, and the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o) CH₂—CO₂R^{A}, or (p) CH₂CH₂—CO₂R^{A};
   wherein the heterocyclyl in (38), (39), (40) or (41) is piperidinyl, piperazinyl, morpholinyl, pyrrolidinyl, or thiomorpholinyl, wherein the heterocyclyl is optionally substituted with oxo, and is also optionally substituted with (1) CO₂R^{A}, (2) CH₂—CO₂R^{A} (3) C(O)(R^{A}), (4) N(R^{A})R^{B}, or (5) (CH₂)₁₋₃—N(R^{A})R^{B};
the O-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (I) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
the heteroaryl is pyridyl, pyrimidinyl, pyrrolyl, furanyl, thienyl, pyrazolyl, imidazolyl, or thiazolyl, and the heteroaryl is optionally substituted with 1 or 2 substitutents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, or (16) CH₂CH₂—CO₂R^{A};
R^{7B} is the R⁷ associated with R⁵ and is H or C₁-C₄ alkyl;
R^{8B} is the R⁸ associated with R⁵ and is H, C₁-C₄ alkyl, cyclopentyl, cyclohexyl, phenyl, CH₂-phenyl, CH₂CH₂-phenyl, or CH(CH₃)-phenyl; wherein
   the C₁-C₄ alkyl is optionally substituted with 2 substituents one of which is phenyl and the other of which is OH, (CH₂)₁₋₂—N(R^{A})R^{B}, or heterocyclyl;
      wherein the heterocyclyl is piperidinyl, piperazinyl, morpholinyl, pyrrolidinyl, or thiomorpholinyl, wherein the heterocyclyl is optionally substituted with oxo, and is also optionally substituted with (a) CO₂R^{A}, (b) CH₂—CO₂R^{A} (c) C(O)(R^{A}), (d) N(R^{A})R^{B}, (e) (CH₂)₁₋₃—N(R^{A})R^{B};
   the phenyl which is or is part of the R^{8B} is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17)NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
alternatively the R^{7B} and R^{8B} together with the N to which both are bonded form heterocycyl which is piperidinyl, piperazinyl, morpholinyl, pyrrolidinyl, or thiomorpholinyl, wherein the heterocyclyl is optionally substituted with oxo and is also optionally substituted with 1 or 2 substituents each of which is independently Cl, Br, F, OH, CH₃, OCH₃, CH₂F, CF₃, OCH₂F, OCF₃, C(O)R^{A}, CO₂R^{A}, CH₂C(O)R^{A}, CH₂CO₂R^{A}, phenyl, CH₂-phenyl, CH₂CH₂-phenyl, CH₂CH₂CH₂-phenyl, or CH(CH₃)-phenyl;
   wherein phenyl which is or is part of a substituent on the heterocyclyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(A)R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
      R⁶ is H; and all other variables are as defined in any one of Embodiments D0 to D6. In an aspect of Embodiment D7, each R^{A} is independently H or C₁-C₆ alkyl; each R^{B} is independently H or C₁-C₆ alkyl; and all other variables are as originally defined in D7. In another aspect of D7, each R^{A} is independently H or C₁-C₄ alkyl, and each R^{B} is independently H or C₁-C₄ alkyl; and all other variables are as originally defined in D7.

Embodiment D8 of the present invention is a compound of Formula I, or a pharmaceutically acceptable salt thereof, as defined in any one of Embodiments D0 to D7, with the proviso (E) that when X is a bond and R² is N(R⁷)R⁸, then R⁷ and R⁸ in the definition of R² do not together with the N form a ring. It is understood that this limitation on N(R⁷)R⁸ applies only to R² and an N(R⁷)R⁸ in any other variable can optionally form such a ring.

Embodiment D9 of the present invention is a compound of Formula I, or a pharmaceutically acceptable salt thereof, as defined in any one of Embodiments D0 to D7, with the proviso (E′) that with respect to any N(R⁷)R⁸ group, R⁷ and R⁸ do not together with the N form a ring. It is understood that this limitation on N(R⁷)R⁸ applies generally to any group that includes one or more N(R⁷)R⁸ groups in its definition.

Embodiment D10 of the present invention is a compound of Formula I as defined in Embodiment D0 above, or a pharmaceutically acceptable salt thereof, with the proviso (F) that when R¹ is O, R³ is OH or NH₂, R⁴ is H, R⁵ is H and R⁶ is H, then XR² is not H. Aspects of Embodiment D10 include each of the foregoing D embodiments other than D0 in which application of proviso F can limit the scope of the embodiment, wherein proviso G is applied thereto.

Embodiment D11 of the present invention is a compound of Formula I as defined in Embodiment D0, or a pharmaceutically acceptable salt thereof, with the proviso (G) that when R¹ is O, R³ is OH, R⁴ is H, R⁵ is H and R⁶ is H, then XR² is not 1,1-dioxido-4H-1,2,4-benzothiadiazin-3-yl. Aspects of Embodiment D11 include each of the foregoing D embodiments other than D0 in which application of proviso G can limit the scope of the embodiment, wherein proviso G is applied thereto.

Embodiment D12 of the present invention is a compound of Formula I, or a pharmaceutically acceptable salt thereof, as defined in any one of the foregoing D embodiments in which application of each of provisos F and G can limit the scope of the embodiment, wherein proviso F and proviso G are applied thereto.

Embodiment D13 of the present invention is a compound of Formula I as defined in Embodiment D0 above, or a pharmaceutically acceptable salt thereof, with the proviso (B′) that when R¹ is O, R³ is H, and R⁴═R⁵═R⁶═H, then XR² is not C(O)O—(C₁-C₆ alkyl). In a first aspect of this embodiment, proviso B′ provides that when R¹ is O, R³ is H, and R⁴═R⁵═R⁶═H, then XR² is not C(O)O—(C₁-C₁₂ alkyl). Other aspects of Embodiment D13 include each of the foregoing D embodiments other than D0 in which application of proviso B′ (as originally defined or as defined in the first aspect of D13) can limit the scope of the embodiment, wherein proviso B′ is applied thereto.

Embodiment D14 of the present invention is a compound, or a pharmaceutically acceptable salt thereof, selected from the group consisting of the compounds set forth in Examples 1-14, 16-59, and 61-268 (alternatively referred to as Compounds 1-14, 16-59, and 61-268) below. In an aspect of this embodiment, the compound is selected from Compounds 17, 44-46, 70, 71, 83-86, 96, 104-167, 169, 170, 172-268, and pharmaceutically acceptable salts thereof. In another aspect of this embodiment, the compound is selected from the group consisting of the compounds in Table 21 below and pharmaceutically acceptable salts thereof.

A class of compounds of the present invention (alternatively referred to herein as Class C1) includes compounds of Formula I and pharmaceutically acceptable salts thereof, wherein:

### R¹ is O;

XR² is (1) H, (2) C(O)O—CH₂CH₃, (3) phenyl optionally substituted with, Cl, OCH₃, or CF₃, (4) CH₂-phenyl, (5) pyridyl, (6) C(O)NH—CH₂-phenyl, (7) C(O)NH—CH₂-pyrrolidinyl, (8) C(O)NH—CH₂-piperidinyl, or (9) C(O)NH—CH₂CF₃;
R³ is OH, methyl, phenyl, HetZ, or N(H)R^{8C}, wherein:
the methyl is:
   (1) substituted with phenyl which is substituted with another phenyl which is substituted by CH₂—N(R^{A})R^{B}, or
   (2) substituted with phenyl which is substituted with (CH₂)₁₋₂-phenyl which is substituted by 1 or 2 substituents each of which is independently Cl, Br, or F;
the phenyl is substituted (i) with CH₂—N(R^{A})R^{B} or (ii) with another phenyl which is substituted by CH₂—N(R^{A})R^{B};
R^{8C} is:
   (1) CH₂-phenyl in which the phenyl is substituted with OCH₃, CH₂NH₂,

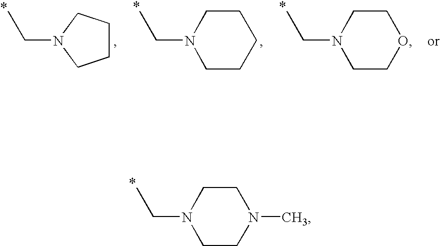

(2) CH(CH₃)-phenyl,
(3) CH₂-pyridyl in which the pyridyl is optionally substituted with

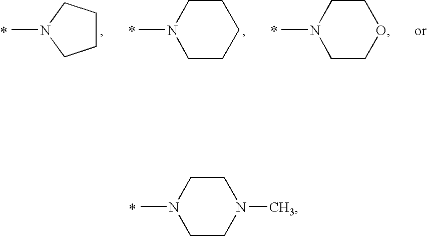

(4) methyl substituted with phenyl and with (CH₂)₁₋₂—N(R^{A})R^{B},

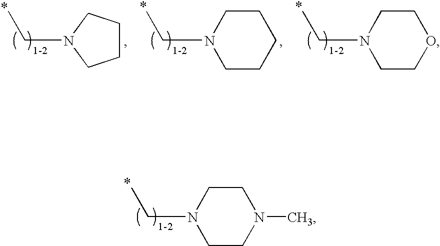

(5) phenyl substituted with phenyl which is optionally substituted with CH₂—N(R^{A})R^{B},

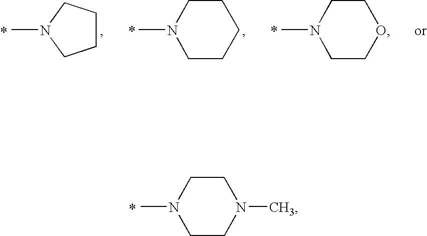

(6) substituted heterocyclyl selected from the group consisting of:

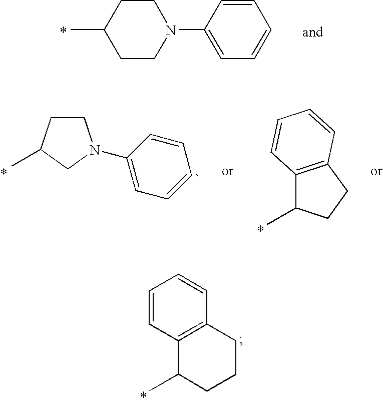

(6)

### HetZ is:

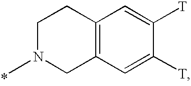 [0186] (1) wherein one T is phenyl, pyridyl, or C(O)OCH₃, and the other T is H,

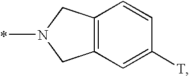

(2) wherein T is phenyl which is optionally substituted with CH₂—N(R^{A})R^{B}, or

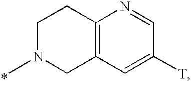

(3) wherein T is phenyl which is optionally substituted with CH₂—N(R^{A})R^{B};
   R⁴ is H, C(O)OCH₃, C(O)OCH₂CH₃, or phenyl which is optionally substituted with Cl, Br, F, OH, CH₃, OCH₃, CF₃, OCF₃, or CH₂—N(R^{A})R^{B};
   R⁵ is H, F, C(O)OCH₃, C(O)OCH₂CH₃, CH₂-phenyl, or phenyl which is optionally substituted with Cl, Br, F, OH, CH₃, OCH₃, CF₃, or OCF₃;

### R⁶ is H;

each R^{A} is independently H, CH₃, or CH₂CH₃; and
each R^{B} is independently H, CH₃, or CH₂CH₃.

Embodiment E1 of the present invention is Compound I, or a pharmaceutically acceptable salt thereof, wherein R¹ is O (i.e., Formula II); and all other variables are as originally defined in Embodiment E0 in the Summary of the Invention.

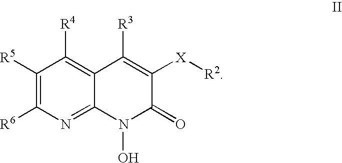

Embodiment E2 of the present invention is a compound of Formula I, or a pharmaceutically acceptable salt thereof, wherein X is a bond, C(O), CH₂, or N(R^{A}); and all other variables are as defined in Embodiment E0 or Embodiment E1. In a first aspect of Embodiment E2, X is a bond; and all other variables are as defined in Embodiment E0 or Embodiment E1. In a second aspect of Embodiment E2, X is C(O); and all other variables are as defined in Embodiment E0 or E1. In a third aspect of Embodiment E2, X is CH₂; and all other variables are as defined in Embodiment E0 or E1.

In any of the D and E embodiments set forth above or below with respect to compounds of Formula I or II and in any classes of compounds defined above or below, the provisos A, B, C and D appearing in Embodiments D0 and E0 of Compound I in the Summary of the Invention apply unless their application is unnecessary. For example, in Embodiment E2, the applicable proviso A is as follows: “and with the proviso that XR² is not C(O)-halo, C(O)—CN, N(R^{A})-halo, N(R^{A})—CN, N(R^{A})—OR⁹, or N(R^{A})—N(R⁷)R⁸″ and proviso B is unchanged. Note, however, that the application of proviso A and proviso B is not necessary in the third aspect of Embodiment E2 because none of the groups excluded by the provisos involve X═CH₂.

Embodiment E3 of the present invention is a compound of Formula I, or a pharmaceutically acceptable salt thereof, wherein R² is H, halo, C₁-C₆ alkyl, C₅-C₇ cycloalkyl, aryl, heteroaryl, N(R⁷)R⁸, or OR⁹, wherein the alkyl, cycloalkyl, aryl, or heteroaryl is optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E2.

In a first aspect of Embodiment E3, R² is H; and all other variables areas defined in any one of Embodiments E0 to E2. In a second aspect of Embodiment E3, R² is halo (e.g., Br or Cl); and all other variables are as defined in any one of Embodiments E0 to E2. In a third aspect of Embodiment E3, R² is C₁-C₆ alkyl; and all other variables are as defined in any one of Embodiments E0 to E2. In a feature of the third aspect of Embodiment E3, R² is C₁-C₄ alkyl; and all other variables areas defined in any one of Embodiments E0 to E2. In another feature of the third aspect of Embodiment E3, R² is methyl, ethyl, n-propyl or n-butyl; and all other variables are as defined in any one of Embodiments E0 to E2.

In a fourth aspect of Embodiment E3, R² is C₅-C₇ cycloalkyl optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E2. In a feature of the fourth aspect of Embodiment E3, R² is cyclopentyl or cyclohexyl; and all other variables are as defined in any one of Embodiments E0 to E2.

In a fifth aspect of Embodiment E3, R² is aryl optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E2. In a feature of the fifth aspect of Embodiment E3, R² is phenyl optionally substituted with 1 to 2 substituents independently selected from halo (e.g., F, Cl or Br), OR^{A}, and CF₃; and all other variables are as defined in any one of Embodiments E0 to E2.

In a sixth aspect of Embodiment E3, R² is heteroaryl optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E2. In a feature of the sixth aspect of Embodiment E3, R² is pyridyl (alternatively referred to as “pyridinyl”) optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; all other variables are as defined in any one of Embodiments E0 to E2.

In a seventh aspect of Embodiment E3, R² is N(R⁷)R⁹ and X is C(O) or SO₂; and all other variables are as defined in any one of Embodiments E0 to E2. In a first feature of the seventh aspect of Embodiment E3, R² is N(R⁷)R⁸ wherein R⁷ is H or C₁-C₆ alkyl; and R⁸ is C₁-C₆ alkyl, C₃-C₆ cycloalkyl, aryl, heteroaryl, or heterocyclyl; wherein the alkyl, cycloalkyl, aryl, heteroaryl or heterocyclyl is optionally substituted with 1 to 2 substituents selected from the group consisting of halo, OR^{A}, OR^{E}, R^{D}, C₁-C₆ alkyl, NO₂, CN, CF₃, NR^{A}CO₂R^{B}, NR^{A}C(O)R^{B}, CO₂R^{A}, and C(O)N(R^{A})R^{B}; and all other variables are as defined in any one of Embodiments E0 to E2. In a second feature of the seventh aspect of Embodiment E3, R² is N(R⁷)R⁸ wherein R⁷ is H or methyl; and R⁸ is C₁-C₃ alkyl, cyclopropyl, phenyl, pyridyl, or piperidinyl; wherein the alkyl, cyclopropyl, phenyl, pyridyl, or piperidinyl is optionally substituted with 1 to 2 substituents selected from the group consisting of halo, OR^{A}, OR^{E}, R^{D}, C₁-C₆ alkyl, CF₃, NR^{A}C(O)R^{B}, CO₂R^{A}, and C(O)N(R^{A})R^{B}; and all other variables are as defined in any one of Embodiments E0 to E2. In a third feature of the seventh aspect of Embodiment E3, R² is N(R⁷)R⁸ wherein R⁷ and R⁸ are taken together with the N atom to which they are bonded to form a 5- to 7-membered saturated, unsaturated non-aromatic, or aromatic heterocyclic ring having 0-2 additional heteroatoms independently selected from N, O and S; and all other variables are as defined in any one of Embodiments E0 to E2. In a fourth feature of the seventh aspect of Embodiment E3, R² is N(R⁷)R⁸ wherein R⁷ and R⁸ are taken together the N atom to which they are bonded to form a piperidinyl ring; and all other variables are as defined in any one of Embodiments E0 to E2.

In an eighth aspect of Embodiment E3, R² is OR⁹ and X is C(O) or SO₂; and all other variables are as defined in any one of Embodiments E0 to E2. In a first feature of the eighth aspect of Embodiment E3, R² is OR⁹ wherein R⁹ is C₁-C₆ alkyl; and all other variables are as defined in any one of Embodiments E0 to E2. In a second feature of the eighth aspect of Embodiment E3, R² is OR⁹ wherein R⁹ is methyl or ethyl; and all other variables are as defined in any one of Embodiments E0 to E2.

Embodiment E4 of the present invention is a compound of Formula I, or a pharmaceutically acceptable salt thereof, wherein R³ is OH, NH₂, halo, SO₂N(R⁷)R⁸, C₁-C₁₂ alkyl, OR⁹, N(R⁷)R⁸, NR^{A}C(O)R⁸, or aryl, wherein the aryl is optionally substituted with 1 to 3 substituents selected from the group consisting of halo, OR^{A}, OR^{E}, SR^{A}, SR^{B}, N(R^{A})R^{B}, R^{D}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(A)R^{B}, NR^{A}CO₂R^{B}, NR^{A}C(O)R^{B}, NR^{A}C(O)N(R^{A})R^{B}, CO₂R^{E}, C(O)R^{A}, and C(O)N(R^{A})R^{B}; and all other variables are as defined in any one of Embodiments E0 to E3. In a first aspect of Embodiment E4, R³ is OH, NH₂, NR^{A}C(O)R⁸, N(R⁷)R⁸, or aryl; and all other variables are as defined in any one of Embodiments E0 to E3. In a second aspect of Embodiment E4, R³ is OH; and all other variables are as defined in any one of Embodiments E0 to E3. In a third aspect of Embodiment E4, R³ is NH₂; and all other variables are as defined in any one of Embodiments E0 to E3. In a fourth aspect of Embodiment E4, R³ is NR^{A}C(O)R⁸; and all other variables are as defined in any one of Embodiments E0 to E3. In a fifth aspect of Embodiment E4, R³ is NR^{A}C(O)R⁸ wherein R^{A} is H and R⁸ is C₁-C₄ alkyl or aryl wherein the alkyl or aryl is optionally substituted with R^{D} wherein R^{D} is aryl; and all other variables are as defined in any one of Embodiments E0 to E3. In a feature of the fifth aspect of Embodiment E4, R³ is NR^{A}C(O)R⁸ wherein R^{A} is H and R⁸ is methyl, phenyl or benzyl; and all other variables are as defined in any one of Embodiments E0 to E3. In a sixth aspect of Embodiment E4, R³ is N(R⁷)R⁸ wherein R⁷ is H or C₁-C₆ alkyl and R⁸ is aryl optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E3. In a feature of the sixth aspect of Embodiment E4, R³ is N(R⁷)R⁸ wherein R⁷ is H or C₁-C₄ alkyl and R⁸ is phenyl; and all other variables are as defined in any one of Embodiments E0 to E3. In a seventh aspect of Embodiment E4, R³ is aryl optionally substituted with 1 to 2 substituents selected from the group consisting of halo, OR^{A}, OR^{E}, R^{D}, C₁-C₆ alkyl, NO₂, CN, CF₃, NR^{A}CO₂R^{B}, NR^{A}C(O)R^{B}, CO₂R^{A}, and C(O)N(R^{A})R^{B}; and all other variables are as defined in any one of Embodiments E0 to E3. In a feature of the seventh aspect of Embodiment E4, R³ is phenyl; and all other variables are as defined in any one of Embodiments E0 to E3.

Embodiment ES of the present invention is a compound of Formula I, or a pharmaceutically acceptable salt thereof, wherein R³ and XR² are taken together to form (A) a 5- or 6-membered heteroaromatic ring containing 1 or 2 heteroatoms independently selected from N, O and S, or (B) a 5 to 7-membered unsaturated but non-aromatic heterocyclic ring containing 1 or 2 heteroatoms independently selected from N, O and S, wherein each N is optionally oxidized and each S is optionally in the form of S(O) or S(O)₂; wherein the heteroaromatic ring of (A) or the heterocyclic ring of (13) is optionally substituted with from 1 to 3 substituents, each of which is independently halo, C₁-C₄ alkyl, aryl, or C₁-C₄ alkyl substituted with aryl; and all other variables are as defined in any one of Embodiments E0 to E4. In an aspect of Embodiment E5, R³ and XR² are taken together to form (A) a 5- or 6-membered heteroaromatic ring containing 1 or 2 N atoms, or (B) a 5 to 7-membered unsaturated but non-aromatic heterocyclic ring containing 1 or 2 N atoms; wherein the heteroaromatic ring of (A) or the heterocyclic ring of (13) is optionally substituted with from 1 or 2 substituents, each of which is independently halo, C₁-C₄ alkyl, aryl, or C₁-C₄ alkyl substituted with aryl and all other variables are as defined in any one of Embodiments E0 to E4. In a second aspect of Embodiment E5, R³ and XR² are taken together to form a pyrazolo ring optionally substituted with C₁-C₄ alkyl; and all other variables are as defined in any one of Embodiments E0 to E4. In a third aspect of Embodiment E5, R³ and XR² are taken together to form a dihydrodiazepino ring substituted with phenyl; and all other variables are as defined in any one of Embodiments E0 to E4. In a fourth aspect of Embodiment E5, R³ and XR² are taken together to form an isoxazolyl optionally substituted with methyl; and all other variables are as defined in any one of Embodiments E0 to E4. In a fifth aspect of Embodiment E5, R³ and XR² are taken together to form thienyl; and all other variables are as defined in any one of Embodiments E0 to E4.

Examples of compounds embraced by Embodiment E5 include:

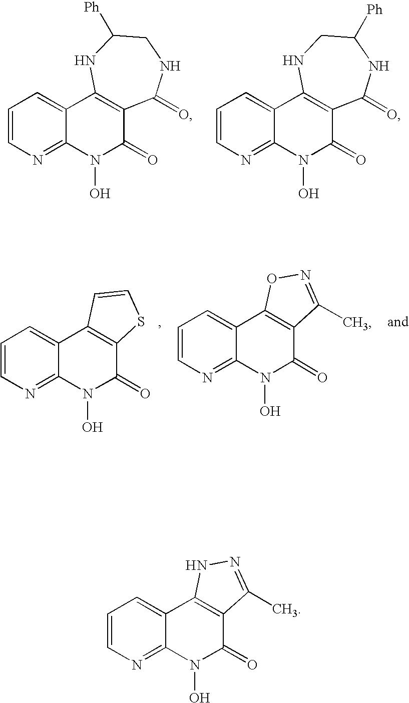

Embodiment E6 of the present invention is a compound of Formula I, or a pharmaceutically acceptable salt thereof, wherein R⁴ is H, aryl, or CO₂R⁹, wherein the aryl is optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E5. In a first aspect of Embodiment E6, R⁴ is H; and all other variables are as defined in any one of Embodiments E0 to E5. In a second aspect of Embodiment E6, R⁴ is phenyl; and all other variables are as defined in any one of Embodiments E0 to E5. In a third aspect of Embodiment E6, R⁴ is CO₂R⁹ wherein R⁹ is C₁-C₆ alkyl; and all other variables are as defined in any one of Embodiments E0 to E5. In a feature of the third aspect of Embodiment E6, R⁴ is CO₂Et; and all other variables are as defined in any one of Embodiments E0 to E5.

Embodiment E7 of the present invention is a compound of Formula I, or a pharmaceutically acceptable salt thereof, wherein R⁵ is H, halo, SO₂N(R⁷)R⁸, C₁-C₁₂ alkyl, C₂-C₁₂ alkenyl, aryl, heteroaryl, OR⁹, CO₂R⁹, or C(O)N(R⁷)R⁸, wherein the alkyl, alkenyl, aryl, or heteroaryl is optionally substituted with 1 to 3 substituents selected from the group consisting of halo, OR^{A}, N(R^{A})R^{B}, N(R^{A})R^{D}, R^{D}, R^{E}, C₁-C₆ alkyl, CN, NR^{A}SO₂R^{B}, and C₁-C₆ alkylene-N(R^{A})R^{B}; and all other variables are as defined in any one of Embodiments E0 to E6. In a first aspect of Embodiment E7, R⁵ is H; and all other variables are as defined in any one of Embodiments E0 to E6. In a second aspect of Embodiment E7, R⁵ is halo; and all other variables are as defined in any one of Embodiments E0 to E6. In a feature of the second aspect of Embodiment E7, R⁵ is F or Br; and all other variables are as defined in any one of Embodiments E0 to E6. In a third aspect of Embodiment E7, R⁵ is C₁-C₁₂ alkyl or C₂-C₁₂ alkenyl wherein the alkyl or alkenyl is optionally substituted with R^{D}, halo or N(A)R^{D}; and all other variables are as defined in any one of Embodiments E0 to E6. In a first feature of the third aspect of Embodiment E7, R⁵ is C₁-C₆ alkyl or C₂-C₆ alkenyl wherein the alkyl or alkenyl is optionally substituted with phenyl (i.e., the alkyl or alkenyl is optionally substituted with R^{D} wherein R^{D} is phenyl), halo or N(R^{A})R^{D} wherein R^{D} is benzyl optionally substituted with halo; and all other variables are as defined in any one of Embodiments E0 to E6. In a second feature of the third aspect of Embodiment E7, R⁵ is methyl, ethyl, bromopropyl (e.g., 2-bromopropyl), benzyl, 2-phenylvinyl (e.g., (E)-2-phenylvinyl), or (chlorobenzyl)amino]ethyl (e.g., 1-[(3-chlorobenzyl)amino]ethyl); and all other variables are as defined in any one of Embodiments E0 to E6.

In the fourth aspect of Embodiment E7, R⁵ is SO₂N(R⁷)R⁸; and all other variables are as defined in any one of Embodiments E0 to E6. In a feature of the fourth aspect of Embodiment E7, R⁵ is SO₂N(R⁷)R⁸ wherein R⁷ is H and R⁸ is phenyl; and all other variables are as defined in any one of Embodiments E0 to E6. In the fifth aspect of Embodiment E7, R⁵ is aryl or heteroaryl wherein the aryl or heteroaryl is optionally substituted with 1 to 2 substituents selected from the group consisting of halo, OR^{A}, N(R^{A})R^{B}, R^{D}, CN, NR^{A}SO₂R^{B}, and C₁-C₆ alkyl optionally substituted with N(R^{A})R^{B}; and all other variables are as defined in any one of Embodiments E0 to E6. In a first feature of the fifth aspect of Embodiment E7, R⁵ is phenyl or naphthyl optionally substituted with 1 to 2 substituents independently selected from F, Cl, Br, CN, OH, OMe, morpholinylmethyl, pyrazolyl, methyl, NH₂, NHSO₂Me, and —CH₂NH₂; and all other variables are as defined in any one of Embodiments E0 to E6. In a second feature of the fifth aspect of Embodiment E7, R⁵ is thienyl or pyridyl; and all other variables are as defined in any one of Embodiments E0 to E6.

In a sixth aspect of Embodiment E7, R⁵ is OR⁹; and all other variables are as defined in any one of Embodiments E0 to E6. In a first feature of the sixth aspect of Embodiment E7, R⁵ is OR⁹ wherein R⁹ is aryl optionally substituted with 1 to 2 substituents selected from the group consisting of halo, OR^{A}, SR^{A}, N(R^{A})R^{B}, C₁-C₆ alkyl, C₁-C₆ haloalkyl, NO₂, CN, CF₃, NR^{A}C(O)R^{B}, CO₂R^{A}, and C(O)N(R^{A})R^{B}; and all other variables are as defined in any one of Embodiments E0 to E6. In a second feature of the sixth aspect of Embodiment E7, R⁵ is OR⁹ wherein R⁹ is phenyl optionally substituted with N(R^{A})R^{B}; and all other variables are as defined in any one of Embodiments E0 to E6. In a seventh aspect of Embodiment E7, R⁵ is CO₂R⁹; and all other variables are as defined in any one of Embodiments E0 to E6. In a feature of the sixth aspect of Embodiment E7, R⁵ is CO₂R⁹ wherein R⁹ is C₁-C₄ alkyl; and all other variables are as defined in any one of Embodiments E0 to E6. In an eighth aspect of Embodiment E7, R⁵ is C(O)N(R⁷)R⁸; and all other variables are as defined in any one of Embodiments E0 to E6. In a first feature of the eighth aspect of Embodiment E7, R⁵ is C(O)N(R⁷)R⁸ wherein R⁷ is H or C₁-C₄ and R⁸ is C₁-C₆ alkyl optionally substituted with R^{D}; and all other variables are as defined in any one of Embodiments E0 to E6. In a second feature of the eighth aspect of Embodiment E7, R⁵ is C(O)N(R⁷)R⁸ wherein R⁷ is H or C₁-C₄ alkyl and R⁸ is C₁-C₆ alkyl optionally substituted with R^{D} wherein R^{D} is phenyl optionally substituted with 1 to 2 substituents selected from the group consisting of halo, OR^{A}, NO₂, CN, CF₃, NR^{A}C(O)R^{B}, CO₂R^{A}, and C(O)N(R^{A})R^{B}; and all other variables are as defined in any one of Embodiments E0 to E6. In a third feature of the eighth aspect of Embodiment E7, R⁵ is C(O)N(R⁷)R⁸ wherein R⁷ and R⁸ are taken together with the N atom to which they are bonded to form a 5- or 6-membered saturated heterocyclic ring having no additional heteroatoms; and all other variables are as defined in any one of Embodiments E0 to E6. In a fourth feature of the eighth aspect of Embodiment E7, R⁵ is C(O)N(R⁷)R⁸ wherein R⁷ and R⁸ are taken together with the N atom to which they are bonded to form a piperidinyl ring substituted with phenylethyl; and all other variables are as defined in any one of Embodiments E0 to E6.

Embodiment E8 of the present invention is a compound of Formula I, or a pharmaceutically acceptable salt thereof, wherein R⁶ is H; and all other variables are as defined in any one of Embodiments E0 to E7.

Embodiment E9 of the present invention is a compound of Formula I, or a pharmaceutically acceptable salt thereof, wherein at least one of R⁴, R⁵ and R⁶ is other than H; and all other variables are as defined in any one of Embodiments E0 to E8.

Embodiment E10 of the present invention is a compound of Formula I, or a pharmaceutically acceptable salt thereof, as defined in any one of Embodiments E0 to E9, with the proviso (E) that when X is a bond and R² is N(R⁷)R⁸, then R⁷ and R⁸ in the definition of R² do not together with the N form a ring. It is understood that this limitation on N(R⁷)R⁸ applies only to R² and an N(R⁷)R⁸ in any other variable can optionally form such a ring.

Embodiment E11 of the present invention is a compound of Formula I, or a pharmaceutically acceptable salt thereof, as defined in any one of Embodiments E0 to E10, with the proviso (E′) that with respect to any N(R⁷)R⁸ group, R⁷ and R⁸ do not together with the N form a ring. It is understood that this limitation on N(R⁷)R⁸ applies generally to any group that includes one or more N(R⁷)R⁸ groups in its definition.

Embodiment E12 of the present invention is a compound of Formula I, or a pharmaceutically acceptable salt thereof, wherein R^{A} is H or C₁-C₆ alkyl; R^{B} is H or C₁-C₆ alkyl; and all other variables are as defined in any one of Embodiments E0 to E11. In a first aspect of Embodiment E12, R^{A} is H or C₁-C₄ alkyl; R^{B} is H or C₁-C₄ alkyl; and all other variables are as defined in any one of Embodiments E0 to E11. In a second aspect of Embodiment E12, R^{A} is H or CH₃; R^{B} is H or CH₃; and all other variables are as defined in any one of Embodiments E0 to E11.

Embodiment E13 of the present invention is a compound of Formula I, or a pharmaceutically acceptable salt thereof, wherein each aryl is phenyl or naphthyl; and all other variables are as defined in any one of Embodiments E0 to E12. It is understood that the references to aryl (whether unsubstituted or substituted with one or more substituents) in any of Embodiments E0 to E12 are replaced with corresponding references to phenyl and naphthyl in Embodiment E13. In an aspect of Embodiment E13, each aryl is phenyl; and all other variables are as defined in any one of Embodiments E0 to E12.

Embodiment E14 of the present invention is a compound of Formula I, or a pharmaceutically acceptable salt thereof, wherein:

(A) each heteroaryl is a 5- or 6-membered heteroaromatic ring containing from 1 to 3 heteroatoms independently selected from N, O and S, and

(B) each heterocyclyl is a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂;

and all other variables are as defined in any one of Embodiments E0 to E13. It is understood that the references to heteroaryl and heterocyclyl (whether unsubstituted or substituted with one or more substituents) in any one of Embodiments E0 to E13 are respectively replaced with corresponding references to the heteroaromatic ring set forth in (A) and the heterocyclic ring set forth in (B) in Embodiment E14.

Embodiment E15 of the present invention is a compound of Formula I, or a pharmaceutically acceptable salt thereof, wherein each aryl is as defined in Embodiment E13 and each heteroaryl and heterocyclyl are as defined in Embodiment E14; and all other variables are as defined in any one of Embodiments E0 to E12.

Embodiment E16 of the present invention is a compound, or a pharmaceutically acceptable salt thereof, selected from the group consisting of the compounds set forth in Examples 1-16, 18-43, 47-69, 72-82, 87-95, 97-103, 168 and 171 (alternatively referred to as Compounds 1-16, 18-43, 47-69, 72-82, 87-95, 97-103, 168 and 171) below.

Embodiment E17 of the present invention is a compound of Formula I as defined in Embodiment E0 above, or a pharmaceutically acceptable salt thereof, with the proviso (F) that when R¹ is O, R³ is OH or NH₂, R⁴ is H, R⁵ is H and R⁶ is H, then XR² is not H.

Aspects of Embodiment E17 include each of Embodiments E1, E2, E3, E4, E6, E7, E8, E10, E11, E12, E13, E14, E1S and E16, wherein proviso F is applied thereto.

Embodiment E18 of the present invention is a compound of Formula I as defined in Embodiment E0, or a pharmaceutically acceptable salt thereof, with the proviso (G) that when R¹ is O, R³ is OH, R⁴ is H, R⁵ is H and R⁶ is H, then XR² is not 1,1-dioxido-4H-1,2,4-benzothiadiazin-3-yl.

Aspects of Embodiment E18 include each of Embodiments E1, E2, E3, E4, E6, E7, E8, E10, E11, E12, E13, E14, E15 and E16, wherein proviso G is applied thereto.

Embodiment E19 of the present invention is a compound of Formula I, or a pharmaceutically acceptable salt thereof, as defined in any one of Embodiments E0, E1, E2, E3, E4, E6, E7, E8, E10, E11, E12, E13, E14, E15 and E16, wherein proviso F as set forth in Embodiment E17 and proviso G as set forth in Embodiment E18 are applied thereto.

Embodiment E20 of the present invention is a compound of Formula I as defined in Embodiment E0 above, or a pharmaceutically acceptable salt thereof, with the proviso (B′) that when R¹ is O, R³ is H, and R⁴═R⁵═R⁶═H, then XR² is not C(O)O—(C₁-C₆ alkyl). In a first aspect of this embodiment, proviso B′ provides that when R¹ is O, R³ is H, and R⁴═R⁵═R⁶═H, then XR² is not C(O)O—(C₁-C₁₂ alkyl).

Aspects of Embodiment E20 include each of Embodiments E1, E2, E3, E6, E7, E8, E10, E11, E12, E13, E14, E15, E16, E17, E18 and E19, wherein proviso B′ (as originally defined or as defined in the first aspect of E 20) is applied thereto.

A class of compounds of the present invention (alternatively referred to herein as Class C2) includes compounds of Formula I and pharmaceutically acceptable salts thereof, wherein:

### R¹ is O;

X is a bond or C(O),
R² is:(i) H, (2) halo, (3) C₁-C₄ alkyl, (4) O—C₁-C₄ alkyl, (5) C₃-C₆ cycloalkyl, (6) phenyl, (7) C₁-C₄ alkylene-phenyl, (8) NR^{7A}R^{8A}, or (9) HetA
wherein phenyl is optionally substituted with a total of from 1 to 3 substituents where:
(i) from zero to 3 of the substituents are selected from the group consisting of halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), and C(O)N(C₁-C₄ alkyl)₂, and
(ii) from zero to 1 of the substituents is phenyl, C₁-C₄ alkylene-phenyl, O—C₁-C₄ alkylene-phenyl, C₁-C₄ alkylene-HetJ, or O—C₁-C₄ alkylene-HetJ;
wherein HetA and HetJ are each independently a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, N—H(C₁-C₄ alkyl), N(C₁-C₄ alkyl)₂, C(O)NH₂, C(O)NH(C₁-C₄ alkyl), or C(O)N(C₁-C₄ alkyl)₂;
   and with the proviso (A) that XR² is not C(O)-halo;
   R^{7A} is H or C₁-C₄ alkyl;
   R^{8A} is: (1) H, (2) C₁-C₄ alkyl, (3) C₁-C₄ fluoroalkyl, (4) C₃-C₆ cycloalkyl, (5) phenyl, (6) C₁-C₄ alkylene-phenyl, (7) HetB, (8) C₁-C₄ alkylene-HetB, (9) HetC, or (10) C₁-C₄ alkylene-HetC;
wherein phenyl is optionally substituted with a total of from 1 to 3 substituents where:
   (i) from zero to 3 of the substituents are selected from the group consisting of halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), and C(O)N(C₁-C₄ alkyl)₂, and
(ii) from zero to 1 of the substituents is phenyl, C₁-C₄ alkylene-phenyl, O—C₁-C₄ alkylene-phenyl, C₁-C₄ alkylene-HetJ, or O—C₁-C₄ alkylene-HetJ, where HetJ is as defined above;
wherein HetB is a 5- to 7-membered saturated heterocyclic ring containing from 1 to 3 heteroatoms selected from 1 to 3 N atoms, zero to 10 atom, and zero to 1 S atom optionally in the form S(O) or S(O)₂, wherein the saturated heterocyclic ring is attached to the rest of the molecule via a ring carbon atom, and wherein the saturated heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl; and
wherein HetC is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), C(O)N(C₁-C₄ alkyl)₂, phenyl, C₁-C₄ alkylene-phenyl or O—C₁-C₄ alkylene-phenyl;
   alternatively, when X is C(O), R^{7A} and R^{8A} together with the N atom to which they are attached form a saturated heterocyclic ring selected from the group consisting of pyrrolidinyl, piperidinyl, piperazinyl, morpholinyl, thiomorpholinyl in which the S atom is optionally in the form S(O) or S(O)₂, and azepanyl, wherein the heterocyclic ring is optionally substituted with from 1 to 3 substituents each of which is independently oxo, C₁-C₄ alkyl, SO₂(C₁-C₄ alkyl), CO₂—C₁-C₄ alkyl, or C(O)—C₁-C₄ alkyl;
   R³ is OH, NH₂, N(H)C(O)—C₁-C₄ alkyl, N(H)C(O)-phenyl, N(H)C(O)—C₁-C₄ alkylene-phenyl, N(H)-phenyl, or phenyl;
   alternatively, R³ and XR² are taken together with the carbon atoms to which each is attached to provide:

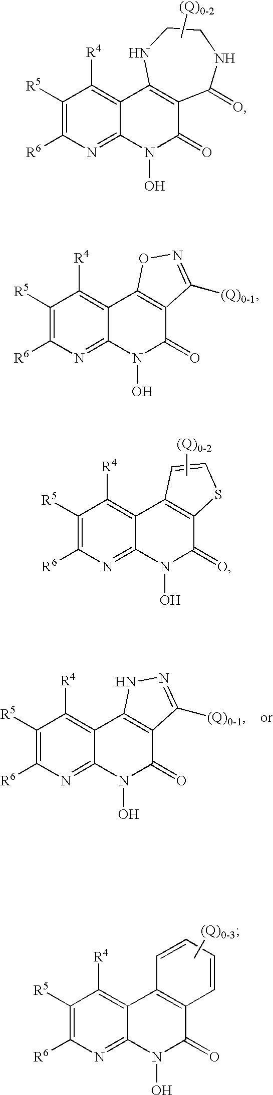

each Q is independently H, C₁-C₄ alkyl, halo, phenyl, or C₁-C₄ alkylene-phenyl;
R⁴ is H, CO₂—C₁-C₄ alkyl, or phenyl, wherein the phenyl is optionally substituted with from 1 to 3 substituents each of which is independently halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), or C(O)N(C₁-C₄ alkyl)₂;
R⁵ is: (1) H, (2) halo, (3) C₁-C₄ alkyl, (4) C₁-C₄ haloalkyl, (5) C(O)O—C₁-C₄ alkyl, (6) phenyl, (7) C₁-C₄ alkylene-phenyl, (8) C₁-C₄ alkenylene-phenyl, (9) O-phenyl, (10) SO₂N(H)-phenyl, (11) SO₂N(C₁-C₄ alkyl)-phenyl, (12) SO₂N(H)—C₁-C₄ alkylene-phenyl, (13) SO₂N(C₁-C₄ alkyl)-C₁-C₄ alkylene-phenyl, (14) naphthyl, (15) C₁-C₄ alkylene-naphthyl, (16) O-naphthyl, (17) HetD, (18) C₁-C₄ alkylene-N(H)—C₁-C₄ alkylene-phenyl, (19) C(O)N(H)—C₁-C₄ alkylene-phenyl, (20) C(O)N(C₁-C₄ alkyl)-C₁-C₄ alkylene-phenyl, or (21) C(O)NR^{7B}R^{8B};
wherein:
phenyl or naphthyl is optionally substituted with from 1 to 3 substituents each of which is independently halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), C(O)N(C₁-C₄ alkyl)₂, phenyl, C₁-C₄ alkylene-phenyl, O—C₁-C₄ alkylene-phenyl, HetK, C₁-C₄ alkylene-HetK, HetL, or C₁-C₄ alkylene-HetL; wherein
   HetK is a 5- to 7-membered saturated heterocyclic ring containing from 1 to 3 heteroatoms selected from N, O and S optionally in the form S(O) or S(O)₂, wherein the saturated heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl;
   HetL is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), or C(O)N(C₁-C₄ alkyl)₂;
HetD is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), C(O)N(C₁-C₄ alkyl)₂, phenyl, C₁-C₄ alkylene-phenyl or O—C₁-C₄ alkylene-phenyl;
   R⁶ is H or C₁-C₄ alkyl;
   R^{7B} is H or C₁-C₄ alkyl;
   R^{8B} is H or C₁-C₄ alkyl; and
   alternatively, R^{7B} and R^{8B} together with the N atom to which they are attached form a saturated heterocyclic ring selected from the group consisting of pyrrolidinyl, piperidinyl, piperazinyl, morpholinyl, thiomorpholinyl in which the S atom is optionally in the form S(O) or S(O)₂, and azepanyl, wherein the heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl.

A first sub-class of Class C2 (Sub-Class SO₂(C₁-C₄) is a compound of Formula I, wherein:

XR² is: (1) H, (2) halo, (3) C₁-C₄ alkyl, (4) C₃-C₆ cycloalkyl, (5) C(O)O—C₁-C₄ alkyl, (6) phenyl, (7) C₁-C₄ alkylene-phenyl, (8) C(O)NR^{7A}R^{8A}, or (9) HetA,
wherein phenyl is optionally substituted with a total of from 1 to 3 substituents where:
   (i) from zero to 3 of the substituents are selected from the group consisting of halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), and C(O)N(C₁-C₄ alkyl)₂, and
   (ii) from zero to 1 of the substituents is phenyl, C₁-C₄ alkylene-phenyl, O—C₁-C₄ alkylene-phenyl, C₁-C₄ alkylene-HetJ, or O—C₁-C₄ alkylene-HetJ;
      and all other variables are as originally defined in Class C2.

A second sub-class of Class C2 (Sub-Class SC2-2) is a compound of Formula I, wherein:

### R¹ is O;

XR² is: (1) H, (2) Cl, Br, or F, (3) C₁-C₄ alkyl, (4) C₃-C₆ cycloalkyl, (5) C(O)OCH₃, (6) C(O)OCH₂CH₃, (6) phenyl, (7) (CH₂)₁₋₂-phenyl, (8) C(O)NR^{7A}R^{8A}, or (9) HetA,
wherein phenyl is optionally substituted with from 1 or 2 substituents selected from the group consisting of Cl, Br, F, OH, CN, CH₃, OCH₃, CF₃, OCF₃, CN, SO₂CH₃, CO₂CH₃, C(O)CH₃, NH₂, NH(CH₃), N(CH₃)₂, N(H)SO₂CH₃, C(O)NH₂, C(O)NH(CH₃), and C(O)N(CH₃)₂, and
HetA is a heteroaromatic ring selected from the group consisting of pyridinyl, pyrimidinyl, and pyrazinyl, wherein the heteroaromatic ring is optionally substituted with 1 or 2 substituents each of which is independently Cl, Br, F, CH₃, OCH₃, CF₃, OCF₃, CN, SO₂CH₃, CO₂CH₃, C(O)CH₃, NH₂, NH(CH₃), N(CH₃)₂, C(O)NH₂, C(O)NH(CH₃), C(O)N(CH₃)₂, phenyl, CH₂-phenyl or OCH₂-phenyl;

### R^{7A} is H or CH₃;

R^{8A} is: (1) H, (2) CH₃, (3) CH₂CF₃, (4) cyclopropyl, (5) phenyl, (6) CH₂-phenyl, (6) CH(CH₃)-phenyl, (7) HetB, (8) CH₂—HetB, (9) HetC, or (10) CH₂—HetC; wherein:
phenyl is optionally substituted with a total of 1 or 2 substituents where:
   (i) from zero to 2 of the substituents are selected from the group consisting of Cl, Br, F, OH, CN, CH₃, OCH₃, CF₃, OCF₃, CN, SO₂CH₃, CO₂CH₃, C(O)CH₃, NH₂, NH(CH₃), N(CH₃)₂, N(H)SO₂CH₃, C(O)NH₂, C(O)NH(CH₃), and C(O)N(CH₃)₂, and
   (ii) from zero to 1 of the substituents is phenyl, CH₂-phenyl, OCH₂-phenyl, CH₂-pyridinyl, or OCH₂-pyridinyl;
HetB is a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the saturated heterocyclic ring is attached to the rest of the molecule via a ring carbon atom, and wherein the saturated heterocyclic ring is optionally substituted with 1 or 2 substituents each of which is independently oxo, CH₃, SO₂CH₃, CO₂CH₃, C(O)CH₃, or CH₂-phenyl; and
HetC is a heteroaromatic ring selected from the group consisting of pyridinyl, pyrimidinyl, and pyrazinyl, wherein the heteroaromatic ring is optionally substituted with 1 or 2 substituents each of which is independently Cl, Br, F, CH₃, OCH₃, CF₃, OCF₃, CN, SO₂CH₃, CO₂CH₃, C(O)CH₃, NH₂, NH(CH₃), N(CH₃)₂, C(O)NH₂, C(O)NH(CH₃), C(O)N(CH₃)₂, phenyl, CH₂-phenyl or OCH₂-phenyl;
   alternatively, R^{7A} and R^{8A} together with the N atom to which they are attached form a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the heterocyclic ring is optionally substituted with oxo, CH₃, SO₂CH₃, CO₂CH₃, or C(O)CH₃;
   R³ is OH, NH₂, N(H)C(O)CH₃, N(H)C(O)-phenyl, N(H)C(O)CH₂-phenyl, N(H)-phenyl, or phenyl;
   alternatively, R³ and XR² are taken together with the carbon atoms to which each is attached to provide:

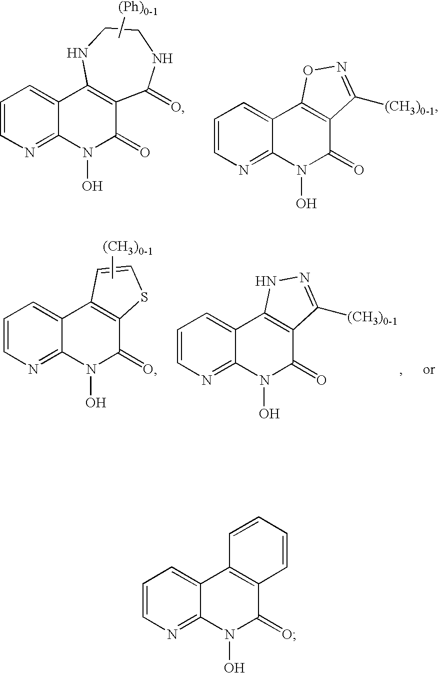

R⁴ is H, CO₂CH₃, CO₂CH₂CH₃, or phenyl;
R⁵ is: (1) H, (2) Cl, Br or F, (3) C₁-C₄ alkyl, (4) CH₂CF₃, (5) CH₂CH(CH₃)Br, (6) C(O)OCH₃, (7) C(O)OCH₂CH₃, (8) phenyl, (9) CH₂-phenyl, (10) CH(CH₃)-phenyl, (11) CH═CH-phenyl, (12) O-phenyl, (13) SO₂N(H)-phenyl, (14) SO₂N(CH₃)-phenyl, (15) SO₂N(H)CH₂-phenyl, (16) SO₂N(CH₃)CH₂-phenyl, (17) naphthyl, (18) CH₂-naphthyl, (19) O-naphthyl, (20) HetD, (21) CH₂N(H)CH₂-phenyl, (22) CH(CH₃)N(H)CH₂-phenyl, (23) C(O)N(H)(CH₂)₁₋₂-phenyl, (24) C(O)N(CH₃)(CH₂)₁₋₂-phenyl, or (25) C(O)NR^{7B}R^{8B}; wherein:
phenyl is optionally substituted with a total of 1 or 2 substituents where:
   (i) from zero to 2 of the substituents are selected from the group consisting of Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), and C(O)N(CH₂CH₃)₂, and
   (ii) from zero to 1 of the substituents is phenyl, CH₂-phenyl, OCH₂-phenyl, HetK, CH₂—HetK, HetL, or CH₂—HetL; wherein
      HetK is a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the saturated heterocyclic ring is attached to the rest of the molecule via a ring carbon atom, and wherein the saturated heterocyclic ring is optionally substituted with 1 or 2 substituents each of which is independently oxo, CH₃, CH₂CH₃, SO₂CH₃, SO₂CH₂CH₃, CO₂CH₃, CO₂CH₂CH₃, C(O)CH₃, C(O)CH₂CH₃, or CH₂-phenyl; and
      HetL is a heteroaromatic ring selected from the group consisting of thienyl, pyrrolyl, pyrazolyl, imidazolyl, pyridinyl, pyrimidinyl, and pyrazinyl, wherein the heteroaromatic ring is optionally substituted with 1 or 2 substituents each of which is independently Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), C(O)N(CH₂CH₃)₂, phenyl, CH₂-phenyl or OCH₂-phenyl;
HetD is a heteroaromatic ring selected from the group consisting of thienyl, pyrrolyl, pyrazolyl, imidazolyl, pyridinyl, pyrimidinyl, and pyrazinyl, wherein the heteroaromatic ring is optionally substituted with 1 or 2 substituents each of which is independently Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), C(O)N(CH₂CH₃)₂, phenyl, CH₂-phenyl or OCH₂-phenyl;

### R^{7B} is H, CH₃, or CH₂CH₃;

### R^{8B} is H, CH₃, or CH₂CH₃; and

alternatively, R^{7B} and R^{8B} together with the N atom to which they are attached form a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the heterocyclic ring is optionally substituted with oxo, CH₃, SO₂CH₃, CO₂CH₃, C(O)CH₃, or (CH₂)₁₋₂-phenyl; and

### R⁶ is H.

A third sub-class of Class C2 (Sub-Class SC2-3) is a compound of Formula I, or a pharmaceutically acceptable salt thereof, wherein R³ is OH; and all other variables are as originally defined in Class C2.

A fourth sub-class of Class C2 (Sub-Class SC2-4) is a compound of Formula I, or a pharmaceutically acceptable salt thereof, wherein R³ is OH; R⁶ is H; and all other variables are as originally defined in Class C2.

A fifth sub-class of Class C2 (Sub-Class SC2-5) is a compound of Formula I, or a pharmaceutically acceptable salt thereof, wherein R³ is OH; and all other variables are as defined in the Sub-Class SC2-2.

A sixth sub-class of Class C2 (Sub-Class SC2-6) is a compound of Formula I as defined in Class C2, or a pharmaceutically acceptable salt thereof, with the proviso (D) that when R³ is OH or NH₂, R⁴ is H, R⁵ is H and R⁶ is H, then XR² is not H. Additional sub-classes of Class C2 include a compound of Formula I as defined in any one of Sub-Classes SC2-1, SC2-2. SC2-3, SC2-4, and SC2-5, wherein proviso D set forth in Sub-Class SC2-6 is applied thereto.

Another embodiment of the present invention is a compound of Formula I, or a pharmaceutically acceptable salt thereof, as defined in any of the foregoing embodiments, aspects, classes, or sub-classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest impurity level is employed. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

The present invention also includes the following embodiments:

(a) A pharmaceutical composition comprising an effective amount of a compound of Formula I′ and a pharmaceutically acceptable carrier.

(b) A pharmaceutical composition which comprises the product prepared by combining (e.g., mixing) an effective amount of a compound of Formula I′ and a pharmaceutically acceptable carrier.

(c) The pharmaceutical composition of (a) or (b), further comprising an effective amount of a second anti-HIV agent (e.g., an anti-HIV-1 agent) other than a compound of Formula I′, selected from the group consisting of HIV antiviral agents, immunomodulators, and anti-infective agents.

(d) The pharmaceutical composition of (c), wherein the second anti-HIV agent is an HIV antiviral (e.g., an HIV-1 antiviral) other than a compound of Formula I′, selected from the group consisting of HIV protease inhibitors, HIV integrase inhibitors, non-nucleoside HIV reverse transcriptase inhibitors, and nucleoside HIV reverse transcriptase inhibitors.

(e) A pharmaceutical combination which is (i) a compound of Formula I′ and (ii) a second anti-HIV agent (e.g., an anti-HIV1 agent) other than a compound of Formula I′ selected from the group consisting of HIV antiviral agents, immunomodulators, and anti-infective agents; wherein the compound of Formula I′ and the anti-HIV agent are each employed in an amount that renders the combination effective for inhibiting HIV integrase and/or HIV reverse transcriptase (e.g., RNase H), for treating or preventing infection by HIV, or for preventing, treating or delaying the onset of AIDS.

(f) The combination of (e), wherein the second anti-HIV agent is an HIV antiviral other than a compound of Formula I′, selected from the group consisting of HIV protease inhibitors, HIV integrase inhibitors, non-nucleoside HIV reverse transcriptase inhibitors and nucleoside HIV reverse transcriptase inhibitors.

(g) A method of inhibiting HIV integrase and/or RNase H (e.g., HIV-1 integrase and/or RNase H) in a subject in need thereof which comprises administering to the subject an effective amount of a compound of Formula I′.

(h) A method of preventing or treating infection by HIV (e.g., HIV-1) in a subject in need thereof which comprises administering to the subject an effective amount of a compound of Formula I′.

(i) The method of (h), wherein the compound of Formula I′ is administered in combination with an effective amount of at least one other HIV antiviral other than a compound of Formula I′, selected from the group consisting of HIV protease inhibitors, HIV integrase inhibitors, non-nucleoside HIV reverse transcriptase inhibitors, and nucleoside HIV reverse transcriptase inhibitors.

(j) A method of preventing, treating or delaying the onset of AIDS in a subject in need thereof which comprises administering to the subject an effective amount of a compound of Formula I′.

(k) The method of 0), wherein the compound is administered in combination with an effective amount of at least one other HIV antiviral other than a compound of Formula I′, selected from the group consisting of HIV protease inhibitors, HIV integrase inhibitors, non-nucleoside HIV reverse transcriptase inhibitors, and nucleoside HIV reverse transcriptase inhibitors.

(l) A method of inhibiting HIV integrase and/or RNase H (e.g., HIV-1 integrase and/or HIV-1 RNase H) in a subject in need thereof which comprises administering to the subject the pharmaceutical composition of (a), (b), (c) or (d) or the combination of (e) or (f).

(m) A method of preventing or treating infection by HIV (e.g., HIV-1) in a subject in need thereof which comprises administering to the subject the pharmaceutical composition of (a), (b), (c) or (d) or the combination of (e) or (f).

(n) A method of preventing, treating or delaying the onset of AIDS in a subject in need thereof which comprises administering to the subject the pharmaceutical composition of (a), (b), (c) or (d) or the combination of (e) or (f).

In the embodiments (a)-(n) just described, the compound of Formula I′ has the same definition as a compound of Formula I as defined in the Summary of the Invention (i.e., as defined in either Embodiment D0 or Embodiment E0), except that proviso B is not applied; i.e., for the purposes of embodiments (a) to (n), suitable compounds of Formula I′ include those in which XR² is C(O)OCH₂CH₃ when R¹ is O and R³═R⁴═R⁵═R⁶═H. In an aspect of each of embodiments (a) to (n), the compound of Formula I′ is a compound of Formula I as defined in the Summary of Invention; i.e., proviso B is applied.

The present invention also includes a compound of Formula I′ (i) for use in, (ii) for use as a medicament for, or (iii) for use in the preparation of a medicament for: (a) inhibiting HIV integrase and/or RNase H, (b) preventing or treating infection by HIV, or (c) preventing, treating or delaying the onset of AIDS. In these uses, the compounds of Formula I′ can optionally be employed in combination with one or more other anti-HIV agents selected from HIV antiviral agents, anti-infective agents, and immunomodulators.

In an aspect of each of embodiments (i) to (iii), the compound of Formula I′ is a compound of Formula I as defined in the Summary of Invention; i.e., proviso B is applied.

Additional embodiments of the invention include the pharmaceutical compositions, combinations and methods set forth in (a)-(n) above and the uses set forth in (i)-(iii) above, wherein the compound of the present invention employed therein is a compound of Formula I as defined in one of the embodiments, aspects, classes, sub-classes, or features of Compound I set forth above. In all of these embodiments, the compound may optionally be used in the form of a pharmaceutically acceptable salt and/or hydrate.

The present invention also includes prodrugs of the compounds of Formula I and I′. The term “prodrug” refers to a derivative of a compound of Formula I (or I′), or a pharmaceutically acceptable salt thereof, which is converted in vivo into Compound I (or I′). Prodrugs of compounds of Formula I (or I′) can exhibit enhanced solubility, absorption, and/or lipophilicity compared to the compounds per se, thereby resulting in increased bioavailability and efficacy. The in vivo conversion of the prodrug can be the result of an enzyme-catalyzed chemical reaction, a metabolic chemical reaction, and/or a spontaneous chemical reaction (e.g., solvolysis). The prodrug can be, for example, a derivative of a hydroxy group such as an ester (—OC(O)R), a carbonate ester (—OC(O)OR), a phosphate ester (—O—P(═O)(OH)₂), or an ether (—OR). Other examples include the following: When the compound of Formula I (or I′) contains a carboxylic acid group, the prodrug can be an ester or an amide, and when the compound of Formula I (or I′) contains a primary amino group or another suitable nitrogen that can be derivatized, the prodrug can be an amide, carbamate, urea, imine, or a Mannich base. One or more functional groups in Compound I (or I′) can be derivatized to provide a prodrug thereof. Conventional procedures for the selection and preparation of suitable prodrug derivatives are described, for example, in Design of Prodrugs, edited by H. Bundgaard, Elsevier, 1985; J. J. Hale et al., J. Med. Chem. 2000, vol. 43, pp. 1234-1241; C. S. Larsen and J. Ostergaard, “Design and application of prodrugs” in: Textbook of Drug Design and Discovery, 3^{rd} edition, edited by C. S. Larsen, 2002, pp. 410-458; and Beaumont et al., Current Drug Metabolism 2003, vol. 4, pp. 461-458; the disclosures of each of which are incorporated herein by reference in their entireties.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical having a number of carbon atoms in the specified range. Thus, for example, “—C₁-C₆ alkylene-” refers to any of the C₁ to C₆ linear or branched alkylenes, and “—C₁-C₄ alkylene-” refers to any of the C1 to C4 linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest is an alkylene selected from the group consisting of —CH₂—, —CH(CH₃)—, and —C(CH₃)₂—. Expressions such as “C₁-C₄ alkylene-phenyl” and “C₁-C₄ alkyl substituted with phenyl” have the same meaning and are used interchangeably.

The term “cycloalkyl” refers to any cyclic ring of an alkane having a number of carbon atoms in the specified range. Thus, for example, “C₃-C₈ cycloalkyl” (or “C₃₋₈ cycloalkyl”) refers to cyclopropyl, cyclobutyl, cyclopentyl, cyclohexyl, cycloheptyl, and cyclooctyl.

The term “alkenylene” refers to any divalent linear or branched chain aliphatic mono-unsaturated hydrocarbon radical having a number of carbon atoms in the specified range.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁-C₆ haloalkyl” (or “C₁₋₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “aryl” refers to (i) phenyl, (ii) 9- or 10-membered bicyclic, fused carbocylic ring systems in which at least one ring is aromatic, and (iii) 11- to 14-membered tricyclic, fused carbocyclic ring systems in which at least one ring is aromatic. Suitable aryls include, for example, phenyl, naphthyl, tetrahydronaphthyl (tetralinyl), indenyl, anthracenyl, and fluorenyl.

The term “heteroaryl” refers to (i) 5- and 6-membered heteroaromatic rings and (ii) 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl

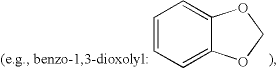

benzopiperidinyl, benzisoxazolyl, benzoxazolyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl

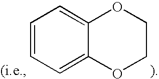

The term “heterocyclyl” refers to (i) 4- to 8-membered, saturated and unsaturated but non-aromatic monocyclic rings containing at least one carbon atom and from 1 to 4 heteroatoms, (ii) 7- to 12-membered bicyclic ring systems containing from 1 to 6 heteroatoms, and (iii) 10- to 18-membered tricyclic ring systems, wherein each ring in (ii) or (iii) is independent of, fused to, or bridged with the other ring or rings and each ring is saturated or unsaturated but nonaromatic, and wherein each heteroatom in (i), (ii), and (iii) is independently selected from N, O and S, wherein each N is optionally in the form of an oxide and each S is optionally oxidized to S(O) or S(O)₂. Suitable 4- to 8-membered saturated heterocyclyls include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, dioxanyl, and azacyclooctyl. Suitable unsaturated heterocyclic rings include those corresponding to the saturated heterocyclic rings listed in the preceding sentence in which a single bond is replaced with a double bond (e.g., a carbon-carbon single bond is replaced with a carbon-carbon double bond). Suitable saturated heterobicyclics include:

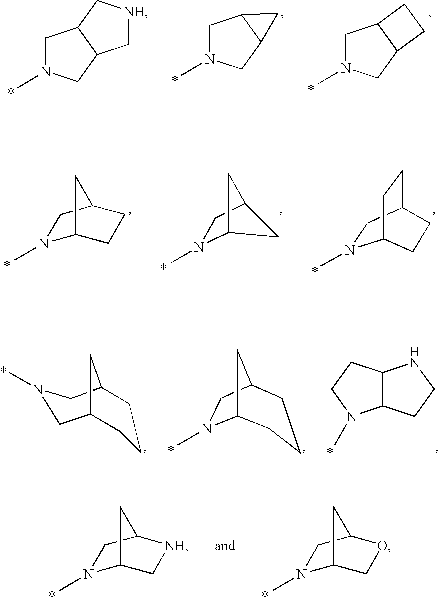

and suitable unsaturated heterobicyclics include those corresponding to the foregoing saturated heterobicyclics in which a single bond is replaced with a double bond. It is understood that the specific rings and ring systems suitable for use in the present invention are not limited to those listed in this and the preceding paragraphs. These rings and ring systems are merely representative.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

When any variable (e.g., R^{A}, R^{B}, R^{C}, R^{D}, and R^{E}) occurs more than one time in any constituent in any formula or embodiment depicting and describing compounds of the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, heteroaryl, cycloalkyl, or heterocyclyl) provided such ring substitution is chemically allowed and results in a stable compound.

Unless expressly stated to the contrary, any of the various carbocyclic and heterocyclic rings and ring systems defined herein may be attached to the rest of the compound at any ring atom (i.e., any carbon atom or any heteroatom) provided that a stable compound results.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

As would be recognized by one of ordinary skill in the art, certain of the compounds of the present invention can exist as tautomers. For the purposes of the present invention a reference herein to a compound of Formula I (or I′) is a reference to the compound per se, or to any one of its tautomers per se, or to mixtures of two or more tautomers. In instances where a hydroxy (—OH) substituent(s) is(are) permitted on a heteroaromatic ring and keto-enol tautomerism is possible, it is understood that the substituent might in fact be present, in whole or in part, in the keto form. Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

The compounds of the present inventions are useful in the inhibition of HIV reverse transcriptase (e.g., HIV-1 RNase H) and/or integrase (e.g., H-1 integrase), the prophylaxis or treatment of infection by human immunodeficiency virus (HIV) and the prophylaxis, treatment or the delay in the onset of consequent pathological conditions such as AIDS. Preventing AIDS, treating AIDS, delaying the onset of AIDS, or preventing or treating infection by HIV is defined as including, but not limited to, treatment of a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV reverse transcriptase (e.g., RNase H) and/or HIV integrase, e.g., by competitive inhibition. Thus the compounds of this invention are commercial products to be sold for these purposes.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating HIV infection or AIDS), “administration” and its variants are each understood to mean that the compound of the invention and the other agent(s) can be administered separately or together, and when administered separately, the dosage form and agent can be given concurrently or at different times (e.g., alternately).

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (e.g., RNase H) and/or HIV integrase and thereby elicit the response being sought (i.e., an “inhibition effective amount”). When the active compound (i.e., active ingredient) is administered as the salt, references to the amount of active ingredient are to the free acid or free base form of the compound.

For the purpose of inhibiting HIV RNase H and/or HIV integrase, preventing or treating HIV infection or preventing, treating or delaying the onset of AIDS, the compounds of the present invention, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in the compositions is provided in Remington's Pharmaceutical Sciences, 18^{th} edition, edited by A. R. Gennaro, Mack Publishing Co., 1990 and in Remington—The Science and Practice of Pharmacy, 21^{st} edition, Lippincott Williams & Wilkins, 2005.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of the HIV RNase H and/or HIV integrase inhibitor compounds of the present invention with one or more anti-HIV agents. An “anti-HIV agent” is any agent which is directly or indirectly effective in the inhibition of HIV integrase or another enzyme required for HIV replication or infection, the treatment or prophylaxis of HIV infection, and/or the treatment, prophylaxis or delay in the onset of AIDS. It is understood that an anti-HIV agent is effective in treating, preventing, or delaying the onset of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS, such as those disclosed in Table 1 of WO 01/38332 or in the Table in WO 02/30930. Suitable HIV antivirals for use in combination with the compounds of the present invention include, for example, those listed in Table A as follows:

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| Capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| Lopinavir | PI |
| lopinavir + ritonavir, Kaletra ® | PI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, Viread ® | nRTI |
| tipranavir, Aptivus ® | PI |

FI = fusion inhibitor; InI = integrase inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., abacavir sulfate, indinavir sulfate, atazanvir sulfate, nelfinavir mesylate.

It is understood that the scope of combinations of the compounds of this invention with anti-HIV agents is not limited to the HIV antivirals listed in Table A and/or listed in the above-referenced Tables in WO 01/38332 and WO 02/30930, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), the 60^{th} edition (2006), or the 61^{st} edition (2007). The dosage ranges for a compound of the invention in these combinations are the same as those set forth above.

Abbreviations employed herein include the following: Ac=acetyl; AIDS=acquired immunodeficiency syndrome; Bn=benzyl; BOC (or Boc)=t-butyloxycarbonyl; DCM=dichloromethane; DIPEA=diisopropylethylamine; DMF=dimethylformamide; DMSO=dimethyl sulfoxide; dppf=1,1′-bis(diphenylphosphino)ferrocene; DTT=dithiothreitol (Cleland's reagent); EDC=1-ethyl-3-(3-dimethylaminopropyl) carbodiimide; EDTA=ethylenediaminetetraacetic acid; EGTA=ethylene glycol bis(2-aminoethyl ether)-N,N,N′,N′-tetraacetic acid; ES MS=electrospray mass spectroscopy; Et=ethyl; EtOAc=ethyl acetate; EtOH=ethanol; FT-ICR-MS=fourier transform ion cyclotron resonance mass spectroscopy; HATU=O-(7-Azabenzotriazol-1-yl)N,N,N′,N′-tetramethyluronium hexafluorophosphate; HOAc=acetic acid; HOAT=1-hydroxy-7-azabenzotriazole; HOBT or HOBt=1-hydroxy benzotriazole; HPLC=high performance liquid chromatography; LC-MS=liquid chromatography-mass spectroscopy; LD₅₀=the dose lethal to 50% of a test population; LiHMDS=lithium hexamethyldisilazide; MCPBA=meta-chloroperoxybenzoic acid; Me=methyl; MeOH=methanol; MS FT-ICR=fourier transform ion cyclotron resonance mass spectroscopy; NMR=nuclear magnetic resonance; PEG=polyethylene glycol; Ph=phenyl; RP-HPLC=reverse phase HPLC; SGC=silica gel column chromatography; TEA=triethylamine; TFA=trifluoroacetic acid; TFAA=trifluoroacetic anhydride; THF=tetrahydrofuran; UHP=urea hydrogen peroxide.

The compounds of the present invention can be tested for inhibition of HIV reverse transcriptase (e.g., RNase H) and HIV integrase activity, as well as for inhibition of HIV replication according to the methods known in the art. A suitable assay for determining RNase H inhibitory activities is the ASH assay, described as follows:

Potency of a substance as an RNase H inhibitors can be determined by measuring its ability prevent RNase H catalyzed cleavage of the RNA strand in a RNA/DNA hybrid duplex substrate. RNase H activity is measured using a substrate generated by annealing the oligoribo-nucleotide 5′-rCrCrUrCrUrCrArArArArArCrArGrGrArGrCrArGrArArArGrArCrArArG (SEQ ID NO:1) to the oligodeoxyribonucleotide 5′-Biotin-GTCTTTCTGCTC (SEQ ID NO:2). Reactions are carried out by mixing HIV-1 reverse transcriptase (3.1 nM, inhibitor, and RNA/DNA hybrid duplex substrate (39.1 nM) in a solution containing 50 mM Tris-HCl, pH 7.8, 80 mM KCl, 6 mM MgCl2, 1 mM DTT, 0.1 mM EGTA, 0.2% PEG 8000 (i.e., polyethylene glycol with an average molecular weight=8000), and 1-10% DMSO. Reactions are incubated at 37° C. for 60 minutes and then quenched by the addition of EDTA to a final concentration of 119 mM. Cleavage of the RNA strand in the duplex results in the dissociation of the 5′-Biotinylated DNA strand. The released 5′-Biotinylated DNA is annealed to a complementary oligodeoxyribonucleotide: 5′-Fluorescein-GAGCAGAAAGAC (SEQ ID NO:3). The resulting double-stranded duplex DNA product is quantitated in an ALPHA screen format using [streptavidin- and anti-fluorescein-coated beads (Packard Bioscience) following the manufacturer's guidelines and reading on a Fusion AlphaScreen instrument. Alternatively, the released 5′-Biotinylated DNA is annealed to a complementary oligodeoxyribonucleotide: 5′-ruthenium-GAGCAGAAAGAC (SEQ ID NO:3). The resulting double-stranded duplex DNA product is quantitated in an ECL screen format using Dynabeads M280 coated with streptavidin (BioVeris Corporation) following the manufacturer's guidelines and reading on a BioVeris M384 Analyzer.

A suitable assay for determining integrase inhibitory activity is the assay measuring the strand transfer activity of integrase as described in WO 02/30930 (and further described in Wolfe, A. L. et al., J. Virol. 1996, 70: 1424-1432, Hazuda et al., J. Virol. 1997, 71: 7005-7011; Hazuda et al., Drug Design and Discovery 1997, 15: 17-24; and Hazuda et al., Science 2000, 287: 646-650).

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. “Ar” in the schemes below refers to optionally substituted aryl.

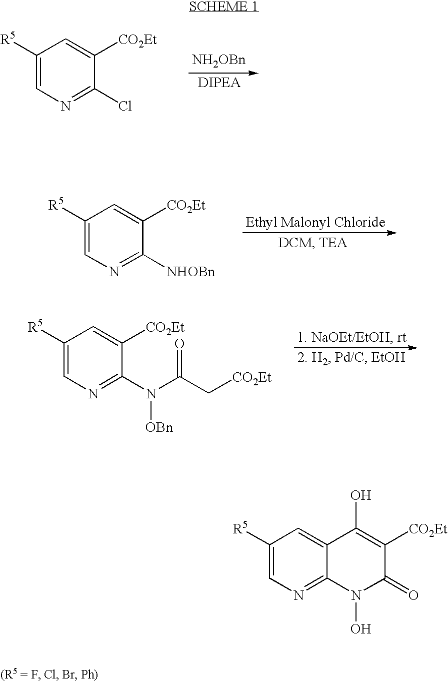

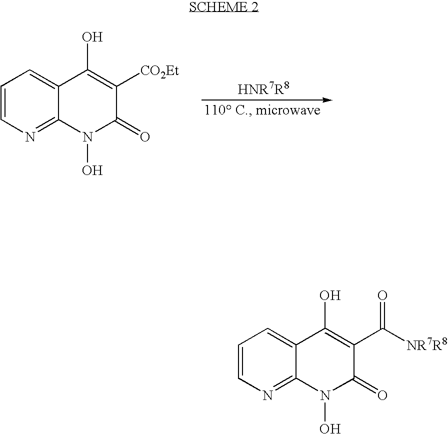

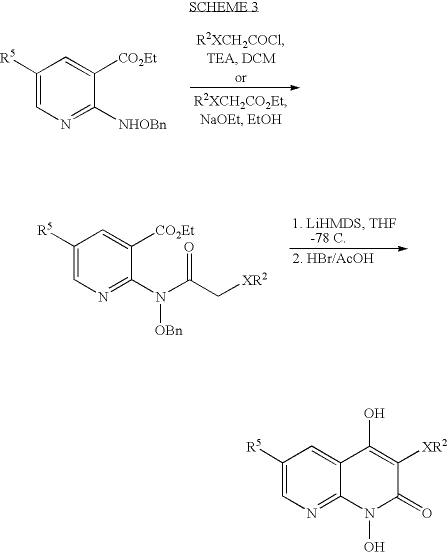

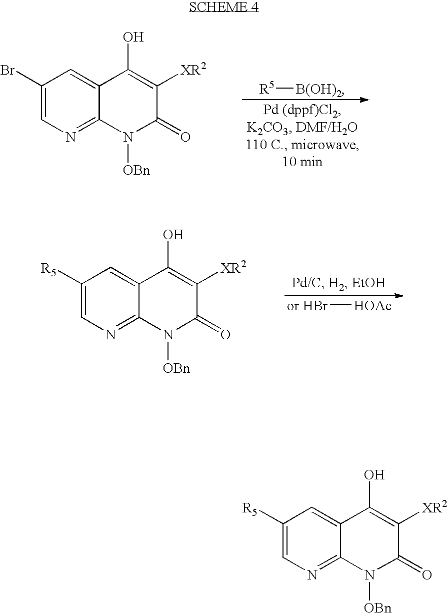

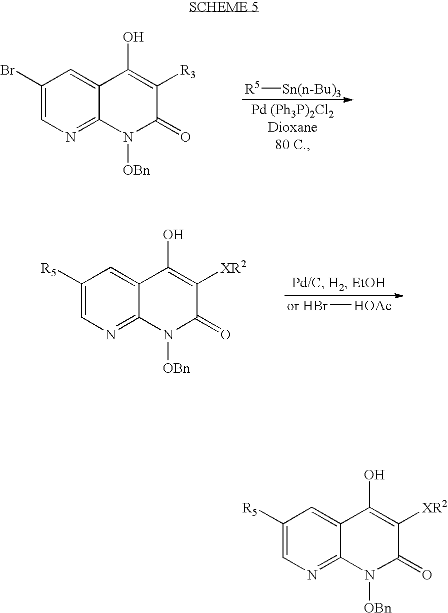

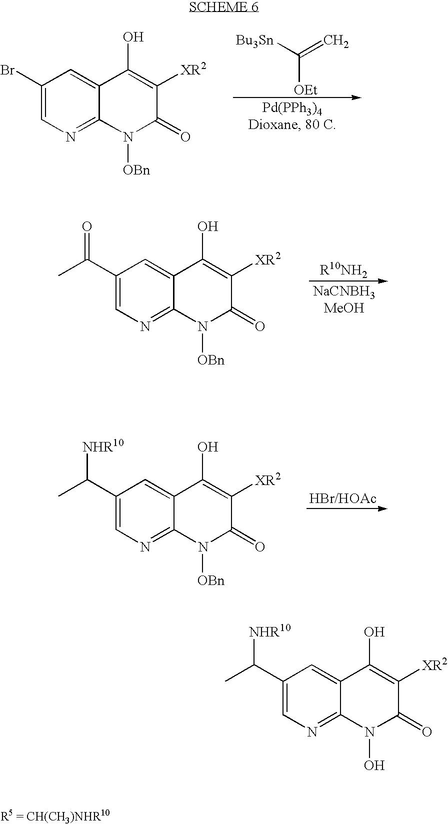

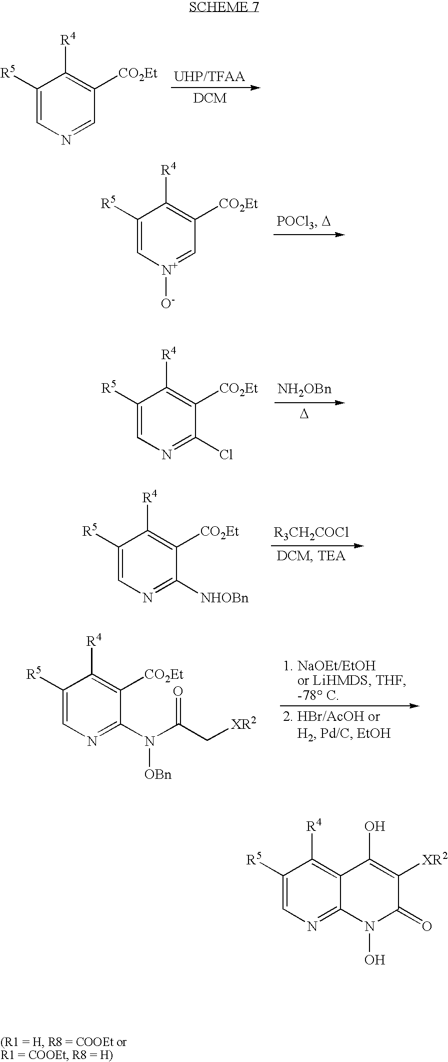

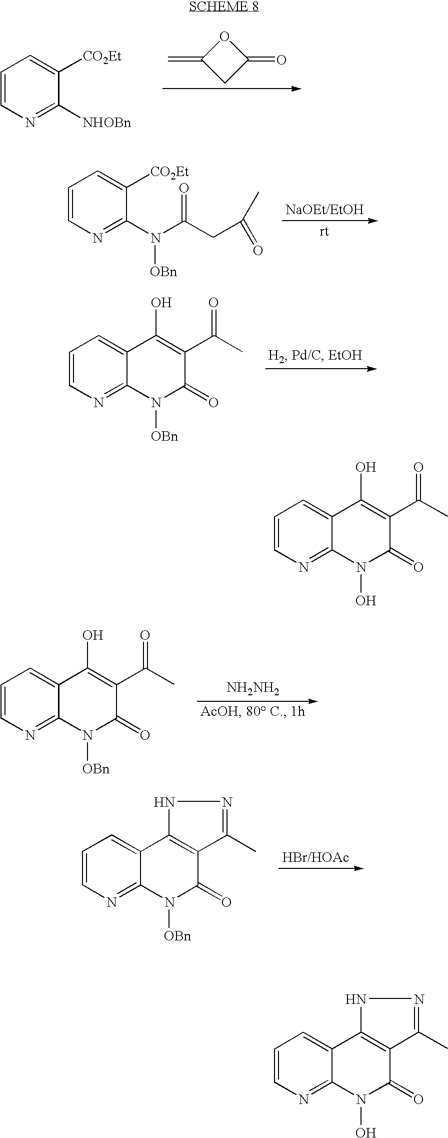

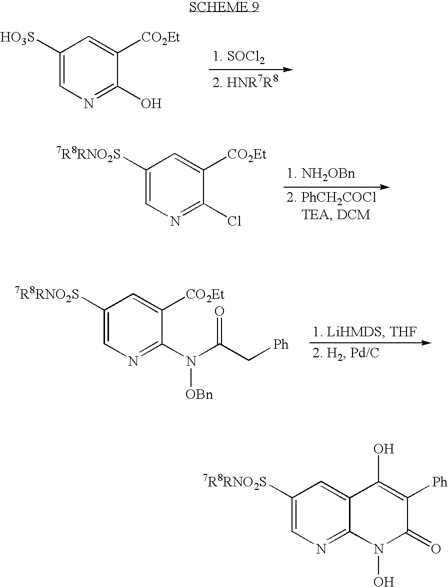

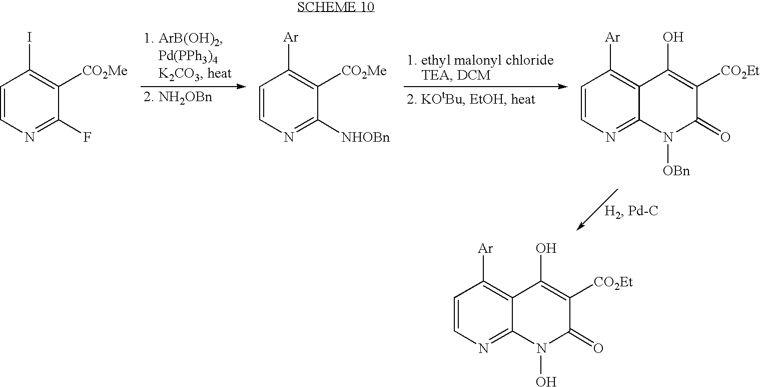

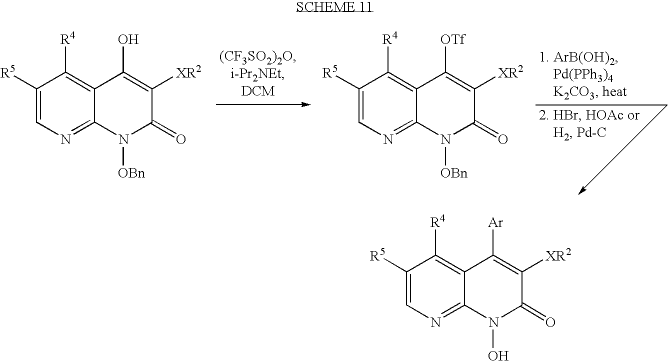

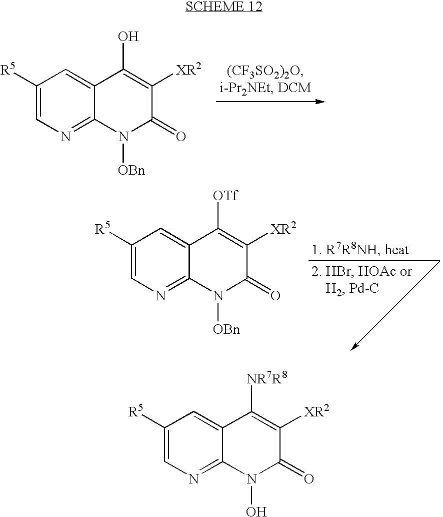

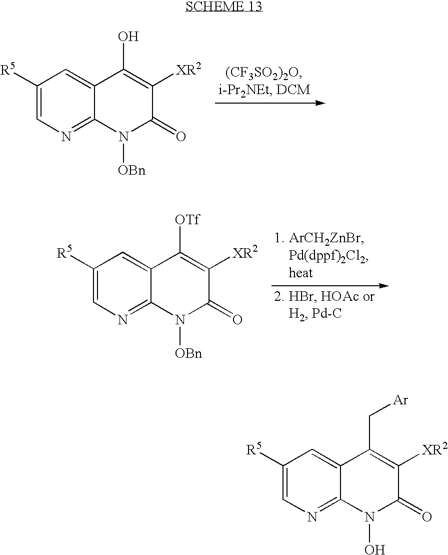

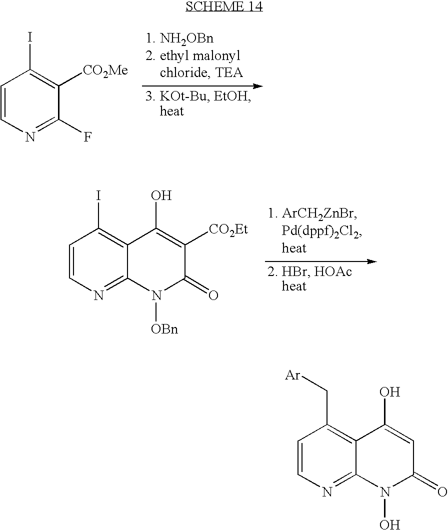

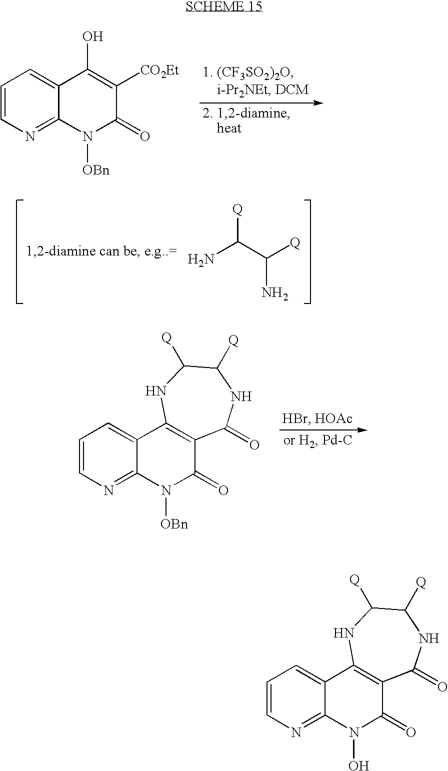

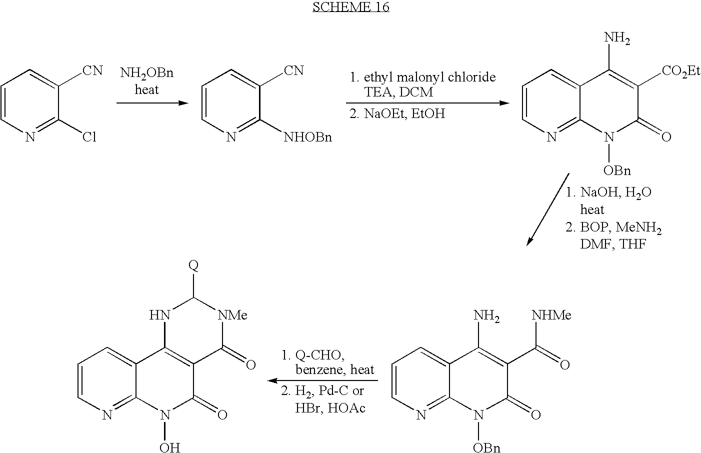

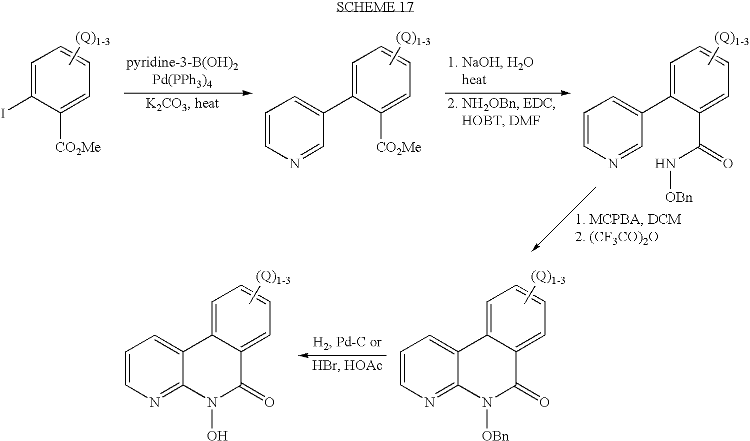

The following examples serve only to illustrate the invention and its practice. The examples are not to be construed as limitations on the scope or spirit of the invention.

### Example 1

### Ethyl 1,4-dihydroxy-2-oxo-1,2-dihydro-1,8-naphthyridine-3-carboxylate

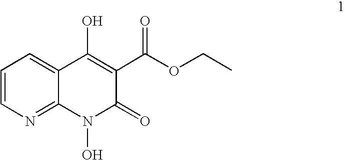

### Step 1: Ethyl 2-[(benzyloxy)(3-ethoxy-3-oxopropanoyl)amino]nicotinate

To a solution of ethyl 2-[(benzyloxy)amino]nicotinate (J. Het. Chem. 1993, 30 (4), 909-912; 7.0 g, 25.7 mmol) and TEA (7.17 mL, 51.4 mmol) in DCM (250 mL) was added dropwise ethyl malonyl chloride (6.62 mL, 51.4 mmol). After 1 hour, the solvent was removed and the solids formed were filtered off. The filtrate was concentrated and the residue was purified by SGC (0%→40% EtOAc/hexanes) to give the title compound as an orange oil. ¹H NMR (400 MHz, d₆-DMSO, ppm): δ 8.71 (d, J=3.9 Hz, 1H), 8.22 (dd, J=1.8, 7.7 Hz, 1H), 7.56 (dd, J=4.8, 7.7 Hz, 1H), 7.36 (m, 5H), 4.99 (s, 2H), 4.24 (q, J=7.1 Hz, 2H), 4.08 (q, J=7.1 Hz, 2H), 3.69 (s, 2H), 1.26 (t, J=7.1 Hz, 3H), and 1.17 (m, 3H). ES MS: m/z=387 (M+1).

### Step 2: Ethyl 1-(benzyloxy)-4-hydroxy-2-oxo-1,2-dihydro-1,8-naphthyridine-3-carboxylate

To a solution of ethyl 2-[(benzyloxy)(3-ethoxy-3-oxopropanoyl)amino]nicotinate (7.0 g, 18.1 mmol) in anhydrous EtOH (200 mL) was added dropwise a solution of sodium ethoxide (21% wt. in EtOH; 16.9 mL, 45.3 mmol). The reaction was stirred at for 18 hours. The reaction solution was brought to pH 4 by the addition of 2N HCl. After 15 minutes, the solids formed were collected by vacuum filtration to give the title compound. The filtrate was concentrated and then diluted with EtOH. The solids formed were collected and combined with the other product to give the title compound. ¹H NMR (400 MHz, d₆-DMSO, ppm): δ 13.2 (br s, 1H), 8.79 (dd, J=1.7, 4.7 Hz, 1H), 8.48-8.46 (m, 1H), 7.63 (dd, J=1.7, 7.8 Hz, 1H), 7.42-7.37 (m, 5H), 5.11 (s, 2H), 4.32 (q, J=7.0 Hz, 2H), and 1.29 (t, J=7.0 Hz, 3H). ES MS: m/z=341 (M+1).

### Step 3: Ethyl 1,4-dihydroxy-2-oxo-1,2-dihydro-1,8-naphthyridine-3-carboxylate

To a solution of ethyl 1-(benzyloxy)-4-hydroxy-2-oxo-1,2-dihydro-1,8-naphthyridine-3-carboxylate (3.0 g, 8.82 mmol) in degassed EtOH (300 mL) was added 10% Pd/C (0.3 g). The reaction mixture was further degassed and purged with N₂ (×3) and was then placed under H₂ balloon and stirred for 1 hour. The mixture was filtered through Celite and washed with degassed hot EtOH. The filtrate was concentrated. The resulting solids were triturated with EtOH and the solids were collected by vacuum filtration to give the title compound. ¹H NMR (400 MHz, d₆-DMSO, ppm): δ 12.9 (br s, 1H), 10.8 (s, 1H), 8.75 (dd, J=4.7 and 1.7 Hz, 1H), 8.43 (dd, J=8.0 and 1.7 Hz, 1H), 7.36 (dd, J=8.0 and 4.7 Hz, 1H), 4.34 (q, J=7.1 Hz, 2H), and 1.31 (t, J=7.1 Hz, 3H). High Resolution MS (FT-ICR): m/z found 251.0664 (M+1); calculated 251.0663 (M+1).

### Example 2

### 1,4-Dihydroxy-1,8-naphthyridin-2(1B)-one

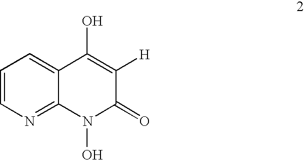

### Step 1: 1-(Benzyloxy)-4-hydroxy-1,8-naphthyridin-2(1H)-one

A stirred solution of ethyl 1-(benzyloxy)-4-hydroxy-2-oxo-1,2-dihydro-1,8-naphthyridine-3-carboxylate (Example 1, Step 2; 4.0 g, 12 mmol) in MeOH (100 mL) and 1 N aqueous NaOH (50 mL, 50 mmol) was heated to boiling. The MeOH was distilled off and the resulting aqueous solution was heated at reflux for 4 hours. The mixture was cooled in an ice-water bath and to the stirred mixture was added conc. HCl dropwise until the solution was pH 1-2. During the addition of the HCl a thick precipitate had formed. The precipitate was collected by filtration and dried for 48 hours to afford the title compound. ¹H NMR (400 MHz, d₆-DMSO, ppm): δ 11.87 (s, 1H), 8.73 (d, J=4.6 Hz, 1H), 8.27 (d, J=7.9 Hz, 1H), 7.66-7.64 (m, 2H), 7.45-7.35 (m, 4H), 5.96 (m, 1H), and 5.14 (s, 2H). ES MS: m/z=269 (M+1).

### Step 2: 1,4-Dihydroxy-1,8-naphthyridin-2(1H)-one

1-(Benzyloxy)-4-hydroxy-1,8-naphthyridin-2(1H)-one (150 mg, 0.56 mmol) was dissolved in a mixture of 33 wt % HBr in HOAc solution (3 mL) and H₂O (1 ml) and heated to 80° C. for two hours. The solvent was removed and the residue was triturated with MeOH. The solids were collected by vacuum filtration to afford the title compound as a white solid. ¹H NMR (400 MHz, d₆-DMSO, ppm): δ 11.7 (br s, 1H), 8.65 (dd, J=1.7, 4.8 Hz, 1H), 8.27 (dd, J=1.7, 7.9 Hz, 1H), 7.32 (dd, J=4.8, 7.9 Hz, 1H), and 5.95 (s, 1H). High Resolution MS: m/z found 179.0444 (M+1), calculated 179.0451 (M+1).

### Example 3

### Ethyl 6-bromo-1,4-dihydroxy-2-oxo-1,2-dihydro-1,8-naphthyridine-3-carboxylate

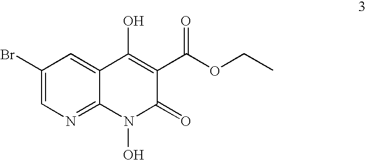

### Step 1: Methyl 2-[(benzyloxy)amino]-5-bromonicotinate

A mixture of methyl-5-bromo-2-chloronicotinate (5 g, 20 mmol) and O-benzylhydroxylamine (10 mL) in a dry flask was stirred at 110° C. overnight. The resulting solution was cooled, treated with aqueous buffer solution (300 mL, pH=4) and extracted with EtOAc (200 mL). The organic layer was washed with H₂O and dried over anhydrous magnesium sulfate. The solvent was removed. The crude product was purified by SGC (10-30% EtOAc/hexane) to give the title compound. ES MS: m/z=337.1 (M+1).

### Step 2: Methyl 2-[(benzyloxy)(3-ethoxy-3-oxopropanoyl)amino]-5-bromonicotinate

To a solution of methyl 2-[(benzyloxy)amino]-5-bromonicotinate (4.0 g, 12 mmol) and TEA (3.8 mL, 25.0 mmol) in DCM (250 mL) was added dropwise ethyl malonyl chloride (3.31 mL, 25.0 mmol). After 1 hour, the solvent was removed and the solids formed were filtered off. The filtrate was concentrated and the residue was purified by SGC (0%→40% EtOAc/hexanes) to give the title compound as an orange oil. ES MS: m/z=451.1 (M+1).

### Step 3: Ethyl 1-(benzyloxy)-6-bromo-4-hydroxy-2-oxo-1,2-dihydro-1,8-naphthyridine-3-carboxylate

To a solution of methyl 2-[(benzyloxy)(3-ethoxy-3-oxopropanoyl)amino]-5-bromonicotinate (4.0 g, 8.1 mmol) in anhydrous EtOH (200 mL) was added dropwise a solution of sodium ethoxide (21% wt. in EtOH; 2.5 mL, 8.1 mmol). The reaction was stirred for 18 hours. The reaction solution was brought to pH 4 by the addition of aqueous 2N HCl. After 15 minutes, the solids formed were collected by vacuum filtration to give the title compound. The filtrate was concentrated and then diluted with EtOH. The solids formed were collected and combined with the other product to give the title compound. ¹H NMR (400 MHz, d₆-DMSO, ppm): δ 10-82 (s, 1H), 8.75 (s, 1H), 8.38 (s, 1H), 7.51 (m, 5H), 5.21 (s, 2H), 4.34 (q, J=7.1 Hz, 2H), and 1.31 (t, J=7.1 Hz, 3H). ES MS: m/z=418.2 (M+1).

### Step 4: Ethyl 6-bromo-1,4-dihydroxy-2-oxo-1,2-dihydro-1,8-naphthyridine-3-carboxylate

To a solution of ethyl 6-bromo-1-(benzyloxy)-4-hydroxy-2-oxo-1,2-dihydro-1,8-naphthyridine-3-carboxylate (0.5 g, 1.2 mmol) in HOAc (3 mL) was added 33% HBr/HOAc (1.0 mL). The reaction mixture was heated to 80° C. and stirred for 1 hour. The solution was concentrated and purified by RP-HPLC (C18 column with H₂O/CH₃CN as mobile phase) to give the title compound. ¹H NMR (400 MHz, d₆-DMSO, ppm): 10.80 (s, 1H), 8.65 (s, 1H), 8.38 (s, 1H), 4.34 (q, J=7.6 Hz, 2H), and 1.31 (t, J=7.6 Hz, 3H). High Resolution MS (FT-ICR): m/z found 328.9776 (M+1); calculated 328.9768 (M+1).

### Example 4

### Ethyl 1,4-dihydroxy-2-oxo-5-phenyl-1,2-dihydro-1,8-naphthylidine-3-carboxylate

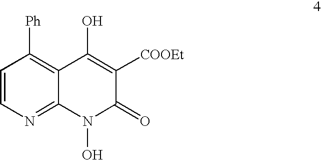

### Step 1: Methyl 2-[(benzyloxy)amino]-4-phenylnicotinate

Methyl 2-fluoro-4-phenylnicotinate (1.0 g, 4.31 mmol)) was taken up in DMSO (10 mL) and O-benzylhydroxylamine (2.0 mL) was added. The mixture was heated at 100° C. overnight. The solution was cooled, diluted with H₂O (50 mL) and extracted with EtOAc (2×50 mL). The organic layers were combined and the solvent removed. The residue was purified by SGC (10-50% EtOAc-hexanes) to give the title compound. ES MS: m/z=335 (M+1).

### Step 2: Methyl 2-[(benzyloxy)(3-ethoxy-3-oxopropanoyl)amino]-4-phenylnicotinate

A solution of methyl 2-[(benzyloxy)amino]-4-phenylnicotinate (1.0 g, 2.9 mmol) in DCM (20 mL) and pyridine (3.0 mL) was treated with ethyl malonyl chloride (0.5 mL, 3.0 mmol) and the mixture stirred at room temperature for 1 hour. Aqueous HCl (1.0 M, 20 mL) was added. The organic layer was separated and concentrated. The residue was purified by SGC (20-100% EtOAc-hexanes) to give the title compound. ES MS: m/z=363.3 (M+1).

### Step 3: Ethyl 1-(benzyloxy)-4-hydroxy-2-oxo-5-phenyl-1,2-dihydro-1,8-naphthyridine-3-carboxylate

Potassium tert-butoxide (50 mg, 0.45 mmol) was added to EtOH (10 mL) and the solution was heated to 80° C. Methyl 2-[(benzyloxy)(3-ethoxy-3-oxopropanoyl)amino]-4-phenylnicotinate (100 mg, 0.22 mmol) was taken up in EtOH (5.0 mL) and the solution was added dropwise to the hot potassium tert-butoxide solution over 5 minutes. The mixture was then cooled and the EtOH was removed. The residue was acidified with aqueous HCl (1.0 M, 5 mL) and extracted into EtOAc (20 mL). The organic layer was dried and concentrated. The residue was recrystallized from EtOAc and hexane to afford the title compound. ¹H NMR (400 MHz, CDCl₃, ppm): δ 8.72 (dd, J=6.4, 6.8 Hz, 1H,), 7.50 (m, 2H), 7.32-7.50 (m, 7H), 7.05 (dd, J=6.2, 6.6 Hz, 1H), 5.32 (s, 2H), 4.48 (q, J=7.3 Hz, 2H), 1.45 (t, J=6.3 Hz, 3H). ES MS: m/z=417.2 (M+1).

### Step 4: Ethyl 1,4-dihydroxy-2-oxo-5-phenyl-1,2-dihydro-1,8-naphthyridine-3-carboxylate

Ethyl 1-(benzyloxy)-4-hydroxy-2-oxo-5-phenyl-1,2-dihydro-1,8-naphthyridine-3-carboxylate (50 mg, 0.12 mmol) was taken up in EtOH (5 mL). The solution was treated with 10% Pd/C (10 mg) and H₂ gas was bubbled through the mixture for 1 minutes. After 1 hour, the solution was filtered through Celite. Concentration of the filtrate afforded the title compound.

¹H NMR (400 MHz, CD₃OD, ppm): δ 8.12 (d, J=6.8 Hz, 1H), 7.50 (m, 2H), 7.32-7.43 (m, 6H), 7.17 (d, J=6.2 Hz, 1H), 4.45 (q, J=7.3 Hz, 2H), and 1.40 (t, J=6.3 Hz, 3H). ES MS: m/z=326.3 (M+1).

### Example 5

### 1,4-Dihydroxy-N,N-dimethyl-2-oxo-1,2-dihydro-1,8-naphthyridine-3-carboxamide

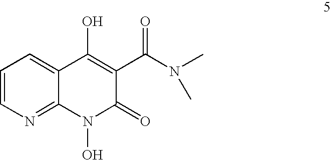

To a solution of ethyl 1,4-dihydroxy-2-oxo-1,2-dihydro-1,8-naphthyridine-3-carboxylate (Example 1, Step 3; 25 mg, 0.1 mmol) in DMF (1.5 mL) was added dimethylamine (2.0 M in MeOH; 0.25 mL, 0.5 mmol). The reaction mixture was stirred in a microwave reactor at 150° C. for 45 minutes. The DMF was removed and the residue was purified by RP-HPLC (C18 column; 5-95% CH₃CN/H₂O with 0.1% TFA) to give the title compound as a yellow solid.

¹H NMR (400 MHz, CD₃OD, ppm): δ 8.69 (d, J=4.2 Hz, 1H), 8.55 (d, J=7.7 Hz, 1H), 7.44 (dd, J=4.9, 7.9 Hz, 1H), 3.07 (s, 6H). High Resolution MS (FT-ICR): m/z found 250.0823 (M+1); calculated 250.0823 (M+1).

TABLE 1
The compounds in the following table were prepared in accordance with the
procedure set forth in Example 5:

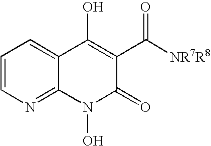

| Ex/Cpd | Name | NR⁷R⁸ | Data |
|---|---|---|---|
| 6 | N-Cyclopropyl-1,4- dihydroxy-2-oxo-1,2- dihydro-1,8- naphthyridine-3- carboxamide | 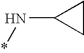 | High Resolution MS: m/z found 262.0819 (M + 1); calculated 262.0823 (M + 1). |
| 7 | N-Benzyl-1,4- dihydroxy-N-methyl-2- oxo-1,2-dihydro-1,8- naphthyridine-3- carboxamide | 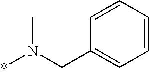 | High Resolution MS: m/z found 326.1137 (M + 1); calculated 326.1136 (M +1). |
| 8 | 1,4-Dihydroxy-3- (piperidin-1- ylcarbonyl)-1,8- naphthyridin-2(1H)-one | 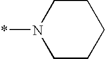 | High Resolution MS: m/z found 290.1138 (M + 1); calculated 290.1136 (M + 1). |
| 9 | tert-Butyl 4-[[1,4- dihydroxy-2-oxo-1,2- dihydro-1,8- naphthyridin-3- yl)carbonyl](methyl) amino]piperidine-1- carboxylate | 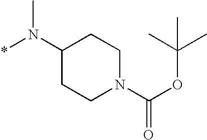 | High Resolution MS: m/z found 419.1926 (M + 1); calculated 419.1925 (M + 1). |
| 10 | tert-Butyl 3-[[(1,4- dihydroxy-2-oxo-1,2- dihydro-1,8- naphthyridin-3- yl)carbonyl](methyl) amino]pyrrolidine-1- carboxylate | 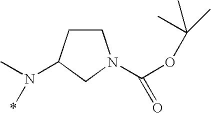 | ES MS: m/z = 305 (M + 1 − Boc). |
| 11 | 1,4-Dihydroxy-N-(2- methoxyphenyl)-2-oxo- 1,2-dihydro-1,8- naphthyridine-3- carboxamide | 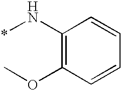 | High Resolution MS: m/z found 328.0930 (M + 1); calculated 328.0928 (M + 1). |
| 12 | 1,4-Dihydroxy-N-(3- methoxyphenyl)-2-oxo- 1,2-dihydro-1,8- naphthyridine-3- carboxamide | 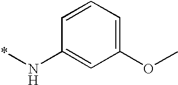 | High Resolution MS: m/z found 328.0928 (M + 1); calculated 328.0928 (M + 1). |
| 13 | 1,4-Dihydroxy-N-(4- methoxyphenyl)-2-oxo- 1,2-dihydro-1,8- naphthyridine-3- carboxamide | 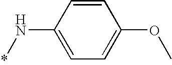 | High Resolution MS: m/z found 328.0921 (M + 1); calculated 328.0928 (M + 1). |
| 14 | 1,4-Dihydroxy-N-(6- methoxypyridine-3-yl)- 2-oxo-1,2-dihydro-1,8- naphthyridine-3- carboxamide | 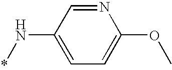 | High Resolution MS: m/z found 329.0875 (M + 1); calculated 329.0881 (M + 1). |
| 15 | 1,4-Dihydroxy-2-oxo- N-[2-(pyridin-2- ylmethoxy)phenyl]-1,2- dihydro-1,8- naphthyridine-3- carboxamide, hydrochloric acid salt | 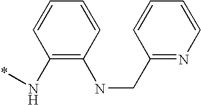 | High Resolution MS: m/z found 405.1204 (M + 1); calculated 405.1194 (M + 1). |
| 16 | 1,4-Dihydroxy-2-oxo- N-pyridin-3-yl-1,2- dihydro-1,8- naphthyridine-3- carboxamide | 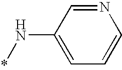 | High Resolution MS: m/z found 299.0775 (M + 1); calculated 299.0775 (M + 1). |
| 17 | 1,4-Dihydroxy-2-oxo- N-(6-phenoxypyridin- 3-yl)-1,2-dihydro-1,8- naphthyridine-3- carboxamide | 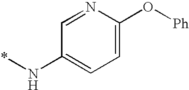 | Resolution MS: m/z found 405.1204 (M + 1); calculated 405.1194 (M + 1) |

### Example 18

### N-(4-Fluorobenzyl)-1,4-dihydroxy-2-oxo-1,2-dihydro-1,8-naphthyridine-3-carboxamide

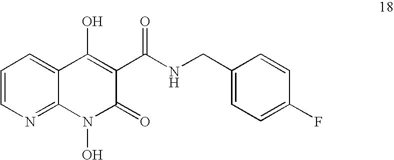

### Step 1: 1-(Benzyloxy)-N-(4-fluorobenzyl)-4-hydroxy-2-oxo-1,2-dihydro-1,8-naphthyridine-3-carboxamide

To a solution of ethyl 1-(benzyloxy)-4-hydroxy-2-oxo-1,2-dihydro-1,8-naphthyridine-3-carboxylate (Example 1, Step 2; 0.20 g, 0.59 mmol) in DMF (1.5 mL) was added 4-fluorobenzylamine (0.34 mL, 2.94 mmol). The reaction was stirred in a microwave reactor at 140° C. for 1 hour. The solvent was removed. The residue was triturated with MeOH and the solids were collected by vacuum filtration to give the title compound as a white solid. ES MS: m/z=420 (M+1).

### Step 2: N-(4-Fluorobenzyl)-1,4-dihydroxy-2-oxo-1,2-dihydro-1,8-naphthyridine-3-carboxamide

A solution of 1-(benzyloxy)-N-(4-fluorobenzyl)-4-hydroxy-2-oxo-1,2-dihydro-1,8-naphthyridine-3-carboxamide (0.22 g, 0.53 mmol) in HBr (33% wt. in HOAc; 5 mL) was heated to 80° C. for 4 hours. H₂O (1 mL) was added and the reaction mixture was stirred at 80° C. for an additional 18 hours. The reaction mixture was allowed to cool to room temperature. The solids formed were collected by vacuum filtration and washed with CH₃CN to give the title compound. ¹H NMR (400 MHz, d₆-DMSO, ppm): δ 10.5 (s, 1H), 8.84 (dd, J=1.7, 4.7 Hz, 1H), 8.48 (dd, J=1.8, 7.9 Hz, 1H), 7.46-7.41 (m, 3H), 7.22-7.17 (m, 2H), and 4.61 (d, J=6.0 Hz, 2H). High Resolution MS: m/z=found 330.0888 (M+1); calculated 330.0885 (M+1).

TABLE 2
The compounds in the following table were prepared in accordance with the
procedures set forth in Example 18:

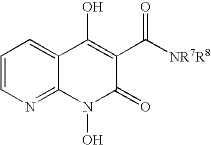

| Ex/cpd | Name | NR⁷R⁸ | Data |
|---|---|---|---|
| 19 | N-benzyl-1,4- dihydroxy-2-oxo-1,2- dihydro-1,8- naphthyridine-3- carboxamide | 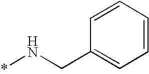 | High Resolution MS: m/z found 312.1005 (M + 1); calculated 312.0979 (M + 1). |
| 20 | 1,4-Dihydroxy-2-oxo- N-phenyl-1,2-dihydro- 1,8-naphthyridine-3- carboxamide | 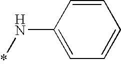 | High Resolution MS: m/z found 298.0848 (M + 1); calculated 298.0823 (M + 1). |
| 21 | N-(1-Benzylpiperidin- 4-yl)-1,4-dihydroxy-2- oxo-1,2-dihydro-1,8- naphthyridine-3- carboxamide, trifluoro- acetic acid salt | 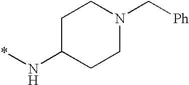 | High Resolution MS: m/z found 395.1724 (M + 1); calculated 395.1714 (M + 1). |
| 22 | 1,4-Dihydroxy-2-oxo- N-(2,2,2- trifluoroethyl)-1,2- dihydro-1,8- naphthyridine-3- carboxamide | 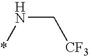 | High Resolution MS: m/z found 304.0545 (M + 1); calculated 304.0540 (M + 1). |
| 23 | 1,4-Dihydroxy-2-oxo- N-(1-phenylethyl)-1,2- dihydro-1,8- naphthyridine-3- carboxamide | 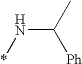 | High Resolution MS: m/z found 326.1147 (M + 1); calculated 326.1136 (M + 1). |
| 24 | 1,4-Dihydroxy-N- methyl-2-oxo-1,2- dihydro-1,8- naphthyridine-3- carboxamide | 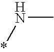 | High Resolution MS: m/z found 236.0664 (M + 1); calculated 236.0666 (M + 1). |
| 25 | 1,4-Dihydroxy-2-oxo- N-(pyrrolidin-3- ylmethyl)-1,2-dihydro- 1,8-naphthyridine-3- carboxamide | 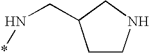 | High Resolution MS: m/z found 305.1251 (M + 1); calculated 305.1245 (M + 1). |
| 26 | 1,4-dihydroxy-N- methyl-2-oxo-N- (piperidin-4-ylmethyl)- 1,2-dihydro-1,8- naphthyridine-3- carboxamide | 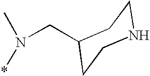 | High Resolution MS: m/z found 319.1405 (M + 1); calculated 319.1401 (M + 1). |

### Example 27

### 1,4-Dihydroxy-3-pyridin-2-yl-1,8-naphthyridin-2(1B)-one

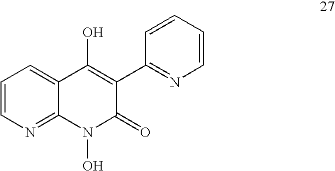

### Step 1: 1-(Benzyloxy)-4-hydroxy-3-pyridin-2-yl-1,8-naphthyridin-2(1H)-one

To a dry round-bottom flask were added ethyl 2-[(benzyloxy)amino]nicotinate ([J. Het. Chem. 1993, 30 (4), 909-912]; 1.0 mmol), ethyl pyridin-2-ylacetate (5.0 mmol) and sodium ethoxide in EtOH (2.5 mmol). The reaction mixture was heated to 80° C. for 48 hours. An aqueous solution of HCl (1 M, 3.0 mmol) was added and the mixture was extracted with EtOAc. The combined organic extracts were washed with H₂O and brine and were then concentrated. The residue was purified by SGC (15% 50% EtOAc/hexanes) to give the title compound. ES MS: m/z=346 (M+1).

### Step 2: 1,4-Dihydroxy-3-pyridin-2-yl-1,8-naphthyridin-2(11)-one

To a solution of 1-(benzyloxy)-4-hydroxy-3-pyridin-2-yl-1,8-naphthyridin-2(1H)-one (0.5 mmol) in HOAc (1 mL) was added HBr (33% wt. in HOAc, 2.0 mL). The reaction mixture was heated to 80° C. for 2 hours. The mixture was concentrated and the residue was triturated with MeOH and EtOAc to give the title compound. ¹H NMR (400 MHz, d₆-DMSO, ppm): δ 9.28 (d, J=6.6 Hz, 1H), 8.62 (2H, m), 8.55 (d, J=6.6 Hz, 1H), 8.24 (t, 6.8 Hz, 1H), 7.51 (t, J=6.4 Hz, 1H), and 7.31 (dd, J=6.2, 8.1 Hz, 1H). ES MS: m/z=256 (M+1).

TABLE 3
The compounds in the following table were prepared in accordance with the
procedures set forth in Example 27:

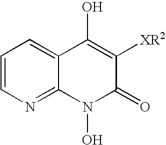

| Ex/cpd | Name | -XR² | Data |
|---|---|---|---|
| 28 | 1,4-Dihydroxy-3-[3- (trifluoromethyl)phenyl]- 1,8-naphthyridin- 2(1H)-one | 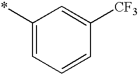 | ¹H NMR (400 MHz, d₆-DMSO) δ 7.12 (d, J = 6.8 Hz, 1H), 7.67-7.81 (m, 4H), 8.41 (d, J = 8.1 Hz, 1H), 8.77 (s, 1H), 10.77 (br s, 1H). ES MS: m/z = 323.3 (M + 1) |
| 29 | 3-(2,6- Difluorophenyl)-1,4- dihydroxy-1,8- naphthyridin-2(1H)- one | 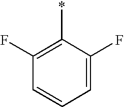 | ¹H NMR (400 MHz, d₆-DMSO) δ 7.17 (m, 2H), 7.32 (dd, J = 5.1, 6.3 Hz, 1H), 7.49 (q, J = 8.1 Hz, 1H), 8.42 (d, J = 8.1 Hz, 1H), 8.71 (d, J = 6 Hz, 1H), 10.77 (br s, 1H), 11.18 (br s, 1H). ES MS: m/z = 291.3 (M + 1) |
| 30 | 1,4-Dihydroxy-3- phenyl-1,8- naphthyridin-2-(1H)- one | 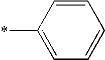 | ¹H NMR (400 MHz, d₆-DMSO) δ 7.26-7.32 (m, 6H), 8.42 (d, J = 7.3 Hz, 1H), 8.61 (d, J = 6.4 Hz, 1H). ES MS: m/z = 255.3 (M + 1) |
| 31 | 1,4-Dihydroxy-3-(4- methoxyphenyl)-1,8- naphthyridin-2(1H)- one | 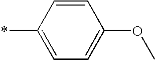 | ¹H NMR (400 MHz, d₆-DMSO) δ 10.68 (br s, 1H), 10.31 (s, 1H), 8.65 (dd, J = 1.7, 4.7 Hz, 1H), 8.38 (dd, J = 1.7, 7.9 Hz, 1H), 7.35-7.30 (m, 3H), 7.02-6.98 (m, 2H), 3.81 (s, 3H). High Resolution MS: m/z found 285.0874 (M + 1); calculated 285.0870 (M + 1) |
| 32 | 3-(2-Fluorophenyl)- 1,4-dihydroxy-1,8- naphthyridin-2(1H)- one | 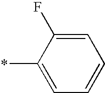 | ¹H NMR (400 MHz, d₆-DMSO): δ 10.77 (br s, 1H) 10.72 (br s, 1H) 8.69 (dd, J = 1.6, 4.6 Hz, 1H), 8.41 (dd, J = 1.5, 8.0 Hz, 1H), 7.47-7.42 (m, 1H), 7.37-7.33 (m, 2H), 7.26 (t, J = 8.1 Hz, 2H). High Resolution MS: m/z found 273.0671 (M + 1); calculated 273.0670 (M + 1) |
| 33 | 3-(3-Chlorophenyl)- 1,4-dihydroxy-1,8- naphthyridin-2(1H)- one | 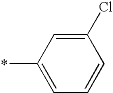 | ¹H NMR (400 MHz, d₆-DMSO): δ 10.71 (br s, 2H), 8.69 (dd, J = 1.7, 7.7 Hz, 1H), 8.42 (dd, J = 1.7, 8.0 Hz, 1H), 7.49-7.40 (m, 3H), 7.37- 7.34 (m, 2H). High Resolution MS: m/z found 289.0379 (M + 1); calculated 289.0375 (M + 1) |
| 34 | 3-(4-Fluorophenyl)- 1,4-dihydroxy-1,8- naphthyridin-2(1H)- one | 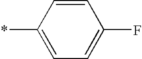 | High Resolution FT-ICR MS: m/z found 273.0673 (M + 1); calculated 273.0670 (M + 1) |
| 35 | 3-(2-Chlorophenyl)- 1,4-dihydroxy-1,8- naphthyridin-2(1H)- one | 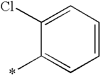 | High Resolution FT-ICR MS: m/z found 289.0377 (M + 1); calculated 289.0375 (M + 1) |
| 36 | 3-(3-Fluorophenyl)- 1,4-dihydroxy-1,8- naphthyridin-2(1H)- one | 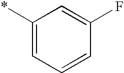 | High Resolution FT-ICR MS: m/z found 273.0672 (M + 1); calculated 273.0670 (M + 1) |
| 37 | 3-(4-Chlorophenyl)- 1,4-dihydroxy-1,8- naphthyridin-2(1H)- one | 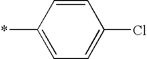 | High Resolution FT-ICR: MS: m/z found 289.0379 (M + 1); calculated 289.0375 (M + 1) |
| 38 | 3-(4-Bromophenyl)- 1,4-dihydroxy-1,8- naphthyridin-2(1H)- one | 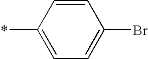 | High Resolution FT-ICR MS: m/z found 332.9879 (M + 1); calculated 332.9870 (M + 1) |
| 39 | 1,4-Dihydroxy-3-(2- methoxyphenyl)-1,8- naphthyridin-2(1H)- one | 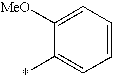 | High Resolution FT-ICR MS: m/z found 285.0869 (M + 1); calculated 285.0820 (M + 1) |
| 40 | 1,4-Dihydroxy-3-(3- methoxyphenyl)-1,8- naphthyridin-2(1H)- one | 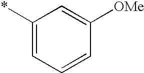 | High Resolution FT-ICR MS: m/z found 285.0874 (M + 1); calculated 285.0870 (M + 1) |
| 41 | 3-Cyclopentyl-1,4- dihydroxy-1,8- naphthyridin-2(1H)- one | 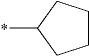 | ES MS: m/z = 247 (M + 1) |
| 42 | 3-Butyl-1,4- dihydroxy-1,8- naphthyridin-2(1H)- one | 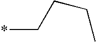 | ES MS: m/z = 235 (M + 1) |
| 43 | 3-(3-Trifluoromethyl phenyl)-1,4- dihydroxy-1,8- naphthyridin-2(1H)- one | 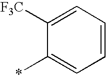 | High Resolution FT-ICR MS: m/z found 323.0648 (M + 1); calculated 323.0638 (M + 1) |
| 44 | 3-(3-bromophenyl)- 1,4-dihydroxy-1,8- naphthyridin-2(1H)- one | 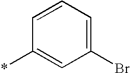 | High Resolution MS (FT-ICR): m/z found 332.9867 (M + 1); calculated 332.987 (M + 1) |
| 45 | 4-(1,4-dihydroxy-2- oxo-1,2-dihydro-1,8- naphthyridin-3- yl)benzonitrile | 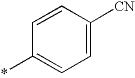 | High Resolution MS (FT-ICR): m/z found 280.0717 (M + 1); calculated 280.0717 (M + 1) |
| 46 | 1,4-dihydroxy-3-(2- hydroxyphenyl)-1,8- naphthyridin-2(1H)- one | 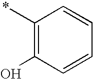 | High Resolution MS (FT-ICR): m/z found 271.0714 (M + 1); calculated 271.0714 (M + 1) |

### Example 47

### 6-Bromo-1,4-dihydroxy-3-phenyl-1,8-naphthyridin-2(1H)-one

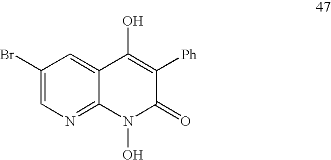

### Step 1: Methyl 2-[(benzyloxy)(phenylacetyl)amino]-5-bromonicotinate

To a solution of methyl 2-[(benzyloxy)amino]-5-bromonicotinate (Example 3, Step 1; 3.0 g, 8.1 mmol) and TEA (3.8 mL, 25.0 mmol) in DCM (250 mL) was added dropwise phenylacetyl chloride (3.6 mL, 12 mmol). After 1 hour, the solids formed were filtered off. The filtrate was concentrated and the residue was purified by SGC (0% 40% EtOAc/hexanes) to give the title compound as brown oil. ES MS: m/z=455.1 (M+1).

### Step 2: 1-(Benzyloxy)-6-bromo-4-hydroxy-3-phenyl-1,8-naphthyridin-2(1H)-one

To a solution of methyl 2-[(benzyloxy)(phenylacetyl)amino]-5-bromonicotinate (2.5 g, 5.5 mmol) in anhydrous THF (20 mL) was added dropwise a solution of lithium hexadimethylsilazide (5.1 mL, 5.5 mmol). The reaction was stirred at −78° C. for 1 hour. The reaction solution was brought to pH 4 by the addition of aqueous 2N HCl. After 15 minutes, the solids formed were collected by vacuum filtration to give the title compound. ¹H NMR (400 MHz, d₆-DMSO, ppm): δ 10.82 (s, 1H), 8.71 (s, 1H), 8.42 (s, 1H), 7.51 (m, 5H), 7.31-7.28 (m, 5H), 5.21 (s, 2H), and 3.61 (s, 3H). ES MS: m/z=423.2 (M+1).

### Step 3: 6-Bromo-1,4-dihydroxy-3-phenyl-1,8-naphthyridin-2(1H)-one

To a solution of 1-(benzyloxy)-6-bromo-4-hydroxy-3-phenyl-1,8-naphthyridin-2(1H)-one (0.33 g, 1.0 mmol) in HOAc (3 mL) was added 33% HBr/HOAc (1.0 mL). The reaction mixture was heated to 80° C. and stirred for 1 hour. The solution was concentrated and purified by RP-HPLC (C18 column eluting with H₂O/CH₃CN) to give the title compound. ¹H NMR (400 MHz, d₆-DMSO, ppm): δ 11.1 (s, 1H), 10.31 (br s, 1H), 8.65 (s, 1H), 8.37 (s, 1H), and 7.31-7.38 (m, 5H). ES MS: m/z=333.2 (M+1).

### Example 48

### 6-Fluoro-1,4-dihydroxy-3-phenyl-1,8-naphthyridin-2(1H)-one

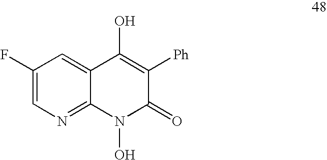

The title compound was prepared from ethyl 2-chloro-5-fluoronicotinate essentially according to the procedures described in Example 47. ¹H NMR (400 MHz, d₆-DMSO, ppm): δ 10.82 (br s, 1H), 10.64 (br s, 1H), 8.72 (d, J=8.0, 1H), 8.25 (m, 1H), and 7.36-7.48 (m, 5H). ES MS: m/z=273.3 (M+1).

### Example 49

### Ethyl 1,4-dihydroxy-2-oxo-6-phenyl-1,2-dihydro-1,8-naphthyridine-3-carboxylate

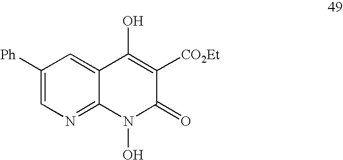

### Step 1: Ethyl 1-(benzyloxy)-4-hydroxy-2-oxo-6-phenyl-1,2-dihydro-1,8-naphthyridine-3-carboxylate

To a solution of ethyl 1-(benzyloxy)-6-bromo-4-hydroxy-2-oxo-1,2-dihydro-1,8-naphthyridine-3-carboxylate (Example 3, Step 3; 100 mg, 0.25 mmol) in DMF (4.0 mL) were added phenyl boronic acid (50 mg, 0.42 mmol), K₂CO₃ (75 mg, 0.61 mmol) and H₂O (1.0 mL). N₂ was bubbled through the solution. Pd(dppf)Cl₂ (25 mg, 0.02 mmol) was added and the reaction vessel sealed. This solution was heated in a microwave reactor at 110° C. for 10 minutes, after which the solution was cooled and partitioned between HCl (1.0 M, 10 mL) and EtOAc (10 mL). The organic layer was separated, dried and concentrated. The residue was purified by SGC (80% EtOAc/hexane) to give the title compound. ES MS: m/z=417.2 (M+1).

### Step 2: Ethyl 1,4-dihydroxy-2-oxo-6-phenyl-1,2-dihydro-1,8-naphthyridine-3-carboxylate

A solution of ethyl 1-(benzyloxy)-4-hydroxy-2-oxo-6-phenyl-1,2-dihydro-1,8-naphthyridine-3-carboxylate (30 mg, 0.07 mmol) in EtOH (5 mL) was treated with 10% Pd/C (10 mg) and the solution was saturated with H₂ and stirred at room temperature. After 1 hour, the solution was filtered through a pad of Celite. The filtrate was concentrated and the residue purified by RP-HPLC (C18 column; H₂O/CH₃CN/0.1% TFA) to yield the title compound. High Resolution MS (FT-ICR): m/z found 327.0990 (M+1); calculated 327.0975 (M+1).

TABLE 4
The compounds in the following table were prepared in accordance with the
procedure set forth in Example 49:

| Ex/cpd | Name | Structure | Data |
|---|---|---|---|
| 50 | 6-[3- (Aminomethyl) phenyl]-1,4- dihydroxy-3- phenyl-1,8- naphthyridin- 2(1H)-one | 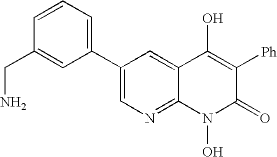 | ¹H NMR (400 MHz, d₆-DMSO, ppm): δ 11.18 (br s, 1H), 10.77 (br s, 1H), 8.71(d, J = 6.0 Hz, 1H), 8.42(d, J = 8.1 Hz, 1H), 7.49-7.32 (m, 6H), 7.23- 7.17 (m, 3H), 4.21 (s, 2H). ES MS: m/z = 291.3 (M + 1) |
| 51 | 1,4-Dihydroxy-3,6- diphenyl-1,8- maphthyridin- 2(1H)-one | 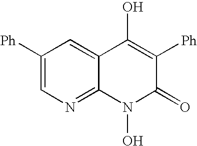 | ¹H NMR (400 MHz, d₆-DMSO, ppm): δ 10.47 (br s, 1H), 8.61 (d, J = 6.5 Hz, 1H), 8.32 (d, J = 7.9 Hz, 1H), 7.41-7.25 (m, 10H). ES MS: m/z = 331.3 (M + 1) |
| 52 | 6-Benzyl-1,4- dihydroxy-3- phenyl-1,8- naphthyridin- 2(1H)-one | 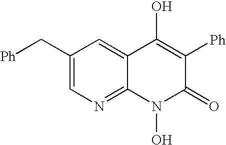 | ¹H NMR (400 MHz, d₆-DMSO, ppm): δ 10.27 (br s, 1H), 8.57 (d, J = 6.5 Hz, 1H), 8.37 (d, J = 7.8 Hz, 1H), 7.38-7.31 (m, 5H), 7.25 (m, 5H), 5.31 (s, 2H). ES MS: m/z = 345.3 (M + 1) |
| 53 | 1,4-Dihydroxy-3- phenyl-6-[2-(1H- pyrazol-1- yl)phenyl]-1,8- naphthyridin- 2(1H)-one | 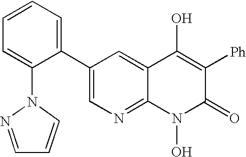 | ¹H NMR (400 MHz, d₆-DMSO, ppm): δ 10.57 (br s, 1H), 8.67 (d, J = 6 Hz, 1H), 8.47 (d, J = 8.1 Hz, 1H), 7.59 (q, J = 8.3 Hz, 1H), 7.41-7.28 (m, 9H), 6.72 (m, 2H). ES MS: m/z = 397.3 (M + 1) |
| 54 | 6-Biphenyl-3-yl- 1,4-dihydroxy-3- phenyl-1,8- naphthyridin- 2(1H)-one | 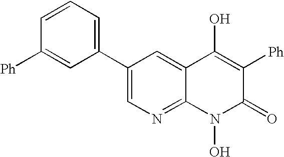 | High Resolution MS (FT-ICR): m/z found 407.1392 (M + 1); calculated 407.139 (M + 1) |
| 55 | 1,4-Dihydroxy-3- phenyl-6-[(E)-2- phenylvinyl]-1,8- naphthyridin- 2(1H)-one | 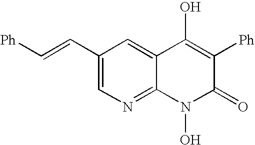 | High Resolution MS (FT-ICR): m/z found 357.1231 (M + 1); calculated 357.1234 (M + 1) |
| 56 | 1,4-Dihydroxy-6- (2-naphthyl)-3- phenyl-1,8- naphthyridin- 2(1H)-one | 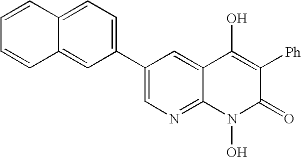 | High Resolution MS (FT-ICR): m/z found 381.1229 (M + 1); calculated 381.1234 (M + 1) |
| 57 | Ethyl 1,4- dihydroxy-6-[3- (morpholin-4- ylmethyl)phenyl]-2- oxo-1,2-dihydro- 1,8-naphthyridine- 3-carboxylate | 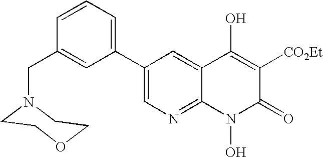 | ¹H NMR (400 MHz, d₆-DMSO, ppm): δ 9.12 (s, 1H), 8.62 (s, 1H), 8.01 (m, 2H), 7.51-7.69 (m, 2H), 4.51 (s, 2H), 4.41 (q, J = 7.1 Hz, 2H), 4.20-3.71 (m, 4H), 3.52-3.31 (m, 4H), 1.42 (t, J = 7.8 Hz, 3H). ES MS: m/z = 354.3 (M + 1) |
| 58 | Ethyl 1,4- dihydroxy-2-oxo-6- pyridin-3-yl-1,2- dihydro-1,8- naphthyridine-3- carboxylate | 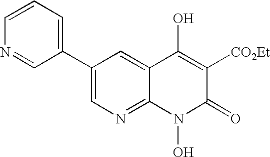 | High Resolution MS (FT-ICR): m/z found 328.0932 (M + 1); calculated 328.0928 (M + 1) |
| 59 | Ethyl 1,4- dihydroxy-6-(3- hydroxyphenyl)-2- oxo-1,2-dihydro- 1,8-naphthyridine- 3-carboxylate | 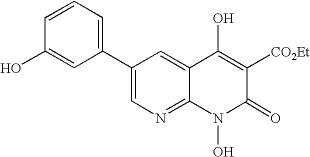 | High Resolution MS (FT-ICR): m/z found 343.0926 (M + 1); calculated 343.0925 (M + 1) |
| 60 | Ethyl 6-(3-cyano phenyl)-1,4- dihydroxy-2-oxo- 1,2-dihydro-1,8- naphthyridine-3- carboxylate | 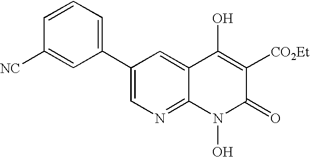 | High Resolution MS (FT-ICR): m/z found 352.094 (M + 1); calculated 352.0928 (M + 1) |
| 61 | Ethyl 1,4- dihydroxy-6-(2- methoxy phenyl)-2- oxo-1,2-dihydro- 1,8-naphthyridine- 3-carboxylate | 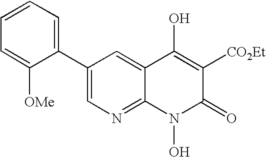 | High Resolution MS (FT-ICR): m/z found 357.1093 (M + 1); calculated 357.1081 (M + 1) |
| 62 | Ethyl 1,4- dihydroxy-6-(3- methoxy phenyl)-2- oxo-1,2-dihydro- 1,8-naphthyridine- 3-carboxylate | 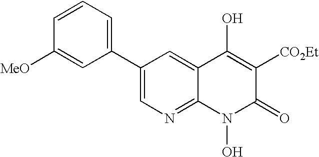 | High Resolution MS (FT-ICR): m/z found 357.1096 (M + 1); calculated 357.1081 (M + 1) |
| 63 | Ethyl 6-benzyl-1,4- dihydroxy-2-oxo- 1,2-dihydro-1,8- naphthyridine-3- carboxylate | 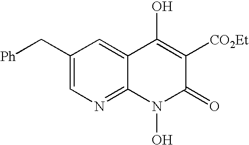 | High Resolution MS (FT-ICR): m/z found 341.1133 (M + 1); calculated 341.1132 (M + 1) |
| 64 | Ethyl 6-biphenyl-3- yl-1,4-dihydroxy-2- oxo-1,2-dihydro- 1,8-naphthyridine- 3-carboxylate | 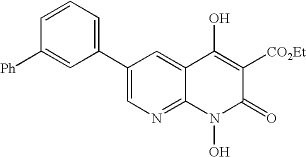 | High Resolution MS (FT-ICR): m/z found 403.129 (M + 1); calculated 403.1289 (M + 1) |
| 65 | Ethyl 6-(3,5- dimethyl phenyl)- 1,4-dihydroxy-2- oxo-1,2-dihydro- 1,8-napthyridine- 3-carboxylate | 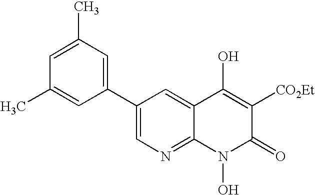 | High Resolution MS (FT-ICR): m/z found 355.1289 (M + 1); calculated 355.1289 (M + 1) |
| 66 | 3,6-Dibenzyl-l,4- dihydroxy-1,8- naphthyridin- 2(1H)-one | 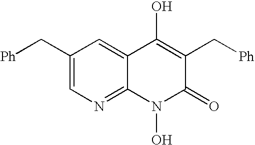 | ¹H NMR (400 MHz, d₆-DMSO, ppm): δ 10.44 (br s, 1H), 8.58 (s, 1H), 8.22 (s, 1H), 7.35-7.22 (m, 10H) 7.12 (m, 1H), 4.09 (s, 2H), 3.92 (s,2H). ES MS: m/z = 273.3 (M + 1) |
| 67 | 3-Benzyl-1,4- dihydroxy-6- phenyl-1,8- naphthyridin- 2(1H)-one | 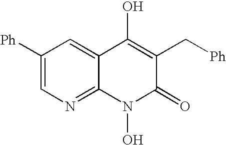 | ¹H NMR (400 MHz, d₆-DMSO, ppm): δ 12.11 (br s, 1H), 10.98 (br s, 1H), 8.99 (s, 1H), 8.67 (s, 1H), 8.25 (m, 1H), 7.85- 7.61 (m, 5H), 7.48-7.16 (m, 5H) 4.12 (m, 2H). ES MS: m/z = 245.3 (M + 1) |
| 68 | 6-(3- Aminophenyl)-1,4- dihydroxy-1,8- naphthyridin- 2(1H)-one | 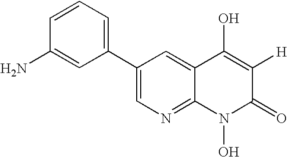 | ES MS: m/z = 270.3 (M + 1) |
| 69 | N-[3-(5,8- Dihydroxy-7-oxo- 7,8-dihydro-1,8- naphthyridin-3- yl)phenyl]methane- sulfonamide | 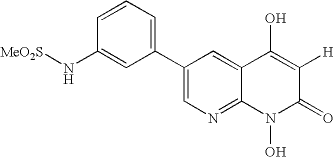 | ES MS: m/z = 348.2 (M + 1). |
| 70 | ethyl 6-acetyl-1,4- dihydroxy-2-oxo- 1,2-dihydro-1,8- naphthyridine-3- carboxylate | 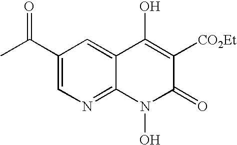 | ES MS: m/z = 294.2 (M + 1). |
| 71 | 6-acetyl-1,4- dihydroxy-3- phenyl-1,8- naphthyridin- 2(1H)-one | 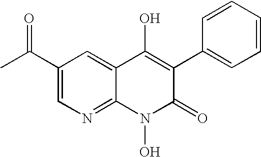 | ES MS: m/z = 297.2 (M + 1). |

### Example 72

### 3-Bromo-1,4-dihydroxy-6-pyridin-4-yl-1,8-naphthyridin-2(1H)-one

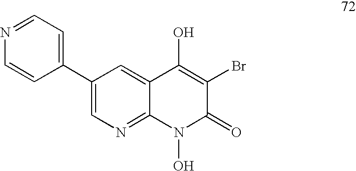

### Step 1: Ethyl 1-(benzyloxy)-4-hydroxy-2-oxo-6-pyridin-4-yl-1,2-dihydro-1,8-naphthyridine-3-carboxylate

The title compound was prepared from ethyl 1-(benzyloxy)-6-bromo-4-hydroxy-2-oxo-1,2-dihydro-1,8-naphthyridine-3-carboxylate (Example 3, Step 3) and pyridin-4-ylboronic acid essentially according to the procedure described in Example 49, Step 1. ES MS: m/z=418.2 (M+1).

### Step 2: 3-Bromo-1,4-dihydroxy-6-pyridin-4-yl-1,8-naphthyridin-2(11)-one

A mixture of ethyl 1-(benzyloxy)-4-hydroxy-2-oxo-6-pyridin-4-yl-1,2-dihydro-1,8-naphthyridine-3-carboxylate (41 mg, 0.10 mmol), 33% HBr—HOAc (2 mL) and H₂O (0.5 mL) was stirred at 80° C. for 1 hour. The solvents were removed and the residue was purified by RP-HPLC (C18 column; 5-95% CH₃CN/H₂O with 0.1% TFA) to give the title compound. High Resolution MS (FT-ICR): m/z found 333.9821 (4+1); calculated 333.9822 (M+1).

### Example 73

### 6-Ethyl-1,4-dihydroxy-3-phenyl-1,8-naphthyridin-2(1H)-one

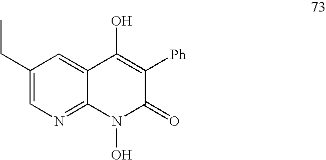

### Step 1: 1-(Benzyloxy)-4-hydroxy-3-phenyl-6-vinyl-1,8-naphthyridin-2(1H)-one

A mixture of 1-(benzyloxy)-6-bromo-4-hydroxy-3-phenyl-1,8-naphthyridin-2(1H)-one (Example 47, Step 2; 50 mg, 0.12 mmol), vinyl tributyltin (0.052 mL, 0.18 mmol) and bis(triphenyl-phosphine)palladium (II) chloride (8.3 mg, 0.012 mmol) in dioxane (7 mL) was heated in a sealed pressure tube at 80° C. for 7.5 hours. Additional vinyl tributyltin (0.069 mL) and Pd catalyst (8 mg) were added, the mixture was purged with N₂ and heated at 100 C for 4.5 hours. The solvent was removed and the residue was purified by SGC (0-80% EtOAc/hexanes) to afford the title compound as an orange foam. ES MS: m/z=371.14 (M+1).

### Step 2: 6-Ethyl-1,4-dihydroxy-3-phenyl-1,8-naphthyridin-2(1H)-one

A solution of 1-(benzyloxy)-4-hydroxy-3-phenyl-6-vinyl-1,8-naphthyridin-2(1H)-one (30 mg, 0.08 mmol) in EtOH (9 mL) was purged with N₂ and treated with 10% Pd/C (1 mg). The mixture was flushed with H₂ (×3) and stirred under H₂ atmosphere at room temperature overnight, resulting in the formation of the intermediate 1-(benzyloxy)-6-ethyl-4-hydroxy-3-phenyl-1,8-naphthyridin-2(1B)-one. The mixture was filtered through a Celite pad and the solvent removed. The residue was dissolved in 33% HBr/HOAc (4 mL) and H₂O (1 mL) and the mixture heated at 80° C. for 1.25 hours. The solvents were removed and the residue dissolved in MeOH. Purification by RP-HPLC (C18 column; 15-100% CH₃CN/H₂O with 0.1% TFA) afforded the title compound. ¹H NMR (400 MHz, d₆-DMSO, ppm): δ 10.46 (br s, 1H), 8.61 (s, 1H), 8.32 (s, 1H), 7.52-7.40 (m, 5H), 2.81 (q, J=7.3, 14.8 Hz, 2H), and 1.32 (t, J=7.5 Hz, 3H). ES MS: m/z=283.3 (M+1).

### Example 74

### 6-(2-Bromopropyl)-1,4-dihydroxy-3-phenyl-1,8-naphthyridin-2(1H)-one

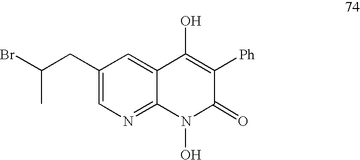

### Step 1: 6-Allyl-1-(benzyloxy)-4-hydroxy-3-phenyl-1,8-naphthyridin-2(1H)-one

The title compound was prepared from 1-(benzyloxy)-6-bromo-4-hydroxy-3-phenyl-1,8-naphthyridin-2(1H)-one (Example 47, Step 2) and allyl tributyltin essentially according to the procedure described in Example 73, Step 1. ES MS: m/z=385.3 (M+1).

### Step 2: 6-(2-Bromopropyl)-1,4-dihydroxy-3-phenyl-1,8-naphthyridin-2(1H)-one

A solution of 6-allyl-1-(benzyloxy)-4-hydroxy-3-phenyl-1,8-naphthyridin-2(1H)-one (10 mg, 0.03 mmol) in 33% HBr/HOAc (2 mL) and H₂O (0.5 mL) was heated at 80° C. for 1 hour. The solvents were removed and the residue dissolved in MeOH and purified by RP-HPLC (C18 column; 15-100% CH₃CN/H₂O with 0.1% TFA) to give the title compound. ¹H NMR (400 MHz, d₆-DMSO, ppm): δ 10.42 (br s, 1H), 8.58 (s, 1H), 8.31 (s, 1H), 7.44-7.36 (m, 5H), 4.58 (br m, 1H), 3.35-3.15 (m, 2H), and 1.73 (d, J=6.0 Hz, 3H). ES MS: m/z=375.2 (M+1).

### Example 75

### 6-(thien-2-yl)-1,4-dihydroxy-3-phenyl-1,8-napthyridin-2(1H)-one

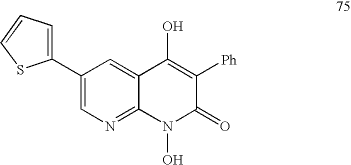

The title compound was prepared essentially according to the procedures described in Example 73, Steps 1 and 2. ¹H NMR (400 MHz, d₆-DMSO, ppm): δ 10.66 (br s, 1H), 9.02 (s, 1H), 8.58 (s, 1H), 7.68-7.65 (m, 2H), 7.45-7.37 (m, 5H), and 7.22 (br s, 1H). ES MS: m/z=337.2 (M+1).

### Example 76

### 6-{1-[(3-Chlorobenzyl)amino]ethyl}-1,4-dihydroxy-3-phenyl-1,8-napthyridin-2(1H)-one

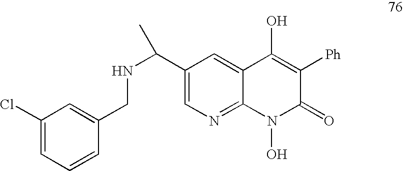

### Step 1: 6-Acetyl-1-(benzyloxy)-4-hydroxy-3-phenyl-1,8-naphthyridin-2(1H)-one

To a solution of 1-(benzyloxy)-6-bromo-4-hydroxy-3-phenyl-1,8-naphthyridin-2(1H)-one (Example 47, Step 2; 0.33 g, 1.0 mmol) in dioxane (5 mL) was added tributyl(1-ethoxyvinyl)tin (0.3 mL). N₂ was bubbled through the solution. Tetrakis(triphenylphosphine)palladium(0) (50 mg, 0.05 mmol) was added and the mixture heated at 80° C. for 1 hour. The solution was cooled and HOAc (1.0 mL) was added followed by EtOAc (20 mL) and brine (20 mL). The organic layer was separated, dried and concentrated. The crude product was purified by SGC (10-60% EtOAc/hexane) to give the title compound. ¹H NMR (400 MHz, d₆-DMSO, ppm): δ 10.41 (br s, 1H), 8.62 (s, 1H), 8.47 (s, 1H), 7.38-7.32 (m, 5H), 5.21 (s, 2H), and 2.42 (s, 3H). ES MS: m/z=387.2 (M+1).

### Step 2: 1-(Benzyloxy)-6-{1-[(3-chlorobenzyl)amino]ethyl}-4-hydroxy-3-phenyl-1,8-naphthyridin-2(1H)-one

A solution of 6-acetyl-1-(benzyloxy)-4-hydroxy-3-phenyl-1,8-naphthyridin-2(1H)-one (50 mg, 0.13 mmol) in MeOH (10 mL) was treated successively with sodium triacetoxyborohydride (100 mg, 0.47 mmol) and 3-chlorobenzylamine (100 mg, 0.71 mmol). The mixture was stirred for 3 hours. The reaction was quenched by addition of saturated sodium carbonate solution (5 mL) and the product was extracted into EtOAc. The organic layer was washed with H₂O, dried and concentrated. The crude product was purified by SGC (30-100% EtOAc/hexane) to give the title compound. ES MS m/z=512.2 (M+1).

### Step 3: (6-{1-[(3-Chlorobenzyl)amino]ethyl}-1,4-dihydroxy-3-phenyl-1,8-napthyridin-2(1H)-one)

A solution of 1-(benzyloxy)-6-{1-[(3-chlorobenzyl)amino]ethyl}-4-hydroxy-3-phenyl-1,8-naphthyridin-2(1H)-one (40 mg, 0.08 mmol) in 33% HBr/HOAc (1.0 mL) was heated at 80° C. for 1 hour. The solution was cooled and the solvent was removed. The crude product was purified by RP-HPLC (C18 column; H₂O/CH₃CN with 0.1% TFA) to give the title compound. ¹H NMR (400 MHz, d₆-DMSO, ppm): δ 10.98(br s, 1H), 8.99 (s, 1H), 8.67 (s, 1H), 7.85-7.61 (m, 2H), 7.48-7.16 (m, 5H) 4.12 (s, 2H), 3.98 (m, 1H), and 3.32 (d, J=7.8 Hz, 3H). ES MS: m/z=422.3 (M+1).

### Example 77

### Ethyl 6-[(benzylamino)carbonyl]-1,4-dihydroxy-2-oxo-1,2-dihydro-1,8-naphthyridine-3-carboxylate

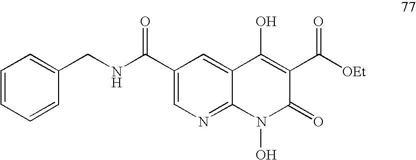

### Step 1: Dimethylpyridine-3,5-dicarboxylate hydrochloride

HCl gas was bubbled through a suspension of pyridine-3,5-dicarboxylic acid (10.0 g, 59.8 mmol) in MeOH (250 mL), resulting in dissolution of all solids. The saturated solution was then stirred overnight at room temperature, resulting in formation of the mono-ester as the major product. Additional HCl was bubbled into the mixture which was then stirred at room temperature overnight. The solvent was removed and the solid residue triturated with MeOH and collected by vacuum filtration to afford the title compound as a white solid. Additional product precipitated from the filtrate and was collected and combined with the first batch. ES MS: m/z=196 (M+1).

### Step 2: Dimethylpyridine-3,5-dicarboxylate 1-oxide

Dimethylpyridine-3,5-dicarboxylate hydrochloride was treated with saturated aqueous sodium bicarbonate. The mixture was extracted with DCM and the organic layer concentrated to afford the free base, dimethylpyridine-3,5-dicarboxylate, as a white solid. This solid (5.0 g, 25.6 mmol) was dissolved in DCM (150 mL) and the solution cooled to 0° C. and treated with urea hydrogen peroxide (5.06 g, 53.8 mmol) followed by trifluoroacetic anhydride (7.2 mL, 51.2 mmol). The reaction mixture was stirred at room temperature overnight and was then treated with additional urea hydrogen peroxide (2.0 g, 21.3 mmol) and trifluoroacetic anhydride (3.1 mL, 22 mmol). The mixture was stirred at room temperature for an additional 3 hours and was then quenched by addition of aqueous sodium dithionite and stirred for 15 minutes. The mixture was then poured into 1 N aqueous HCl and extracted with DCM. The combined organic extracts were dried, filtered and concentrated. The residue was purified by SCG (0-5% MeOH/DCM) to give the title compound as a light yellow solid. ¹H NMR (400 MHz, d₆-DMSO, ppm): δ 8.73 (m, 2H), 8.08 (m, 1H), and 3.92 (s, 6H). ES MS: m/z=212 (M+1).

### Step 3: Dimethyl 2-chloropyridine-3,5-dicarboxylate

A mixture of dimethylpyridine-3,5-dicarboxylate 1-oxide (5.15 g, 24.4 mmol) and phosphorus oxychloride (7.5 mL, 80 mmol) was heated at 90° C. for 5 d. The volatiles were removed to give a brown residual oil which was pipetted into MeOH (40 mL). The solvent was removed and the residue purified by SGC (0-60% EtOAc/hexanes) to give the title compound as an off-white solid. ¹H NMR (400 MHz, d₆-DMSO, ppm): δ 9.05 (m, 1H), 8.65 (m, 1H), and 3.92 (s, 6H). ES MS: m/z=230 (M+1).

### Step 4: Dimethyl 2-[(benzyloxy)amino]pyridine-3,5-dicarboxylate

A mixture of dimethyl 2-chloropyridine-3,5-dicarboxylate (618 mg, 2.7 mmol) and O-benzylhydroxylamine (663 mg, 5.4 mmol) in MeOH (20 mL) was heated at 80° C. overnight. The solvent was removed and the residue was purified by SGC (0-20% EtOAc/hexanes) to give title compound as an orange-yellow oil. ES MS: m/z=317 (M+1).

### Step 5: Dimethyl 2-[(benzyloxy)(3-ethoxy-3-oxopropanoyl)amino]pyridine-3,5-dicarboxylate

A solution of dimethyl 2-[(benzyloxy)amino]pyridine-3,5-dicarboxylate (740 mg, 2.3 mmol) and TEA (0.65 mL, 4.7 mmol) in DCM (10 mL) was treated dropwise with ethyl malonyl chloride (0.60 mL, 4.7 mmol) at room temperature. The mixture was stirred for 1 hour and was then partitioned between H₂O and DCM. The layers were separated and the aqueous layer extracted twice more with DCM. The combined organic extracts were dried, filtered and concentrated. The residue was purified by SGC (0-30% EtOAc/hexanes) to give the title compound as a yellow solid. ¹H NMR (400 MHz, d₆-DMSO, ppm): δ 8.9.12 (s, 1H), 8.51 (m, 1H), 7.39-7.33 (m, 5H), 5.00 (s, 2H), 4.05 (q, J=7.1 Hz, 2H), 3.90 (s, 2H), 3.76 (s, 3H), 3.72 (s, 3H), and 1.17-1.10 (m, overlap with residual EtOAc peak). ES MS: m/z=431 (M+1).

### Step 6: 3-Ethyl 6-methyl 1-(benzyloxy)-4-hydroxy-2-oxo-1,2-dihydro-1,8-naphthyridine-3,6-dicarboxylate and Diethyl 1-(benzyloxy)-4-hydroxy-2-oxo-1,2-dihydro-1,8-naphthyridine-3,6-dicarboxylate

A solution of dimethyl 2-[(benzyloxy)(3-ethoxy-3-oxopropanoyl)amino]pyridine-3,5-dicarboxylate (678 mg, 1.6 mmol) in EtOH (6 mL) was treated with a solution of sodium ethoxide in EtOH (21 wt %, 1.2 mL, 3.2 mmol), resulting in the precipitation of yellow solids. The thick mixture was stirred at room temperature for 3 hours and the solvent was then removed. The residue was partitioned between 0.5 M aqueous HCl and EtOAc. The layers were separated and the aqueous layer was extracted twice more with EtOAc. The combined organic extracts were dried, filtered and concentrated. The residue was triturated with EtOAc and the solids collected by vacuum filtration to afford a 1:1 mixture of the title compounds as a white solid. 3-Ethyl 6-methyl 1-(benzyloxy)-4-hydroxy-2-oxo-1,2-dihydro-1,8-naphthyridine-3,6-dicarboxylate: ES MS: m/z=399 M+1). Diethyl 1-(benzyloxy)-4-hydroxy-2-oxo-1,2-dihydro-1,8-naphthyridine-3,6-dicarboxylate: ES MS: m/z=413 (M+1).

### Step 7 8-(Benzyloxy)-6-(ethoxycarbonyl)-5-hydroxy-7-oxo-7,8-dihydro-1,8-naphthyridine-3-carboxylic acid

A mixture of 3-ethyl 6-methyl 1-(benzyloxy)-4-hydroxy-2-oxo-1,2-dihydro-1,8-naphthyridine-3,6-dicarboxylate and diethyl 1-(benzyloxy)-4-hydroxy-2-oxo-1,2-dihydro-1,8-naphthyridine-3,6-dicarboxylate (50 mg) in EtOH (2 mL) was treated with 1 N aqueous NaOH (0.13 mL, 0.13 mmol). After 10 minutes at room temperature, white solids precipitated from the initially homogeneous solution. The mixture was heated to 60° C. for 1 hour and was then treated with additional 1 N NaOH (0.13 mL) and heated overnight at 60° C. Additional 1 N NaOH (0.13 mL) was added and the mixture heated for 1 hour. The solvent was then removed and the residue partitioned between H₂O (acidified with 1 N aqueous HCl) and EtOAc. The layers were separated and the aqueous layer extracted twice more with EtOAc. The combined organic extracts were dried, filtered and concentrated the title compound as a white solid. ¹H NMR (400 MHz, d₆-DMSO, ppm): δ 9.23 (d, J=2.0 Hz, 1H), 8.84 (d, J=2.1 Hz, 1H), 7.67-7.65 (m, 2H), 7.47-7.39 (m, 3H), 5.17 (s, 2H), 4.36 (q, J=7.0 Hz, 2H), and 1.33 (t, J=7.2 Hz, 3H). ES MS: m/z=385 (M+1).

### Step 8: Ethyl 6-[(benzylamino)carbonyl]-1-(benzyloxy)-4-hydroxy-2-oxo-1,2-dihydro-1,8-naphthyridine-3-carboxylate

BOP reagent (115 mg, 0.26 mmol) was added to a solution of 8-(benzyloxy)-6-(ethoxycarbonyl)-5-hydroxy-7-oxo-7,8-dihydro-1,8-naphthyridine-3-carboxylic acid (50 mg, 0.13 mmol) in DMF (2 mL). The mixture was stirred for 10 minutes and was then treated with benzylamine (0.03 mL, 0.26 mmol). The mixture was stirred at room temperature for 1.5 hours and the solvent was then removed. The residue was partitioned between H₂O and EtOAc, the layers separated and the aqueous layer extracted twice more with EtOAc. The combined organic extracts were dried, filtered and concentrated. The residue was triturated with CH₃CN and the solids collected by vacuum filtration to afford the title compound as a white solid. Additional title compound was recovered by concentration of the filtrate. ES MS: m/z=474 (M+1).

### Step 9: Ethyl 6-[(benzylamino)carbonyl]-1,4-dihydroxy-2-oxo-1,2-dihydro-1,8-naphthyridine-3-carboxylate

Ethyl 6-[(benzylamino)carbonyl]-1-(benzyloxy)-4-hydroxy-2-oxo-1,2-dihydro-1,8-naphthyridine-3-carboxylate (32 mg, 0.07 mmol) was dissolved in EtOH (10 mL) and the solution was purged with N₂. 10% Pd/C (7.2 mg) was added and the mixture stirred under H₂ atmosphere (balloon) for 30 minutes. The reaction mixture was filtered through a Celite plug under N₂, rinsing the Celite with degassed EtOH. The filtrate was then passed through a Nylon 0.2 μm Millipore Milex-GN cartridge to remove any residual catalyst. The filtrate was concentrated and the residue triturated with EtOH. Collection of the resulting solids by vacuum filtration afforded the title compound as a yellow solid. ¹H NMR (400 MHz, d₆-DMSO, ppm): δ 12.9 (br s, 1H), 11.0 (br s, 1H), 9.39 (t, J=5.8 Hz, 1H), 9.20 (d, J=2.2 Hz, 1H), 8.90 (d, J=2.2 Hz, 1H), 7.36-7.24 (m, 5H), 4.53 (d, J=5.8 Hz, 2H), 4.34 (q, J=7.1 Hz, 2H), 1.31 (t, J=7.1 Hz, 3H). High Resolution MS (FT-ICR): m/z found 384.1195 (M+1); calculated 384.1190 (M+1).

TABLE 5
The following compounds were prepared from 8-(benzyloxy)-6-(ethoxycarbonyl)-
5-hydroxy-7-oxo-7,8-dihydro-1,8-naphthyridine-3-carboxylic acid (Example 77, Step 7)
essentially according to the methods described in Example 77, Steps 8-9 above:

| Ex/cpd | Name | Structure | Data |
|---|---|---|---|
| 78 | Ethyl 1,4-dihydroxy-6- {[3- methoxybenzyl)amino] carbonyl}-2-oxo-1,2-dihydro- 1,8-naphthyridine-3- carboxylate | 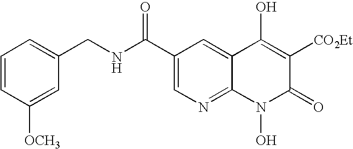 | High Resolution MS (FT-ICR): m/z found 414.1297 (M + 1); calculated 414.1296 (M + 1) |
| 79 | Ethyl 1,4-dihydroxy-6- {[2- methoxybenzyl)amino] carbonyl}-2-oxo-1,2-dihydro- 1,8-naphthyridine-3- carboxylate | 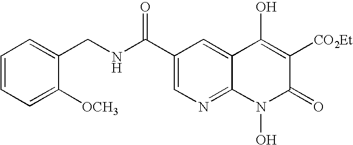 | High Resolution MS (FT-ICR): m/z found 414.1302 (M + 1); calculated 414.1296 (M + 1) |
| 80 | Ethyl 6-{[benzyl(methyl) amino]carbonyl}-1,4- dihydroxy-2-oxo-1,2- dihydro-1,8- naphthyridine-3- carboxylate | 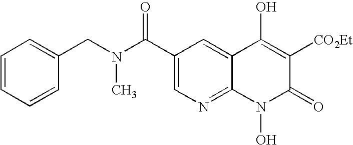 | High Resolution MS (FT-ICR): m/z found 398.1345 (M + 1); calculated 398.1347 (M + 1) |
| 81 | Ethyl 1,4-dihydroxy-6- {[methyl(2-phenylethyl) amino]carbonyl}-2-oxo- 1,2-dihydro-1,8- naphthyridine-3- carboxylate | 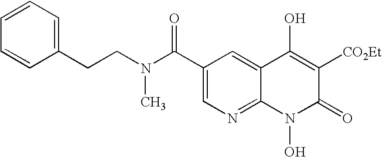 | High Resolution MS (FT-ICR): m/z found 412.1502 (M + 1); calculated 412.1503 (M + 1) |
| 82 | Ethyl 1,4-dihydroxy-2- oxo-6-{[2-(2-phenylethyl) piperidin-1-yl]carbonyl}- 1,2-dihydro-1,8- naphthyridine-3- carboxylate | 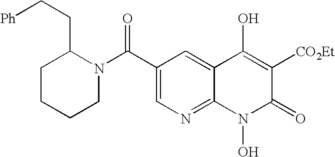 | High Resolution MS (FT-ICR): m/z found 466.1957 (M + 1); calculated 466.1973 (M + 1) |
| 83 | Ethyl 1,4-dihydroxy-2- oxo-6-{[4-(3- phenylpropyl)piperidin-1- yl]carbonyl}-1,2-dihydro- 1,8-naphthyridine-3- carboxylate | 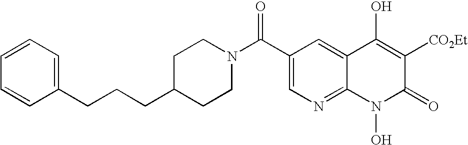 | High Resolution MS: m/z found 480.2131 (M + 1); calculated 480.2129 (M + 1) |
| 84 | Ethyl 1,4-dihydroxy-2- oxo-6-{[4-(2- phenylethyl)piperidin-1- yl]carbonyl}-1,2-dihydro- 1,8-naphthyridine-3- carboxylate | 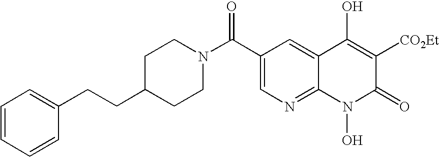 | High Resolution MS: m/z found 466.1965 (M + 1); calculated 466.1973 (M + 1) |
| 85 | Ethyl 6-[(3- benzylpyrrolidin-1- yl)carbonyl]-1,4- dihydroxy-2-oxo-1,2- dihydro-1,8- naphthyridine-3- carboxylate | 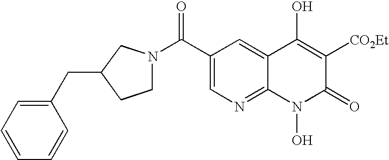 | High Resolution MS: m/z found 438.1651 (M + 1); calculated 438.1660 (M + 1) |
| 86 | ethyl-6- [(cyclohexylamino) carbonyl]-1,4-dihydroxy-2- oxo-1,2-dihydro-1,8- naphthyridine-3- carboxylate | 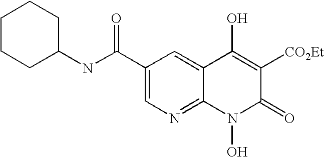 | High Resolution MS (FT-ICR): m/z found 376.1519 (M + 1); calculated 376.1503 (M + 1) |

### Example 87

### N,N′-Dibenzyl-1,4-dihydroxy-2-oxo-1,2-dihydro-1,8-naphthyridine-3,6-dicarboxamide

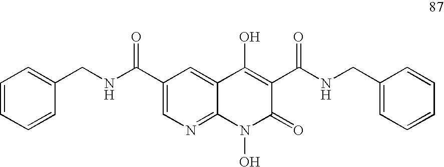

### Step 1: N,N-dibenzyl-1-(benzyloxy)-4-hydroxy-2-oxo-1,2-dihydro-1,8-naphthyridine-3,6-dicarboxamide

A solution of ethyl 6-[(benzylamino)carbonyl]-1-(benzyloxy)-4-hydroxy-2-oxo-1,2-dihydro-1,8-naphthyridine-3-carboxylate (Example 77, Step 8; 20 mg, 0.04 mmol) and benzylamine (0.5 mL, 4.6 mmol) in DMF (1.5 mL) was heated at 140° C. in a microwave. The solvent was removed and the residue was purified by RP-HPLC (C18 column; 0-75% CH₃CN/H₂O with 0.1% TFA) to give the title compound as a white solid. ES MS: m/z=535 (M+1)

### Step 2: N,N′-dibenzyl-1,4-dihydroxy-2-oxo-1,2-dihydro-1,8-naphthyridine-3,6-dicarboxamide

A mixture of N,N-dibenzyl-1-(benzyloxy)-4-hydroxy-2-oxo-1,2-dihydro-1,8-naphthyridine-3,6-dicarboxamide (11 mg, 0.02 mmol), 33 wt % HBr-HOAc (2 mL, 0.02 mmol) and H₂O (1 mL) was heated at 80° C. for 1 hour. The solvent was removed and the residue triturated with CH₃CN. The solids were collected by vacuum filtration to afford the title compound. High resolution MS (FT-ICR): m/z found 445.1513 (M+1); calculated 445.1507 (M+1).

### Example 88

### Ethyl 5,8-dihydroxy-7-oxo-7,8-dihydro-1,8-naphthyridine-4-carboxylate

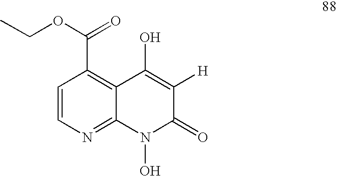

### Step 1: Diethyl pyridine-3,4-dicarboxylate 1-oxide

Urea hydrogen peroxide (4.42 g, 47.0 mmol) was added to a stirred solution of diethyl pyridine-3,4-dicarboxylate (5.00 g, 22.4 mmol) in DCM (150 mL) at 0° C. Trifluoroacetic anhydride (6.32 mL, 44.8 mmol) was added slowly to the mixture while maintaining the temperature below 5° C. Upon complete addition, the reaction mixture was allowed to warm to room temperature and stirred for 3 d. The mixture was then quenched by addition of aqueous sodium dithionite (250 mL) followed by stirring for 15 minutes. The mixture was then poured into aqueous 1 N HCl and extracted with DCM (×2). The combined organic extracts were dried, filtered and concentrated. The residue was purified by SGC (0-5% MeOH/DCM) to give the title compound. ES MS: m/z=240.3.

### Step 2: Diethyl 2-chloropyridine-3,4-dicarboxylate

A mixture of diethyl pyridine-3,4-dicarboxylate 1-oxide (1.00 g, 4.18 mmol) and phosphorus oxychloride (6.60 mL) was heated at 90° C. overnight. The volatiles were removed to afford a brown oil which was pipetted into MeOH (40 mL) and the mixture stirred for 30 minutes. The solvent was removed and the residue was pipetted into stirred saturated aqueous NaHCO₃ solution. The mixture was extracted with DCM (×3) and the combined organic layers were dried, filtered and concentrated. The residue was purified by SGC (0-50% EtOAc-hexanes) to give the regioisomeric by-product, diethyl 6-chloropyridine-3,4-dicarboxylate as the first component to elute, followed by the title compound. Title compound ES MS: m/z=258.3 (M+1).

### Step 3: Diethyl 2-[(benzyloxy)amino]pyridine-3,4-dicarboxylate

A mixture of diethyl 2-chloropyridine-3,4-dicarboxylate (400 mg, 1.55 mmol) and O-benzylhydroxylamine (382 mg, 3.10 mmol) in EtOH (15 mL) was heated at 80° C. overnight. No conversion had occurred and the mixture was treated with additional O-benzylhydroxylamine (764 mg, 6.20 mmol). After 4 hours and no conversion, the EtOH was removed the residue dissolved in diisopropylethylamine (20 mL). The mixture was heated at 130° C. for 6 d, at which point most of the solvent had evaporated and formation of the title compound was observed by LCMS. Additional heating at 130° C. for 1 more day did not result in further conversion. The crude material was purified by SGC (0-30% EtOAc-hexanes) to give the title compound. ES MS: m/z=345.3 (M+1).

### Step 4: Diethyl 2-[acetyl(benzyloxy)amino]pyridine-3,4-dicarboxylate

Acetic anhydride (33 μL, 0.35 mmol) was added dropwise to a mixture of diethyl 2-[(benzyloxy)amino]pyridine-3,4-dicarboxylate (60 mg, 0.17 mmol) and TEA (48 μL, 0.35 mmol) in DCM (2 mL) at room temperature. No conversion had occurred after 5.5 hours. The mixture was treated with additional acetic anhydride and TEA and stirring continued for 5 d. The mixture was then heated at 50° C. for 2 hours and treated with acetyl chloride (25 μL, 0.35 mmol), but with no further conversion. The mixture was partitioned between H₂O and DCM. The layers were separated and the aqueous layer further extracted with DCM (×2). The combined organic layers were dried, filtered and concentrated. The residue was purified by SGC (0-5% MeOH/DCM) to give the title compound. ES MS: m/z=345.3 (M+1-42),387.3 (M+1).

### Step 5: Ethyl 8-(benzyloxy)-5-hydroxy-7-oxo-7,8-dihydro-1,8-naphthyridine-4-carboxylate

A solution of lithium hexamethyldisilazide (1 M in THF, 0.32 mL, 0.32 mmol) was added dropwise to a cold (−78° C.) solution of diethyl 2-[acetyl(benzyloxy)amino]pyridine-3,4-dicarboxylate (50 mg, 0.13 mmol) in anhydrous THF (1 mL) while maintaining the temperature below −75° C. The mixture was stirred for 15 minutes at −78° C. and was then allowed to warm to room temperature and quenched by addition of aqueous 1 M HCl. The mixture was extracted with EtOAc (×3) and the combined organic extracts were washed with brine, dried, filtered and concentrated to afford the title compound. ES MS: m/z=341.2 (M+1).

### Step 6: Ethyl 5,8-dihydroxy-7-oxo-7,8-dihydro-1,8-naphthyridine-4-carboxylate

A solution of ethyl 8-(benzyloxy)-5-hydroxy-7-oxo-7,8-dihydro-1,8-naphthyridine-4-carboxylate (40 mg, 0.12 mmol) in EtOH (5 mL) was purged with N₂. 10% Pd/C (13 mg) was added and the mixture was stirred under H₂ atmosphere for 2.5 hours. The mixture was then filtered through a pad of Celite, washing with EtOH. The filtrate was concentrated and the residue purified by RP-HPLC (C18 column; 0-95% CH₃CN—H₂O with 0.1% TFA) to give the title compound. ¹H NMR (400 MHz, d₆-DMSO, ppm): δ 11.97 (s, 1H), 8.70 (d, J=4.7 Hz, 1H), 7.25 (d, J=4.7 Hz, 1H), 5.93 (s, 1H), 4.34 (q, J=7.1 Hz, 2H), and 1.30 (t, J=7.1 Hz, 3H). High resolution MS: m/z found 251.0663 (M+1), calculated 251.0662 (M+1).

### Example 89

### Ethyl 6-(4-aminophenoxy)-1,4-dihydroxy-2-oxo-1,2-dihydro-1,8-naphthyridine-3-carboxylate

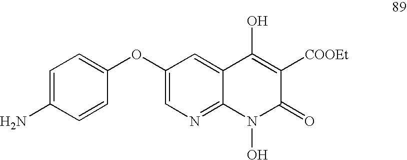

### Step 1: Methyl 5-(4-nitrophenoxy)nicotinate

The title compound was prepared from 5-hydroxynicotinic acid methyl ester (available from TCI-US) and 1-fluoro-4-nitrobenzene essentially according to the method described in Khire, U. R. et al Bioorg. Med. Chem. Lett. 2004, 14, 783-786, substituting cesium carbonate for sodium hydride as the base.

### Step 2: Methyl 5-(4-nitrophenoxy)nicotinate 1-oxide

The title compound was prepared from methyl 5-(4-nitrophenoxy)nicotinate (2.35 g, 8.6 mmol) essentially according to the procedure described in Example 77, Step 2 and was isolated as a pale yellow solid. ¹H NMR (400 MHz, d₆-DMSO, ppm): δ 8.63 (m, 1H), 8.46 (m, 1H), 8.32 (d, J=0.7 Hz, 2H), 8.30 (m, 1H), 8.29 (d, J=0.7 Hz, 2H), and 3.88 (s, 3H). ES MS: m/z=291 (M+1).

### Step 3: Methyl 2-chloro-5-(4-nitrophenoxy)nicotinate

The title compound was prepared from methyl 5-(4-nitrophenoxy)nicotinate-1-oxide (1.0 g, 3.4 mmol) essentially according to the procedure described in Example 77, Step 3. Purification of the crude reaction product by SGC (0-20% EtOAc-hexanes) afforded a 1:1 mixture of the title compound and the regioisomeric methyl 6-chloro-5-(4-itrophenoxy)nicotinate as a pale yellow oil. ES MS: m/z/z=309 (M+1).

### Step 4: Methyl 2-[(benzyloxy)amino]-5-(4-nitrophenoxy)nicotinate

The title compound was prepared from a 1:1 mixture of methyl 2-chloro-5-(4-nitrophenoxy)nicotinate and methyl 6-chloro-5-(4-nitrophenoxy)nicotinate (865 mg, 2.80 mmol) essentially according to the procedure described in Example 77, Step 4. Purification of the crude product mixture by RP-HPLC (C18 column; 0-95% CH₃CN—H₂O with 0.1% TFA) afforded a 1:1 mixture of the title compound and the regioisomeric methyl 6-[(benzyloxy)amino]-5-(4-nitrophenoxy)nicotinate as an orange yellow-oil. ES MS: m/z=396 (M+1).

### Step 5: Methyl 2-[(benzyloxy)(3-ethoxy-3-oxopropanoyl)amino]-5-(4-nitrophenoxy)nicotinate

The title compound was prepared from a 1:1 mixture of ethyl 2-[(benzyloxy)amino]-5-(4-nitrophenoxy)nicotinate and methyl 6-[(benzyloxy)amino]-5-(4-nitrophenoxy)nicotinate (251 mg, 0.64 mmol) essentially according to the procedure described in Example 77, Step 5. Purification of the crude product mixture by SGC (0-60% EtOAc-hexanes) afforded separation of the title compound (yellow oil) from the unreacted methyl 6-[(benzyloxy)amino]-5-(4-nitrophenoxy)nicotinate starting material. ¹H NMR (400 MHz, d₆-DMSO, ppm): δ 8.67 (s, 1H), 8.32 (m, 2H), 8.00 (s, 1H), 7.38-7.32 (m, 7H), 5.04 (s, 2H), 4.10 (q, J=6.9 Hz, 2H), 3.78 (s, 3H), 3.70 (s, 2H), and 1.19-1.15 (m). ES MS: m/z=510 (M+1).

### Step 6: Ethyl 1-(benzyloxy)-4-hydroxy-6-(4-nitrophenoxy)-2-oxo-1,2-dihydro-1,8-naphthyridine-3-carboxylate

The title compound was prepared from methyl 2-[(benzyloxy)(3-ethoxy-3-oxopropanoyl)amino]-5-(4-nitrophenoxy)nicotinate (110 mg, 0.22 mmol) essentially according to the procedure described in Example 77, Step 6 and was isolated as a yellow solid. ES MS: m/z=478 (M+1).

### Step 7: Ethyl 6-(4-aminophenoxy)-1,4-dihydroxy-2-oxo-1,2-dihydro-1,8-naphthyridine-3-carboxylate

The title compound was prepared from ethyl 1-(benzyloxy)-4-hydroxy-6-(4-nitro phenoxy)-2-oxo-1,2-dihydro-1,8-naphthyridine-3-carboxylate (44 mg, 0.09 mmol) essentially according to the procedure described in Example 77, Step 9, omitting the filtration through a Nylon 0.2 gm Millipore Milex-GN cartridge. The crude product was purified by RP-HPLC (C18 column; 0-95% CH₃CN—H₂O with 0.1% TFA) to give the title compound as an orange solid. ¹H NMR (400 MHz, CD₃OD, ppm) δ 8.16 (d, J=2.2 Hz, 1H), 8.14 (d, J=2.5 Hz, 1H), 7.44 (d, J=8.6 Hz, 2H), 7.26 (d, J=8.4 Hz, 2H), 4.49 (q, J=7.1 Hz, 2H), and 1.43 (t, J=7.0 Hz, 3H). High Resolution MS (FT-ICR): m/z found 358.1045 (M+1); calculated 358.1034 (M+1).

### Example 90

### Ethyl 6-[4-(diethylamino)phenoxy]-1,4-dihydroxy-2-oxo-1,2-dihydro-1,8-naphthyridine-3-carboxylate

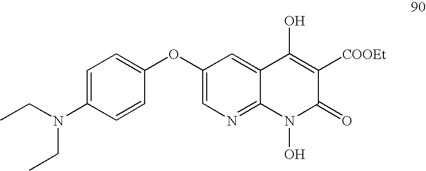

A solution of ethyl 6-(4-aminophenoxy)-1,4-dihydroxy-2-oxo-1,2-dihydro-1,8-naphthyridine-3-carboxylate (Example 89, Step 9; 29 mg, 0.08 mmol) in DMF (1 mL) and EtOH (1 mL) was treated successively with HOAc (14 μL, 0.24 mmol), acetaldehyde (14 μL, 0.24 mmol) and sodium cyanoborohydride (15 mg, 0.24 mmol). The mixture was stirred at room temperature for 2 hours. The solvent was then removed and the residue was purified by RP-HPLC (C18 column; 0-95% CH₃CN—H₂O with 0.1% TFA) to give the title compound as a yellow solid. ¹H NMR (400 MHz, d₆-DMSO, ppm) δ 13.6 (bs, 1H), 10.9 (bs, 1H), 8.64 (s, 1H), 7.91 (bs, 1H), 7.23-7.04 (m, 4H), 4.32 (q, J=7.1 Hz, 2H), 3.60-3.28 (m, 4H), 1.29 (t, J=7.0 Hz, 3H), and 1.05 (m, 6H). High Resolution MS (FT-ICR): m/z found 414.1676 (N+1); calculated 414.1660 (M+1).

### Example 91

### Methyl 6-[(benzylamino)carbonyl]-5,8-dihydroxy-7-oxo-7,8-dihydro-1,8-naphthyridine-3-carboxylate

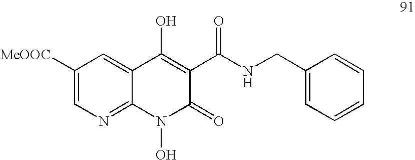

### Step 1: Methyl 6-[(benzylamino)carbonyl]-8-(benzyloxy)-5-hydroxy-7-oxo-7,8-dihydro-1,8-naphthyridine-3-carboxylate and Ethyl 6-[(benzylamino)carbonyl]-8-(benzyloxy)-5-hydroxy-7-oxo-7,8-dihydro-1,8-naphthyridine-3-carboxylate

A mixture of 3-ethyl 6-methyl 1-(benzyloxy)-4-hydroxy-2-oxo-1,2-dihydro-1,8-naphthyridine-3,6-dicarboxylate and diethyl 1-(benzyloxy)-4-hydroxy-2-oxo-1,2-dihydro-1,8-naphthyridine-3,6-dicarboxylate (Example 77, Step 6; 61 mg, 0.08 mmol) in DMF (3 mL) was treated with benzylamine (1 mL, 9.2 mmol). The mixture was heated in a microwave at 140° C. for 45 minutes. The crude mixture was then purified by RP-HPLC (C18 column; 0-85% CH₃CN/H₂O with 0.1% TFA) to afford the title compounds. Methyl 6-[(benzylamino)carbonyl]-8-(benzyloxy)-5-hydroxy-7-oxo-7,8-dihydro-1,8-naphthyridine-3-carboxylate: white solid. ES MS: m/z=460 (M+1). Ethyl 6-[(benzylamino)carbonyl]-8-(benzyloxy)-5-hydroxy-7-oxo-7,8-dihydro-1,8-naphthyridine-3-carboxylate: White solid (36 mg). ES MS: m/z=474 (M+1).

### Step 2: Methyl 6-[(benzylamino)carbonyl]-5,8-dihydroxy-7-oxo-7,8-dihydro-1,8-naphthyridine-3-carboxylate

A solution of methyl 6-[(benzylamino)carbonyl]-8-(benzyloxy)-5-hydroxy-7-oxo-7,8-dihydro-1,8-naphthyridine-3-carboxylate (15 mg, 0.03 mmol) in MeOH (5 mL) was purged with N₂ gas and treated with 10% Pd/C (3.5 mg). The reaction mixture was stirred under H₂ atmosphere (balloon) for 30 minutes and was then purged with N₂ and filtered through a plug of Celite, rinsing with degassed MeOH. The filtrate was then passed through a Nylon 0.2 μm Millipore Milex-GN cartridge to remove any residual catalyst. The filtrate was concentrated to afford the title compound as a yellow solid. ¹H NMR (400 MHz, d₆-DMSO, ppm) δ 11.3 (bs, 1H), 10.4 (bs, 1H), 9.22 (bs, 1H), 8.08 (d, J=2.0 Hz, 1H), 7.37-7.26 (m, 6H), 4.61 (d, J=5.3 Hz, 2H), and 3.92 (s, 3H). ES MS: m/z=370 (M+1).

### Example 92

### Ethyl 6-[(benzylamino)carbonyl]-5,8-dihydroxy-7-oxo-7,8-dihydro-1,8-naphthyridine-3-carboxylate

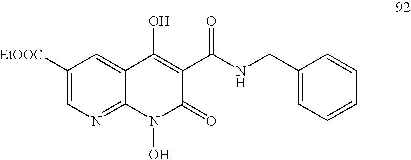

The title compound was prepared from ethyl 6-[(benzylamino)carbonyl]-8-(benzyloxy)-5-hydroxy-7-oxo-7,8-dihydro-1,8-naphthyridine-3-carboxylate (Example 91, Step 1; 15 mg) essentially according to the procedure described in Example 91, Step 2 and was isolated as a yellow solid. ES MS: m/z=384 (M+1).

### Example 93

### 3-acetyl-1,4-dihydroxy-1,8-naphthyridin-2(1H)-one

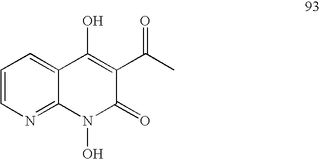

### Step 1 Ethyl 2-{acetoacetyl(benzyloxy)amino]nicotinate

A mixture of ethyl 2-[(benzyloxy)amino]nicotinate ([J. Het. Chem. 1993, 30 (4), 909-912]; 300 mg, 1.1 mmol) and diketene (0.5 mL) was heated in a microwave at 100° C. for 30 minutes. The solution was cooled and purified by SGC (20-100% EtOAc-hexanes) to give the title compound. ¹H NMR (400 MHz, CDCl₃, ppm): δ 8.67 (d, J=8.3 Hz, 1H), 8.21 (d, J=7.8 Hz, 1H), 7.31-7.35 (m, 6H), 5.21 (s, 2H), 4.21 (q, J=5.8 Hz, 2H), 3.57 (s, 2H), 2.12 (s, 3E1), and 1.21 (t, J=6.2 Hz, 3H). ES MS: m/z=357.2.

### Step 2: 3-Acetyl-1-(benzyloxy)-4-hydroxy-1,8-napthyridin-2-(1H)-one

A solution of ethyl 2-{acetoacetyl(benzyloxy)amino]nicotinate (287 mg, 0.80 mmol) in EtOH (5 mL) was treated with potassium tert-butoxide (220 mg, 1.96 mmol) and the mixture stirred at room temperature for 30 minutes. The mixture was then acidified with aqueous HCl (1.0 M, 5 mL) and extracted with EtOAc (25 mL). The organic layer was concentrated and the residue recrystallized from EtOAc and hexane to afford the title compound. ES MS: m/z=311 (M+1).

### Step 3: 3-acetyl-1,4-dihydroxy-1,8-naphthyridin-2(1H)-one

A solution of 3-acetyl-1-(benzyloxy)-4-hydroxy-1,8-napthyridin-2-(1H)-one (50 mg, 0.16 mmol) was taken up in 33% HBr/HOAc solution (1 mL) and heated at 80° C. for 1 hour. The solution was then cooled and concentrated. The residue was purified by RP-HPLC (C18 column; CH₃CN/H₂O with 0.1% TFA to give the title compound. ¹H NMR (400 MHz, d₆-DMSO, ppm): δ 10.81 (s, 1H), 8.78 (d, J=6.2 Hz, 1H), 8.46 (d, J=8.5 Hz, 1H), 7.36 (dd, J=6.2, 7.1 Hz, 1H), 6.49 (br s, 1H), and 2.76 (s, 3H). ES MS: t/z=221.2 (M+1).

### Example 94

### 5-Hydroxy-3-methyl-1,5-dihydro-4H-pyrazolo[4,3-c]-1,8-naphthyridin-4-one

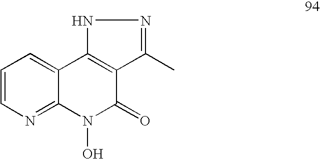

3-Acetyl-1-(benzyloxy)-4-hydroxy-1,8-napthyridin-2-(1H)-one (Example 93, Step 2; 50 mg, 0.16 mmol) was taken up in HOAc (1.0 mL). Sulfuric acid (2 drops) and hydrazine (0.5 mL) were added and the mixture heated at 80° C. for 3 hours. The solution was cooled and treated with 33% HBr in HOAc (3.0 mL). Heating was continued at 80° C. for 1 hour. The solvents were removed and the residue purified by RP-HPLC (C18 column; CH₃CN and H₂O with 0.1% TFA) to give the title compound. ¹H NMR (400 MHz, d₆-DMSO, ppm): δ 10.81 (s, 1H), 8.78 (d, J=6.2 Hz, 1H), 8.66 (d, J=8.5 Hz, 1H), 7.36 (br s, 1H), and 2.66 (s, 3H). ES MS: m/z=217.2 (M+1).

### Example 95

### 5,8-Dihydroxy-7-oxo-N,6-diphenyl-7,8-dihydro-1,8-naphthyridine-3-sulfonamide

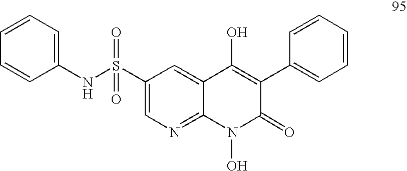

### Step 1: Ethyl 2-chloro-5-(chlorosulfonyl)nicotinate

Thionyl chloride (18 mL, 247 mmol) was added to a stirred solution of 5-(ethoxy carbonyl)-6-hydroxypyridine-3-sulfonic acid ([Org. Proc. Res. Dev. 2002, 6, 767-772]; 12.0 g, 48 mmol) in toluene (50 mL). DMF (2 mL) was added and the resulting suspension was refluxed for 3 hours, yielding a yellow solution. The solvents were removed and the residue partitioned between EtOAc and saturated aqueous NaHCO₃/brine. The layers were separated and the organic layer was concentrated to give the crude title compound which was used directly in the next step.

### Step 2: Ethyl 5-(anilinosulfonyl)-2-chloronicotinate

A solution of ethyl 2-chloro-5-(chlorosulfonyl)nicotinate (1.0 g, 3.5 mmol) in toluene (3.5 mL) was cooled to −10° C. A solution of aniline (320 μL, 3.5 mmol) and TEA (1.1 mL, 7.0 mmol) in toluene (3 mL) was added slowly dropwise while maintaining the temperature below 10° C. Upon complete addition the mixture was allowed to warm to room temperature and was then washed with H₂O and brine. The organic layer was concentrated and the residue was purified by SGC (0-30% EtOAc-hexanes) to give the title compound co-eluted with a by-product, ethyl 2-anilino-5-(anilinosulfonyl)nicotinate. The mixture was re-purified by SGC (0-10% EtOAc/DCM) to give title compound as white crystals. The ethyl 2-anilino-5-(anilinosulfonyl)nicotinate by-product was collected separately. Title compound ES MS: m/z=341.2 (M+1).

### Step 3: Ethyl 5-(anilinosulfonyl)-2-[(benzyloxy)amino]nicotinate

A mixture of ethyl 5-(anilinosulfonyl)-2-chloronicotinate (1.0 g, 2.9 mmol), O-benzylhydroxylamine (680 μL, 5.9 mmol) and diisopropylethylamine (1.0 mL, 5.9 mmol) was heated at 90° C. for 1 hour. The mixture was diluted with DCM (1 mL) and purified by SGC (0-30% EtOAc-hexanes), followed by RP-HPLC (C18 column; 15-100% CH₃CN/H₂O with 0.1% TFA) to give the title compound. ES MS: m/z=428.3 (M+1).

### Step 4: Ethyl 5-(anilinosulfonyl)-2-[benzoyl(benzyloxy)amino]nicotinate

A mixture of ethyl 5-(anilinosulfonyl)-2-[(benzyloxy)amino]nicotinate (361 mg, 0.844 mmol) and phenylacetyl chloride (1.1 mL, 8.4 mmol) was stirred at room temperature for 1 hour. Pyridine (137 μL, 1.69 mmol) was added and stirring continued for an additional hours. The mixture was diluted with DCM and washed with aqueous 1 N HCl. The aqueous layer was further extracted with DCM (×3) and the combined organic extracts were washed with brine, dried filtered and concentrated. The residue was purified by SGC (30-50% EtOAc-hexanes) to afford the title compound. ES MS: m/z=546.1 (M+1)

### Step 5: 8-(Benzyloxy)-5-hydroxy-7-oxo-N,6-diphenyl-7,8-dihydro-1,8-naphthyridine-3-sulfonamide

Ethyl 5-(anilinosulfonyl)-2-[benzoyl(benzyloxy)amino]nicotinate (230 mg, 0.422 mmol) was azeotroped twice with anhydrous DMF. The residue was dissolved in THF (4.2 mL) and the stirred solution cooled to −78 C— Lithium hexamethyldisilazide (2 M in THF, 0.63 mL, 1.3 mmol) was added dropwise and the mixture was then allowed to warm to room temperature. The solvent was removed and the residue purified by SGC (0-30-50-100% EtOAc-hexanes) to give the title compound. ES MS: m/z=500.2 (M+1)

### Step 6: 5,8-Dihydroxy-7-oxo-N,6-diphenyl-7,8-dihydro-1,8-naphthyridine-3-sulfonamide

A degassed solution of 8-(benzyloxy)-5-hydroxy-7-oxo-N,6-diphenyl-7,8-dihydro-1,8-naphthyridine-3-sulfonamide (85 mg, 0.17 mmol) in EtOH (15 mL) was treated with 10% Pd/C (18 mg). The mixture was flushed with H₂ (×3) and then stirred under H₂ atmosphere for 4 hours. The mixture was then filtered through a pad of Celite. The filtrate was concentrated and the residue dissolved in MeOH and purified by RP-HPLC (C18 column; 15-100% CH₃CN/H₂O with 0.1% TFA) to give the title compound. ¹H NMR (400 MHz, d₆-DMSO, ppm): δ 10.94 (br s, 1H), 10.44 (s, 1H), 8.85 (d, J=2.2 Hz, 1H), 8.68 (d, J=2.2 Hz, 1H), 7.46-7.34 (m, 5H), 7.29-7.25 (m, 2H), and 7.14-7.06 (m, 2H). ES MS: m/z=410.01 (M+1).

### Example 96

### N-benzyl-5,8-dihydroxy-7-oxo-6-phenyl-7,8-dihydro-1,8-naphthyridine-3-sulfonamide

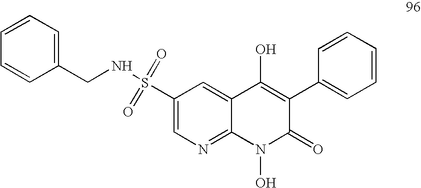

The above compound was prepared in accordance with the procedures set forth in Example 95. High Resolution MS (FT-ICR): m/z found 424.2 (M+1); calculated 423.4418 (M+1)

### Example 97

### Ethyl 4-amino-1-hydroxy-2-oxo-1,2-dihydro-1,8-naphthyridine-3-carboxylate

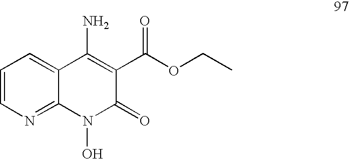

### Step 1: 2-[(Benzyloxy)amino]nicotinonitrile

A mixture of 2-chloronicotinonitrile (5.0 g, 36.1 mmol), O-benzylhydroxylamine hydrochloride (6.91 g, 43.3 mmol), and DIEA (12.6 mL, 72.2 mmol) was stirred and heated in a sealed flask at 160° C. for 18 hours. H₂O and EtOAc were added and the layers were separated. The aqueous layer was extracted with EtOAc (3×) and the combined organic extracts were washed with saturated brine, dried, filtered and concentrated. The crude residue was purified by SGC (0-50% EtOAc/hexanes) to give the title compound as an orange solid. ES MS: m/z/z=226 (M+1).

### Step 2: Ethyl 3-[(benzyloxy)(3-cyanopyridin-2-yl)amino]-3-oxopropanoate

To a solution of 2-[(benzyloxy)amino]nicotinonitrile (2.5 g, 11.1 mmol) and TEA (2.32 mL, 16.6 mmol) in DCM (30 mL) was added dropwise ethyl 3-chloro-3-oxopropanoate (2.14 mL, 16.6 mmol). The reaction mixture was stirred at for 2 hours. The solvent was removed and the residue was triturated with EtOAc. The solids were filtered off and the filtrate was purified by SGC (0-50% EtOAc/hexanes) to give the title compound as an orange oil. ES MS: m/z=340 (M+1).

### Step 3: Ethyl 4-amino-1-(benzyloxy)-2-oxo-1,2-dihydro-1,8-naphthyridine-3-carboxylate

To a solution of ethyl 3-[(benzyloxy)(3-cyanopyridin-2-yl)amino]-3-oxopropanoate ((1.87 g, 5.5 mmol) in anhydrous EtOH (40 mL) was added a solution of sodium ethoxide (21% wt. in EtOH; 4.11 mL, 11.0 mmol). The reaction turned darker orange. After 45 minutes, the EtOH was removed. EtOAc and H₂O were added and the solution was brought to pH 3 with the addition of 1 N HCl. The layers were separated and the aqueous layer was extracted with EtOAc (3×). The combined organic extracts were dried, filtered and concentrated to give the title compound as an orange solid. ES MS: m/z=340 (M+1).

### Step 4: Ethyl 4-amino-1-hydroxy-2-oxo-1,2-dihydro-1,8-naphthyridine-3-carboxylate

To a solution of ethyl 4-amino-1-(benzyloxy)-2-oxo-1,2-dihydro-1,8-naphthyridine-3-carboxylate (0.10 g, 0.30 mmol) in degassed EtOH (5 mL) was added 10% Pd/C (10 mg). The reaction mixture was further degassed and purged with N₂ (3 times) and was then placed under H₂ balloon and stirred for 18 hours. The mixture was filtered through Celite and washed with degassed MeOH. The filtrate was concentrated. The resulting residue was purified by RP-HPLC (C18 column; 5-65% CH₃CN/H₂O with 0.1% TFA) to give the title compound. ¹H NMR (400 MHz, d₆-DMSO, ppm): δ 8.67 (d, J=4.6 Hz, 1H), 8.63 (dd, J=1.3, 8.1 Hz, 1H), 8.23 (s, 2H), 7.29 (dd, J=4.7, 8.1 Hz, 1H), 4.26 (q, J=7.1 Hz, 2H), and 1.28 (t, J=7.1 Hz, 3H). High Resolution MS (FT-ICR): m/z found 250.0822 (M+1); calculated 250.0823 (M+1).

### Example 98

### Ethyl 4-amino-1-hydroxy-2-oxo-6-phenyl-1,2-dihydro-1,8-naphthyridine-3-carboxylate

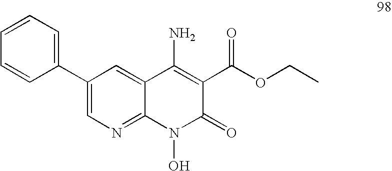

The title compound was prepared from 2-chloro-5-phenylnicotinonitrile essentially according to the procedures described in Example 97. High Resolution MS (FT-ICR): m/z found 326.1166(M+1); calculated 326.1136 (M+1).

### Example 99

### 4-Amino-1-hydroxy-1,8-naphthyridin-2(1H)-one

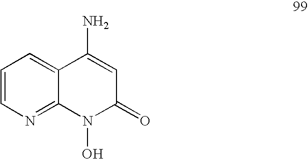

### Step 1: 4-Amino-1-(benzyloxy)-1,8-naphthyridin-2(1H)-one

To a solution of ethyl 4-amino-1-(benzyloxy)-2-oxo-1,2-dihydro-1,8-naphthyridine-3-carboxylate (Example 9, Step 3; 1.0 g, 3.0 mmol) in MeOH (30 mL) was added aqueous NaOH solution (2 M, 8.84 mL, 17.7 mmol). The reaction mixture was heated to reflux. After 2 hours, additional NaOH (0.35 g, 8.84 mmol) and H₂O (10 mL) were added and the mixture was stirred at reflux for an additional 18 hours. The reaction mixture was allowed to cool to room temperature. The solids that formed in the reaction mixture were collected by vacuum filtration to give the title compound. ES MS: m/z=268 (M+1).

### Step 2: 4-Amino-1-hydroxy-1,8-naphthyridin-2(1H)-one

The solution of 4-amino-1-(benzyloxy)-1,8-naphthyridin-2(1H)-one (79 mg, 0.30 mmol) in HBr (33% wt. in HOAc; 3 mL) was heated to 50° C. for 2 hours. The reaction mixture was allowed to cool to room temperature and the solvent was removed. The residue was triturated with CH₃CN and the solids were collected by vacuum filtration to give the title compound. ¹H NMR (400 MHz, d₆-DMSO, ppm): δ 8.66-8.62 (m, 2H), 7.65 (br s, 2H) 7.45-7.42 (m, 2H), 5.70 (s, 1H). High Resolution MS (FT-ICR): m/z found 178.0613 (M+1); calculated 178.0611 (M+1).

### Example 100

### N-(1-Hydroxy-2-oxo-1,2-dihydro-1,8-naphthyridin-4-yl)acetamide

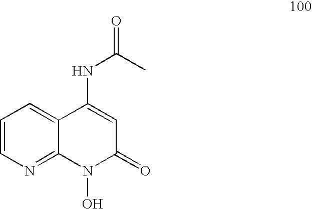

### Step 1: N-[1-(Benzyloxy)-2-oxo-1,2-dihydro-1,8-naphthyridin-4-yl]acetamide

To a solution of 4-amino-1-(benzyloxy)-1,8-naphthyridin-2(1H)-one (Example 99, Step 1; 75 mg, 0.28 mmol) and pyridine (34 uL, 0.42 mmol) in anhydrous DCM (3 mL) was added acetyl chloride (24 μL, 0.34 mmol). After 1 hour, additional pyridine (34 μL, 0.42 mmol) and acetyl chloride (24 μL, 0.34 mmol) were added and the reaction was stirred at room temperature for an additional 18 hours. The reaction was concentrated and the crude residue was purified by RP-HPLC (C18 column; 5-95% CH₃CN/H₂O with 0.1% TFA) to give the title compound. ES MS: m/z=310 (M+1).

### Step 2: N-(1-Hydroxy-2-oxo-1,2-dihydro-1,8-naphthyridin-4-yl)acetamide

The solution of N-[1-(benzyloxy)-2-oxo-1,2-dihydro-1,8-naphthyridin-4-yl]acetamide (62 mg, 0.20 mmol) in HBr (33% wt. in HOAc; 2 mL) was heated to 60° C. for 2 hours. The solvent was removed and the residue was triturated with MeOH. The solids formed were collected by vacuum filtration to give the title compound as a yellow solid. ¹H NMR (400 MHz, d₆-DMSO, ppm): δ 9.96 (m, 1H), 8.59-8.55 (m, 3H), 7.35 (m, 2H), 5.65 (s, 1H), 2.20 (s, 3H). High Resolution MS (FT-ICR): m/z found 220.0718 (M+1); calculated 220.0717 (M+1).

TABLE 6
The compounds in the following table were prepared in accordance with the
procedures set forth in Example 100:

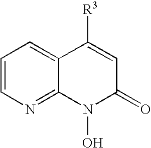

| Ex/cpd | Name | R³ | Data |
|---|---|---|---|
| 101 | N-(1-Hydroxy-2-oxo- 1,2-dihydro-1,8- naphthyridin-4-yl)-2- phenylacetamide | 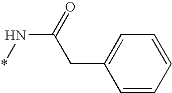 | High Resolution MS (FT-ICR): m/z found 496.1030 (M + 1); calculated 296.1030 (M + 1) |
| 102 | N-(1-Hydroxy-2-oxo- 1,2-dihydro-1,8- naphthyridin-4- yl)benzamide | 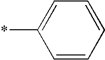 | High Resolution MS: m/z found 282.0867 (M + 1); calculated 282.0873 (M + 1) |

### Example 103

### 4-Anilino-1-hydroxy-1,8-naphthyridin-2(1H)-one

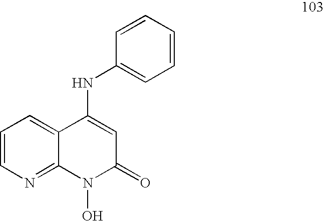

### Step 1: 1-(Benzyloxy)-2-oxo-1,2-dihydro-1,8-naphthyridin-4-yl trifluoromethanesulfonate

A solution of 1-(benzyloxy)-4-hydroxy-1,8-naphthyridin-2(1H)-one (Example 2, Step 1; 276 mg, 1.03 mmol) and TEA (0.29 mL, 2.06 mmol) in DCM (5 mL) was cooled to 0° C. and treated dropwise with trifluoromethanesulfonic anhydride (0.35 mL, 2.06 mmol). The cooling bath was removed and the mixture stirred at room temperature for 1 hour. The crude reaction mixture was SGC (0 to 40% EtOAc-hexanes) to give the title compound. ES MS: m/z=401 (M+1).

### Step 2: 4-Anilino-1-(benzyloxy)-1,8-naphthyridin-2(1H)-one

A mixture of 1-(benzyloxy)-2-oxo-1,2-dihydro-1,8-naphthyridin-4-yl trifluoromethane-sulfonate (50 mg, 0.12 mmol) and aniline (0.5 mL, 5.48 mmol) in DMF (1.5 mL) was heated in a microwave at 140° C. for 45 minutes. The crude reaction mixture was purified by RP-HPLC (C18 column; 95:5 to 5:95 H₂O:CH₃CN with 0.1% TFA) to give the title compound as a pale yellow solid. ES MS: m/z=344 (M+1).

### Step 3: 4-anilino-1-hydroxy-1,8-naphthyridin-2(1B)-one

A mixture of 4-anilino-1-(benzyloxy)-1,8-naphthyridin-2(1H)-one (22 mg, 0.06 mmol) in 33 wt % HBr-HOAc (2 mL, 0.06 mmol) and H₂O (1 mL) was heated at 80° C. for 1 hour. The solvents were removed and the residue was triturated with CH₃CN. The solids were collected by vacuum filtration to afford the title compound as a bright yellow-orange solid. ¹H NMR (400 MHz, d₆-DMSO, ppm) δ 8.90 (s, 1H), 8.72 (d, J=8.0 Hz, 1H), 8.29 (d, J=4-7 Hz, 1H), 7.48-7.41 (m, 3H), 7.34 (m, 2H), 7.21 (t, J=7.0 Hz, 1H), and 5.88 (s, 1H). High Resolution MS: m/z found 254.0920 (M+1); calculated 254.0924 (M+1).

TABLE 7
The compounds in the following table were prepared in accordance with the
procedures set forth in Example 103:

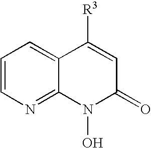

| Ex/cpd | Name | R³ | Data |
|---|---|---|---|
| 104 | 4-(Biphenyl-2- ylamino)-1-hydroxy- 1,8-naphthyridin- 2(1H)-one | 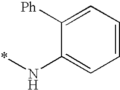 | High Resolution MS: m/z found 330.1260 (M + 1); calculated 330.1237 (M + 1) |
| 105 | 4-(Biphenyl-3- ylamino)-1-hydroxy- 1,8-naphthyridin- 2(1H)-one | 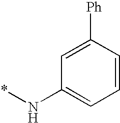 | High Resolution MS: m/z found 659.2388 (2M + 1); calculated 659.2401 (2M + 1) |
| 106 | 4-(Biphenyl-4- ylamino)-1-hydroxy- 1,8-naphthyridin- 2(1H)-one | 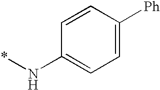 | High Resolution MS: m/z found 330.1248 (M + 1); calculated 330.1237 (M + 1) |
| 107 | 1-Hydroxy-4-[(2- morpholin-4-yl-1- phenylethyl)amino]- 1,8-naphthyridin- 2(1H)-one | 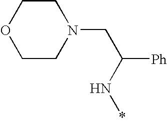 | High Resolution MS: m/z found 367.1752 (M + 1); calculated 367.1765 (M + 1) |
| 108 | 4-[(1-Benzylpiperidin- 4-yl)amino]-1-hydroxy- 1,8-naphthyridin- 2(1H)-one | 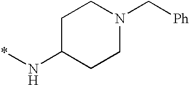 | High Resolution MS: m/z found 351.1807 (M + 1); calculated 351.1816 (M + 1) |
| 109 | Ethyl 4-[(1-hydroxy-2- oxo-1,2-dihydro-1,8- naphthyridin-4- yl)amino]piperidine-1- carboxylate | 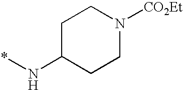 | High Resolution MS: m/z found 333.1550 (M + 1); calculated 333.1558 (M + 1) |
| 110 | 1-Hydroxy-4-[(2- morpholin-4-yl-2- phenylethyl)amino]- 1,8-naphthyridin- 2(1H)-one | 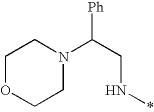 | High Resolution MS: m/z found 367.1764 (M + 1); calculated 367.1765 (M + 1) |
| 111 | 1-Hydroxy-4-{[4- (morpholin-4- ylmethyl)benzyl]amino}- 1,8-naphthyridin- 2(1H)-one | 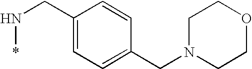 | High Resolution MS: m/z found 367.1754 (M + 1); calculated 367.1765 (M + 1) |
| 112 | 4-[(1-Benzylpyrrolidin- 3-yl)amino]-1-hydroxy- 1,8-naphthyridin- 2(1H)-one | 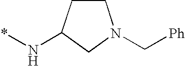 | High Resolution MS: m/z found 337.1645 (M + 1); calculated 337.1659 (M + 1) |
| 113 | 4-{[(3R)-1-Benzylpyrrolidin-3- yl]amino}-1-hydroxy- 1,8-naphthyridin- 2(1H)-one | 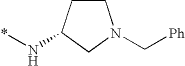 | High Resolution MS: m/z found 337.1651 (2M + 1); calculated 337.1659 (M + 1) |
| 114 | 4-{[(3S)-1- Benzylpyrrolidin-3- yl]amino}-1 -hydroxy- 1,8-naphthyridin- 2(1H)-one | 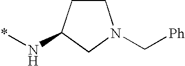 | High Resolution MS: m/z found 337.1648 (M + 1); calculated 637.1659 (M + 1) |
| 115 | 1-hydroxy-4-{[(6- morpholin-4- ylpyridin- 2-yl)methyl]amino}- 1,8-naphthyridin- 2(1H)-one | 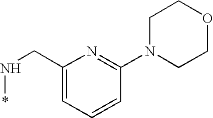 | High Resolution MS (FT- ICR): m/z found 354.1544 (M + 1); calculated 354.1561 (M + 1) |
| 116 | 1-hydroxy-4- {methyl[3-(1H)-1,2,4- triazol-1- ylmethyl)benzyl] amino}1,8-nahthyridin- 2(1H)-one | 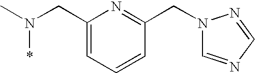 | High Resolution MS (FT- ICR): m/z found 363.1545 (M + 1); calculated 363.1564 (M + 1) |
| 117 | 4-[(2R)-2- (fluoromethyl)pyrrolidin- 1-yl]-1-hydroxy-1,8- naphthyridin-2(1H)-one | 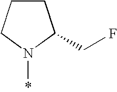 | Resolution MS (FT-ICR): m/z found 264.1134 (M + 1); calculated 264.1143 (M + 1) |
| 118 | 4-(3-fluoropiperidin-1- yl)-1-hydroxy-1,8- naphthyridin-2(1H)-one | 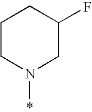 | High Resolution MS (FT- ICR): m/z found 264.1133 (M + 1); calculated 264.1143 (M + 1) |
| 119 | 4-(3,4- dihydroisoquinolin- 2(1H)-yl)-1-hydroxy- 1,8-naphthyridin- 2(1H)-one | 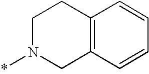 | High Resolution MS (FT- ICR): m/z found 294.3 (M + 1); calculated 293.328 (M + 1) |
| 120 | 2-(1-hydroxy-2-oxo- 1,2-dihydro-1,8- naphthyridin-4-yl)- 1,2,3,4- tetrahydroisoquinoline- 7-carbonitrile | 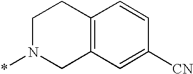 | High Resolution MS (FT- ICR): m/z found 319.3 (M + 1); calculated 318.338 (M + 1) |
| 121 | 4-[6- (methoxycarbonyl)-3,4- dihydroisoquinolin- 2(1H)-yl]-1-hydroxy- 1,8-naphthyridin- 2(1H)-one | 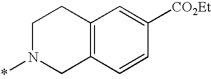 | High Resolution MS (FT- ICR): m/z found 352.3 (M + 1); calculated 351.365 (M + 1) |
| 122 | 4-[7- (methoxycarbonyl)-3,4- dihydroisoquinolin- 2(1H)-yl]-1-hydroxy- 1,8-naphthyridin- 2(1H)-one | 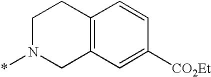 | High Resolution MS (FT- ICR): m/z found 352.4 (M + 1); calculated 351.365 (M + 1) |
| 123 | 4-(7,8-dihydro-1,6- naphthyridin-6(5H)-yl)- 1-hydroxy-1,8- naphthyridin-2(1H)-one | 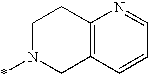 | High Resolution MS (FT- ICR): m/z found 296.3 (M + 1); calculated 295.324 (M + 1) |
| 124 | 1-hydroxy-4-{[(1S)-1- phenylethyl]amino}- 1,8-naphthyridin- 2(1H)-one | 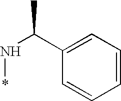 | High Resolution MS (FT- ICR): m/z found 282.4 (M + 1); calculated 281.317 (M + 1) |
| 125 | 1-hydroxy-4-{[(1R)-1- phenylethyl]amino}- 1,8-naphthyridin- 2(1H)-one | 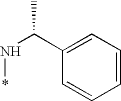 | High Resolution MS (FT- ICR): m/z found 282.4 (M + 1); calculated 281.317 (M + 1) |
| 126 | 4-(benzylamino)-1- hydroxy-1,8- naphthyridin-2(1H)-one | 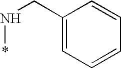 | ES MS: m/z = 268.1 (M + 1). |

TABLE 8
The following were made in a similar manner to Example 103 except that ethyl 1-
(benzyloxy)-4-hydroxy-2-oxo-1,2-dihydro-1,8-naphthyridine-3-carboxylate (Example 1,
Step 2) was used in place of 1-(benzyloxy)-4-hydroxy-1,8-naphthyridin-2(1H)-one in Step 1:

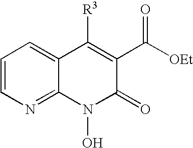

| Ex/cpd | Name | R³ | Data |
|---|---|---|---|
| 127 | Ethyl 4-anilino-1- hydroxy-2-oxo-1,2- dihydro-1,8- naphthyridine-3- carboxylate | 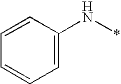 | Resolution MS: m/z found 326.1125 (M + 1); calculated 326.1135 (M + 1) |
| 128 | Ethyl 4-(benzylamino)- 1-hydroxy-2-oxo-1,2- dihydro-1,8- naphthyridine-3- carboxylate | 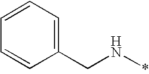 | High Resolution MS: m/z found 340.1285 (M + 1); calculated 340.1292 (M + 1) |
| 129 | Ethyl 1-hydroxy-4- {[(1R)-2-hydroxy-1- phenylethyl]amino}-2- oxo-1,2-dihydro-1,8- naphthyridine-3- carboxylate | 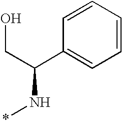 | High Resolution MS: m/z found 370.1391 (M + 1); calculated 370.1398 (M + 1) |

### Example 130

### 4-[benzyl(methyl)amino]-1-hydroxy-1,8-naphthyridin-2(1H)-one

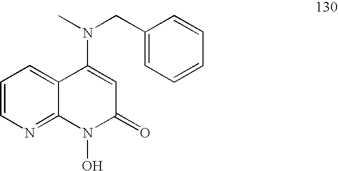

Step 1 was carried out in accordance with the procedures set forth in Example 103

### Step 2: 4-[benzyl (methyl)amino]-1-(benzyloxy)-1,8-naphthyridin-2(11)-one

The 1-(benzyloxy)-2-oxo-1,2-dihydro-1,8-naphthyridin-4-yl trifluoromethanesulfonate (70 mg, 0.175 mmol) and N-methylbenzylamine (0.5 ml, 3.87 mmol) were dissolved in DMF (0.5 ml). The solution was irradiated for 20 minutes. at 140° C. in a microwave tube. The residue was purified by RP-HPLC (C18 column; 5-100% CH₃CN/H₂O with 0.1%.TFA) to give the title compound. ES MS: m/z=282.1 (M+1)

### Step 3: 4-[benzyl(methyl)amino]-1-hydroxy-1,8-naphthyridin-2(11)-one

A mixture of the 4-[benzyl (methyl)amino]-1-(benzyloxy)-1,8-naphthyridin-2(1H)-one (20 mg, 0.054 mmol) in MeOH (4 ml) was evacuated and purged with N₂. Palladium hydroxide (7.56 mg, 0.054 mmol) was added to the reaction mixture. The mixture stirred at room temperature under 1 atm of H₂. After 1 hour, the solution was filtered through a pad of celite. The solvents were removed and the residue purified by RP-HPLC (C18 column; 5-100% CH₃CN/H₂O with 0.1% TFA) to give the title compound as a yellow solid. ¹H NMR (400 MHz, CD₃OD): δ 8.59 (s, 1H), 8.35 (s, 1H), 7.28 (m, 6H), 6.17 (s, 1H), 4.45 (s, 2H), 2.83 (s, 3H). ES MS: m/z=282.1 (M+1).

TABLE 9
The following were made in a similar manner to Example 103. Specifically, Step
1 was carried out in the same fashion and Steps 2 and 3 were carried in
accordance with Example 130 above.

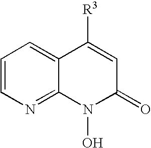

| Ex/cpd | Name | R³ | Data |
|---|---|---|---|
| 131 | 1-hydroxy-4-(4- phenylpiperazin-1-yl)- 1,8-naphthyridin- 2(1H)-one | 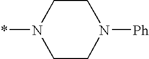 | ES MS: m/z = 323.2 (M + 1). |
| 132 | 1-hydroxy-4-[(2- phenylethyl)amino]- 1,8-naphthyridin- 2(1H)-one | 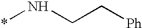 | ES MS: m/z = 283.0 (M + 1). |
| 133 | 4-(4-benzylpiperidin-1- yl)-1-hydroxy-1,8- naphthyridin-2(1H)-one | 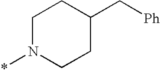 | ES MS: m/z = 336.2 (M + 1). |
| 134 | 4-(2,3-dihydro-1H- inden-1-ylamino)-1- hydroxy-1,8- naphthyridin-2(1H)-one | 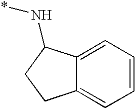 | ES MS: m/z = 294.1 (M + 1). |
| 135 | 1-hydroxy-4-(1,2,3,4- tetrahydronaphthalen-1- ylamino)-1,8- naphthyridin-2(1H)-one | 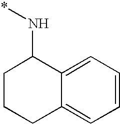 | ES MS: m/z = 308.2 (M + 1). |
| 136 | 4-(4-benzylpiperazin-1- yl)-1-hydroxy-1,8- naphthyridin-2(1H)-one | 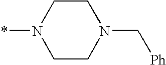 | ES MS: m/z = 337.2 (M + 1). |
| 137 | 1-hydroxy-4-[(2- pyridin-3- ylethyl)amino]-1,8- naphthyridin-2(1H)-one | 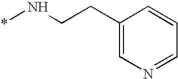 | ES MS: m/z = 284.0 (M + 1). |

### Example 138

### 1-hydroxy-4-[4-(4-morpholinyl)-1-piperidinyl]-1,8-naphthyridin-2(1H-one)

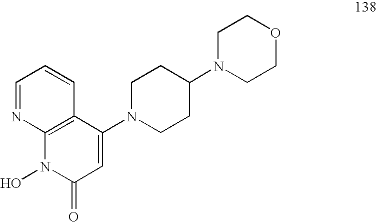

### Step 1: 1-(benzyloxy)-4-[4-(4-morpholinyl)-1-piperidinyl]-1,8-naphthyridin-2(1-H-one)

To a solution of 1-(benzyloxy)-4-hydroxy-1,8-naphthyridin-2(1H)-one (Example 2, Step 1; 60 mg, 0.150 mmol) was added 4-morpholinopiperidine (213 mg, 0.749 mmol). The reaction mixture was stirred in a microwave reactor at 120° C. for 25 minutes. The reaction was purified by RP-HPLC (C18 column; H₂O/CH₃CN with 0.1% TFA) to afford the title compound. ES MS: m/z 421 (M+1).

### Step 2: 1-hydroxy-4-[4-(4-morpholinyl)-1-piperidinyl]-1,8-naphthyridin-2(1-H-one)

1-(benzyloxy)-4-[4-(4-morpholinyl)-1-piperidinyl]-1,8-naphthyridin-2(1-H-one) (64 mg, 0.115 mmol) was dissolved in degassed MeOH and then Pd(OH)₂ was added and the reaction degassed again and then allowed to stir at room temperature for 30 minutes. At the end of 30 minutes, the reaction was degassed and then filtered through a pad of celite and washed with copious amounts of MeOH. The solution was concentrated and purified by RP-HPLC (C18 column; H₂O/CH₃CN with 0.1% TFA) to afford the title compound. High Resolution MS (FT-ICR): m/z found 331.1739 (M+1); calculated 331.1692 (M+1).

TABLE 10
The compounds in the following table were prepared in accordance with the
procedures set forth in Example 138:

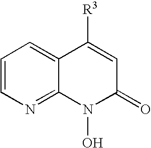

| Ex/cpd | Name | R³ | Data |
|---|---|---|---|
| 139 | 1-hydroxy-4-{[2-(1- piperidinyl)pheny] amino}-1,8-naphthyridin- 2(1H-one) | 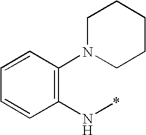 | High Resolution MS: m/z found 337.1665 (M + 1); calculated 337.1586 (M + 1) |
| 140 | 1-hydroxy-4-[(4- methoxybenzyl)amino]- 1,8-naphthyridin- 2(1H-one) | 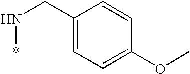 | High Resolution MS: m/z found 298.1200 (M + 1); calculated 298.1113 (M + 1) |
| 141 | 4-[(2- chlorobenzyl)amino]-1- hydroxy-1,8- naphthyridin- 2(1H-one) | 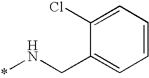 | Resolution MS: m/z found 302.0704 (M + 1); calculated 302.0618 (M + 1) |
| 142 | 1-hydroxy-4-[(2- methoxybenzyl)amino]- 1,8-naphthyridin- 2(1H-one) | 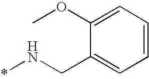 | High Resolution MS: m/z found 298.1200 (M + 1); calculated 298.1113 (M + 1) |
| 143 | 1-hydroxy-4-[(4- methylbenzyl)amino]- 1,8-naphthyridin- 2(1H-one) | 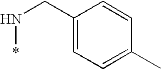 | High Resolution MS: m/z found 282.1164 (M + 1); calculated 282.1164 (M + 1) |
| 144 | 1-hydroxy-4-[3-(4- morpholinylmethyl)-1- piperidinyl]-1,8- naphthyridin- 2(1H-one) | 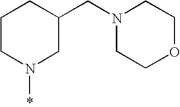 | High Resolution MS: m/z found 345.1924 (2M + 1); calculated 345.18481 (2M + 1) |
| 145 | 1-hydroxy-4-[(3- methoxybenzyl)amino]- 1,8-naphthyridin- 2(1H-one) | 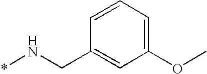 | LCMS: 298.0 (M + 1) |
| 146 | 4-[(3- fluorobenzyl)amino]-1- hydroxy-1,8- naphthyridin-2(1H- one) | 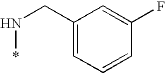 | High Resolution MS: m/z found 286.0961 (M + 1); calculated 286.0914 (M + 1) |
| 147 | 1-hydroxy-4-{[2-(4- morpholinyl)phenyl] amino}-1,8-naphthyridin- 2(1H-one) | 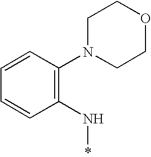 | High Resolution MS: m/z found 339.1456 (M + 1); calculated 339.1379 (M + 1) |
| 148 | 1-hydroxy-4-(4-methyl- 3-oxo-1-piperazinyl)- 1,8-naphthyridin- 2 (1H-one) | 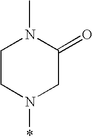 | High Resolution MS: m/z found 275.1134 (M + 1); calculated 275.1066 (M + 1) |
| 149 | 1-hydroxy-4-[(2- pyridinylmethyl)amino]- 1,8-naphthyridin- 2(1H-one) | 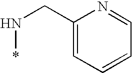 | High Resolution MS: m/z found 269.0956 (M + 1); calculated 269.0960 (M + 1) |
| 150 | 1-hydroxy-4-({3-[(4- methyl-1- piperazinyl)methyl] benzyl}amino)-1,8- naphthyridin-2(1H-one) | 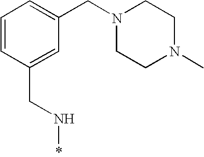 | High Resolution MS: m/z found 380.2070 (M + 1); calculated 380.2008 (M + 1) |
| 151 | 1-hydroxy-4-{[3-(4- morpholinylmethyl) benzyl]amino}-1,8- naphthyridin- 2(1H-one) | 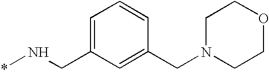 | High Resolution MS: m/z found 367.1753 (M + 1); calculated 367.1692 (M + 1) |
| 152 | 4-[(4- fluorobenzyl)amino]-1- hydroxy-1,8- naphthyridin-2(1H)- one) | 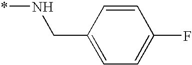 | High Resolution MS: m/z found 286.0979 (M + 1); calculated 286.0914 (M + 1) |
| 153 | 1-hydroxy-4-{[3-(1- piperidinylmethyl)benzyl amino}-1,8- naphthyridin-2(1H- one) | 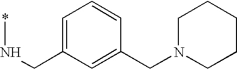 | High Resolution MS: m/z found 365.1961 (M + 1); calculated 365.1899 (M + 1) |
| 154 | 1-hydroxy-4-[(1- phenyl-4- piperidinyl)amino]-1,8- naphthyridin-2(1H-one) | 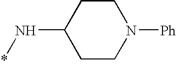 | High Resolution MS: m/z found 337.1651 (M + 1); calculated 337.1586 (M + 1) |
| 155 | 4-[(2- fluorobenzyl)amino]-1- hydroxy-1,8- naphthyridin-2(1H- one) | 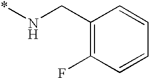 | High Resolution MS: m/z found 286.0980 (M + 1); calculated 286.0914 (M + 1) |
| 156 | 1-hydroxy-4-{4-[1-(4- morpholinyl)ethyl]-1- piperidinyl}-1,8- naphthyridin-2(1H- one) | 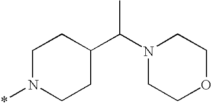 | High Resolution MS: m/z found 359.2067 (M + 1); calculated 359.2005 (M + 1) |
| 157 | 1-hydroxy-4-[(1- phenylethyl)amino]-1,8- naphthyridin-2(1H-one) | 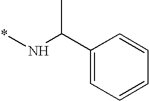 | High Resolution MS (FT-ICR): m/z found 282.1230 (M + 1); calculated 282.1164 (M + 1) |
| 158 | 4-{[3-(dimethylamino)- 1-phenylpropyl]amino}- 10hydroxy-1,8- naphthyridin-2(1H- one) | 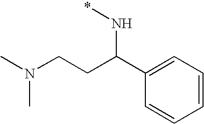 | High Resolution MS (FT-ICR): m/z found 339.1814 (M + 1); calculated 339.1743 (M + 1) |
| 159 | 4-{[3- (aminomethyl)benzyl] amino}-1-hydroxy-1,8- naphthyridin-2(1H- one) | 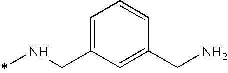 | High Resolution MS (FT-ICR): m/z found 297.1346 (M + 1); calculated 297.1273 (M + 1) |
| 160 | 1-hydroxy-4-[(4- pyridinylmethyl)amino]- 1,8-naphthyridin-2 (1H-one) | 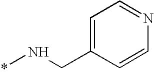 | High Resolution MS (FT-ICR): m/z found 269.1034 (M + 1); calculated 269.0960 (M + 1) |
| 161 | 1-hydroxy-4-{[(2-(4- morpholinyl)ethyl] amino}-1,8-naphthyridin- 2(1H-one) | 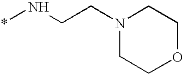 | High Resolution MS: m/z found 291.1451 (M + 1); calculated 291.1379 (M + 1) |
| 162 | 1-hydroxy-4-[(3- pyrindinylmethyl)amino]- 1,8-naphthyridin-2 (1H-one) | 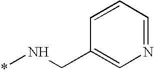 | High Resolution MS: m/z found 269.0625 (M + 1); calculated 269.0960 (M + 1) |
| 163 | 4-{[2- (aminomethyl)benzyl] amino}-1-hydroxy-1, 8-naphthyridin-2 (1H-one) | 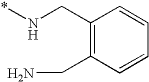 | High Resolution MS: m/z found 297.1346 (M + 1); calculated 297.1273 (M + 1) |
| 164 | ethyl[4-(1-hydroxy-2- oxo-1,2-dihydro-1,8- naphthyridin-4-yl)-1- piperazinyl]acetate | 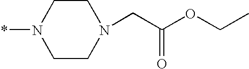 | High Resolution MS: m/z found 333.1563 (M + 1); calculated 333.1485 (M + 1) |
| 165 | 1-hydroxy-4-({3-[(4- methyl-1- piperidinyl)methyl] benzyl}amino)-1,8- naphthyridin-2 (1H-one) | 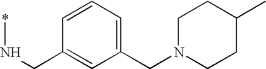 | High Resolution MS: m/z found 379.2116 (M + 1); calculated 379.2056 (M + 1) |
| 166 | 1-hydroxy-4-{[1- phenyl-2-(1- piperidinyl)ethyl]amino}- 1,8-naphthyridin-2 (1H-one) | 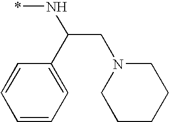 | High Resolution MS: m/z found 365.1959 (M + 1); calculated 365.1899 (M + 1) |
| 167 | 1-hydroxy-4-{[(2- phenyl-1-imidazole-5- yl)methyl]amino}-1,8- naphthyridin-2 (1H-one) | 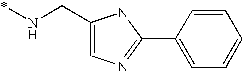 | High Resolution MS: m/z found 334.1226 (M + 1); calculated 334.1226 (M + 1) |

### Example 168

### (2S)-7-Hydroxy-2-phenyl-3,4-dihydro-1H-[1,4]diazepino[6,5-c]-1,8-naphthyridine-5,6(2H,7H)-dione and (3S)-7-Hydroxy-3-phenyl-3,4-dihydro-1H-[1,4]diazepino[6,5-c]-1,8-naphthyridine-5,6(2H,7H)-dione

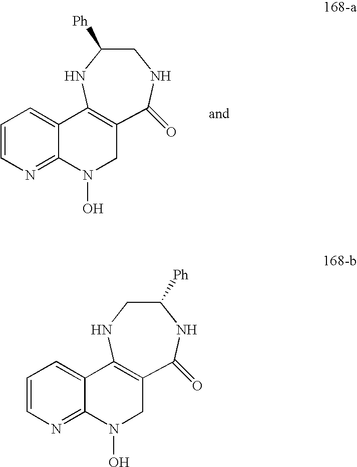

### Step 1: (2S)-7-(Benzyloxy)-2-phenyl-3,4-dihydro-1H-[1,4]diazepino[6,5-c]-1,8-naphthyridine-5,6(2H,7H)-dione and (3S)-7-(Benzyloxy)-3-phenyl-3,4-dihydro-1H-[1,4]diazepino[6,5-c]-1,8-naphthyridine-5,6(2H,7H)-dione

A mixture of 1-(benzyloxy)-2-oxo-1,2-dihydro-1,8-naphthyridin-4-yl trifluoromethanesulfonate (Example 103, Step 1; 50 mg, 0.11 mmol) and (1S)-1-phenylethane-1,2-diamine (50 mg, 0.37 mmol) in DMF (2 mL) was heated in a microwave at 140° C. for 45 minutes, then at 150° C. for 90 minutes. The crude reaction mixture was purified by RP-HPLC (C18 column; 5-95% CH₃CN/H₂O with 0.1% TFA) to afford a mixture of the title compounds. ES MS: m/z=413 (M+1).

### Step 2: (2S)-7-Hydroxy-2-phenyl-3,4-dihydro-1H-[1,4]diazepino[6,5-c]-1,8-naphthyridine-5,6(2H,7H)-dione and (3S)-7-Hydroxy-3-phenyl-3,4-dihydro-1H-[1,4]diazepino[6,5-c]-1,8-naphthyridine-5,6(2H,7H)-dione

A mixture of (2S)-7-(benzyloxy)-2-phenyl-3,4-dihydro-1H-[1,4]diazepino[6,5-c]-1,8-naphthyridine-5,6(2H,7H)-dione and (3S)-7-(benzyloxy)-3-phenyl-3,4-dihydro-1H-[1,4]diazepino[6,5-c]-1,8-naphthyridine-5,6(2H,7H)-dione from the previous step (25 mg, 0.06 mmol) in 33% HBr—HOAc (1 mL, 0.06 mmol) and H₂O (0.3 mL) was heated at 80° C. for 1 hour. The solvents were removed and the residue purified by RP-HPLC (C18 column; 100-80% H₂O/CH₃CN with 0.1% TFA) to afford the title compounds: (2S)-7-hydroxy-2-phenyl-3,4-dihydro-1H-[1,4]diazepino[6,5-c]-1,8-naphthyridine-5,6(2H,71)-dione as a yellow solid (5 mg): ¹H NMR (600 MHz, d₆-DMSO, ppm): δ 8.65 (m, 1H), 8.55 (m, 1H), 8.00 (br s, 1H), 7.65 (br s, 1H), 7.39-7.37 (m, 2H), 7.32-7.29 (m, 4H), 5.00 (br s, 1H), 3.63-3.60 (m, 1H), 3.55-3.50 (m, 1H). ES MS: t/z=323.3 (M+1) and (3S)-7-hydroxy-3-phenyl-3,4-dihydro-1H-[1,4]diazepino[6,5-c]-1,8-naphtyridine-5,6(2H,7H)-dione: ¹H NMR (600 MHz, d₆-D MSO): δ 11.06 (br signal, 1H), 9.62 (br s, 1H), 8.84 (br s, 1H), 8.74 (m, 1H), 8.47 (m, 1H), 7.42-7.26 (m, 6H), 5.05 (br s, 1H), 4.15-4.11 (m, 1H), and 3.78-3.75 (m, 1H). ES MS: m/z=323.3 (M+1).

TABLE 11
The compounds in the following table were prepared in accordance with the
procedures set forth in Example 168:

| Ex/ | | | |
|---|---|---|---|
| cpd | Name | Structure | Data |
| 169 | (8aS, 12aS)-5-hydroxy- 8,8a,9,10,11,12,12a,13- octahydro-5H- [1,8]naphthyridino[4,3- b][1,5]benzodiazepine- 6,7-dione | 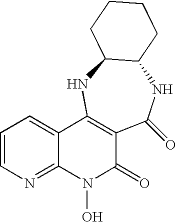 | High Resolution MS: m/z found 301.1294 (M + 1); calculated 301.1295 (M + 1) |
| 170 | (2S, 3R)-7-hydroxy- 2,3-diphenyl-3,4- dihydro-1H- [1,4]diazepino[6,5-c]- 1,8-naphthyridine- 5,6(2H, 7H)-dione | 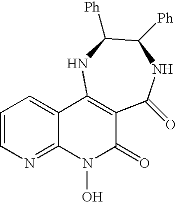 | High Resolution MS: m/z found 399.1450 (M + 1); calculated 399.1452 (M + 1) |

### Example 171

### 1-Hydroxy-4-phenyl-1,8-naphthyridin-2(H)-one

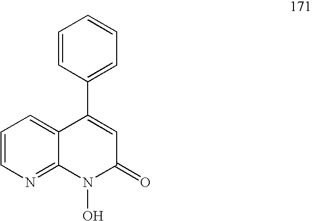

A mixture of 1-(benzyloxy)-2-oxo-1,2-dihydro-1,8-naphthyridin-4-yl trifluoromethanesulfonate (Example 103, Step 1; 40 mg, 0.10 mmol), phenylboronic acid (14.6 mg, 0.12 mmol), sodium carbonate (21 mg, 0.20 mmol) and Pd(PPh₃)₄ (5.8 mg, 0.005 mmol) in dioxane (3 mL) was heated to 100° C. overnight. Loss of the benzyl protecting group from the initially formed 1-benzyloxy-4-phenyl-1,8-napthyridin-2-(1H)-one (observed by LCMS) was noted after overnight heating, and the solvent had evaporated. The residue was diluted with MeOH and purified by RP-HPLC (C18 column; 95:5 to 5:95 H₂O:CH₃CN with 0.1% TFA), followed by a second RP-HPLC purification (85:15 H₂O:CH₃CN with 0.1% TFA) to give the title compound as a yellow solid. High Resolution MS (FT-ICR): m/z found 239.0815 (M+1); calculated 239.0815 (M+1).

TABLE 12
The compounds in the following table were prepared in accordance with the
procedures set forth in Example 171:

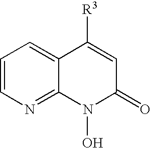

| Ex/ | | | |
|---|---|---|---|
| cpd | Name | R³ | Data |
| 172 | 4-[3- (aminomethyl)phenyl]- 1-hydroxy-1,8- naphthyridin-2(1H)-one | 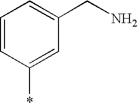 | ES MS: m/z = 267.9 (M + 1). |
| 173 | 1-hydroxy-4-(2- naphthyl)-1,8- naphthyridin-2(1H)-one | 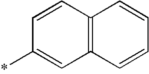 | ES MS: m/z = 288.9 (M + 1). |
| 174 | 1-hydroxy-4-[4-(1- morpholin-4- ylethyl)phenyl]-1,8- naphthyridin-2(1H)-one | 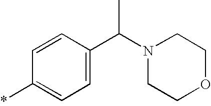 | ES MS: m/z = 352.0 (M + 1). |
| 175 | N-[3-(1-hydroxy-2- oxo-1,2-dihydro-1,8- naphthyridin-4- yl)phenyl]methanesulfo- namide | 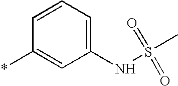 | ES MS: m/z = 332.1 (M + 1). |
| 176 | 4-[3-(3,5-dimethyl- 1H)-pyrazol-1- yl)phenyl]-1-hydroxy- 1,8-naphthyridin- 2(1H)-one | 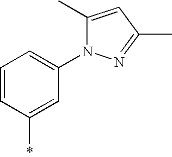 | ES MS: m/z = 333.3 (M + 1). |
| 177 | N-[3′-(1-hydroxy-2- oxo-1,2-dihydro-1,8- naphthyridin-4- yl)biphenyl-3- yl]methanesulfonamide | 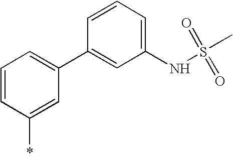 | ES MS: m/z = 408.1 (M + 1). |
| 178 | 1-hydroxy-4-[4′-(1- morpholin-4- ylethyl)biphenyl-3-yl]- 1,8-naphthyridin- 2(1H)-one | 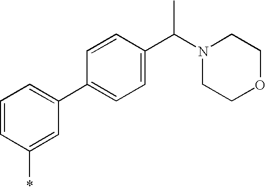 | High Resolution MS: m/z found 428.1967 (M + 1); calculated 427.1896 (M + 1) |
| 179 | 1-hydroxy-4-[3- (morpholin-4- ylcarbonyl)phenyl]-1,8- naphthyridin-2(1H)-one | 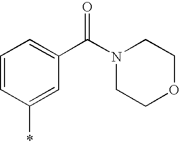 | High Resolution MS: m/z found 352.1286 (M + 1); calculated 351.1219 (M + 1) |
| 180 | 3-(1-hydroxy-2-oxo- 1,2-dihydro-1,8- naphthyridin-4-yl)-N- methylbenzamide | 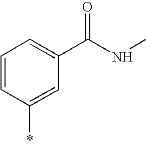 | High Resolution MS: m/z found 296.1 (M + 1); calculated 295.0957 (M + 1) |
| 181 | 3-(1-hydroxy-2-oxo- 1,2-dihydro-1,8- naphthyridin-4-yl)- N,N- dimethylbenzamide | 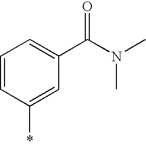 | High Resolution MS: m/z found 310.1177 (M + 1); calculated 309.1113 (M + 1) |
| 182 | N-(tert)-butyl)-3-(1- hydroxy-2-oxo-1,2- dihydro-1,8- naphthyridin-4- yl)benzamide | 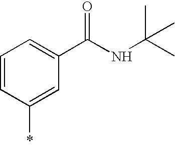 | High Resolution MS: m/z found 338.1489 (M + 1); calculated 337.1426 (M + 1) |
| 183 | 1-hydroxy-4-[3- (hydroxymethyl)phenyl]- 1,8-naphthyridin- 2(1H)-one | 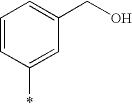 | High Resolution MS: m/z found 269.0914 (M + 1); calculated 268.0848 (M + 1) |
| 184 | 1-hydroxy-4-quinolin- 6-yl-1,8-naphthyridin- 2(1H)-one | 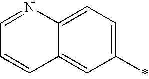 | High Resolution MS: m/z found 290.0914 (M + 1); calculated 289.0851 (M + 1) |
| 185 | 1-hydroxy-4-(2- methoxy-5-pyridin-4- ylphenyl)-1,8- naphthyridin-2(1H)-one | 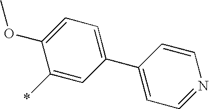 | High Resolution MS: m/z found 346.1179 (M + 1); calculated 345.1113 (M + 1) |
| 186 | 1-hydroxy-4-(1H-indol- 6-yl)-1,8-naphthyridin- 2(1H)-one | 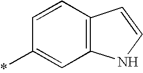 | High Resolution MS: m/z found 278.0916 (M + 1); calculated 277.0851 (M + 1) |
| 187 | methyl-3-[3′-(1- hydroxy-2-oxo-1,2- dihydro-1,8- naphthyridin-4- yl)biphenyl-3-yl]- propanoate | 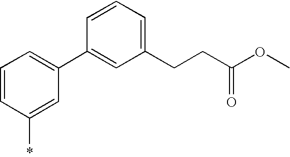 | ES MS: m/z = 401.1 (M + 1). |
| 188 | 1-hydroxy-4-[4-(1H- pyrazol-5-yl)phenyl]- 1,8-naphthyridin- 2(1H)-one | 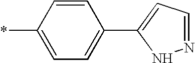 | High Resolution MS: m/z found 305.1007 (M + 1); calculated 304.0960 (M + 1) |
| 189 | N-[4-(1-hydroxy-2- oxo-1,2-dihydro-1,8- naphthyridin-4- yl)benzyl]methanesulfo- namide | 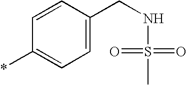 | High Resolution MS: m/z found 346.0831 (M + 1); calculated 345.0783 (M + 1) |
| 190 | 4-{5-[3- (aminomethyl)phenyl] pyridin-3-yl}-1-hydroxy- 1,8-naphthyridin- 2(1H)one | 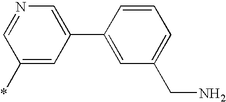 | ES MS: m/z = 345.3 (M + 1). |
| 191 | 4-(3′-acetylbiphenyl-3- yl)-1-hydroxy-1,8- naphthyridin-2(1H)-one | 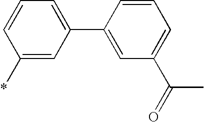 | High Resolution MS: m/z found 357.1229 (M + 1); calculated 356.1161 (M + 1) |
| 192 | 3-(1-hydroxy-2-oxo- 1,2-dihydro-1,8- naphthyridin-4- yl)benzenesulfonamide | 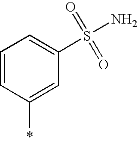 | High Resolution MS: m/z found 318.0543 (M + 1); calculated 317.0470 (M + 1) |
| 193 | 3-(1-hydroxy-2-oxo- 1,2-dihydro-1,8- naphthyridin-4-yl)-N- methylbenzenesulfona- mide | 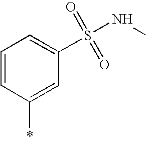 | High Resolution MS: m/z found 332.0717 (M + 1); calculated 331.0627 (M + 1) |
| 194 | N-[3-(1-hydroxy-2- oxo-1,2-dihydro-1,8- naphthyridin-4- yl)phenyl]acetamide | 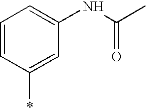 | High Resolution MS: m/z found 296.1040 (M + 1); calculated 295.0957 (M + 1) |
| 195 | 1-hydroxy-4-[3- (pyrrolidin-1- ylcarbonyl)phenyl]-1,8- naphthyridin-2(1H)-one | 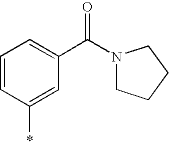 | High Resolution MS: m/z found 336.1375 (M + 1); calculated 335.1270 (M + 1) |
| 196 | 3-(1-hydroxy-2-oxo- 1,2-dihydro-1,8- naphthyridin-4- yl)benzamide | 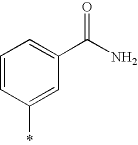 | High Resolution MS: m/z found 282.0869 (M + 1); calculated 281.0800 (M + 1) |
| 197 | 1-hydroxy-4-pyrimidin- 5-yl-1,8-naphthyridin- 2(1H)-one | 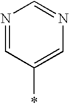 | High Resolution MS: m/z found 241.0717 (M + 1); calculated 240.0647 (M + 1) |
| 198 | 1-hydroxy-4-(1H- pyrazol-5-yl)-1,8- naphthyridin-2(1H)-one | 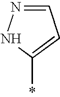 | High Resolution MS: m/z found 229.0717 (M + 1); calculated 228.0647 (M + 1) |
| 199 | 1-hydroxy-4-[3- (methylsulfonyl)phenyl]- 1,8-naphthyridin- 2(1H)-one | 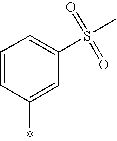 | High Resolution MS: m/z found 317.0587 (M + 1); calculated 316.0518 (M + 1) |
| 200 | 1-hydroxy-4-{6-[(2- morpholin-4- ylethyl)amino]pyridin- 3-yl}-1,8-naphthyridin- 2(1H)-one | 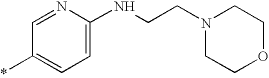 | High Resolution MS: m/z found 368.1711 (M + 1); calculated 367.1644 (M + 1) |
| 201 | N-[2-(1-hydroxy-2- oxo-1,2-dihydro-1,8- naphthyridin-4- yl)phenyl]methanesulfo- namide | 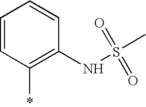 | High Resolution MS: m/z found 332.0693 (M + 1); calculated 331.0627 (M + 1) |
| 202 | 3′-(1-hydroxy-2-oxo- 1,2-dihydro-1,8- naphthyridin-4- yl)biphenyl-3- carbonitrile | 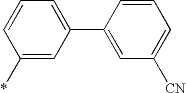 | High Resolution MS: m/z found 340.1082 (M + 1); calculated 339.1008 (M + 1) |
| 203 | 1-hydroxy-4-pyridin-4- yl-1,8-naphthyridin- 2(1H)-one | 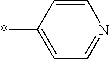 | ES MS: m/z = 239.9 (M + 1). |
| 204 | 4-[3′- (aminomethyl)biphenyl- 3-yl]-1-hydroxy-1,8- naphthyridin-2(1H)-one | 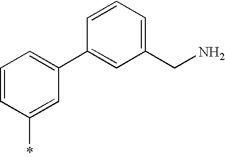 | ES MS: m/z = 344.2 (M + 1). |

### Example 205

### 4-{3′-[(benzylamino)methyl]biphenyl-3-yl}-1-hydroxy-1,8-naphthyridin-2(H)-one

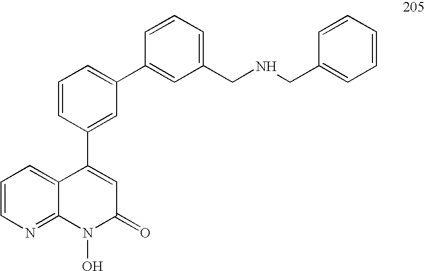

The above compound, Example 205, was prepared in accordance with the procedures set forth in Example 171 (Step 1) with an additional Step 2

### Step 2: 4-{3′-[(benzylamino)methyl]biphenyl-3-yl}-1-hydroxy-1,8-naphthyridin-2(H)-one

To a solution of the aldehyde (50 mg, 0.116 mmol) in anhydrous THF (5 ml) was added TEA (0.097 ml, 0.694 mmol) and the benzylamine (0.038 ml, 0.347 mmol). After stirring at room temperature for 1 hour, sodium triacetoxyborohydride (73.5 mg, 0.347 mmol) and HOAc (0.013 ml, 0.231 mmol) were added to the mixture. After 1 hour, the solvents were removed and the residue was purified by RP-HPLC (C18 column; 5-100% CH₃CN/H₂O with 0.1% TFA) to give the title compound as a white solid. ¹H NMR (500 MHz, CDCl₃): δ 8.71 (s, 1H), 7.88 (d, J=7.5, 1H), 7.74 (d, J=7.2H), 7.65 (m, 2H), 7.53 (m, 3H), 7.40 (m, 5H), 7.19 (m, 1H), 6.74 (s, 1H), 3.92 (s, 2H), 3.87 (s, 2H). ES MS: m/z=524.2 (M+1).

### Example 206

### 4-{3-[(4-benzyl-1-piperazinyl)methyl]phenyl}-1-hydroxy-1,8-naphthyridin-2(1H-one)

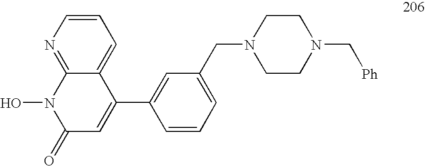

### Step 1: 3-[1-(benzyloxy)-2-oxo-1,2-dihydro-1,8-naphthyridin-4-yl]benzaldehyde

A mixture of 1-(benzyloxy)-2-oxo-1,2-dihydro-1,8-naphthyridin-4-yl trifluoromethanesulfonate (Example 103, Step 1; 40 mg, 0.10 mmol), 3-formylphenylboronic acid (14.6 mg, 0.12 mmol), sodium carbonate (21 mg, 0.20 mmol) and Pd(PPh₃)₄ (5.8 mg, 0.005 mmol) in dioxane (3 mL) was heated to 120° C. for 20 minutes in a sealed microwave vial. The reaction was then purified by RP-HPLC (C18 column; 5:95 H₂O:CH₃CN with 0.1% TFA) to give the title compound as a yellow solid. ES MS: m/z=357 (M+1).

### Step 2: 1-(benzyloxy)-4-{3-[(4-benzyl-1-piperazinyl)methyl]phenyl}-1,8-naphthyridin-2(1H)-one

A mixture of 3-(1-hydroxy-2-oxo-1,2-dihydro-1,8-naphthyridin-4-yl)benzaldehyde (70 mg, 0.196 mmol), 1-benzylpiperazine (45 mg, 0.255 mmol), sodium triacetoxyborohydride (210 mg, 0.590 mmol), and HOAc (25 uL, 0.395 mmol) was heated to 130° C. for 10 minutes in a microwave. The reaction was mixture was then concentrated and taken on to the step 3 without any purification. ES MS: t/z=517 (M+1).

### Step 3: 4-{3-[(4-benzyl-1-piperazinyl)methyl]phenyl}-1-hydroxy-1,8-naphthyridin-2(1H -one)

A mixture of 1-(benzyloxy)-4-{3-[(4-benzyl-1-piperazinyl)methyl]phenyl}-1,8-naphthyridin-2(1H)-one (100 mg, 0.213 mmol), 33% HBr in AcOH (2.5 mL), and H₂O (0.5 mL) was heated for 10 minutes at 100° C. in a microwave. The reaction was then purified by RP-HPLC (C18 column; 95:5 to 5:95 H₂O:CH₃CN with 0.1% TFA) to give the title compound as a yellow solid. High Resolution MS (FT-ICR): m/z found 427.2127 (M+1); calculated 427.2056 (M+1).

TABLE 13
The compounds in the following table were prepared in accordance with the
procedures set forth in Example 206:

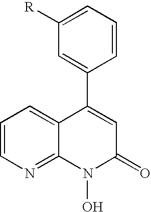

| Ex/ | | | |
|---|---|---|---|
| cpd | Name | R | Data |
| 207 | 4-[3-({4-[(4- chlorophenyl)(phenyl) methyl]-1- piperazinyl}methyl) phenyl-1-hydroxy-1,8- naphthyridin-2(1H- one) | 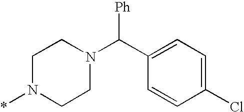 | High Resolution MS (FT-ICR): m/z found 537.2047 (M + 1); calculated 537.1979 (M + 1) |
| 208 | 1-hydroxy-4-[3-({4-[2- oxo-2-(1- pyrrolidinyl)ethyl]-1- piperazinyl}methyl) phenyl]-1,8-naphthyridin- 2(1H-one) | 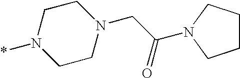 | High Resolution MS (FT-ICR): m/z found 448.2336 (M + 1); calculated 448.2770 (M + 1) |
| 209 | 2-{[3-(1-hydroxy-2- oxo-1,2-dihydro-1,8- naphthyridin-4- yl)benzyl]amino}-N,N- dimethylacetamide | 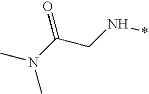 | High Resolution MS (FT-ICR): m/z found 353.1599 (M + 1); calculated 353.1535 (M + 1) |
| 210 | 1-hydroxy-4-(3-{[4-(1- phenylethyl)-1- piperazinyl]methyl} phenyl)-1,8-naphthyridin- 2(1H-one) | 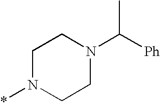 | High Resolution MS (FT-ICR): m/z found 441.2284 (M + 1); calculated 441.2212 (M + 1) |
| 211 | 1-hydroxy-4-[3-({4-[2- (4-morpholinyl)-2- oxoethyl]-1- piperazinyl}methyl) phenyl]-1,8- naphthyridin-2(1H- one) | 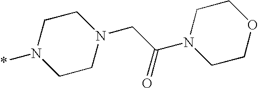 | High Resolution MS: m/z found 464.2284 (M + 1); calculated 464.2220 (M + 1) |
| 212 | 4-[3-({4-[3- (dimethylamino)propyl]-1- piperazinyl}methyl) phenyl]-1-hydroxy-1,8- naphthyridin-2(1H- one) | 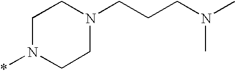 | High Resolution MS: m/z found 422.2541 (M + 1); calculated 422.2478 (M + 1) |
| 213 | 4-{3-[(4-acetyl-1- piperazinyl)methyl] phenyl}-1-hydroxy-1,8- naphthyridin-2(1H-one) | 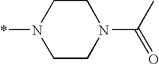 | High Resolution MS: m/z found 379.1750 (M + 1); calculated 379.1692 (M + 1) |

### Example 214

### 1,4-Dihydroxy-2-oxo-N-phenyl-1,2-dihydro-1,8-naphthyridine-3-carboxamide

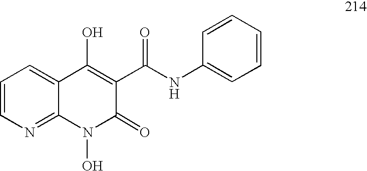

### Step 1: 1-(Benzyloxy)-4-hydroxy-2-oxo-N-phenyl-1,2-dihydro-1,8-naphthyridine-3-carboxamide

To a solution of 1-(benzyloxy)-4-hydroxy-1,8-naphthyridin-2(1R)-one (Example 2, Step 1; 30 mg, 0.11 mmol) in nitrobenzene (0.4 mL) were added phenyl isocyanate (18 μL, 0.17 mmol) and TEA (16 μL, 0.111 mmol). The reaction mixture was stirred in a microwave reactor at 160° C. for 3 hours. The reaction was purified by RP-HPLC (C18 column; H₂O/CH₃CN with 0.1% TFA) to afford the title compound. ES MS: m/z=388 (M+1).

### Step 2: 1,4-Dihydroxy-2-oxo-N-phenyl-1,2-dihydro-1,8-naphthyridine-3-carboxamide

1-(Benzyloxy)-4-hydroxy-2-oxo-N-phenyl-1,2-dihydro-1,8-naphthyridine-3-carboxamide (23 mg, 0.06 mmol) was heated at 85° C. for 1 hour in 33% HBr/HOAc (2 mL). The solution was concentrated and purified by RP-HPLC (C18 column; H₂O/CH₃CN with 0.1% TFA) to afford the title compound. ¹H NMR (400 MHz, d6-DMSO, ppm): δ 12.2 (bs, 1H), 8.80 (d, J=3.8 Hz, 1H), 8.62 (d, J=7.7 Hz, 1H), 7.69 (d, J=7.6 Hz, 2H), 7.47 (dd, J=7.5 and 4.7 Hz, 1H), 7.39 (t, J=7.2 Hz, 2H) and 7.18 (t, J=7.1 Hz, 3H). High Resolution MS (FT-ICR): m/z found 298.0848 (M+1); calculated 298.0823 (M+1).

### Example 215

### 1,4-Dihydroxy-N-methyl-2-oxo-N-pyrrolidin-3-yl-1,2-dihydro-1,8-naphthyridine-3-carboxamide

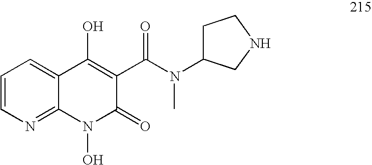

### Step 1: 1,4-Dihydroxy-N-methyl-2-oxo-N-pyrrolidin-3-yl-1,2-dihydro-1,8-naphthyridine-3-carboxamide

tert-Butyl 3-[[(1,4-dihydroxy-2-oxo-1,2-dihydro-1,8-naphthyridin-3-yl)carbonyl](methyl)amino]pyrrolidine-1-carboxylate (Table 1, Cmpd 10; 25 mg, 0.062 mmol) was dissolved in DCM (2 ml) and TFA (0.048 ml, 0.618 mmol) was added. The reaction was stirred overnight at room temperature. The solvent was removed and the residue was purified by RP-HPLC (C18 column; H₂O/CH₃CN with 0.1% TFA) to afford the title compound as the TFA salt. ¹H NMR (400 MHz, d₆-DMSO, ppm): 810.3 (bs, 1H), 8.85 (dd, J=4.6 and 1.8 Hz, 1H), 8.71 (bs, 2H), 8.47 (dd, J=8.0 and 1.8 Hz, 1H), 7.45 (dd, J=8.0 and 4.6 Hz, 1H), 3.49 (m, 3H), 3.28 (m, 2H), 3.15 (m, 1H), 2.94 (m, 1H), 2.61 (m, 1H), 2.05 (m, 1H) and 1.69 (m, 1H). High Resolution MS (FT-ICR): m/z found 305.1247 (M+1); calculated 305.1245 (M+1).

### Example 216

### 6-Hydroxy-3-methyl-2-phenyl-2,3-dihydropyrimido[5,4-c]-1,8-naphthyridine-4,5(1H, 6H)-dione

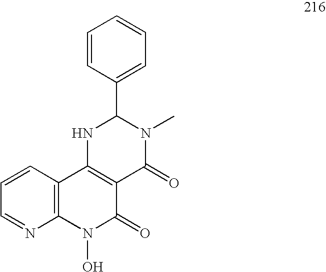

### Step 1: Sodium 4-amino-1-(benzyloxy)-2-oxo-1,2-dihydro-1,8-naphthyridine-3-carboxylate

1N NaOH (5.89 ml, 5.89 mmol) was added to ethyl 4-amino-1-(benzyloxy)-2-oxo-1,2-dihydro-1,8-naphthyridine-3-carboxylate (Example 97, Step 3; 1 g, 2.95 mmol) in EtOH (20 ml) and the solution was heated at 50° C. for 3 hours. The reaction was cooled and the solids were collected to afford the title compound. ¹H NMR (400 MHz, d₆-DMSO, ppm): δ 8.8 (bs, 2H), 8.64 (dd, J=4.7 and 1.3 Hz, 1H), 8.51 (dd, J=8.0 and 1.3 Hz, 1H), 7.68 (m, 2H), 7.44-7.38 (m, 3H), 7.30 (dd, J=8.0 and 4.7 Hz, 1H) and 5.10 (s, 2H). ES MS: m/z=312 (M+1).

### Step 2: 4-Amino-1-(benzyloxy)-N-methyl-2-oxo-1,2-dihydro-1,8-naphthyridine-3-carboxamide

Sodium 4-amino-1-(benzyloxy)-2-oxo-1,2-dihydro-1,8-naphthyridine-3-carboxylate (50 mg, 0.15 mmol), BOP reagent (133 mg, 0.30 mmol), and 2M methylamine in THF (0.150 ml, 0.30 mmol) were combined in DMF (1 ml) at room temperature. The reaction was stirred overnight at room temperature The reaction was partitioned between aqueous sodium hydrogen carbonate and DCM. The layers were separated and the product was extracted from the aqueous. layer twice more with DCM. The combined organic extracts were dried, filtered and concentrated to afford the title compound. ¹H NMR (400 MHz, d₆-DMSO, ppm): δ 10.9 (bs, 1H), 9.98 (d, J=4.6 Hz, 1H), 8.79 (d, J=4.6 Hz, 1H), 8.70 (d, J=8.0 Hz, 1H), 8.40 (bs, 1H), 7.66 (m, 2H), 7.56-7.40 (m, 4H), 5.14 (s, 2H) and 2.83 (d, J=4.6 Hz, 3H). ES MS: m/z=325 (M+1).

### Step 3: 6-(Benzyloxy)-3-methyl-2-phenyl-2,3-dihydropyrimido[5,4-c]-1,8-naphthyridine-4,5(1H, 6H)-dione

4-Amino-1-(benzyloxy)-N-methyl-2-oxo-1,2-dihydro-1,8-naphthyridine-3-carboxamide (23 mg, 0.071 mmol), benzaldehyde (65 μl, 0.43 mmol), and toluenesulfonic acid (13 mg, 0.071 mmol) were combined in benzene (2 mL) and heated to 80° C. for 2 hours. The solvent was removed and the residue was purified by RP-HPLC (C18 column; H₂O/CH₃CN with 0.1% TFA) to afford the title. ES MS: m/z=413 (M+1).

### Step 4: 6-Hydroxy-3-methyl-2-phenyl-2,3-dihydropyrimido[5,4-c]-1,8-naphthyridine-4,5(1H, 6H)-dione

To a solution of 6-(benzyloxy)-3-methyl-2-phenyl-2,3-dihydropyrimido[5,4-c]-1,8-naphthyridine-4,5(1H, 6H)-dione (16 mg, 39 μmol) was heated at 85° C. for 2 hours in 33% HBr/HOAc (1.5 mL) and H₂O (0.5 mL). The solution was concentrated and purified by RP-HPLC (C18 column; H₂O/CH₃CN with 0.1% TFA) to afford the title compound. ¹H NMR (400 MHz, d₆-DMSO, ppm): 9.25 (d, J=3.7 Hz, 1H), 8.65 (d, J=4.0 Hz, 1H), 8.51 (d, J=7.9 Hz, 1H), 7.41-7.33 (m, 5H), 7.29 (dd, J=7.9 and 4.7 Hz, 1H), 6.05 (d, J=3.9 Hz, 1H) and 2.94 (s, 3H). High Resolution MS (FT-ICR): m/z found 323.1132 (M+1); calculated 323.1139 (M+1).

TABLE 14
The compounds in the following table were prepared in accordance with the
procedures set forth in Example 216:

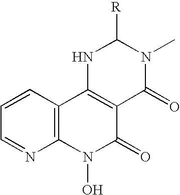

| Ex/ | | | |
|---|---|---|---|
| cpd | Name | R | Data |
| 217 | 2-Biphenyl-2-yl-6- hydroxy-3-methyl-2,3- dihydropyrimido[5,4- c]-1,8-naphthyridine- 4,5(1H, 6H)dione | 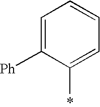 | High Resolution MS (FT-ICR): m/z found 399.1447 (M + 1); calculated 399.1452 (M + 1) |
| 218 | 6-Hydroxy-3-methyl-2- (3-phenoxyphenyl)-2,3- dihydropyrimido[5,4- c]-1,8-naphthyridine- 4,5(1H, 6H)dione | 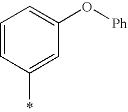 | High Resolution MS (FT-ICR): m/z found 415.1399 (M + 1); calculated 415.1401 (M + 1) |
| 219 | 6-Hydroxy-3-methyl-2- [3-(morpholin-4- ylmethyl)phenyl]-2,3- dihydropyrimido[5,4- c]-1,8-naphthyridine- 4,5(1H, 6H)dione | 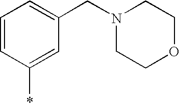 | High Resolution MS (FT-ICR): m/z found 422.1826 (M + 1); calculated 422.1823 (M + 1) |
| 220 | 2-[3- (Aminomethyl)phenyl]- 6-hydroxy-3-methyl- 2,3- dihydropyrimido[5,4- c]-1,8-naphthyridine- 4,5(1H, 6H)dione | 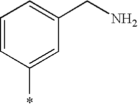 | High Resolution MS (FT-ICR): m/z found 352.1387 (M + 1); calculated 352.1404 (M + 1) |

### Example 221

### 4-Amino-1-hydroxy-N-methyl-2-oxo-1,2-dihydro-1,8-naphthyridine-3-carboxamide

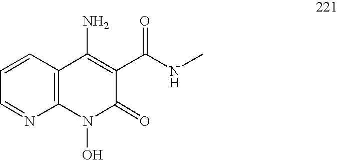

### Step 1: 4-Amino-1-hydroxy-N-methyl-2-oxo-1,2-dihydro-1,8-naphthyridine-3-carboxamide

To a solution of 4-amino-1-(benzyloxy)-N-methyl-2-oxo-1,2-dihydro-1,8-naphthyridine-3-carboxamide (Example 216, Step 2; 175 mg, 0.54 mmol) was heated at 85° C. for 2 hours in 33% HBr/HOAc (2.5 mL) and H₂O (0.5 mL). The residue was triturated with MeOH and the solids were collected by vacuum filtration to give the title compound as the HBr salt. ¹H NMR (400 MHz, d₆-DMSO, ppm): 10.1 (bs, 1H), 8.72-8.68 (m, 2H), 7.38 (dd, J=8.0 and 4.6 Hz, 1H), 6.6 (vbs, 3H) and 2.81 (s, 3H). High Resolution MS (FT-ICR): m/z found 235.0833 (M+1); calculated 235.0826 (M+1).

### Example 222

### 2-[2-(Benzyloxy)phenyl]-6-hydroxy-2,3-dihydropyrimido[5,4-c]-1,8-naphthyridine-4,5(1H, 6H)dione

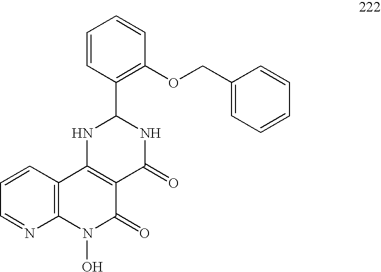

### Step 1: 2-[2-(Benzyloxy)phenyl]-6-hydroxy-2,3-dihydropyrimido[5,4-c]-1,8-naphthyridine-4,5 (1H, 6H)dione

4-Amino-1-hydroxy-N-methyl-2-oxo-1,2-dihydro-1,8-naphthyridine-3-carboxamide (Example 221, Step 1; 25 mg, 0.079 mmol) was heated overnight at 80° C. with 2-benzyloxybenzaldehyde (0.044 ml, 0.278 mmol) and toluenesulfonic acid (45.3 mg, 0.238 mmol) in a solution of benzene (2 ml) and DMF (0.5 ml). The solvent was removed and the residue was purified by RP-HPLC (C18 column; H₂O/CH₃CN with 0.1% TFA) to afford the title compound. High Resolution MS (FT-ICR): m/z found 429.1540 (M+1); calculated 429.1558 (M+1).

### Example 223

### Ethyl (1,4-dihydroxy-2-oxo-1,2-dihydro-1,8-naphthyridin-3-yl)acetate

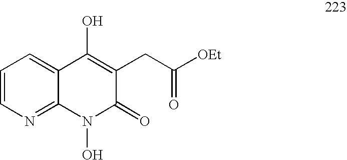

### Step 1: Ethyl 2-[(benzyloxy)(4-ethoxy-4-oxobutanoyl)aminomnicotinate

To a solution of ethyl 2-[(benzyloxy)amino]nicotinate (J. Het. Chem. 1993, 30 (4), 909-912; 2.0 g, 7.34 mmol) and pyridine (1.19 mL, 14.7 mmol) in dry toluene (20 mL) was added dropwise ethyl succinyl chloride (2.10 mL, 14.7 mmol). The solution was refluxed for 4 hours. The reaction was concentrate and the residue was purified by SGC (EtOAc/hexane gradient) to afford the title compound. ¹H NMR (400 MHz, d6-DMSO, ppm): δ 8.71 (dd, J=4.8 and 1.8 Hz, 1H), 8.20 (dd, J=7.8 and 1.8 Hz, 1H), 7.54 (dd, J=7.8 and 4.8 Hz, 1H), 7.36 (m, 5H), 5.03 (s, 2H), 4.21 (q, J=7.1 Hz, 2H), 4.04 (m, 2H), 2.54-2.41 (m, 4H), 1.24 (t, J=7.1 Hz, 3H), and 1.17 (t, J=7.1 Hz, 3H). ES MS: m/z=401 (M+1).

### Step 2: Ethyl [1-(benzyloxy)-4-hydroxy-2-oxo-1,2-dihydro-1,8-naphthyridin-3-yl]acetate and [1-(Benzyloxy)-4-hydroxy-2-oxo-1,2-dihydro-1,8-naphthyridin-3-yl]acetic acid

To a solution of ethyl 2-[(benzyloxy)(4-ethoxy-4-oxobutanoyl)amino]nicotinate (100 mg, 0.25 mmol) in dry toluene (2 mL) was added 30 wt % potassium hydride in mineral oil (33 mg, 0.25 mmol). The solution was heated overnight at 70° C. The reaction was partitioned between 10% aqueous. H₂SO₄ and DCM. The layers were separated and the product was extracted from the aqueous. layer twice more with DCM. The combined organic extracts were dried, filtered and concentrated. The crude product was purified by SGC (EtOAc/hexane gradient) to afford the title compounds. ES MS: m/z=355 (M+1).

### Step-3: Ethyl (1,4-dihydroxy-2-oxo-1,2-dihydro-1,8-naphthyridin-3-yl)acetate

To a solution of ethyl [1-(benzyloxy)-4-hydroxy-2-oxo-1,2-dihydro-1,8-naphthyridin-3-yl]acetate (22 mg, 62 μmol) in degassed EtOH (2 mL) was added 10% Pd/C (5 mg). The reaction mixture was further degassed and purged with N₂ (×3) and was then placed under H₂ balloon and stirred for 1 hour at room temperature. The mixture was filtered through Celite and washed with degassed EtOH. The filtrate was concentrated to afford the title compound. ¹H NMR (400 MHz, d6-DMSO, ppm): δ 10.9 (bs, 1H), 8.65 (d, J=3.3 Hz, 1H), 8.37 (d, J=7.9 Hz, 1H), 7.34 (dd, J=7.8 and 4.8 Hz, 1H), 4.07 (q, J=7.1 Hz, 2H), 3.65 (s, 2H) and 1.19 (t, J=7.1 Hz, 3H) ES MS: t/z=265 (M+1).

### Example 224

### N-[3-(Aminomethyl)benzyl]-2-[1-(benzyloxy)-4-hydroxy-2-oxo-1,2-dihydro-1,8-naphthyridin-3-yl]acetamide

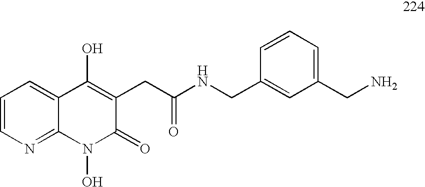

### Step 1: tert-Butyl {3-[({[1-(enzyloxy)-4-hydroxy-2-oxo-1,2-dihydro-1,8-naphthyridin-3-yl]acetyl)amino)methyl]benzyl}carbamate

[1-(Benzyloxy)-4-hydroxy-2-oxo-1,2-dihydro-1,8-naphthyridin-3-yl]acetic acid (Example 223, Step 2; 28 mg, 86 μmol), tert-butyl N-[3-(aminomethyl)benzyl]carbamate (30 mg, 0.13 mmol), EDC (25 mg, 0.13 mmol), and HOAT (18 mg, 0.13 mmol) were combined in DMF (1 ml). The reaction was stirred overnight at room temperature The solvent was removed and the residue was purified by RP-HPLC (C18 column; H₂O/CH₃CN with 0.1% TFA) to afford the title compound. ES MS: m/z=545 (M+1).

### Step 2: N-[3-(Aminomethyl)benzyl]-2-[1-(benzyloxy)-4-hydroxy-2-oxo-1,2-dihydro-1,8-naphthyridin-3-yl]acetamide

tert-Butyl {3-[({[1-(benzyloxy)-4-hydroxy-2-oxo-1,2-dihydro-1,8-naphthyridin-3-yl]acetyl}amino)methyl]benzyl}carbamate (35 mg, 64 μmol) was stirred in a solution of DCM (2 mL) and TFA (0.5 mL) for 2 hours at room temperature. The solvent was removed and the residue was dissolved in degassed MeOH (2 mL). To the solution was added 10% Pd/C (5 mg). The reaction mixture was further degassed and purged with N₂ (×3) and was then placed under H₂ balloon and stirred for 1 hour at room temperature. The mixture was filtered through Celite and washed with degassed MeOH. The solvent was removed and the residue was purified by RP-HPLC (C18 column; H₂O/CH₃CN with 0.1% TFA) to afford the title compound as the TFA salt. ¹H NMR (400 MHz, d6-DMSO, ppm): δ 11.6 (bs, 1H), 8.75 (t, J=5.7 Hz, 1H), 8.66 (dd, J=4.7 1.6 Hz, 1H), 8.36 (dd, J=7.8 and 1.6 Hz, 1H), 8.20 (bs, 3H), 7.42-7.28 (m, 5H), 4.30 (d, J=5.7 Hz, 2H), 4.03 (m, 2H) and 3.68 (s, 2H). ES MS: m/z=355 (M+1).

### Example 225

### ethyl-5-(3-bromophenyl)-1,4-dihydroxy-2-oxo-1,2-dihydro-1,8-naphthyridine-3-carboxylate and 5-(3-bromophenyl)-1,4-dihydroxy-1,8-naphthyridin-2(1H)-one

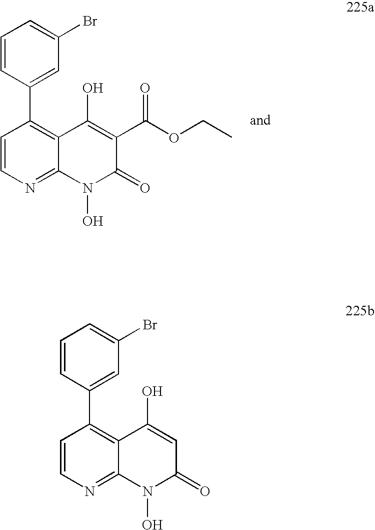

### Step 1: Methyl 4-(3-bromophenyl)-2-fluoronicotinate

To a solution of methyl 2-fluoro-4-iodonicotinate (0.500 g, 1.779 mmol) in toluene (4 mL), EtOH (0.50 mL), and H₂O (0.50 mL) was added 3-bromophenyl boronic acid (0.357 g, 1.779 mmol), potassium carbonate (0.369 g, 2.67 mmol), and tetrakis (0.514 g, 0.445 mmol) while N₂ was bubbled through the solution. The reaction vessel was sealed and the reaction heated at 110° C. for 1.5 hours. The solution was cooled to room temperature, diluted with aqueous NaOH (1N, 10 mL), and extracted into EtOAc (3×10 mL). The organic layers were combined, dried, filtered, and concentrated. The residue was purified by SGC (0-25% EtOAc/hexane) to afford the title compound. ES MS: m/z=310 (M), 312 (M+2).

### Step 2: Methyl 2-[(1-benzyloxy)amino]-4-(3-bromophenyl)nicotinate

To a solution of methyl 4-(3-bromophenyl)-2-fluoronicotinate (0.2546 g, 0.821 mmol) in DMSO (5 mL) in a microwave tube was added o-benzylhydroxylamine (0.337 mL, 2.87 mmol). After sealing the tube, the reaction mixture was stirred at 110° C. overnight. The solution was cooled to room temperature, diluted with aqueous HCl (1N, 12 mL), and extracted into EtOAc (3×12 mL). The organic layers were combined, dried, filtered, and concentrated. The residue was purified by SGC (0-25% EtOAc/hexane) to afford the title compound. ES MS: m/z=413 (M), 415 (M+2).

### Step 3: Methyl 2-[(benzyloxy) (3-ethoxy-3-oxopropanoyl)amino]-4-(3-bromophenyl)nicotinate

A solution of Methyl 2-[(benzyloxy)amino]-4-(3-bromophenyl)nicotinate (0.1991 g, 0.482 mmol) in DCM (10 mL) and TEA (0.134 mL, 0.964 mmol) was treated dropwise with ethyl malonyl chloride (0.124 mL, 0.964 mmol). The mixture was stirred at room temperature for 1 hour. Aqueous HCl (0.5M, mL) was added. The organic layer was separated and extracted 2× more with DCM. The organic layers were combined, dried, filtered and concentrated. The residue was purified by SGC (0-50% EtOAc/hexane) to afford the title compound. ES MS: m/z=527 (M), 529 (M+2).

### Step 4: Ethyl 1-(benzyloxy)-5-(3-bromophenyl)-4-hydroxy-2-oxo-1,2-dihydro-1,8-naphthyridine-3-carboxylate

Potassium tert-butoxide (0.085 g, 0.755 mmol) was added to EtOH (6 mL) and the solution was refluxed (80° C.) under N₂ for ˜20 minutes. Methyl 2-[(benzyloxy) (3-ethoxy-3-oxopropanoyl)amino]-4-(3-bromophenyl)nicotinate (0.1992 g, 0.378 mmol) was taken up in EtOH (6 mL) and the solution was added dropwise to the hot potassium tert-butoxide solution over 5 minutes. The resulting solution was refluxed for an additional 20 minutes then cooled to room temperature. The EtOH was removed. The residue was acidified with aqueous HCl (0.5 M) and extracted into EtOAc (3×12 mL). The organic layers were combined, dried, filtered, and concentrated to afford the title compound. ES MS: m/z=495 (M), 497 (M+2).

### Step 5: Ethyl 5-(3-bromophenyl)-1,4-dihydroxy-2-oxo-1,2-dihydro-1,8-naphthyridine-3-carboxylate and 5-(3-bromophenyl)-1,4-dihydroxy-1,8-naphthyridin-2(1H)-one

A solution of Ethyl 1-(benzyloxy)-5-(3-bromophenyl)-4-hydroxy-2-oxo-1,2-dihydro-1,8-naphthyridine-3-carboxylate (0.040 g, 0.081 mmol) in HBr (33 wt. % in AcOH, 2 mL) and H₂O (0.5 mL) was heated to 80° C. for 0.5 hour. The solvent was removed. After concentration, the decarboxylated product is seen by LC/MS in addition to the desired product. The residue was purified by RP-HPLC (C18 column; H₂O/CH₃CN with 0.1% TFA) to afford separation of the title compounds. Compound A: ¹H NMR (400 MHz, d₆-DMSO, ppm): δ 13.02 (bs, 1H), 11.85 (bs, 1H), 8.72 (d, J=4.6 Hz, 1H), 7.62-7.60 (m, 2H), 7.11 (d, J=4.8 Hz, 1H), 4.29 (q, J=7.1 Hz, 2H), 1.27 (t, J=7.1 Hz, 3H). High Resolution MS (FT-ICR): m/z found 405.0069 (M+1); calculated 405.0081 (M+1). Compound B: ¹H NMR (400 MHz, d₆-DMSO, ppm): δ 11.19 (s, 1H), 11.65 (bs, 1H), 8.62 (d, J=4.4 Hz, 1H), 7.58 (d, J=3.9 Hz, 1H), 7.56 (s, 1H), 7.35 (s, 2H), 7.05 (d, J=4.4 Hz, 1H) 5.83 (s, 1H). High Resolution MS (FT-ICR): m/z found 332.9870 (M+1); calculated 332.9870 (M+1).

### Example 226

### 1,4-dihydroxy-5-(3-hydroxyphenyl)-1,8-naphthyridin-2(1H)-one

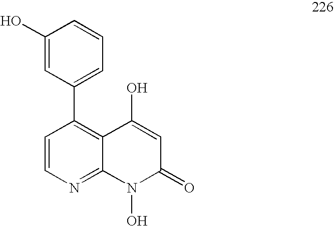

The above compound was prepared in accordance with the procedures set forth in Example 225. High Resolution MS (FT-ICR): m/z found 271.0714 (M+1); calculated 271.0714 (M+1).

### Example 227

### 5-[3′-(aminomethyl)biphenyl-3-yl]-1,4-dihydroxy-1,8-naphthyridin-2-(1H)-one

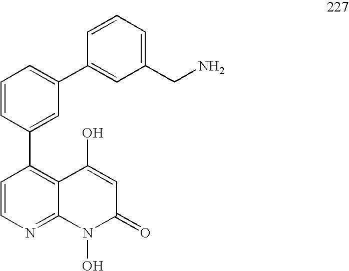

### Step 1: tert-butyl ({3′-[8-benzyloxy)-5-hydroxy-7-oxo-7,8-dihydro-1,8-naphthyridin-4-yl]biphenyl-3-yl}methyl)carbamate

The Ethyl 1-(benzyloxy)-5-(3-bromophenyl)-4-hydroxy-2-oxo-1,2-dihydro-1,8-naphthyridine-3-carboxylate (Example 225, Step 4, 0.100 g, 0.202 mmol) was dissolved in DMF (5.0 mL) and H₂O (1.0 mL). To this was added 3-(N-BOC-aminomethyl)phenylboronic acid (0.101 g, 0.404 mmol), potassium carbonate (0.084 g, 0.606 mmol), and the Pd dppf (DCM adduct) catalyst (0.008 g, 0.010 mmol) while N₂ was bubbled through the solution. The reaction vessel was sealed and the reaction heated in a microwave at 100° C. for 0.5 hour. The solution was cooled to room temperature, diluted with H₂O (6 mL), and extracted into EtOAc (3×10 mL). The organic layers were combined, dried, filtered, and concentrated. The residue was purified by SGC (0-50% EtOAc/hexane) to afford the title compound. ES MS: m/z=622 (M+1).

### Step 2: 5-[3′-(aminomethyl)biphenyl-3-yl]-1,4-dihydroxy-1,8-naphthyridin-2-(1H)-one

A solution of tert-butyl ({3′-[8-benzyloxy)-5-hydroxy-7-oxo-7,8-dihydro-1,8-naphthyridin-4-yl]biphenyl-3-yl}methyl) carbamate (0.1272 g, 0.205 mmol) in HBr (33 wt. % in AcOH, 3 mL) and H₂O (0.75 mL) was heated to 80° C. for 0.5 hour. The solvent was removed. The residue was purified by RP-HPLC (C18 column; H₂O/CH₃CN with 0.1% TFA) to afford the title compound. ¹H NMR (400 MHz, d₆-DMSO, ppm): δ 11.59 (s, 1H), 10.64 (bs, 1H), 8.65 (d, J=4.7 Hz, 1H), 8.17 (bs, 3H), 7.84 (s, 1H), 7.74 (t, J=9.1 Hz, 2H), 7.65 (s, 1H), 7.52 (t, J=7.6 Hz, 2H), 7.44 (d, J=7.5 Hz, 1H), 7.38 (d, J=7.3 Hz, 1H), 7.12 (d, J=4.7 Hz, 1H), 5.85 (s, 1H), 4.11 (d, J=5.2 Hz, 2H). High Resolution MS (FT-ICR): m/z found 360.1342 (M+1); calculated 360.1343 (M+1).

TABLE 15
The compounds in the following table were prepared in accordance with the
procedures set forth in Example 227:

| Ex/ | | | |
|---|---|---|---|
| cpd | Name | Structure | Data |
| 228 | 5-[4′- (aminomethyl)biphenyl- 3-yl]-1,4-dihydroxy- 1,8-naphthyridin- 2(1H)-one | 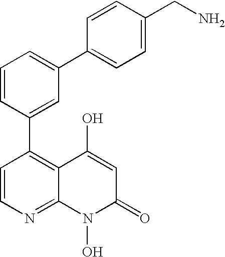 | Resolution MS (FT-ICR): m/z found 360.1325 (M + 1); calculated 360.1343 (M + 1) |
| 229 | 5-[3-(3,5-dimethyl-1H- pyrazol-1-yl)phenyl]- 1,4-dihydroxy-1,8- naphthyridin-2(1H)-one | 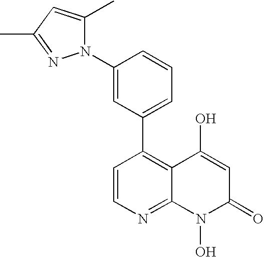 | ES MS: m/z = 348.1 (M + 1). |
| 230 | ethyl 5-{3- [(dimethylamino)methyl] phenyl}-1,4- dihydroxy-2-oxo-1,2- dihydro-1,8- naphthyridine-3- carboxylate | 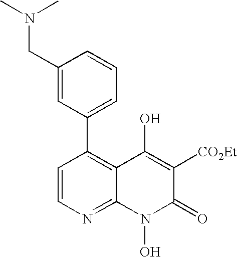 | ES MS: m/z = 384.1 (M + 1). |

### Example: 231

### 4-{[3′-(aminomethyl)biphenyl-3-yl]methyl}-1-hydroxy-1,8-naphthyridin-2(1 H)-one

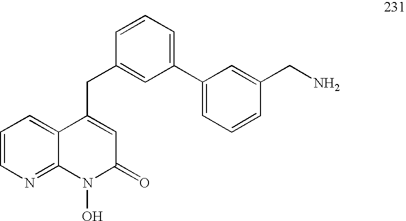

### Step 1: 1-(benzyloxy)-4-(3-bromobenzyl)-1,8-naphthyridin-2(1H)-one

N₂ was bubbled through a solution of 1-(Benzyloxy)-2-oxo-1,2-dihydro-1,8-naphthyridin-4-yl trifluoromethanesulfonate (Example 103, Step 1; 0.250 g, 0.624 mmol) in anhydrous THF (5 mL). After the addition the tetrakis (0.036 g, 0.031 mmol) the reaction vessel was sealed. To this was added, 3-bromobenzylzinc bromide (0.5M solution in THF, 2.498 mL, 1.249 mmol) via syringe. The reaction was heated in a microwave at 110° C. for 10 minutes. The solution was cooled to room temperature, diluted with aqueous HCl (1N, 8 mL), and extracted into EtOAc (10 mL). The organic layer was dried, filtered, and concentrated. ES MS: m/z=421 (M), 423 (M+2).

### Step 2: tert-butyl[(3-{[1-(benzyloxy)-2-oxo-1,2-dihydro-1,8-naphthyridin-4-yl]methyl}biphenyl-3-yl)methyl]carbamate

The 1-(benzyloxy)-4-(3-bromobenzyl)-1,8-naphthyridin-2(1H)-one (0.150 g, 0.356 mmol) was dissolved in DMF (5.0 mLs) and H₂O (1.0 mL). To this was added 3-(N-BOC-aminomethyl)phenylboronic acid (0.179 g, 0.712 mmol), potassium carbonate (0.148 g, 1.068 mmol), and the Pd dppf (DCM adduct) catalyst (0.015 g, 0.018 mmol) while N₂ was bubbled through the solution. The reaction vessel was sealed and the reaction heated in a microwave at 100° C. for 10 minutes. The solution was cooled to room temperature, diluted with aqueous HCl (1N, 6 mL), and extracted into EtOAc (10 mL) The organic layer was dried, filtered, and concentrated. ES MS: m/z=548 (M+1).

### Step 3: 4-{[3′-(aminomethyl)biphenyl-3-yl]methyl}-1-hydroxy-1,8-naphthyridin-2(1H)-one

A solution of tert-butyl [(3′-{[1-(benzyloxy)-2-oxo-1,2-dihydro-1,8-naphthyridin-4-yl]methyl}biphenyl-3-yl)methyl]carbamate (0.4343 g, 0.743 mmol) in HBr (33 wt. % in AcOH, 3 mL) and H₂O (0.75 mL) was heated to 80° C. for 0.5 hour. The solvent was removed. The residue was purified by RP-HPLC (C18 column; H₂O/CH₃CN with 0.1% TFA) to afford the title compound. ¹H NMR (400 MHz, d₆-DMSO, ppm): δ 10.90 (bs, 1H), 8.66 (d, J=3.6 Hz, 1H), 8.36 (d, J=6.7 Hz, 1H), 8.18 (bs, 3H), 7.77 (s, 1H), 7.67 (d, J=9.2 Hz, 2H), 7.59-7.43 (m, 4H), 7.34-7.32 (m, 2H), 6.55 (s, 1H), 4.33 (s, 2H), 4.11 (d, J=3.3 Hz, 2H). High Resolution MS (FT-ICR): m/z found 358.1555 (M+1); calculated 358.155 (M+1).

TABLE 16
The compounds in the following table were prepared in accordance with the
procedures set forth in Example 231:

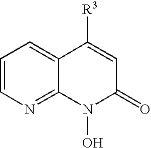

| Ex/ | | | |
|---|---|---|---|
| cpd | Name | R³ | Data |
| 232 | 4-{[4′- (aminomethyl)biphenyl- 3-yl]methyl}-1- hydroxy-1,8- naphthyridin-2(1H)-one | 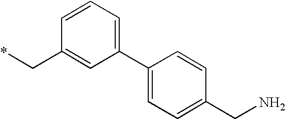 | High Resolution MS (FT-ICR): m/z found 358.1556 (M + 1); calculated 358.155 (M + 1) |
| 233 | 4-[(3′-aminobiphenyl- 3-yl)methyl]-1- hydroxy-1,8- naphthyridin-2(1H)-one | 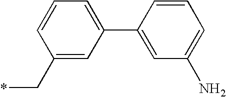 | High Resolution MS (FT-ICR): m/z found 344.1391 (M + 1); calculated 344.1394 (M + 1) |
| 234 | 4-[(4′-aminobiphenyl- 3-yl)methyl]-1- hydroxy-1,8- naphthyridin-2(1H)-one | 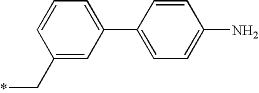 | Resolution MS (FT-ICR): m/z found 344.1393 (M + 1); calculated 344.1394 (M + 1) |
| 235 | 4-{[4′- (aminomethyl)biphenyl- 4-yl]methyl}-1- hydroxy-1,8- naphthyridin-2(1H)-one | 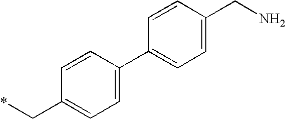 | High Resolution MS (FT-ICR): m/z found 358.1547 (M + 1); calculated 358.155 (M + 1) |
| 236 | 4-{4-[2-(2,4- difluorophenyl)ethyl] benzyl}-1-hydroxy-1,8- naphthyridin-2(1H)-one | 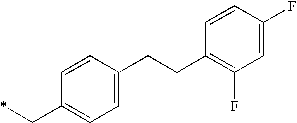 | High Resolution MS (FT-ICR): m/z found 393.1401 (M + 1); calculated 393.1409 (M + 1) |

### Example 237

### 1-hydroxy-4-(3-hydroxyphenyl)-1,8-naphthyridin-2(1H)-one

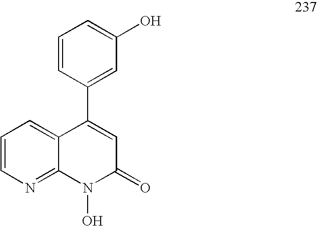

### Step 1: 1-(benzyloxy)-4-(3-hydroxyphenyl)-1,8-naphthyridin-2(1H)-one

The 1-(Benzyloxy)-2-oxo-1,2-dihydro-1,8-naphthyridin-4-yl trifluoromethanesulfonate (Example 103, Step 1; 0.150 g, 0.375 mmol) was dissolved in DMF (5.0 mLs). To this was added 3-hydroxyphenylboronic acid (0.054 g, 0.393 mmol), aqueous sodium carbonate (2 M; 0.375 mL, 0.749 mmol), and tetrakis (0.022 g, 0.019 mmol) while N₂ was bubbled through the solution. The reaction vessel was sealed and the reaction heated at 80° C. for 1 hour. The solvent was removed. The residue was diluted with aqueous HCl (1N, 10 mL), and extracted into EtOAc (3×10 mL). The organic layers were combined, dried, filtered, and concentrated. The residue was purified by SGC (0-10% MeOH/DCM) to afford the title compound. ES MS: m/z=345 (M+1).

### Step 2: 1-hydroxy-4-(3-hydroxyphenyl)-1,8-naphthyridin-2(1 B)-one

A solution of 1-(benzyloxy)-4-(3-hydroxyphenyl)-1,8-naphthyridin-2(1H)-one (0.0592 g, 0.172 mmol) in HBr (33 wt. % in AcOH, 2 mL) and H₂O (0.5 mL) was heated to 80° C. for 0.5 hour. The solvent was removed. The residue was purified by RP-HPLC (C18 column; H₂O/CH₃CN with 0.1% TFA) to afford the title compound. ¹H NMR (400 MHz, d₆-DMSO, ppm): δ 11.07 (s, 1H), 9.77 (s, 1H), 8.70 (dd, J=4.5, 1.5 Hz, 1H), 7.92 (dd, J=8.0, 1.5 Hz, 2H), 7.37-7.31 (m, 2H), 6.94-6.88 (m, 2H), 6.85 (d, J=1.7 Hz, 1H), 6.66 (s, 1H). High Resolution MS (FT-ICR): m/z found 255.0787 (M+1); calculated 255.0764 (M+1).

TABLE 17
The compounds in the following table were prepared in accordance with the
procedures set forth in Example 237:

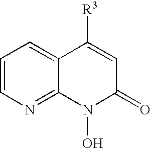

| Ex/ | | | |
|---|---|---|---|
| cpd | Name | R³ | Data |
| 238 | 4-[3′-(1- aminocyclopropyl)bi- phenyl-4-yl]-1-hydroxy- 1,8-naphthyridin- 2(1H)-one | 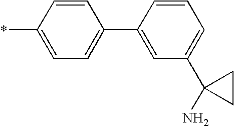 | High Resolution MS (FT-ICR): m/z found 370.1551 (M + 1); calculated 370.155 (M + 1) |
| 239 | 4-[4-(4- aminobenzyl)phenyl]- 1-hydroxy-1,8- naphthyridin-2(1H)-one | 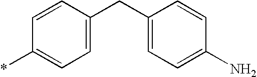 | High Resolution MS (FT-ICR): m/z found 344.1395 (M + 1); calculated 344.1394 (M + 1) |
| 240 | ethyl 1-hydroxy-2-oxo- 4-(2-phenylethyl)-1,2- dihydro-1,8- naphthyridine-3- carboxylate | 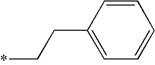 | ES MS: m/z = 339.1 (M + 1). |

### Example 241

### Ethyl 5-[4′-(aminomethyl)biphenyl-4-yl]-8-hydroxy-7-oxo-7,8-dihydro-1,8-naphthyridine-4-carboxylate

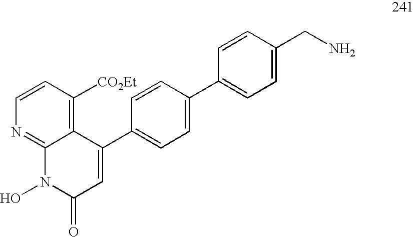

### Step 1: Ethyl 8-(benzyloxy)-7-oxo-5-{[trifluoromethyl)sulfonyl]oxy}-7,8-dihydro-1,8-naphthyridine-4-carboxylate

A solution of Ethyl 8-(benzyloxy)-5-hydroxy-7-oxo-7,8-dihydro-1,8-naphthyridine-4-carboxylate (Example 88, Step 4; 0.150 g, 0.441 mmol) and TEA (0.123 mL, 0.881 mmol) in DCM (6 mL) was cooled to 0° C. and treated dropwise with trifluoromethanesulfonic anhydride (0.119 mL, 0.705 mmol). The cooling bath was removed after 30 minutes and the mixture stirred at room temperature for 1 hour. The solvent was removed. The residue was purified by SGC (0-50% EtOAc/hexane) to give the title compound. ES MS: m/z=473 (M+1).

### Step 2: Ethyl 8-(benzyloxy)-5-(4′-{[tert-butoxycarbonyl)amino]methyl}biphenyl-4-yl)-7-oxo-7,8-dihydro-1,8-naphthyridine-4-carboxylate

The Ethyl 8-(benzyloxy)-7-oxo-5-{[trifluoromethyl)sulfonyl]oxy}-7,8-dihydro-1,8-naphthyridine-4-carboxylate (0.050 g, 0.106 mmol) was dissolved in DMF (2.0 mL). To this was added tert-butyl {[4′-(4,4,5,5-tetramethyl-1,3,2-dioxaborolan-2-yl)biphenyl-4-yl]methyl}carbamate (0.045 g, 0.111 mmol), aqueous sodium carbonate (2 M; 0.106 mL, 0.212 mmol), and tetrakis (0.0011 g, 0.907 μmol) while N₂ was bubbled through the solution. The reaction vessel was sealed and the reaction heated in a microwave at 100° C. for 10 minutes. The solvent was removed. The residue was partioned between aqueous HCl (1N, 6 mL), and EtOAc (6 mL). The organic layer was separated, dried, filtered, and concentrated to afford the title compound. ES MS: m/z=606 (M+1).

### Step 3: Ethyl 5-[4′-(aminomethyl)biphenyl-4-yl]-8-hydroxy-7-oxo-7,8-dihydro-1,8-naphthyridine-4-carboxylate

A solution of Ethyl 8-(benzyloxy)-5-(4′-{[tert-butoxycarbonyl)amino]methyl}biphenyl-4-yl)-7-oxo-7,8-dihydro-1,8-naphthyridine-4-carboxylate (0.1755 g, 0.290 mmol) in HBr (33 wt. % in AcOH, 3 mL) and H₂O (0.75 mL) was heated to 80° C. for 0.5 hour. The solvent was removed. The residue was purified by RP-HPLC (C18 column; H₂O/CH₃CN with 0.1% TFA) to afford the title compound. ¹H NMR (400 MHz, d₆-DMSO, ppm): δ 11.27 (bs, 1H), 8.83 (d, J=4.7 Hz, 1H), 8.21 (bs, 1H), 7.82-7.80 (m, 4H), 7.59 (d, J=8.0 Hz, 2H), 7.47 (d, J=8.1 Hz, 2H), 7.40 (d, J=4.7 Hz, 1H), 6.78 (s, 1H), 4.12 (s, 2H), 3.44 (q, J=7.1 Hz, 2H), 1.03 (t, J=7.1 Hz, 3H). High Resolution MS IT-ICR): m/z found 416.1613 (M+1); calculated 416.1605 (M+1).

### Example 242

### 4-[4′-(aminomethyl)biphenyl-4-yl]-6-fluoro-1-hydroxy-3-phenyl-1,8-naphthyridin-2-(1H)-one

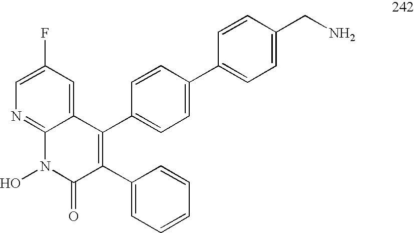

### Step 1: 1-(benzyloxy)-6-fluoro-2-oxo-3-phenyl-1,2-dihydro-1,8-naphthyridin-4-yl trifluoromethanesulfonate

A solution of 1-(benzyloxy)-6-fluoro-4-hydroxy-3-phenyl-1,8-naphthyridin-2(1H)-one (the o-benzylated precursor to Example 48; (0.150 g, 0.414 mmol) and TEA (0.112 mL, 0.662 mmol) in DCM (6 mL) was cooled to 0° C. and treated dropwise with trifluoromethanesulfonic anhydride (0.115 mL, 0.828 mmol). The cooling bath was removed after 30 minutes and the mixture stirred at room temperature for 1 hour. The solvent was removed. The residue was purified by SGC (0-50% EtOAc/hexane) to give the title compound. ES MS: m/z=495 (M+1).

### Step 2: 4-[4′-(aminomethyl)biphenyl-4-yl]-1-(benzyloxy)-6-fluoro-3-phenyl-1,8-naphthyridin-2(1H)-one

The 1-(benzyloxy)-6-fluoro-2-oxo-3-phenyl-1,2-dihydro-1,8-naphthyridin-4-yl trifluoromethanesulfonate (0.050 g, 0.101 mmol) was dissolved in DMF (2.0 mLs). To this was added tert-butyl {[4′-(4,4,5,5-tetramethyl-1,3,2-dioxaborolan-2-yl)biphenyl-4-yl]methyl}carbamate (0.043 g, 0.106 mmol), aqueous sodium carbonate (2 M; 0.101 mL, 0.202 mmol), and tetrakis (0.0011 g, 0.907 μmol) while N₂ was bubbled through the solution. The reaction vessel was sealed and the reaction heated in a microwave at 100° C. for 10 minutes. The solvent was removed. The residue was partioned between aqueous HCl (1N, 10 mL), and EtOAc (10 mL). The organic layer was separated, dried, filtered, and concentrated the title compound. ES MS: m/z=628 (M+1).

### Step 3: 4-[4′-(aminomethyl)biphenyl-4-yl]-6-fluoro-1-hydroxy-3-phenyl-1,8-naphthyridin-2-(1H)-one

A solution of 4-[4′-(aminomethyl)biphenyl-4-yl]-1-(benzyloxy)-6-fluoro-3-phenyl-1,8-naphthyridin-2(1H)-one (0.2241 g, 0.357 mmol) in HBr (33 wt. % in AcOH, 3 mL) and H₂O (0.75 mL) was heated to 80° C. for 0.5 hour. The solvent was removed. The residue was purified by RP-HPLC (C18 column; H₂O/CH₃CN with 0.1% TFA) to afford the title compound. ¹H NMR (400 MHz, d₆-DMSO, ppm): δ 11.32 (bs, 1H), 8.77 (d, J=2.7 Hz, 1H), 8.20 (bs, 1H), 7.77 (d, J=8.2 Hz, 2H), 7.70 (d, J=8.2 Hz, 2H), 7.53 (d, J=8.1 Hz, 2H), 7.33-7.29 (m, 3H), 7.24-7.17 (m, 5H), 4.09 (d, J=5.6 Hz, 2H). High Resolution MS (FT-ICR): m/z found 438.1625 (M+1); calculated 438.1613 (M+1).

### Example 243

### Ethyl 4-[4′-(aminomethyl)biphenyl-4-yl]-1-hydroxy-2-oxo-1,2-dihydro-1,8-naphthyridine-3-carboxylate

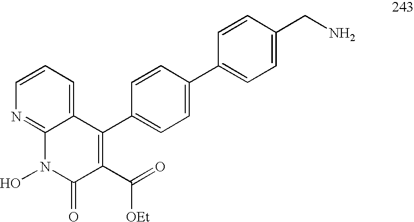

### Step 1: Ethyl 4-[4′-(aminomethyl)biphenyl-4-yl]-1-(benzyloxy)-2-oxo-1,2-dihydro-1,8-naphthyridine-3-carboxylate

The ethyl 1-(benzyloxy)-2-oxo-4-{[(trifluoromethyl)sulfonyl]oxy}-1,2-dihydro-1,8-naphthyridine-3-carboxylate (0.100 g, 0.212 mmol) was dissolved in DME (2.0 mL). To this was added tert-butyl {[4′-(4,4,5,5-tetramethyl-1,3,2-dioxaborolan-2-yl)biphenyl-4-yl]methyl}carbamate (0.091 g, 0.222 mmol), aqueous sodium carbonate (2 M; 0.212 mL, 0.423 mmol), and tetrakis (0.012 g, 10.58,mol) while N₂ was bubbled through the solution. The reaction vessel was sealed and the reaction heated at 80° C. for 2 hours. The solvent was removed. The residue was partioned between aqueous HCl (1N, 5 mL), and EtOAc (5 mL). The organic layer was separated, dried, filtered, and concentrated to afford the title compound. ES MS: m/z=606(M+1).

### Step 2: Ethyl 4-[4′-(aminomethyl)biphenyl-4-yl]-1-hydroxy-2-oxo-1,2-dihydro-1,8-naphthyridine-3-carboxylate

A solution of Ethyl 4-[4′-(aminomethyl)biphenyl-4-yl]-1-(benzyloxy)-2-oxo-1,2-dihydro-1,8-naphthyridine-3-carboxylate (0.3569 g, 0.589 mmol) in HBr (33 wt. % in AcOH, 3 mL) and H₂O (0.75 mL) was heated to 80° C. for 0.5 hour. The solvent was removed. The residue was purified by RP-HPLC (C18 column; H₂O/CH₃CN with 0.1% TFA). ¹H NMR (400 MHz, d₆-DMSO, ppm): δ 8.77 (dd, J=4.6, 1.7, 1H), 7.88 (d, J=8.3 Hz, 1H), 7.82 (d, J=8.2 Hz, 1H), 7.73 (dd, J=8.1, 1.6 Hz, 1H), 7.58 (d, J=8.1 Hz, 1H), 7.49 (d, J=8.3 Hz, 1H), 7.36 (q, J=4.2 Hz, 1H), 4.08 (s, 2H), 4.05 (q, J=7.1 Hz, 2H), 0.91 (t, J=7.1 Hz, 3H). High Resolution MS (FT-ICR): m/z found 416.1631 (M+1); calculated 416.1605 (M+1).

### Example 244

### 1-Hydroxy-4-(pyrazol-4-yl)-1,8-naphthyridin-2-(1H)-one

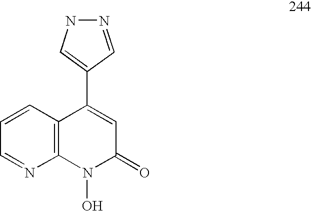

### Step 1: 1-(benzyloxy)-4-(1H-pyrazol-4-yl)-1,8-naphthyridin-2(1H)-one

A mixture of 1-(benzyloxy)-2-oxo-1,2-dihydro-1,8-naphthyridin-4-yl trifluoromethanesulfonate (50 mg, 0.125 mmol), tert-butyl 4-(4,4,5,5-tetramethyl-1,3,2-dioxaborolan-2-yl)-1H-pyrazole-1-carboxylate (74 mg, 0.25 mmol), 2M sodium carbonate (187 uL, 0.375 mmol), and Pd(PPh3)₄ (7.2 mg, 6.24 umol) in 1.5 mL DME was microwaved at 120° C. for 25 minutes. Reaction was filtered through Celite, washing with DCM. The solvent was evaporated and the residue was purified by SGC (0-5% MeOH:CHCl₃) to give 22 mg of an oil.

### Step 2: 1-hydroxy-4-(1H-pyrazol-4-yl)-1,8-naphthyridin-2(1H)-one hydrobromide

1-(benzyloxy)-4-(1H-pyrazol-4-yl)-1,8-naphthyridin-2(1H)-one (22 mg, 0.069 mmol) was dissolved in 300 uL 30% HBr/HOAc. Add 90 uL H₂O and heat at 80° C. for 1 hour. Concentrated to give a solid. Triturate with ether and filter off solids. Dry under vacuum to give 18 mg of a solid. ¹H NMR (400 MHz, d6 DMSO): 8.70 (d, J=3.7 Hz, 1H), 8.35 (d, J=7.7 Hz, 1H), 8.13 (s, 2H), 7.37 (dd, J=4.7, 7.9 Hz, 1H), 6.81 (s, 1H). High Resolution MS (FT-ICR): m/z found 229.0752 (M+1); calculated 229.0720 (M+1).

### Example 245

### 1-hydroxy-4-(1H-pyrrolo[2,3-b]pyridin-5-yl)-1,8-naphthyridin-2(1H)-one bistrifluoroacetate

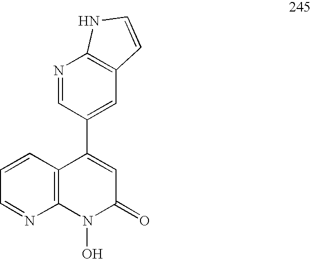

The above compound was prepared in accordance with the procedures set forth in Example 244 with the exception that the final compound was purified by RP-HPLC (C18 column; H₂O/CH₃CN with 0.1% TFA). High Resolution MS (FT-ICR): m/z found 279.0907 (M+1); calculated 279.0887 (M+1)

### Example 246

### 4-(3,4-dihydronaphthalen-2-yl)-1-hydroxy-1,8-naphthyridin-2(1H)-one

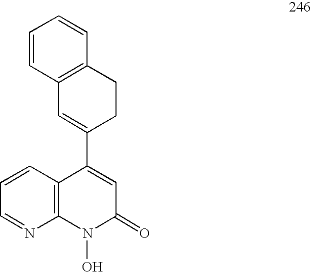

### Step 1: 1,4-dihydronaphthalen-2-yl trifluoromethanesulfonate

3,4-dihydronaphthalen-2(1H)-one (1 g, 6.84 mmol) was dissolved in dry DCM (15 ml) and cooled to −78° C. N-diisopropylethylamine (5.97 ml, 34.2 mmol) was added and the mixture to stir for 10 minutes. Trifluoromethanesulfonic anhydride (1.4 ml, 8.21 mmol) was added drop-wise, followed by slow warming to room temperature overnight. The mixture was then washed with H₂O and 10% citric acid solution (2×) and dried and the solvent removed. The residue was purified by SGC (0-5% EtOAc/Hexane) to give the title compound. ¹H NMR (400 MHz, CDCl₃, ppm): B 7.15 (m, 4H), 6.47 (s, 1H), 3.57 (t, J=8.2, 3H), 3.12 (t, J=8.4 3H).

### Step 2: 1-(benzyloxy)-4-(3,4-dihydronaphthalen-2-yl)-1,8-naphthyridin-2(1H)-one

A flask charged with 1,4-dihydronaphthalen-2-yl trifluoromethanesulfonate (100 mg, 0.359 mmol), bis(pinacolato)diboron (100 mg, 0.395 mmol), potassium acetate (106 mg, 1.078 mmol) and PdCl₂(dppf) (7.89 mg, 0.011 mmol) in DMF (2 ml) was flushed with N₂. The reaction mixture was stirred at 80° C. for 2 hours. After cooling to room temperature, 1-(benzyloxy)-2-oxo-1,2-dihydro-1,8-naphthyridin-4-yltrifluoromethanesulfonate (288 mg, 0.719 mmol), PdCl₂(dppf) (7.89 mg, 0.011 mmol), and Na₂CO₃ (0.898 ml, 1.797 mmol, 2M in H₂O) were added. The mixture was then stirred at 80° C. (oil bath) under N₂ overnight. The reaction was cooled to room temperature and the product was extracted with Et₂O. The organics were washed with H₂O, brine, dried and concentrated. The residue was purified on SGC (5% EtOAc/hexane) to give the title compound: ES MS: m/z=381.3 (M+1).

### Step 3: 4-(3,4-dihydronaphthalen-2-yl)-1-hydroxy-1,8-naphthyridin-2(1H)-one

1-(benzyloxy)-4-(3,4-dihydronaphthalen-2-yl)-1,8-naphthyridin-2(1H)-one (33 mg, 0.087 mmol) was dissolved in 15 ml of EtOH. While bubbling with N₂ (g), 10% Pd/C (˜8 mg) was added. The reaction was then flushed with H₂ (g) (3×) and was allowed to stir under H₂ (g) for 3 hours. Upon completion it was filtered and purified by RP-HPLC (10-100% H₂O/CH₃CN) to give the title compound. ¹H NMR (400 MHz, CD₃OD, ppm): [symbol] \tilde{□} □ [symbol] ddJ □~₁□ [symbol] and 4.94 □z, 1H), 8.40 (dd, J=1.47 and 8.06 Hz, 1H), 7.43 (dd, J=4.76 and 8.06 Hz, 1H), 7.18 (m, 4H), 6.78 (s, 1H), 6.75 (s, 1H), 3.02 (t, J=7.69, 3H), 2.69 (t, J=7.14, 3H). ES MS: m/z=291.3 (M+1).

### Example 247

### 4-(3,4-dihydroisoquinolin-2(1H)-ylcarbonyl)-1-hydroxy-1,8-naphthyridin-2(1H)-one

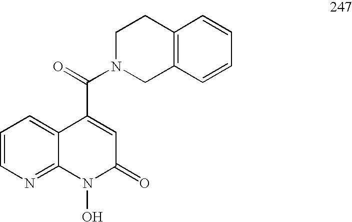

### Step 1: 1-(benzyloxy)-2-oxo-1,2-dihydro-1,8-naphthyridine-4-carboxylic acid

In an oven-dried glass liner of a Parr pressure vessel, a solution of 1-(benzyloxy)-2-oxo-1,2-dihydro-1,8-naphthyridin-4-yl trifluoromethanesulfonate (Example 103, step 1, 400 mg, 1.0 mmol) and N,N-dicyclohexylmethylamine (0.4 mL, 1.87 mmol) in anhydrous DMF (10 mL) and anhydrous MeOH (5 mL) was bubbled with N₂ gas for 10 minutes. Bis(tri-t-butylphosphine)palladium(0) (34 mg, 0.067 mmol) was added and the pressure vessel was pressurized with CO(g) to 300 psi. The vessel was heated at 70° C. for 18 hours. The vessel was then cooled and depressurized. The reaction was diluted with MeOH, filtered, and concentrated. The crude product was purified by SGC (0-100% EtOAc/hexane) to afford a white powder. This solid was dissolved in MeOH (20 mL), NaOH (1N, 1.1 eq) was added and the solution was stirred at room temperature for 30 minutes. The organics were removed and the residue was acidified with 1N HCl and extracted into EtOAc. The combined organics were dried, filtered, and concentrated to afford the title compound as a white solid. ES MS m/z=297.1 (M+1).

### Step 2: 4-(3,4-dihydroisoquinolin-2(1H)-ylcarbonyl)-1-hydroxy-1,8-naphthyridin-2(1H)-one

To a solution of 1,2,3,4-tetrahydroisoquinoline (32 uL, 0.25 mmol), diisopropylethylamine (65 uL, 0.37 mmol), and 1-(benzyloxy)-2-oxo-1,2-dihydro-1,8-naphthyridine-4-carboxylic acid (50 mg, 0.17 mmol) in DMF (0.50 mL) was added (1H-1,2,3-benzotriazol-1-yloxy)[tris(dimethylamino)]phosphonium hexafluorophosphate (90 mg, 0.20 mmol) and the resulting solution was allowed to stir for 2 days at room temperature. The reaction was diluted with H₂O and extracted into EtOAc. The combined organics were washed with brine, dried, filtered, and concentrated. The residue was dissolved into a 2:1 EtOAc/EtOH solution (2 mL) and bubbled with N₂ gas. Pearlman's catalyst (23 mg) was added and a balloon of H₂ gas was attached to the flask. After 2 hours of stirring, the reaction was filtered through a pad of celite and the filtrate concentrated. The crude product was purified by RP-HPLC (C18 column; H₂O/CH₃CN with 0.1% TFA) to give the title compound. High Resolution MS (FT-ICR): m/z found 322.1186 (M+1); calculated 322.1178 (M+1).

### Example 248

### ethyl 4-[4′-(aminomethyl)biphenyl-3-yl]-1-hydroxy-6-(2-methoxyphenyl)-2-oxo-1,2-dihydro-1,8-naphthyridine-3-carboxylate

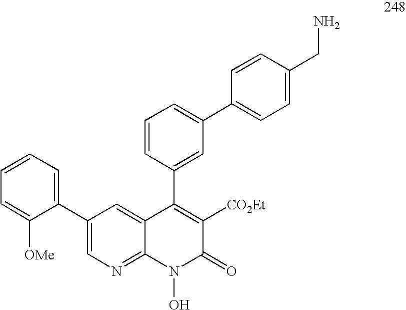

### Step 1: ethyl 1-(benzyloxy)-4-hydroxy-6-(2-methoxyphenyl)-2-oxo-1,2-dihydro-1,8-naphthyridine-3-carboxylate

Ethyl 1-(benzyloxy)-6-bromo-4-hydroxy-2-oxo-1,2-dihydro-1,8-naphthyridine-3-carboxylate (420 mgs, 1 mmol) was taken up in DMF (5 mL) and 2-methoxyphenylboronate (170 mgs, 1.1 mmol) and sodium carbonate solution (1 mL, 2 M) was added under N₂ followed by Pd(ddpf)Cl₂ (70 mgs, 0.1 mmol) and heated at 80° C. for 1 hour. LC-MS indicated completion of the reaction. EtOAc (10 mL) was added and the organic layer was washed with H₂O (5 mL), dried and concentrated to give the title compound at greater than 90% purity. LC-MS: Calc. 446.1 found 447.2 (M+H).

### Step 2: Ethyl 1-(benzyloxy)-6-(2-methoxyphenyl)-2-oxo-4-{[(trifluoromethyl)sulfonyl]oxy}-1,2-dihydro-1,8-naphthyridine-3-carboxylate-Ethyl 1-(benzyloxy)-4-hydroxy-6-(2-methoxyphenyl)-2-oxo-1,2-dihydro-1,8-naphthyridine-3-carboxylate (420 mgs, 0.9 mmol) from Step 1 was taken up in DCM and TEA (0.5 mL) and trifluoromethanesulfonicanhydride (0.5 mL) were added. The solution was stirred for 1 hour. LC-MS indicated completion of reaction. H₂O (10 mL) was added and the organic layer was separated, dried and concentrated to give the title compound at greater than 85% pure. LC-MS: Calc. 578.1 found 579.1 (M+H).

### Step 3: ethyl 4-[4′-(aminomethyl)biphenyl-3-yl]-1-hydroxy-6-(2-methoxyphenyl)-2-oxo-1,2-dihydro-1,8-naphthyridine-3-carboxylate

Ethyl 1-(benzyloxy)-6-(2-methoxyphenyl)-2-oxo-4-{[(trifluoromethyl)sulfonyl]oxy}-1,2-dihydro-1,8-naphthyridine-3-carboxylate (500 mgs, 0.8 mmol) from step 2 was taken up in anhydrous THF (5 mL) and (4′-{[(tert-butoxycarbonyl)amino]methyl}biphenyl-3-yl)boronic acid (350 mgs, 1.1 mmol) was added followed by sodium carbonate solution (0.5 mL, 2.0 M). The solution was heated at 80° C. for 30 minutes. The solution was cooled, and EtOAc (20 mL) was added and the organic layer was separated, dried and concentrated. The crude intermediate was taken up in HOAc (1.0 mL) and 33% HBr in HOAc (0.5 mL) and heated at 80° C. for 1 hour. The solution was cooled, the HOAc was removed and the crude product was purified by RP-HPLC (C18 column; H₂O/CH₃CN). Yield (50 mgs, 20% yield). ¹H NMR (400 MHz, d₆-DMSO, ppm): δ 8.77 (dd, J=5.1, 1.7, 1H), 7.88 (s, 1H), 7.73 (m, 2H), 7.49-7.36 (m, 8H), 7.29-7.22 (m, 4H), 4.08 (s, 2H), 4.05 (q, J=7.6 Hz, 2H), 0.91 (t, J=7.1 Hz, 3H). LC-MS: Calc. 521.1 found 522.2 (M+H).

### Example 249

### ethyl 5-{[3′-(aminomethyl)biphenyl-3-yl]methyl}-1,4-dihydroxy-2-oxo-1,2-dihydro-1,8-naphthyridine-3-carboxylate

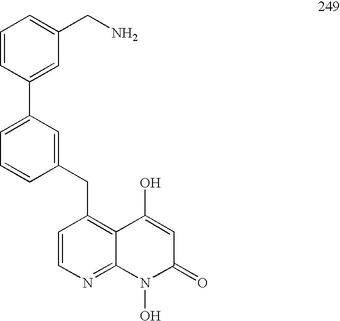

### Step 1: ethyl 1-(benzyloxy)-5-(3-bromobenzyl)-4-hydroxy-2-oxo-1,2-dihydro-1,8-naphthyridine-3-carboxylate

The ethyl 1-(benzyloxy)-4-hydroxy-5-iodo-2-oxo-1,2-dihydro-1,8-naphthyridine-3-carboxylate (0.220 g, 0.500 mmol) was dissolved in DME (5.0 mL). To this was added bromobenzylzinc bromide (1 mL, 1.0 M solution in THF)), and Pd (dppf)₂Cl₂ (0.044 g, 0.01 mmol) while N₂ was bubbled through the solution. The reaction vessel was sealed and the reaction heated at 80° C. for 1 hour. The solvent was removed. The residue was diluted with aqueous HCl (1N, 10 mL), and extracted into EtOAc (3×10 mL). The organic layers were combined, dried, filtered, and concentrated. The crude product (200 mg) was carried on. ES MS: m/z=509.1 (M+1).

### Step 2: ethyl 1-(benzyloxy)-5-[(3′-{[(tert-butoxycarbonyl)amino]methyl}biphenyl-3-yl)methyl]-4-hydroxy-2-oxo-1,2-dihydro-1,8-naphthyridine-3-carboxylate

Ethyl 1-(benzyloxy)-5-(3-bromobenzyl)-4-hydroxy-2-oxo-1,2-dihydro-1,8-naphthyridine-3-carboxylate: from step 1 (0.200 g, 0.41 mmol) was dissolved in DMF (5.0 mL) and H₂O (1.0 mL). To this was added 3-(N-BOC-aminomethyl)phenylboronic acid (0.101 g, 0.404 mmol), K₂CO₃ (0.084 g, 0.606 mmol), and the Pd dppf (DCM adduct) catalyst (0.008 g, 0.010 mmol) while N₂ was bubbled through the solution. The reaction vessel was sealed and the reaction heated in a microwave at 100° C. for 0.5 hour. The solution was cooled to room temperature, diluted with H₂O (6 mL), and extracted into EtOAc (3×10 mL). The organic layers were combined, dried, filtered, and concentrated. The residue was purified by SGC (0-100% EtOAc/hexane) to afford the title compound (150 mgs). ES MS: m/z=636.1 (M+1).

### Step 3: ethyl 5-{[3′-(aminomethyl)biphenyl-3-yl]methyl}-1,4-dihydroxy-2-oxo-1,2-dihydro-1,8-naphthyridine-3-carboxylate

Ethyl 1-(benzyloxy)-5-[(3′-{[(tert-butoxycarbonyl)amino]methyl}biphenyl-3-yl)methyl]-4-hydroxy-2-oxo-1,2-dihydro-1,8-naphthyridine-3-carboxylate (0.0592 g, 0.172 mmol) in HBr (33 wt. % in AcOH, 2 mL) and H₂O (0.5 mL) was heated to 80° C. for 0.5 hour. The solvent was removed. The residue was purified by RP-HPLC (C18 column; H₂O/CH₃CN with 0.1% TFA) to afford the title compound. LC-MS: 446.1 (M+1) Calculated: 445.1

### Example 250

### 6-amino-1,4-dihydroxy-3-phenyl-1,8-naphthyridin-2(1H)-one

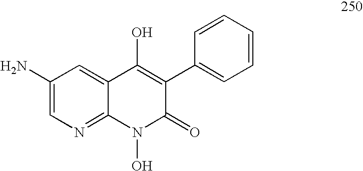

### Step 1: 1-(benzyloxy)-4-hydroxy-6-nitro-3-phenyl-1,8-naphthyridin-2(1H)-one

ethyl 2-[(benzyloxy)amino]-5-nitronicotinate (1 gm, 0.33 mmol), ethyl phenylacetate (1 mL), sodium ethoxide (400 mgs, 0.66 mmol) were added in EtOH and refluxed overnight. The solution was acidified with HCl (2.0 mL, 1.0 M) and extracted into EtOAc. The organic layer was separate, dried, and concentrated. The product was recrystallized from EtOAc and hexanes (150 mgs, 12% yield).

### Step 2: 6-amino-1,4-dihydroxy-3-phenyl-1,8-naphthyridin-2(1H)-one

1-(Benzyloxy)-4-hydroxy-6-nitro-3-phenyl-1,8-naphthyridin-2(1H)-one (50 mgs, 0.12 mmol) from Step 1 was taken up in EtOH (10 mL) under N₂. TFA (0.5 mL) and 10% Pd/C (20 mgs) were added and hydrogenated at room temperature using a H₂ balloon. After 1 hour, the solution was filtered through celite, and concentrated. The product was triturated with the addition of diethyl ether (10 mL). ¹H NMR (400 MHz, d₆-DMSO, ppm): δ 8.71 (s 11H), 7.88 (s, 1H), 7.73-7.61 (m, 5H), LC-MS: Calc. 269.1 found 270.2 (M+H).

### Example 251

### 4-[7-(3-aminophenyl)-3,4-dihydroisoquinolin-2(1H)-yl]-1-hydroxy-1,8-naphthyridin-2(1H)-one

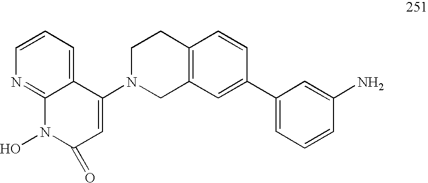

### Step 1: 1-(benzyloxy)-4-(7-bromo-3,4-dihydroisoquinolin-2(1H)-yl)-1,8-naphthyridin-2(1)-one

1-(benzyloxy)-2-oxo-1,2-dihydro-1,8-naphthyridin-4-yltrifluoromethanesulfonate (Example 2, Step 1: 500 mg, 1.249 mmol) and 7-bromo-1,2,3,4-tetrahydroisoquinoline (1007 mg, 4.75 mmol) in DMF (10 ml) was heated at 110° C. and stirred for 90 minutes. The crude mixture was dissolved in DCM and purified by SGC (30-100% EtOAc-hexanes) to give the title compound. MS: m/z=462.3 (M), 464.3 (M+2).

### Step 2: 4-[7-(3-aminophenyl)-3,4-dihydroisoquinolin-2(1H)-yl]-1-(benzyloxy)-1,8-naphthyridin-2(1H)-one

1-(benzyloxy)-4-(7-bromo-3,4-dihydroisoquinolin-2(1H)-yl)-1,8-naphthyridin-2(1)-one (50 mg, 0.324 mmol), 3-aminophenylboronic acid (89 mg, 0.649 mmol), PdCl₂(dppf)-DCM (13.25 mg, 0.016 mmol), and K₂CO₃ (224 mg, 1.622 mmol) in DMF (2 ml) and H₂O (0.5 ml) were degassed with N₂. The reaction mixture was stirred 120° C. in a microwave for 10 minutes. The crude mixture was diluted in EtOAc and washed with brine, dried and then concentrated. The residue was purified by SGC (50-100% EtOAc-hexane) to give the title compound ES MS: m/z-475.4 (M+1).

### Step 3: 4-[7-(3-aminophenyl)-3,4-dihydroisoquinolin-2(1H)-yl]-1-hydroxy-1,8-naphthyridin-2(1H)-one

4-[7-(3-aminophenyl)-3,4-dihydroisoquinolin-2(1H)-yl]-1-(benzyloxy)-1,8-naphthyridin-2(1H)-one (10 mg, 0.211 mmol) was dissolved in EtOH (10 ml). After degassing the reaction mixture with N₂ for 5 minutes, 10% Pd/C (20 mg) was added. The reaction vessel was primed with H₂ with a H₂ balloon 3×. The reaction mixture was stirred under a H₂ balloon for 2 hours. The Pd catalyst was filtered and the reaction mixture was purified using RP-HPLC (C18 column; 5-95% CH2CN/H2O with 0.1% TFA) to give the title compound. ¹H NMR (400 MHz, d₆-DMSO, ppm): δ 8.66 (d, J=3.3, 1H), 8.21 (d, J=6.6H, 1H), 7.37 (m, 7H), 7.02 (d, J=7.7, 11H), 6.21 (s, 1H), 4.40 (s, 2H), 3.49 (t, J=5.8, 2H), 3.12 (t, J=5.4, 2H). ES MS: m/z=385.4 (M).

TABLE 18
The compounds in the following table were prepared in accordance with the
procedures set forth in Example 251:

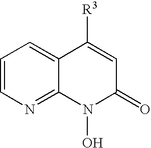

| Ex/ | | | |
|---|---|---|---|
| cpd | Name | R³ | Data |
| 252 | 4-[7-(3- aminomethylphenyl)- 3,4-dihydroisoquinolin- 2(1H)-yl]-1-hydroxy- 1,8-naphthyridin-2(1H)- one | 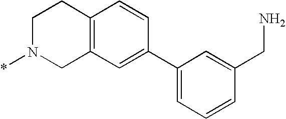 | ES MS: m/z = 399.4 (M). |
| 253 | 1-hydroxy-4-(7-pyridin- 4-yl-3,4- dihydroisoquinolin- 2(1H)-yl)-1,8- naphthyridin-2(1H)-one | 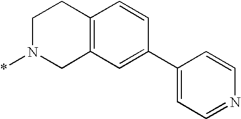 | ES MS: m/z = 371.4 (M). |
| 254 | 4-[7-(4-aminophenyl)- 3,4-dihydroisoquinolin- 2(1H)-yl]-1-hydroxy- 1,8-naphthyridin-2(1H)- one | 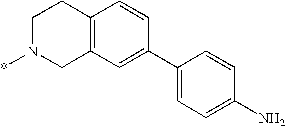 | ES MS: m/z = 385.4 (M). |
| 255 | 1-hydroxy-4-(7-pyridin- 3-yl-3,4- dihydroisoquino1in- 2(1H)-yl)-1,8- naphthyridin-2(1H)-one | 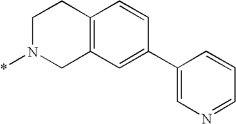 | ES MS: m/z = 371.4 (M). |
| 256 | 1-hydroxy-4-(5-phenyl- 1,3-dihydro-2H- isoindol-2-yl)-1,8- naphthyridin-2(1H)-one | 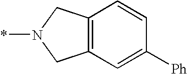 | ES MS: m/z = 356.3 (M + 1). |
| 257 | 4-{5-[4- (aminomethyl)phenyl]- 1,3-dihydro-2H- isoindol-2-yl}-1- hydroxy-1,8- naphthyridin-2(1H)-one | 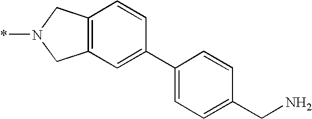 | ES MS: m/z = 386.4 (M + 1). |

TABLE 19
The compounds in the following table were prepared in accordance with the
procedures set forth in Example 251 except ethyl 1-hydroxy-2-oxo-4-
{[(trifluoromethyl)sulfonyl]oxy}-1,2-dihydro-1,8-naphthyridine-3-carboxylate was used as the
starting material:

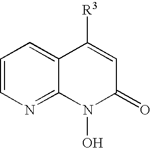

| Ex/ | | | |
|---|---|---|---|
| cpd | Name | R³ | Data |
| 258 | ethyl 4-[7-(4- fluorophenyl)-3,4- dihydroisoquinolin- 2(1H)-yl]-1-hydroxy-2- oxo-1,2-dihydro-1,8- naphthyridine-3- carboxylate | 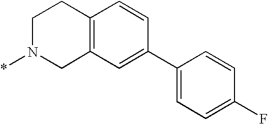 | ES MS: m/z = 460.4 (M + 1). |
| 259 | ethyl 4-[7-(3- fluorophenyl)-3,4- dihydroisoquinolin- 2(1H)-yl]-1-hydroxy-2- oxo-1,2-dihydro-1,8- naphthyridine-3- carboxylate | 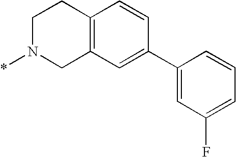 | ES MS: m/z = 460.4 (M + 1). |

### Example 260

### 4-[7-(phenylethylaminocarbonyl)-3,4-dihydroisoquinolin-2(1H)-yl]-1-hydroxy-1,8-naphthyridin-2(1H)-one

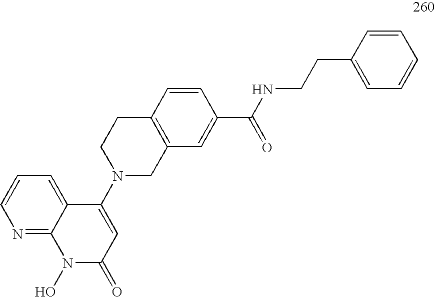

### Step 1: Methyl 2-[1-(benzyloxy)-2-oxo-1,2-dihydro-1,8-naphthyridin-4-yl]-1,2,3,4-tetrahydroisoquinoline-7-carboxylate

1-(benzyloxy)-2-oxo-1,2-dihydro-1,8-naphthyridin-4-yltrifluoromethanesulfonate (Example 2, Step 1: 400 mg, 1.249 mmol) and methyl 1,2,3,4-tetrahydroisoquinoline-7-carboxylate (382 mg, 2.00 mmol) in DMF (5 ml) was heated at 110° C. and stirred for 90 minutes. The crude mixture was dissolved in DCM and purified by SGC (0-30% EtOAc-hexanes) to give the title compound. MS: m/z=442.4 (M+1).

### Step 2: 2-[1-(benzyloxy)-2-oxo-1,2-dihydro-1,8-naphthyridin-4-yl]-1,2,3,4-tetrahydroisoquinoline-7-carboxylic acid

To a solution of methyl 2-[1-(benzyloxy)-2-oxo-1,2-dihydro-1,8-naphthyridin-4-yl]-1,2,3,4-tetrahydroisoquinoline-7-carboxylate (90 mg, 0.204 mmol) in THF (19 ml), added KOTMS (78.5 mg, 0.612 mmol). The reaction mixture was stirred overnight at room temperature. The reaction mixture was diluted with EtOAc, washed with H₂O, brine and then dried and concentrated to give the crude title compound which was used directly in the next step. ES MS: m/z=428.3 (M+1).

### Step 3: 2-[1-(benzyloxy)-2-oxo-1,2-dihydro-1,8-naphthyridin-4-yl]-N-(2-phenylethyl)-1,2,3,4-tetrahydroisoquinoline-7-carboxamide

To a solution of 2-[1-(benzyloxy)-2-oxo-1,2-dihydro-1,8-naphthyridin-4-yl]-1,2,3,4-tetrahydroisoquinoline-7-carboxylic acid (45 mg, 0.105 mmol) in DMF (1.5 ml), TEA (13.25 mg, 0.016 mmol), HATU (80 mg, 0.211 mmol) were added. 2-phenylethanamine (25.5 mg, 0.211 mmol) was then added to the reaction mixture under N₂. The reaction mixture was stirred at room temperature overnight. The crude mixture was diluted in EtOAc and washed with saturated aqueous solution of Na₂CO₃, dried and then concentrated to give the crude title compound which was used directly in the next step. ES MS: m/z=531.4 (M+1).

### Step 4: 4-[7-(3-aminophenyl)-3,4-dihydroisoquinolin-2(1H)-yl]-1-hydroxy-1,8-naphthyridin-2(1H)-one

2-[1-(benzyloxy)-2-oxo-1,2-dihydro-1,8-naphthyridin-4-yl]-N-(2-phenylethyl)-1,2,3,4-tetrahydroisoquinoline-7-carboxamide (50 mg, 0.094 mmol) was dissolved in EtOH (10 ml). After degassing the reaction mixture with N₂ for 5 minutes, Pd(OH)₂ (5 mg) was added. The reaction vessel was primed with H2 with a H₂ balloon 3×. The reaction mixture was stirred under a H₂ balloon for 3 hours. The Pd catalyst was filtered and the reaction mixture was purified using RP-HPLC (C18 column; 5-95% CH₃CN/H₂O with 0.1% TFA) to give the title compound. ¹H NMR (400 MHz, d₆-DMSO, ppm): δ 8.65 (broad s, 1H), 8.52 (broad s, 1H), 8.20 (d, J=7.14, 11H), 7.68 (m, 2H), 7.28 (m, 7H), 6.20 (s, 1H), 4.36s, 2H), 3.47 (broad s, 4H), 3.11 (broad s, 2H), 2.85 (broad s, 2H). ES MS: m/z=441.4 (M+1).

### Example 261

### 4-[6-(benzylaminocarbonyl)-3,4-dihydroisoquinolin-2(1H)-yl]-1-hydroxy-1,8-naphthyridin-2(1H)-one

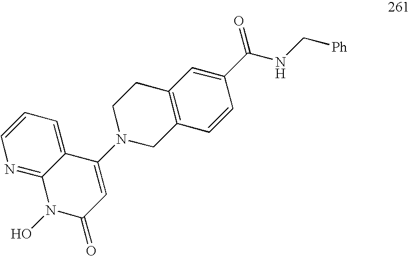

The above compound was prepared in accordance with the procedures set forth in Example 260. ES MS: m/z=427.4 (M+1).

### Example 262

### Ethyl 4-[4-(2-pyridin-4-ylethyl)phenyl]-hydroxy-2-oxo-1,2-dihydro-1,8-naphthyridin-3-carboxylate

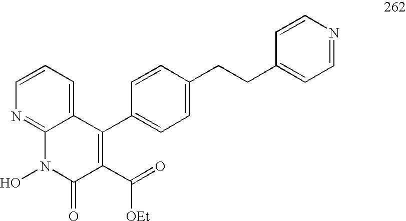

### Step 1: ethyl 1-(benzyloxy)-2-oxo-4-[4-(2-pyridin-4-ylethyl)phenyl]-1,2-dihydro-1,8-naphthyridine-3-carboxylate

To a solution of 1-(benzyloxy)-2-oxo-1,2-dihydro-1,8-naphthyridin-4-yl trifluoromethanesulfonate (Example 103, Step 1; 75 mg, 0.159 mmol) in THF (2 ml), 4-{2-[4-(4,4,5,5-tetramethyl-1,3,2-dioxaborolan-2-yl)phenyl]ethyl}pyridine (98 mg, 0.318 mmol), K₂CO₃ (65.8 mg, 0.476 mmol) and H₂O (1 ml) were added. N₂ was bubbled through the solution Pd(dppf)Cl₂ (12.97 mg, 0.016 mmol) was added and the reaction vessel sealed. This solution was heated in a microwave reactor at 120° C. for 20 minutes, after which the solution was cooled and partitioned between HCl (1.0 M, 10 mL) and EtOAc (10 mL). The organic layer was separated, dried and concentrated. The residue was purified by SGC (50-100% EtOAc-hexane) to give title compound ES MS: m/z=505.8 (M).

### Step 2: Ethyl 4-[4-(2-pyridin-4-ylethyl)phenyl]1-hydroxy-2-oxo-1,2-dihydro-1,8-naphthyridin-3-carboxylate

A mixture of ethyl 1-(benzyloxy)-2-oxo-4-[4-(2-pyridin-4-ylethyl)phenyl]-1,2-dihydro-1,8-naphthyridine-3-carboxylate (50 mg, 0.06 mmol) in 33 wt % HBr—HOAc (3 mL, 18.23 mmol) and H₂O (1 mL) was heated at 80° C. for 0.5 hour. The solvents were removed and the residue was purified using RP-HPLC (C18 column; 5-95% CH₃CN/H₂O with 0.1% TFA) to give the title compound. ¹H NMR (400 MHz, d₆-DMSO, ppm) δ 8.81 (broad s, 2H), 7.91 (broad s, 2H), 7.62 (s, 1H), 7.37 (m, 6H), 3.99 (broad s, 2H), 3.23 (broad s, 2H), 3.11 (broad s, 2H) 0.87 (broad s, 3H). ES MS: m/z=415.8 (M+1).

TABLE 20
The compounds in the following table were prepared in accordance with the
procedures set forth in Example 262:

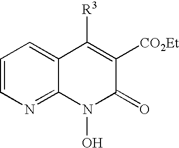

| Ex/ | | | |
|---|---|---|---|
| cpd | Name | R³ | Data |
| 263 | ethyl 4-[4′- aminomethyl-5-(2- fluorophenethyl)bi- phenyl-3-yl]1-hydroxy-2- oxo-1,2-dihydro-1,8- naphthyridine-3- carboxylate | 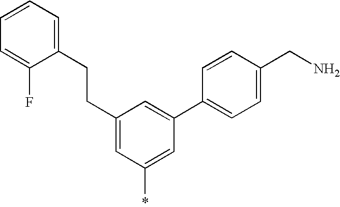 | ES MS: m/z = 537.9 (M). |
| 264 | ethyl 4-{4-[2-(4- aminophenyl)ethyl] phenyl}1-hydroxy-2-oxo- 1,2,-dihydro-1,8- naphthyridin-3- carboxylate | 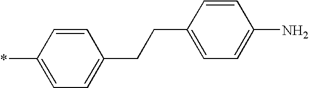 | ES MS: m/z = 429.8 (M). |

### Example 265

### 5-Hydroxybenzo[c]-1,8-naphthyridin-6(5B)-one

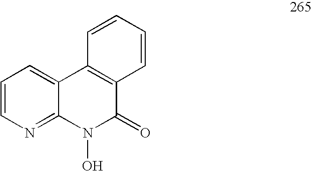

### Step 1: Ethyl 2-pyridin-3-ylbenzoate

Ethyl 2-bromobenzoate (1.4 ml, 8.7 mmol), pyridine-3-boronic acid (1.6 g, 13 mmol), Tetrakis (0.5 g, 0.44 mmol), and K₂CO₃ (3.6 g, 26 mmol) were combined in Toluene (20 ml) and heated at reflux for 3 hours. The reaction was filtered through a fritted syringe to remove the solids, washing with EtOAc. The filtrate was concentrated and residue was purified by RP-HPLC (C18 column; H₂O/CH₃CN with 0.1% TFA) to afford the title compound. ES MS: m/z=228 (M+1).

### Step 2: 2-Pyridin-3-ylbenzoic Acid

Ethyl 2-bromobenzoate (600 mg, 2.6 mmol) was stirred in a solution of NaOH (5.2 ml, 5.2 mmol) and MeOH (10 ml) at 50° C. overnight. The solvent was removed and the residue was purified by RP-HPLC (C18 column; H₂O/CH₃CN with 0.1% TFA) to afford the title compound. ¹H NMR (400 MHz, d₆-DMSO, ppm): δ 8.72 (m, 2H), 8.10 (d, J=7.8 Hz, 1H), 7.95 (d, J=7.8 Hz, 1H), 7.74-7.67 (m, 2H), 7.59 (m, 1H) and 7.46 (d, J=7.6 Hz, 1H). ES MS: m/z=200 (M+1).

### Step 3: N-(Benzyloxy)-2-pyridin-3-ylbenzamide

Ethyl 2-bromobenzoate (380 mg, 1.9 mmol), O-hydroxylbenzylamine (280 mg, 2.3 mmol), EDC (440 mg, 2.3 mmol), and HOBT (350 mg, 2.3 mmol) were combined in DMF (2 ml) and stirred over the 2 days at room temperature. The reaction was purified by RP-HPLC (C18 column; H₂O/CH₃CN with 0.1% TFA) to afford the title compound. ¹H NMR (400 MHz, d₆-DMSO, ppm): δ 11.6 (s, 1H), 8.75 (s, 1H), 8.72 (d, J=4.8 Hz, 1H), 8.07 (d, J=8.0 Hz, 1H), 7.73 (t, J=6.6 Hz, 1H), 7.63 (t, J=7.3 Hz, 1H), 7.57-7.51 (m, 3H), 7.44-7.34 (m, 5H) and 4.76 (s, 2H). ES MS: m/z=305 (M+1).

### Step 4: To N-(Benzyloxy)-2-pyridin-3-ylbenzamide

(340 mg, 1.1 mmol) in DCM (10 ml) at 0° C. was added mCPBA (290 mg, 1.7 mmol). The reaction was stirred at room temperature and more mCPBA was added each hours until the reaction was completed. The reaction was purified by RP-HPLC (C18 column; H₂O/CH₃CN with 0.1% TFA) to afford the title compound. ES MS: m/z=321 (M+1).

### Step 5: 5-(Benzyloxy)benzo[c]-1,8-naphthyridin-6(5B)-one

Trifluoroacetic anhydride (0.07 ml, 0.49 mmol) was added to a solution of N-(benzyloxy)-2-(1-oxidopyridin-3-yl)benzamide (79 mg, 0.25 mmol) in DCM (2 ml) at 0° C. The solution was allowed to stir at room temperature for 1 hour. Another batch of TFAA (0.07 ml, 0.49 mmol) was added and the reaction was stirred overnight. The solvent was removed and the residue was purified by RP-HPLC (C18 column; H₂O/CH₃CN with 0.1% TFA) to afford the title compound. ES MS: m/z=303 (M+1).

### Step 6: 5-Hydroxybenzo[c]-1,8-naphthyridin-6(5H)-one

5-(Benzyloxy)benzo[c]-1,8-naphthyridin-6(5H)-one (30 mg, 0.10 mmol) was dissolved in a mixture of 33 wt % HBr in HOAc solution (1.5 mL) and H₂O (0.5 ml) and heated to 80° C. overnight. The solvent was removed and the residue was purified by RP-HPLC (C18 column; H₂O/CH₃CN with 0.1% TFA) to afford the title compound. ¹H NMR (400 MHz, d6-DMSO, ppm): δ 8.0 (d, J=8.0 Hz, 1H), 8.66 (d, J=4.7 Hz, 1H), 8.61 (d, J=8.1 Hz, 1H), 8.39 (d, J=8.0 Hz, 1H), 7.92 (t, J=8.1, 1H), 7.74 (t, J=7.8 Hz, 1H), and 7.45 (dd, J=7.8 and 4.7 Hz, 1H). High Resolution MS (FT-ICR): m/z found 213.0651 (M+1): calculated 213.0659 (M+1).

### Example 266

### 5-Hydroxy-N-methyl-6-oxo-5,6-dihydrobenzo[c]-1,8-naphthyridine-9-carboxamide

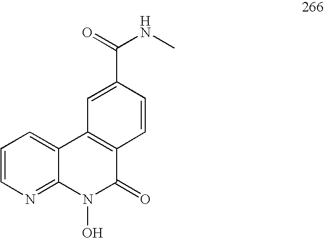

### Step 1: Dimethyl 2-pyridin-3-ylterephthalate

Dimethyl iodoterephthalate (1.5 g, 4.7 mmol), pyridine-3-boronic acid (0.63 g, 5.2 mmol), tetrakis (0.27 g, 0.23 mmol), and Cs₂CO₃ (3.0 g, 9.4 mmol) were heated to 130° C. overnight in DMF (25 ml). The solvent was removed and the residue was partitioned between H₂O and EtOAc. The layers were separated and the product was extracted from the aqueous. layer twice more with EtOAc. The combined organic extracts were dried over Na₂SO₄, filtered and conc. The crude product was purified by SGC (0-50% EtOAc/hexane) to afford the product. ¹H NMR (400 MHz, d6-DMSO, ppm): δ 8.61 (dd, J=4.8 and 1.5 Hz, 1H), 8.53 (d, J=2.4 Hz, 1H), 8.11 (dd, J=7.8 and 1.5 Hz, 1H), 8.00 (d, J=8.1 Hz, 1H), 7.95 (d, J=1.7 Hz, 1H), 7.80 (m, 1H), 7.48 (m, 1H), 3.90 (s, 3H) and 3.66 (s, 3H). ES MS: m/z=272 (M+1).

### Step 2: 4-(Methoxycarbonyl)-3-pyridin-3-ylbenzoic acid

Dimethyl 2-pyridin-3-ylterephthalate (340 mg, 1.3 mmol) was heated to 50° C. in a solution of MeOH (10 ml) and 1N NaOH (1.3 ml, 1.3 mmol) overnight. The solvent was removed and the residue was purified by RP-HPLC (C18 column; H₂O/CH₃CN with 0.1% TFA) to afford the title compound. ES MS: m/z=258 (M+1).

### Step 3: Methyl 4-[(methylamino)carbonyl]-2-pyridin-3-ylbenzoate

4-(Methoxycarbonyl)-3-pyridin-3-ylbenzoic acid (380 mg, 1.0 mmol), 2M methylamine in THF (1.0 ml, 2.0 mmol), EDC (390 mg, 2.0 mmol), and HOBT (310 mg, 2.0 mmol) were combined in DMF (7 ml) at room temperature. The reaction was stirred overnight then the solvent was removed. The residue was partitioned between H₂O and DCM, the layers were separated, and the product was extracted from the aqueous. layer twice more with DCM. The combined organic extracts were dried over Na₂SO₄, filtered and conc. The crude product was purified by SGC (0-5% MeOH/DCM) to afford the title compound ¹H NMR (400 MHz, d₆-DMSO, ppm): δ 8.68 (d, J=4.1 Hz, 1H), 8.60 (d, J=4.8 Hz, 1H), 8.55 (s, 1H), 7.96 (m, 2H), 7.90 (s, 1H), 7.79 (d, J=7.8 Hz, 1H), 7.48 (m, 1H), 3.65 (s, 3H) and 2.80 (d, J=4.5 Hz, 3H). ES MS: m/z=271 (M+1).

### Step 4: 5-Hydroxy-N-methyl-6-oxo-5,6-dihydrobenzo[c]-1,8-naphthyridine-9-carboxamide

In a similar manner to Example 265 (Steps 2 to 6), the title compound was prepared from methyl 4-[(methylamino)carbonyl]-2-pyridin-3-ylbenzoate. ¹H NMR (400 MHz, d₆-DMSO, ppm): δ 11.1 (bs, 1H), 8.96 (m, 2H), 8.82 (d, J=4.1 Hz, 1H), 8.69 (d, J=4.5 Hz, 1H), 8.45 (d, J=8.2 Hz, 1H), 8.12 (d, J=8.3 Hz, 1H), 7.51 (m, 1H), and 2.89 (d, J=4.2 Hz, 3H). High Resolution MS (FT-ICR): m/z found 270.0871 (M+1); calculated 270.0873 (M+1).

### Example 267

### 5-Hydroxy-9-phenylbenzo[c]-1,8-naphthyridin-6(5.H)-one

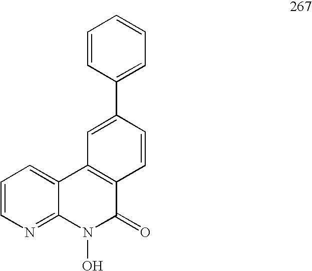

### Step 1: Methyl 4-chloro-2-pyridin-3-ylbenzoate

In a similar manner to Example 265 (Step 1), methyl 4-chloro-2-iodobenzoate was Suzuki coupled with pyridine 3-boronic acid to afford the title compound after SGC (0-50% EtOAc/hexane. ES MS: m/z=248 (M+1).

### Step 2: Methyl 3-pyridin-3-ylbiphenyl-4-carboxylate

Methyl 4-chloro-2-pyridin-3-ylbenzoate (2.5 g, 10 mmol), Pd(OAc)₂ (45 mg, 0.20 mmol), phenylbornic acid (1.85 g, 15 mmol), CsF (4.6 g, 30 mmol), and 2-dicyclohexylphosphino-2′-(N,N-dimethylamino)biphenyl (0.119 g, 0.303 mmol) were combined in degassed dioxane (30 ml) and stirred at 85° C. overnight. The reaction was filtered through a thin pack of celite, washing with dioxane and DMF. The solvent was removed and the residue was purified by SGC (0-50% EtOAc/hexane) to afford the title compound. ES MS: m/z=290 (M+1).

### Step 3: Methyl 3-(1-oxidopyridin-3-yl)biphenyl-4-carboxylate

m-CPBA (9.0 g, 52 mmol) was added to a 0° C. solution of methyl 3-pyridin-3-ylbiphenyl-4-carboxylate (3.0 g, 10.4 mmol) in DCM (100 ml). After stirring for 4 hours, the reaction was poured into NaHCO₃(aqueous) and extracted (4×) with DCM. The combined organic extracts were dried over Na₂SO₄, filtered and concentrated. The residue was purified by SGC (2-20% MeOH/DCM) to afford the title compound. ES MS: m/z=306 (M+1).

### Step 4: 3-(1-Oxidopyridin-3-yl)biphenyl-4-carboxylic acid

1N NaOH (17 ml, 17 mmol) was added to a solution of methyl 3-(1-oxidopyridin-3-yl)biphenyl-4-carboxylate (3.5 g, 11.3 mmol) in MeOH (57 ml) at 50° C. overnight. 1N HCl (17 mL, 17 mmol) was added to the cooled reaction and the solvent was removed. ES MS: m/z=292 (M+1).

### Step 5: 5-(Benzyloxy)-9-phenylbenzo[c]-1,8-naphthyridin-6(5H)-one

A mixture of 3-(1-oxidopyridin-3-yl)biphenyl-4-carboxylic acid (30 mg, 0.10 mmol), O-benzylhydroxylamine (38 mg, 0.31 mmol), EDC (59 mg, 0.31 mmol), and HOBT (32 mg, 0.21 mmol) in DMF (1.0 mL) was stirred at room temperature for 1 hour. Acetic anhydride (97 μl, 1.0 mmol) was added to the reaction and stirring was continued for 1 hour. The reaction was purified by RP-HPLC (C18 column; H₂O/CH₃CN with 0.1% TFA) to afford the title compound. ES MS: m/z=379 (M+1).

### Step 6: 5-Hydroxy-9-phenylbenzo[c]-1,8-naphthyridin-6(5H)-one

In a similar manner to Example 265 (Step 6), 5-(benzyloxy)-9-phenylbenzo[c]-1,8-naphthyridin-6(5H)-one was deprotected to afford the title compound. ¹H NMR (400 MHz, d₆-DMSO, ppm): δ 9.21 (m, 1H), 8.87 (s, 1H), 8.68 (d, J=4.6 Hz, 1H), 8.46 (d, J=8.2 Hz, 1H), 8.05 (m, 1H), 7.96 (d, J=7.7 Hz, 2H), 7.58 (m, 2H), and 7.52-7.47 (m, 2H). High Resolution MS (FT-ICR): m/z found 289.0970 (M+1); calculated 289.0972 (M+1).

### Example 268

### 5-Hydroxy-8-phenylbenzo[c]-1,8-naphthyridin-6(5H)-one

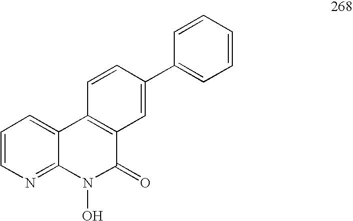

¹H NMR (400 MHz, d₆-DMSO, ppm): δ 9.03 (d, J=7.7 Hz, 1H), 8.72-8.67 (m, 2H), 8.62 (m, 1H), 8.24 (m, 1H), 8.05 (m, 1H), 7.86 (d, J=7.5 Hz, 2H), 7.56 (m, 2H), and 7.51-7.45 (m, 2H). High Resolution MS (FT-ICR): m/z found 289.0972 (M+1); calculated 289.0972 (M+1).

### Example 269

Representative compounds of the present invention exhibit inhibition of the HIV integrase or of HIV RNase H or of both. For example, compounds 1-268 were tested in the ASH assay as described above (using the alternative 5′-biotinylated DNA annealed to the complementary oligodeoxyribonucleotide 5′-ruthenium-GAGCAGAAAGAC (SEQ ID NO:3) and reading on a BioVeris M384 analyzer) and all were found to have IC₅₀ values of less than 100 micromolar. Compounds 1-268 were also tested in the integrase strand transfer assay (STA) as described above. The compounds of Examples 1-92, 94-162, 164-234, 236-257, and 260-268 were found to have IC₅₀ values of less than 50 micromolar, and the compounds of Examples 93, 163,235, 258, and 259 were found to have IC₅₀ values greater than 50 micromolar in the STA assay.

### Example 270

### Assay A for Inhibition of HIV Replication

An assay for measuring the inhibition of acute HIV infection with HeLa P4-2 cells in a single cycle infectivity assay (SCIA-A) was conducted in accordance with Joyce, J. G., et al., J. Biol. Chem., 2002, 277, 45811, Hazuda, D. J. et al., Science, 2000, 287, 646, and Kimpton, J. et al, J. Virol. 1992, 66, 2232. Infectious virus was produced by transfecting 293T cells with HIV proviral DNA in which the integrase gene was derived from a IIIB isolate and the remainder of the HIV genome was derived from the NL4-3 isolate. Compounds 1-16, 18-43, 47-69, 72-82, 87-95, 97-103, 168 and 171 were found to have antiviral IC₅₀ values of less than 100 micromolar in this assay.

### Assay B for Inhibition of HIV Replication

This assay B for measuring the inhibition of acute HIV infection with HeLa P4-2 cells in a single cycle infectivity assay (SCIA-B) is essentially the same as Assay A described above, except that HXB2 virus is employed instead of the IIIb isolate. Compounds 1-14, 16-59, and 61-268 were found to have antiviral IC₅₀ values of less than 100 micromolar, and the compounds of Examples 15 and 60 were found to have IC₅₀ values greater than 100 micromolar in this assay.

### Example 271

### Cytotoxicity Test A

The P4/R5 cell line used in the single-cycle HIV infectivity assays is a HeLa cell derived line containing a stably integrated LTR-LacZ reporter gene cassette. In the absence of virus infection, these cells express a low but measurable level of the reporter enzyme beta-galactosidase. Levels of reporter expression in the absence of virus and in the presence of varying concentrations of drug are measured using a chemiluminescent substrate for beta-galactosidase. The toxicity value assigned to a given compound, the MTC value, is the lowest concentration of the compound that results in a significant reduction in the basal beta-galactosidase expression levels in the absence of virus. Representative compounds of the present invention that were tested in the single cycle infectivity assay (see Assay A in Example 270) were examined for cytotoxicity up to a concentration of 100 micromolar, and were found to exhibit cytotoxicity only at concentrations significantly higher than concentrations providing an antiviral effect. In particular, Compounds 1-16, 18-43, 47-69, 72-82, 87-95, 97-103, 168 and 171 were tested in this assay. Most of those compounds did not exhibit cytotoxicity in this assay, and those that exhibited a cytotoxicity had MTC values that were at least three times higher than their IC₅₀ values for antiviral activity as measured in the Assay A of Example 270.

### Cytotoxicity Test B

The HeLa P4-2 cell line used in the single cycle HIV infectivity Assay B of Example 270 was also used to determine compound cytotoxicity in the absence of viral infection. The cytotoxicity of a compound was determined by using the nontoxic colorimetric-based assay, Alamar Blue (Biosource, Camarillo, Calif.), according to manufacturer's protocol, wherein the results are reported as LD₅₀ values. This assay was found to be a more sensitive measure of cytotoxicity than Test B above. Compounds 1-268 were examined for cytotoxicity up to a concentration of 100 micromolar. A majority of the compounds did not exhibit cytotoxicity in this test; i.e., no cytotoxicity was observed at concentrations ≦100 μM. The remaining compounds did exhibit cytotoxicity in the test. All of the compounds except for Compounds 15 and 60 were found to have LD₅₀ values that were at least five-fold greater than their antiviral IC₅₀ values determined in Assay B of Example 270.

The values obtained for certain of the compounds in the RNase H mediated RNA cleavage assay (ASH, Example 269), the integrase strand-transfer assay (STA, Example 269), the single-cycle HIV infectivity assay B (SCIA-B, Example 270), and the cytotoxicity test B (Example 271) are presented in Table 21.

TABLE 21

| | ASH | STA | SCIA-B | Cytotoxicity |
|---|---|---|---|---|
| Compound | (IC₅₀, μM) | (IC₅₀, μM) | (IC₅₀, μM) | (LD₅₀, μM) |
| 4 | 0.26 | 0.24 | 0.53 | 50 |
| 21 | 0.18 | 0.25 | 0.47 | >100 |
| 24 | 0.18 | 0.55 | 0.22 | >100 |
| 27 | 0.10 | 0.06 | 0.50 | 6.2 |
| 28 | 0.12 | 0.26 | 0.22 | >100 |
| 33 | 0.047 | 0.18 | 0.66 | >100 |
| 40 | 0.11 | 0.62 | 0.67 | >100 |
| 48 | 0.20 | 0.023 | 0.14 | 31 |
| 57 | 0.52 | 0.069 | 0.10 | 36 |
| 59 | 0.15 | 0.022 | 0.05 | 20 |
| 66 | 0.49 | 0.02 | 0.38 | 34 |
| 81 | 0.27 | 0.027 | 0.06 | >100 |
| 106 | 0.088 | 0.22 | 0.43 | 12 |
| 111 | 0.046 | 1.8 | 0.12 | >100 |
| 115 | 0.18 | 1.9 | 0.11 | 8.6 |
| 121 | 0.15 | 0.60 | 0.26 | 16 |
| 123 | 0.11 | 2.4 | 0.42 | 8.7 |
| 124 | 0.23 | 0.66 | 0.16 | 50 |
| 134 | 0.091 | 0.36 | 0.034 | 23 |
| 135 | 0.061 | 0.19 | 0.50 | >100 |
| 140 | 0.11 | 0.22 | 0.09 | 5 |
| 142 | 0.084 | 0.56 | 0.09 | 4.4 |
| 147 | 0.017 | 0.22 | 0.20 | 7.7 |
| 149 | 0.14 | 0.70 | 0.06 | 5.2 |
| 150 | 0.15 | 1.7 | 0.07 | 14 |
| 151 | 0.16 | 0.23 | 0.13 | 15 |
| 154 | 0.20 | 1.7 | 0.27 | 7.2 |
| 158 | 0.36 | 6.5 | 0.24 | 16 |
| 163 | 0.18 | >50 | 0.14 | 50 |
| 172 | 0.14 | 0.22 | 0.26 | 5.5 |
| 230 | 0.11 | 2.3 | 0.77 | >100 |
| 231 | 0.34 | 1.1 | 0.29 | 10 |
| 235 | 0.046 | >50 | 0.13 | 7.8 |
| 236 | 0.12 | 0.050 | 0.26 | 21 |
| 241 | 0.29 | 6.2 | 0.32 | 3.8 |
| 242 | 0.18 | 7.4 | 0.25 | 310 |
| 255 | 0.12 | 1.8 | 0.17 | 5.1 |
| 257 | 0.033 | 46 | 0.35 | 49 |

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 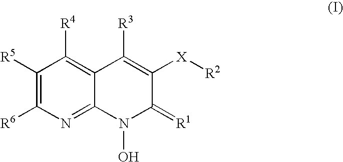
wherein:
R¹ is O, S, or N—R^{A};
X is a bond, C(O), SO₂, C₁-C₆ alkylene, O, N(R^{A}), or S;
R² is H, halo, CN, C₁-C₁₂ alkyl, C₃-C₈ cycloalkyl, aryl, heteroaryl, N(R⁷)R⁸, or OR⁹; wherein:
the alkyl is optionally substituted with from 1 to 3 substituents each of which is independently selected from the group consisting of halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A} SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, and C(O)N(R^{A})—C₁-C₆ alkylene-AryB;
wherein AryB is phenyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₈ cycloalkyl, CN, SO₂(C₁-C₆ alkyl), S(O)(C₁-C₆ alkyl), N(R^{A})R^{B}, NR^{A}SO_{R}^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-N(R^{A})R^{B}, C₁-C₆ alkylene-CO₂R^{A}, C₁-C₆ alkylene-C(O)R^{A}, or C₁-C₆ alkylene-C(O)N(R^{A})R^{B},
the cycloalkyl, aryl, or heteroaryl is optionally substituted with from 1 to 3 substituents each of which is independently selected from the group consisting of halo, OR^{A}, SR^{A}, C₁-C₆ alkyl, C₁-C₆ haloalkyl, N(R^{A})R^{B}, C₁-C₆ alkylene-N(R^{A})R¹, CO₂R^{B}, C₁-C₆ alkylene-CO₂R^{A}, NR^{B} SO₂R^{B}, C₁-C₆ alkylene-NR^{A} SO₂R^{B}, C(O)N(R^{A})R^{B}, C₁-C₆ alkylene-C(O)N(R^{A})R^{B}, C₁-C₆ alkylene-OR^{A}, C₁-C₆ alkylene-SR^{A}, SO₂N(R^{A})R^{B}, SO₂(C₁-C₆ alkyl), S(O)(C₁-C₆ alkyl), C(O)R^{A}, C₁-C₆ alkylene-C(O)R^{A}, NR^{A}CO₂R^{B}, NR^{A}C(O)R^{B}, NR^{A}C(O)N(R^{A})R^{B}CN, R^{C} and NO₂;
the alkyl or cycloalkyl is optionally also substituted with an oxo group; and
any two adjacent substituents of the cycloalkyl are optionally taken together with the ring atoms to which they are attached to form a ring fused to the cycloalkyl which is (i) a 5- to 7-membered unsaturated but non-aromatic carbocyclic ring, (ii) a benzene ring, (iii) a 5- or 6-membered heteroaromatic ring containing from 1 to 3 heteroatoms independently selected from N, O and S, or (iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂; and wherein the ring fused to the cycloalkyl is optionally substituted with from 1 to 3 substituents each of which is independently selected from the group consisting of halo, OR^{A}, SR^{A}, N(R^{A})R^{B}, R^{C}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A} SO₂R^{B}, SO₂N(R^{A})R^{A},NR^{B}CO₂R^{B}, NR^{A}C(O)R^{B}, NR^{A}C(O)N(R^{A})R^{B}CO₂R^{A}, C(O)R^{A}, and C(O)N(R^{A})R^{B};
and with the proviso (A) that XR² is not C(O)-halo, C(O)—CN, SO₂-halo, SO₂—CN, O-halo, O—CN, O—OR⁹, N(R^{A})-halo, N(R^{A})—CN, N(R^{A})—OR⁹, N(R^{A})—N(R⁷)R⁸, S-halo, S—CN, S—OR⁹, S—N(R⁷)R⁸, N(R^{A})-heteroaryl when the heteroaryl is attached to the N via a ring heteroatom, or S-heteroaryl when the heteroaryl is attached to the S via a ring heteroatom;
R³ is H, OH, halo, SO₂N(R⁷)R⁸, C₁-C₁₂ alkyl, OR⁹, N(R⁷)R⁸, NR^{A}C(O)R⁸, aryl, heteroaryl other than HetZ, HetZ, or C(O)-heteroaryl; wherein
the alkyl is optionally substituted with from 1 to 3 substituents each of which is independently selected from the group consisting of halo, OR^{A}, OR^{E}, SR^{A}, SR^{E}, N(R^{A})R^{B}, R^{D}, C₁-C₆ alkyl, C₁-C₆ haloalkyl, NO₂, CN, 502(C₁-C₆ alkyl), S(O)(C₁-C₆ alkyl), NR^{A} SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B};
the aryl or heteroaryl is optionally substituted with 1 to 3 substituents each of which is independently selected from the group consisting of halo, OR^{A}, OR^{E}, SR^{E}, SR^{E}, N(R^{A})R^{B}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}—C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}O, C₁-C₆ alkylene-N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{A}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{B}, C₁-C₆ alkylene-C(O)R^{A}, C₁-C₆ alkylene-C(O)N(R^{A})R^{B}, N(R^{A})—C₁-C₆ alkylene-C(O)N(R^{A})R^{B}, C(O)N(R^{A})R^{B}, C(O)—HetX, N(R^{A})—C₁-C₆ alkylene-HetX, and C₁-C₆ alkylene-HetX; and wherein HetX independently has the same definition as HetY; and
the HetZ is a fused bicyclic heteroaryl selected from the group consisting of: 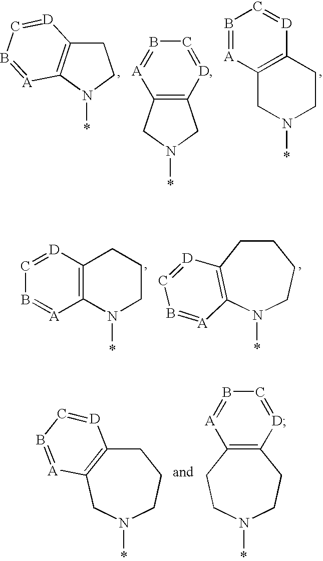
wherein A, B, C and D are each independently N or C-T, with the proviso that no more than two of A, B, C and D is N; and wherein each T is independently H, halo, CN, CO₂R^{A}, OR^{A}, SR^{A}, N(R^{A})R^{B}, N(R^{A})SO₂R^{B}, N(R^{A})CO₂R^{B}, N(R^{A})C(O)R^{B}, N(R^{A})C(O)N(R^{A})R^{B}, NO₂, CN, SO₂(C₁-C₆ alkyl), S(O)(C₁-C₆ alkyl), SO₂N(R^{A})(R^{B}), NR^{A}SO₂R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkyl, C₁-C₆ haloalkyl, C₁-C₆ alkylene-OR^{A}, C₁-C₆ alkylene-SR^{A}, C₁-C₆ alkylene-N(R^{A})R^{B}, C₁-C₆ alkylene-N(R^{A})SO₂R^{B}, C₁-C₆ alkylene-N(R^{A})CO₂R^{B}, C₁-C₆ alkylene-N(R^{A})C(O)R^{B}, C₁-C₆ alkylene-N(R^{A})C(O)N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-SO₂N(R^{A})(R^{B}), C₁-C₆ alkylene-NR^{A}SO₂R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, C₃-C₈ cycloalkyl, O—C₃-C₈ cycloalkyl, O—C₁-C₆ alkylene-C₃-C₈ cycloalkyl, S—C₃-C₈ cycloalkyl, S—C₁-C₆ alkylene-C₃-C₉ cycloalkyl, aryl, O-aryl, O—C₁-C₆ alkylene-aryl, S-aryl, S—C₁-C₆ alkylene-aryl, N(R^{A})—C₁-C₆ alkylene-aryl, C(O)N(R^{A})—C₁-C₆ alkylene-aryl, heteroaryl, O-heteroaryl, O—C₁-C₆ alkylene-heteroaryl, S-heteroaryl, S—C₁-C₆ alkylene-heteroaryl, N(R^{A})—C₁-C₆ alkylene-heteroaryl, or C(O)N(R^{A})—C₁-C₆ alkylene-heteroaryl, wherein
wherein in each T which is or contains C₃-C₈ cycloalkyl, the C₃-C₈ cycloalkyl is optionally and independently substituted with 1 to 3 substituents each of which is independently halogen, C₁-C₆ alkyl, C₁-C₆ haloalkyl, C₁-C₆ hydroxyalkyl, OR^{A}, N(R^{A})R^{B}, N(R^{A})R^{C}, N(R^{A})R^{E}, N(R^{A})SO₂R^{B}, N(R^{A})CO₂R^{B}, N(R^{A})C(O)R^{B}, N(R^{A})C(O)N(R^{A})R^{B}; NO₂, CN, SO₂(C₁-C₆ alkyl), S(O)(C₁-C₆ alkyl), SO₂N(R^{A})(R^{B}), NR^{A}SO₂R^{B}, NR^{A}CO₂R^{B}, NR^{A}C(O)R^{B}, NR^{A}C(O)N(R^{A})R^{B}, CO₂R^{A}, C(O)R^{A}, or C(O)N(R^{A})R^{B};
wherein in each T which is or contains aryl or heteroaryl, the aryl or heteroaryl is optionally substituted with 1 to 3 substituents each of which is independently selected from the group consisting of halo, OR^{A}, OR^{E}, SR^{A}, SR^{E}, N(R^{A})R^{B}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}-C₁-C₆alkylene-C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A} SO₂R^{B}, C₁-C₆ alkylene-SO₂N(R^{A})R^{B}, C₁-C₆ alkylene-NR^{A}CO₂R^{A}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, C(O)—HetY, and C₁-C₆ alkylene-HetY;
and wherein each HetY is independently a 4- to 7-membered saturated heterocyclyl containing a total of 1 or 2 heteroatoms selected from 1 or 2 N, zero or 10, and zero or 1S, wherein the heterocyclyl is optionally substituted with from 1 to 3 substituents each of which is independently halo, OH, O—C₁-C₆ alkyl, C₁-C₆ alkyl, O—C₁-C₆ haloalkyl, C₁-C₆ haloalkyl, C(O)R^{A}, CO₂R^{A}, or oxo;
alternatively, XR² and R³ are taken together with the carbon atoms to which each is attached to form:
(i) a 5- to 7-membered unsaturated but non-aromatic carbocyclic ring,
(ii) a benzene ring,
(iii) a 5- or 6-membered heteroaromatic ring containing from 1 to 3 heteroatoms independently selected from N, O and S, wherein each N is optionally oxidized,
(iv) a 5- to 7-membered unsaturated but non-aromatic heterocyclic ring containing from 1 to 3 heteroatoms independently selected from N, O and S, wherein each N is optionally oxidized and each S is optionally in the form of S(O) or S(O)₂, or
(v) a 5- to 7-membered unsaturated but non-aromatic heterocyclic ring having a 5- to 7-membered carbocyclic ring fused thereto via two adjacent carbon atoms in the heterocyclic ring, wherein the heterocyclic ring contains from 1 to 3 heteroatoms independently selected from N, O and S, wherein each N is optionally oxidized and each S is optionally in the form of S(O) or S(O)₂;
wherein:
the carbocyclic ring of (i), the benzene ring of (ii), the heteroaromatic ring of (iii), the heterocyclic ring of (iv) is fused to the naphthyridine ring to provide a fused tricyclic ring system, or the heterocylic ring of (v) is fused to the naphthyridine ring to provide a fused tetracyclic ring system;
the carbocyclic ring of (i), the benzene ring of (ii), the heteroaromatic ring of (iii), or the heterocyclic ring of (iv) is optionally substituted with from 1 to 4 substituents each of which is independently halo, OR^{A}, SR^{A}, N(R^{A})R^{A}, R^{A}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{A}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-OR^{A}, C₁-C₆ alkylene-SO₂, C₁-C₆ alkylene-N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{A}, C₁-C₆ alkylene-SO₂N(R^{A})R^{B}, C₁-C₆ alkylene-OR^{A}R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B} or phenyl,
wherein each phenyl is independently and optionally substituted with 1 to 3 substituents each of which is independently halo, C₁-C₆ alkyl, C₁-C₆ haloalkyl, CN, CO₂R^{A}, OR^{A}, SR^{A}, N(R^{A})R^{B}, N(R^{A})SO₂R^{B}, N(R^{A})CO₂R^{B}, N(R^{A})C(O)R^{B}, N(R^{A})C(O)N(R^{A})R^{B}, NO₂, SO₂(C₁-C₆ alkyl), S(O)(C₁-C₆ alkyl), SO₂N(R^{A})(R^{B}), NR^{A} SO₂R^{A}, NR^{A}CO₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆ C₁-C₆ alkylene-N(R^{A})R^{B}, C₁-C₆ alkylene-N(R^{A})C(O)R^{B}, C₁-C₆ alkylene-N(R^{A})CO₂R^{B}, C₁-C₆ alkylene-N(R^{A})C(O)R^{B}, C₁-C₆ alkylene-N(R^{A})C(O)N(R^{A})R^{B}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-SO₂N(R^{A})(R^{B}), C₁-C₆ alkylene-NR^{A}SO₂R^{B}, C₁-C₆ alkylene-NR²CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, C₁-C₆ alkylene-C(O)N(R^{A})R^{B}, C₃-C₈ cycloalkyl, AryC, O-AryC, O—C₁-C₆ alkylene-AryC, heteroaryl, HetW, C₁-C₆ alkylene-HetW; wherein:
each AryC independently has the same definition as AryA;
each HetW independently has the same definition as HetY; and
each heteroaryl is a 5- or 6-membered heteroaromatic ring containing from 1 to 4 heteroatoms selected from N, O and S, wherein the heteroaromatic ring is optionally substituted with 1 to 3 substituents each of which is independently halo, C₁-C₆ alkyl, C₁-C₆ haloalkyl, CO₂R^{A}, OR^{A}, SR^{A}, N(R^{A})R^{B}, CO₂R^{A}, C(O)R^{A}, C(O)N(R^{A})R^{B}, C₁-C₆ alkylene-OR^{A}, C₁-C₆ alkylene-N(R^{A})R^{B}, C₁-C₆ alkylene-CO₂R^{A}, C₁-C₆ alkylene-C(O)R^{A}, or C₁-C₆ alkylene-C(O)N(R^{A})R^{B};
the carbocyclic ring of (i), the heterocyclic ring of (iv), or the heterocyclic ring of (v) is optionally also substituted with 1 or 2 oxo groups; and
the carbocyclic ring fused to the heterocyclic ring of (v) is optionally substituted with 1 to 3 substituents each of which is independently halogen, OH, C₁-C₆ alkyl, O-C₁-C₆ alkyl, C₁-C₆ haloalkyl, O—C₁-C₆ haloalkyl, N(R^{A})R^{B}, or C₁-C₆ alkylene-N(R^{A})R^{B}, and wherein the heterocyclic ring of (v), in addition to being fused to the carbocyclic ring, is optionally substituted with 1 to 3 substituents each of which is independently OR^{A}, N(R^{A})R^{B}, C₁-C₆ alkyl, C₁-C₆ haloalkyl, SO₂(C₁-C₆ alkyl), S(O)(C₁-C₆ alkyl), NR^{A} SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-OR^{A}, C₁-C₆ alkylene-N(R^{A})R^{B}, C₁-C₆ alkylene-CO₂R^{A}, C₁-C₆ alkylene-C(O)R^{A}, C₁-C₆ alkylene-C(O)N(R^{A})R^{B}, or oxo; R⁴, R⁵, and R⁶ are each independently H, OH, halo, C₁-C₁₂ alkyl, C₂-C₁₂ alkenyl, aryl, heteroaryl, C(O)N(R⁷)R⁸, N(R⁷)R⁸, C(O)N(R⁷)R⁸, SO₂N(R⁷)R⁸, C₃-C₈ cycloalkyl, heterocyclyl, OR⁹, CO₂R⁹, or C(O)R¹⁰; wherein:
the alkyl, alkenyl, cycloalkyl, or heterocyclyl is optionally substituted with 1 to 3 substituents each of which is independently selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NA BC(O)R^{B}, NR^{A}C(O)N(R^{A})R^{B}, CO₂R^{A}, C(O)R^{A}, C(O)N(R^{A})R^{A}, C(O)N(R^{A})R^{D}, and C₁-C₆ alkylene-N(R^{A})R^{B};
the alkyl, cycloalkyl, or heterocyclyl is optionally also substituted with an oxo group; and
the aryl or heteroaryl is optionally substituted with 1 to 3 substituents each of which is independently selected from the group consisting of halo, OR^{A}, SR^{A}, N(R^{A})R^{B}, N(A)D, R^{D}, R^{E}, C₁-C₆ alkyl, C₁-C₆ haloalkyl, NO₂, CN, —C6 alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}—C₁-C₆ alkylene-C(O)N(R^{A})R^{B}, CO₂R^{A}, C(O)R^{A}, C(O)N(R^{A})R^{B}, C(O)N(R^{A})R^{D}, C₁-C₆ alkylene-N(R^{A})R^{B}, C₁-C₆ alkylene-OR^{A}, C₁-C₆ alkylene-SR^{A}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{B}, C₁-C₆ alkylene-C(O)R^{A}, C₁-C₆ alkylene-C(O)N(R^{A})R^{B}, and C(O)—HetS; wherein each HetS independently has the same definition as HetY;
alternatively, R⁴ and R⁵ taken together with the carbons to which each is attached form:
(i) a 5- to 7-membered unsaturated but non-aromatic carbocyclic ring,
(ii) a benzene ring,
(iii) a 5- or 6-membered heteroaromatic ring containing from 1 to 3 heteroatoms independently selected from N, O and S, or
(iv) a 5 to 7-membered unsaturated but non-aromatic heterocyclic ring containing from 1 to 3 heteroatoms independently selected from N, O and S, wherein each N is optionally oxidized and each S is optionally in the form of S(O) or S(O)₂,
wherein the carbocyclic ring of (i), the benzene ring of (ii), the heteroaromatic ring of (iii), or the heterocyclic ring of (iv) is fused to the naphthyridine ring to provide a fused tricyclic ring system,
wherein the carbocyclic ring of (i), the benzene ring of (ii), the heteroaromatic ring of (iii), or the heterocyclic ring of (iv) is optionally substituted with from 1 to 4 substituents each of which is independently C₁-C₆ alkyl, C₃-C₇ cycloalkyl, aryl, or heteroaryl, wherein the alkyl, cycloalkyl, aryl or heteroaryl is optionally substituted with from 1 to 3 substituents each of which is independently halo, OR^{A}, SR^{A}, N(R^{A})R^{B}, R^{C}, C₁-C₆ alkyl, C₁-C₆ haloalkyl, NO₂, CN, SO₂(C₁-C₆ alkyl), S(O)(C₁-C₆ alkyl), NR^{A} SO₂R^{A}, SO₂N(R^{A})R^{B}, NR^{A}CO₂R^{A}, NR^{A}C(O)R^{B}, NR^{A}C(O)N(R^{A})R^{B}, CO₂R^{B}, C(O)R^{A}, or C(O)N(R^{A})R^{B}, and
wherein the carbocyclic ring of (i) or the heterocyclic ring of (iv) is optionally also substituted with 1 or 2 oxo groups;
each R⁷ is independently H or C₁-C₁₂ alkyl, wherein the alkyl is optionally substituted with 1 to 3 substituents each of which is independently selected from the group consisting of oxo, halo, OR^{A}, SR^{A}, N(R^{A})R^{A}, R^{A}, C₁-C₆ alkyl, C₁-C₆ haloalkyl, NO₂, CN, SO₂(C₁-C₆ alkyl), S(O)(C₁-C₆ alkyl), NR^{B}SO₂R^{A}, SO₂N(R^{A})R^{A}, NR^{A}CO₂R^{A}, NR^{A}C(O)R^{B}, NR^{A}C(O)N(R^{A})R^{B}, C(2R^{A}, C(O)R^{A}, and C(O)N(R^{A})R^{B};
each R⁸ is independently H, C₁-C₁₂ alkyl, C₃-C₈ cycloalkyl, C₁-C₆ alkylene-C3-C₈ cycloalkyl, aryl, C₁-C₆ alkylene-aryl, heteroaryl, C₁-C₆ alkylene-heteroaryl, heterocyclyl, or C₁-C₆ alkylene-heterocyclyl; wherein:
the alkyl, cycloalkyl, aryl, heteroaryl, or heterocyclyl which is or is a part of R⁸ is optionally substituted with 1 to 3 substituents each of which is independently halo, OR^{A}, OR^{E}, SR^{A}, SR^{E}, N(R^{A})R^{B}, R^{E}, R^{D}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, C₁-C₆ alkylene-NR^{A}SO₂R^{B}, SO₂N(R^{A})R^{B}, NR^{A}CO₂R^{B}, NR^{A}C(O)R^{B}, NR^{A}-C₁-C₆ alkylene-C(O)R^{B}, NR^{A}C(O)N(R^{A})R^{B}, NR^{A}—C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-O-C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{A}, C₁-C₆ alkylene-CO₂R^{A}, C₁-C₆ alkylene-C(O)R^{A}, C₁-C₆ alkylene-C(O)N(R^{A})R^{B}, O-AryC, or O—C₁-C₆ alkylene-AryC, wherein AryC is aryl which is optionally substituted with from 1 to 3 substituents each of which is independently halo, OH, C₁-C₆ alkyl, C₁-C₆ haloalkyl, O—C₁-C₆ alkyl, O—C₁-C₆ haloalkyl, N(R^{A})R^{B}, CO₂R^{A}, or C(O)N(R^{A})R^{B}; and
the alkyl, cycloalkyl or heterocyclyl is optionally also substituted with an oxo group;
or R⁷ and R⁸ are optionally taken together with the N atom to which they are attached to form a 5- to 7-membered saturated heterocyclic ring, an unsaturated non-aromatic heterocyclic ring, or an aromatic heterocyclic ring, wherein the heterocyclic ring has from zero to 2 heteroatoms independently selected from N, O and S in addition to the N atom to which the R⁷ and R⁸ are attached; wherein each S atom in the saturated or unsaturated non-aromatic ring is optionally in the form S(O) or S(O)₂; and wherein the ring is optionally substituted with from 1 to 4 substituents each of which is independently halo, OR^{A}, SR^{A}, N(R^{A})R^{B}, C₁-C₆ alkyl, C₁-C₆ haloalkyl, NO₂, CN, SO₂(C₁-C₆ alkyl), S(O)(C₁-C₆ alkyl), CO₂R^{A}, C(O)R^{A}, C(O)N(R^{A})R^{B}, C₁-C₆ alkylene-SR^{A}, C₁-C₆ alkylene-SR^{A}, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-CO₂R^{A}, C₁-C₆ alkylene-C(O)R^{A}, C₁-C₆ alkylene-C(O)N(R^{A})R^{B}, oxo, aryl, C₁-C₆ alkylene-aryl, HetV, C₁-C₆ alkylene-HetV, with the proviso that no more than one substituent on the ring is aryl, C₁-C₆ alkylene-aryl, HetV, or C₁-C₆ alkylene-HetV; wherein:
HetV independently has the same definition as HetY; and
in any substituent of the heterocyclic ring formed from R⁷ and R⁸ taken together which is or contains aryl, the aryl is optionally substituted with from 1 to 3 substituents each of which is independently halo, OH, SH, S—C₁-C₆ alkyl, N(R^{A})R^{B}, C₁-C₆ alkyl, O—C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, C₁-C₆ alkylene-NR^{A}C(O)R^{B}, NR^{A}—C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-OH, C₁-C₆ alkylene-O—C₁-C₆ alkyl, C₁-C₆ alkylene-SH, C₁-C₆ alkylene-S—C₁-C₆ alkyl, C₁-C₆ alkylene-N(R^{A})R^{B}, C₁-C₆ alkylene-O—C₁-C₆ haloalkyl, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-CO₂R^{A}, C₁-C₆ alkylene-C(O)R^{A}, or C₁-C₆ alkylene-C(O)N(R^{A})R^{B};
each R⁹ is independently C₁-C₁₂ alkyl or aryl, wherein the aryl is optionally substituted with 1 to 3 substituents each of which is independently selected from the group consisting of halo, OR^{A}, SR^{A}, N(R^{A})R^{B}, N(R^{A})R^{D}, R^{D}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}—C₁-C₆ alkylene-C(O)N(R^{A})R^{B}, CO₂R^{A}, C(O)R^{A}, C(O)N(R^{A})R^{B}, C(O)N(R^{A})R^{D}, C₁-C₆ alkylene-N(R^{A})R^{B}, C₁-C₆ alkylene-OR^{A}, C₁-C₆ alkylene-SR^{A}, C₁-C₆ alkylene-NO₂, C₁-C₆ alkylene-CN, C₁-C₆ alkylene-SO₂(C₁-C₆ alkyl), C₁-C₆ alkylene-S(O)(C₁-C₆ alkyl), C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆-alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, or C₁-C₆ alkylene-C(O)N(R^{A})R^{B};
R¹⁰ is H or C₁-C₆ alkyl;
R^{A} is H, C₁-C₆ alkyl, C₁-C₆ haloalkyl, or C₃-C₈ cycloalkyl;
R^{B} is H, C₁-C₆ alkyl, C₁-C₆ haloalkyl, or C₃-C₈ cycloalkyl;
R^{C} is aryl or C₁-C₆ alkyl substituted with aryl;
R^{D} is aryl, C₁-C₆ alkyl substituted with aryl, heterocyclyl, C₁-C₆ alkyl substituted with heterocyclyl, heteroaryl, C₁-C₆ alkyl substituted with heteroaryl, C₃-C₇ cycloalkyl, or C₁-C₆ alkyl substituted with C₃-C₇ cycloalkyl, wherein:
in any substituted alkyl set forth in R^{D}, the alkyl is optionally substituted with 1 to 3 substituents each of which is independently selected from the group consisting of halo, OR^{A}, SR^{A}, N(R^{A})R^{B}, R^{C}, R^{E}, C₁-C₆ alkyl, C₁-C₆ haloalkyl, NO₂, CN, SO₂(C₁-C₆ alkyl), S(O)(C₁-C₆ alkyl), NR^{A} SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, and C(O)N(R^{A})R^{B}; and
in any R^{D} which is or contains cycloalkyl or heterocyclyl, the cycloalkyl or heterocyclyl is optionally substituted with 1 to 3 substituents each of which is independently selected from the group consisting of halo, OR^{A}, SR^{A}, N(R^{A})R^{A}, R^{E}, C₁-C₆ alkyl, C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, CO₂R^{A}, C(O)R^{A}, C(O)N(R^{A})R^{B}, C₁-C₆ alkylene-OR^{A}, C₁-C₆ alkylene-SR^{A}, C₁-C₆ alkylene-N(R^{A})R^{B}, C₁-C₆ alkylene-NR^{A}SO₂R^{B}, C₁-C₆ alkylene-SO₂N(R^{A})R^{B}, C₁-C₆ alkylene-NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{B}, C₁-C₆ alkylene-C(O)R^{A}, C₁-C₆ alkylene-C(O)N(R^{A})R^{B}, AryA, C₁-C₆ alkylene-AryA, C₁-C₆ alkylene-HetU, C(O)—HetU, C₁-C₆ alkylene-C(O)—HetU, C₁-C₆ alkylene-(AryA)₁₋₂, and oxo;
in any R^{D} which is or contains aryl or heteroaryl, the aryl or heteroaryl is optionally substituted with 1 to 3 substituents each of which is independently selected from the group consisting of halo, OR^{A}, SR^{A}, N(R^{A})R^{B}, R^{C}, R^{E}, C₁-C₆ alkyl, C₁-C₆ haloalkyl, O—C₁-C₆ haloalkyl, NO₂, CN, SO₂(C₁-C₆ alkyl), S(O)(C₁-C₆ alkyl), NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}-C₁-C₆ alkylene-C(O)N(R^{A})R^{B}, CO₂R^{A}, C(O)R^{A}, C(O)N(R^{A})R^{B}, C₁-C₆ alkylene-OR^{A}, C₁-C₆-alkylene-SR^{A}, C₁-C₆ alkylene-N(R^{A})R^{B}, C₁-C₆ alkylene-NR^{A}SO₂R^{B}, C₁-C₆ alkylene-SO₂N(R^{A})R^{B}, C₁-C₆, C₁-C₆ alkylene-NR^{A}C(O)R^{B}, C₁-C₆ alkylene-NR^{A}(O)N(R^{A})R^{B}, C₁-C₆ alkylene-CO₂, C₁-C₆ alkylene-C(O)R^{A}, C₁-C₆ alkylene-C(O)N(R^{A})R^{B}, CycA, AryA, C₁-C₆ alkylene-AryA, HetU, C(O)—HetU, C₁-C₆ alkylene-HetU, C₁-C₆ alkylene-C(O)—HetU, C₁-C₆ alkylene-CO₂R^{A}, C₁-C₆ alkylene-C(O)R^{A}, C₁-C₆ alkylene-C(O)N(R^{A})R^{B}, C₁-C₆ alkylene-AryA and C₁-C₆ alkylene-RF;
wherein:
each AryA is independently phenyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₈ cycloalkyl, CN, SO₂(C₁-C₆ alkyl), S(O)(C₁-C₆ alkyl), N(R^{A})R^{B}, NR^{A}SO₂R^{B}, SO₂N(R^{A})R^{B}, NR^{A}CO₂R^{B}, NR^{A}C(O)R^{B}, NR^{A}C(O)N(R^{A})R^{B}, NR^{A}—C₁-C₆ alkylene-C(O)N(R^{A})R^{B}, CO₂R^{A}, C(O)R^{A}, C(O)N(R^{A})R^{B}, C₁-C₆ alkylene-OH, C₁-C₆ alkylene-N(R^{A})R^{B}, C₁-C₆ alkylene-NR^{A}SO₂R^{B}, C₁-C₆ alkylene-N(R^{A})R^{B}SO₂N(R^{A})R^{B}, C₁-C₆ alkylene-N(R^{A})R^{B}NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, or C₁-C₆ alkylene-C(O)N(R^{A})R^{B};
CycA is C₃-C₈ cycloalkyl which is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, N(R^{A})R^{B}, or C₁-C₆ alkylene-N(R^{A})R^{B};
RF is C(O)-aryl, N(R^{A})-aryl, N(R^{A})—C₁-C₆ alkylene-aryl, C(O)N(R^{A})-aryl, S-aryl, SO₂-aryl, C(O)-heteroaryl, N(R^{A})-heteroaryl, C(O)N(R^{A})-heteroaryl, S-heteroaryl, or SO₂-heteroaryl, wherein the aryl or heteroaryl is optionally substituted with from 1 to 3 substituents each of which is independently halo, OH, C₁-C₆ alkyl, O—C₁-C₆ alkyl, C₁-C₆ haloalkyl, O—C₁-C₆ haloalkyl, C₁-C₆ alkenyl, C₃-C₈ cycloalkyl, CN, SO₂(C₁-C₆ alkyl), S(O)(C₁-C₆ alkyl), N(R^{A})R^{B}, NR^{A}SO₂R^{A}, SO₂N(R^{A})R^{B}, NR^{A}CO₂R^{A}, NR^{A}C(O)R^{B}, NR^{A}C(O)N(R^{A})R^{B}, CO₂R^{A}, C(O)R^{A}, C(O)N(R^{A})R^{B}, or C₁-C₆ alkylene-OH, C₁-C₆ alkylene-N(R^{A})R^{B}, C₁-C₆ alkylene-N(R^{A})R^{B}NR^{A}SO₂R^{B}, C₁-C₆ alkylene-N(R^{A})R^{B}SO₂N(R^{A})R^{B}, C₁-C₆ alkyleneCN(R^{A})R^{B}NR^{A}CO₂R^{B}, C₁-C₆ alkylene-NR^{A}C(O)R^{B}, C₁-C₆ alkylene-NR^{A}C(O)N(R^{A})R^{B}, C₁-C₆ alkylene-CO₂R^{A}, C₁-C₆ alkylene-C(O)R^{A}, or C₁-C₆ alkylene-C(O)N(R^{A})R^{B};
each HetU independently has the same definition as HetY; and
R^{E} is heteroaryl or C₁-C₆ alkyl substituted with heteroaryl;
and with the provisos that:
(B) when R¹ is O, R³ is H, and R⁴═R⁵═R⁶═H, then XR² is not C(O)OCH₂CH₃;
(C) when R¹ is O, XR² is C(O)N(R⁷)R⁸, R⁴═R⁵═R⁶═H, then R⁸ is not (pyridin-2-ylmethoxy)phenyl; and
(D) when R¹ is O, XR² is C(O)OR⁹, R⁴═R⁶═H, and R⁹ is ethyl, then R⁵ is not 3-cyanophenyl.

2. The compound of Formula I according to claim 1, or a pharmaceutically acceptable salt thereof, wherein R¹ is O.

3. The compound of according to claim 2, or a pharmaceutically acceptable salt thereof, wherein:
each R^{A} is independently H or C₁-C₆ alkyl;
each R^{B} is independently H or C₁-C₆ alkyl;
at least one of R⁴ and R⁵ is H; and
R⁶ is H, OH, or NH₂.

4. The compound of claim 3, or a pharmaceutically acceptable salt thereof, wherein XR² is H, Cl, Br, F, C₁-C₄ alkyl, C(O)O—C₁-C₄ alkyl, C(O)—C₁-C₄ alkyl, cyclopentyl, cyclohexyl, phenyl, CH₂-phenyl, pyridyl, pyrimidinyl, C(O)N(R^{7A})R^{8A}, or O—C₁-C₄ alkyl; wherein:
the C₁-C₄ alkyl is optionally substituted with C(O)O—C₁-C₄ alkyl or C(O)N(H)CH₂-phenyl, wherein the phenyl is optionally substituted with 1 or 2 substituents each of which is independently Cl, Br, F, OH, CH₃, OCH₃, CF₃, OCF₃, N(R^{A})R^{B}, or (CH₂)₁₋₂—N(R^{A})R^{B};
the phenyl or the phenyl which is part of CH₂-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
R^{7A} is the R⁷ associated with R² and is H or methyl;
R^{8A} is the R⁸ associated with R² and is H, C₁-C₄ alkyl, CH₂CF₃, CH₂CH₂CF₃, cyclopropyl, phenyl, CH₂-phenyl, CH(CH₃)-phenyl, heteroaryl, heterocyclyl, or CH₂-heterocyclyl, wherein:
the phenyl or the phenyl in CH₂-phenyl or CH(CH₃)-phenyl is optionally substituted with 1 or 2 substituents each of which is independently Cl, Br, F, OH, methyl, CN, OCH₃, CF₃, OCF₃, C(O)CH₃, N(H)C(O)CH₃, CO₂CH₃, C(O)NH₂, C(O)N(H)CH₃, or C(O)N(CH₃)₂;
the heteroaryl is pyridyl, pyrimidinyl, pyrrolyl, thienyl, furanyl, pyrazolyl, imidazolyl, oxazolyl, or thiazolyl, wherein the heteroaryl is optionally substituted with O-phenyl or OCH₂-phenyl, and is optionally also substituted with 1 or 2 substituents each of which is independently Cl, Br, F, OH, methyl, OCH₃, CF₃, OCF₃, C(O)CH₃, CO₂CH₃, C(O)NH₂, C(O)N(H)CH₃, or C(O)N(CH₃)₂, wherein the total number of substituents ranges from zero to 2;
the heterocyclyl or the heterocyclyl in CH₂-heterocyclyl is pyrrolidinyl, piperidinyl, piperazinyl, morpholinyl, or thiomorpholinyl, wherein the heterocyclyl is optionally substituted with oxo and is optionally also substituted with C₁-C₄ alkyl, C(O)O—C₁-C₄ alkyl or CH₂-phenyl;
alternatively the R^{7A} and R^{8A} are optionally taken together with the N atom to which they are bonded to form a saturated heterocyclic ring selected from the group consisting of piperidinyl, piperazinyl, pyrrolidinyl, morpholinyl, and thiomorphinyl, wherein the heterocyclic ring is optionally substituted with 1 to 3 substituents each of which is independently halo, OH, methyl, OCH₃, CF₃, OCF₃, C(O)R^{A}, CO₂R^{A}, C(O)N(R^{A})R^{B}, and oxo;
each R^{A} is independently H or C₁-C₄ alkyl; and
each R^{B} is independently H or C₁-C₄ alkyl.

5. The compound of claim 4, or a pharmaceutically acceptable salt thereof, wherein R³ is OH, NH₂, methyl, phenyl, naphthyl, 3,4-dihydronaphthyl, heteroaryl other than HetZ, HetZ, C(O)—HetZ, NR^{A}C(O)R^{8C}, or N(R^{7C})R^{8C}, wherein:
the methyl is substituted with phenyl or (CH₂)₁₋₂-phenyl, wherein either phenyl is further substituted by (i) another phenyl or (ii) another (CH₂)₁₋₂-phenyl, wherein the phenyl in (i) or (ii) is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{a}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) CH₂-phenyl, (32) CH(CH₃)-phenyl, (33) CH₂CH₂-phenyl, (34) heteroaryl, (35) CH₂-heteroaryl, (36) CH₂CH₂-heteroaryl, (37) CH(CH₃)-heteroaryl, (38) heterocyclyl, (39) CH₂-heterocyclyl, (40) CH(CH₃)-heterocyclyl, or (41) C(O)-heterocyclyl;
wherein the phenyl in (30), (31), (32), or (33) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (J) OCF₃, (k) N(R^{a})R^{B}, (l) CH₂—N(R^{A})R^{B}, (m) CH₂CH₂—N(R^{A})R^{B}, (n) CO₂R¹, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{a})C(O)R^{B}, (bb) N(R^{A})CH₂C(O)N(R^{A})R^{B}, (cc) CN, (dd) cyclopropyl optionally substituted with N(R^{A})R^{B}, (ee) CH₂—N(R^{A})CH₂-phenyl, (ff) heterocyclyl (gg) C(O)-heterocyclyl, (hh) CH₂-heterocyclyl, or (ii) CH(CH₃)-heterocyclyl; wherein the heterocyclyl in (ff), (gg), (hh) or (ii) is piperidinyl, piperazinyl (optionally substituted with C₁-C₄ alkyl), morpholinyl, pyrrolidinyl, or thiomorpholinyl;
wherein the heteroaryl in (34), (35), (36), or (37) is pyridyl, pyrimidinyl, pyrrolyl, thienyl, furanyl, pyrazolyl, imidazolyl, oxazolyl, or thiazolyl, and the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (J) OCF₃, (k) N(R^{A})R^{B}, (l) CH₂—N(R^{A})R^{B}, (m) CH₂CH₂—N(R^{a})R^{B}, (n) CO₂R^{A}, (O)CH₂—CO₂R^{A}, or (p) CH₂CH₂—CO₂R¹;
wherein the heterocyclyl in (38), (39), (40), or (41) is piperidinyl, piperazinyl, morpholinyl, pyrrolidinyl, or thiomorpholinyl, wherein the heterocyclyl is optionally substituted with oxo, and is also optionally substituted with (a) CO₂R^{A}, (b) CH₂—CO₂R^{A} (c) C(O)(R^{A}), (d) N(R^{A})R^{B}, (e) (CH₂)₁₋₃—N(R^{A})R^{B}, (f) C(O)N(R^{A})R^{B}, (g) (CH₂)₁₋₃—C(O)N(R^{A})R^{B}, (h) CH₂C(O)-heterocyclyl, (i) phenyl, (J) CH₂-phenyl, (k) CH(CH₃)-phenyl, (l) CH(phenyl)₂, wherein the heterocyclyl in (h) is piperidinyl, piperazinyl (optionally substituted with C₁-C₄ alkyl), morpholinyl, pyrrolidinyl, or thiomorpholinyl, and wherein the phenyl in (i), (J), (k), or (L) is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R¹;
the heteroaryl is
(A) pyridyl, pyrimidinyl, pyrrolyl, thienyl, furanyl, pyrazolyl, imidazolyl, oxazolyl, or thiazolyl, any of which is optionally substituted with 1 or 2 substitutents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) C(O)R^{A}, (18) CH₂—C(O)R^{A}, (19) SO₂(C₁-C₄ alkyl), (20) SO₂N(R^{A})R^{B}, (21) NHSO₂CH₃, (22) CH₂NHSO₂CH₃, (23) C(O)N(R^{A})R^{B}, (24) CH₂C(O)N(R^{A})R^{B}, (25) CH₂OH, (26) CH₂CH₂OH, (27) CN, (28) phenyl, (29) CH₂-phenyl, (30) CH(CH₃)-phenyl, (31) CH₂CH₂-phenyl, or (32) N(R^{A})(CH₂)₁₋₂-heterocyclyl;
wherein the phenyl in (28), (29), (30) or (31) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN; and
wherein the heterocyclyl in (32) is piperidinyl, piperazinyl (optionally substituted with C₁-C₄ alkyl), morpholinyl, pyrrolidinyl, or thiomorpholinyl; or 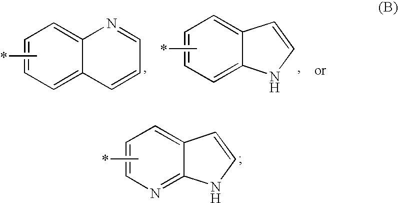
the HetZ is: 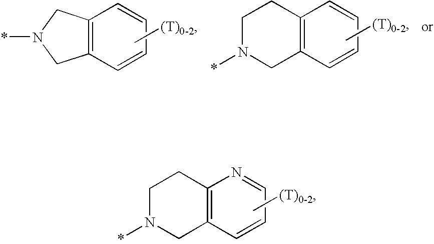
wherein each T is independently (1) H, (2) Cl, (3) Br, (4) F, (5) OH, (6) CH₃, (7) OCH₃, (8) CH₂F, (9) CF₃, (10) OCH₂F, (11) OCF₃, (12) N(R^{A})R^{B}, (13) CH₂—N(R^{A})R^{B}, (14) CH₂CH₂—N(R^{A})R^{B}, (15) CO₂R^{A}, (16) CH₂—CO₂R^{A}, (17) CH₂CH₂—CO₂R^{A}, (18) CN, (19) pyridyl, (20) pyrimidinyl, (21) phenyl, or (22) C(O)NH(CH₂)₁₋₂-phenyl;
wherein the phenyl in (21) or (22) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
R^{7C} is the R⁷ associated with R³ and is H or C₁-C₄ alkyl;
R^{8C} is the R⁸ associated with R³ and is C₁-C₄ alkyl, phenyl, CH₂-phenyl, CH₂CH₂-phenyl, CH(CH₃)-phenyl, indenyl, dihydroindenyl, 1,2,3,4-tetrahydronaphthyl, heteroaryl, CH₂-heteroaryl, CH(CH₃)-heteroaryl, CH₂CH₂-heteroaryl, heterocyclyl, CH₂-heterocyclyl, CH₂CH₂-heterocyclyl, or CH(CH₃)-heterocyclyl; wherein:
the C₁-C₄ alkyl is optionally substituted with 2 substituents one of which is phenyl and the other of which is OH, (CH₂)₁₋₂—N(R^{A})R^{B}, piperidinyl, piperazinyl (optionally substituted with C₁-C₄ alkyl), morpholinyl, pyrrolidinyl, or thiomorpholinyl;
the phenyl which is or is part of the R^{8C} is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) heteroaryl, (32) heterocyclyl, or (33) CH₂-heterocyclyl;
wherein the phenyl in (30) is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
wherein the heteroaryl in (31) is which is pyridyl, pyrimidinyl, pyrrolyl, thienyl, furanyl, pyrazolyl, imidazolyl, oxazolyl, thiazolyl, or triazolyl, and wherein the heteroaryl is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
wherein the heterocyclyl in (32) or (33) is piperidinyl, piperazinyl, morpholinyl, pyrrolidinyl, or thiomorpholinyl and is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A};
the heteroaryl which is or is part of R^{8C} is pyridyl, pyrimidinyl, pyrrolyl, thienyl, furanyl, pyrazolyl, imidazolyl, oxazolyl, or thiazolyl, and is optionally substituted with phenyl, CH₂-phenyl, heterocyclyl, or CH₂-heterocyclyl in which the heterocyclyl is piperidinyl, piperazinyl (optionally substituted with C₁-C₄ alkyl), morpholinyl, pyrrolidinyl, or thiomorpholinyl;
the heterocyclyl which is or is part of the R^{8C} is piperidinyl, piperazinyl, morpholinyl, pyrrolidinyl, or thiomorpholinyl, wherein the heterocyclyl is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, CO₂R^{A}, phenyl, or CH₂-phenyl;
alternatively the R^{7C} and R^{8C} together with the N to which both are bonded form a heterocycyl which is piperidinyl, piperazinyl, morpholinyl, pyrrolidinyl, or thiomorpholinyl, wherein the heterocyclyl is optionally substituted with oxo and is also optionally substituted with from 1 to 3 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) C(O)R^{A}, (12) CO₂R^{A}, (13) CH₂C(O)R^{A}, (14) CH₂CO₂R^{A}, (15) phenyl, (16) CH₂-phenyl, (17) CH(CH₃)-phenyl, (18) heterocyclyl, (19) CH₂-heterocyclyl, or (20) CH(CH₃)-heterocyclyl;
wherein the phenyl in (15), (16), or (17) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN; and
wherein the heterocyclyl in (18), (19) or (20) is piperidinyl, piperazinyl, morpholinyl, pyrrolidinyl, or thiomorpholinyl, wherein the heterocyclyl is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A}.

6. The compound according to claim 5, or a pharmaceutically acceptable salt thereof, wherein alternatively XR² and R³ are taken together with the carbon atoms to which each is attached to provide: 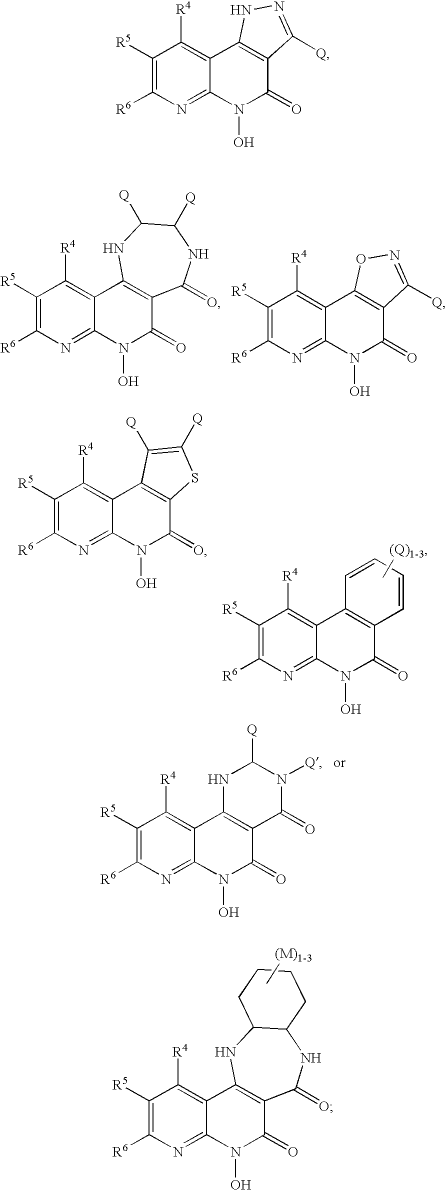
wherein:
each M is independently H, OH, Cl, Br, F, C₁-C₄ alkyl, N(R^{A})R^{B}, or (CH₂)₁₋₂—N(R^{A})R^{B},
each Q is independently H, Cl, Br, F, C₁-C₄ alkyl, C(O)N(R^{A})R^{B}, (CH₂)₁₋₂—C(O)N(R^{A})R^{B}, N(R^{A})R^{B}, (CH₂)₁₋₂—N(R^{A})R^{B}, or phenyl, wherein:
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) O-phenyl, (32) (CH₂)₁₋₂-phenyl, (33) O—(CH₂)₁₋₂-phenyl, (34) heteroaryl, (35) heterocyclyl, or (36) (CH₂)₁₋₂-heterocyclyl,
wherein the phenyl in (30), (31), (32), or (33) is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
wherein the heteroaryl in (34) is pyridyl, pyrimidinyl, pyrrolyl, thienyl, furanyl, pyrazolyl, imidazolyl, oxazolyl, thiazolyl, or triazolyl, and wherein the heteroaryl is optionally substituted with 1 or 2 substituents each of which is independently Cl, Br, F, OH, CH₃, OCH₃, CH₂F, CF₃, OCH₂F, OCF₃, N(R^{A})R^{B}, CH₂—N(R^{A})R^{B}, CH₂CH₂—N(R^{A})R^{B}, CO₂R^{A}, CH₂—CO₂R^{A}, or CH₂CH₂—CO₂R^{A};
wherein the heterocyclyl in (35) or (36) is piperidinyl, piperazinyl, morpholinyl, pyrrolidinyl, or thiomorpholinyl and is optionally substituted with oxo and also optionally substituted with 1 or 2 substituents each of which is independently Cl, Br, F, OH, CH₃, OCH₃, CH₂F, CF₃, OCH₂F, OCF₃, C(O)R^{A}, or CO₂R^{A}; and
Q′ is H or C₁-C₄ alkyl.

7. The compound according to claim 6, or a pharmaceutically acceptable salt thereof, wherein:
R⁴ is H, phenyl, CH₂-phenyl, or C(O)O—C₁-C₄ alkyl wherein:
the phenyl or the phenyl in CH₂-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN; (30) phenyl, (31) CH₂-phenyl, (32) CH(CH₃)-phenyl, (33) CH₂CH₂-phenyl, or (34) heteroaryl;
wherein the phenyl in (30), (31), (32), or (33) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
wherein the heteroaryl in (34) is pyridyl, pyrimidinyl, pyrrolyl, thienyl, furanyl, pyrazolyl, imidazolyl, oxazolyl, or thiazolyl, and wherein the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, or (p) CH₂CH₂—CO₂R^{A};
R⁵ is H, Cl, Br, F, C₁-C₄ alkyl, C₂-C₄ alkenyl, phenyl, O-phenyl, naphthyl, heteroaryl, NH₂, C(O)N(R^{7B})R^{8B}, SO₂N(R^{7B})R^{8B}, C(O)O—C₁-C₄ alkyl, C(O)H, or C(O)—C₁-C₄ alkyl, wherein:
the C₁-C₄ alkyl is optionally substituted with 1 or 2 substituents each of which is independently (1) Cl, (2) Br, (3) F, (4) OH, (5) OCH₃, (6) CH₂F, (7) CF₃, (8) OCH₂F, (9) OCF₃, (10) N(R^{A})R^{B}, (11) phenyl, or (12) N(R^{A})CH₂-phenyl;
wherein the phenyl in (11) or (12) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (O)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
the C₂-C₄ alkenyl is optionally substituted with (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, or (12) phenyl;
the phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, (29) CN, (30) phenyl, (31) CH₂-phenyl, (32) CH(CH₃)-phenyl, (33) CH₂CH₂-phenyl, (34) heteroaryl, (35) CH₂-heteroaryl, (36) CH₂CH₂-heteroaryl, (37) CH(CH₃)-heteroaryl, (38) heterocyclyl, (39) CH₂-heterocyclyl, (40) CH(CH₃)-heterocyclyl, or (41) C(O)-heterocyclyl;
wherein the phenyl in (30), (31), (32), or (33) is optionally substituted with 1 or 2 substituents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂—CO₂R^{A}, (p) CH₂CH₂—CO₂R^{A}, (q) C(O)R^{A}, (r) CH₂—C(O)R^{A}, (s) SO₂(C₁-C₄ alkyl), (t) SO₂N(R^{A})R^{B}, (u) NHSO₂CH₃, (v) CH₂NHSO₂CH₃, (w) C(O)N(R^{A})R^{B}, (x) CH₂C(O)N(R^{A})R^{B}, (y) CH₂OH, (z) CH₂CH₂OH, (aa) N(R^{A})C(O)R^{B}, (bb) N(R^{A})CH₂C(O)N(R^{A})R^{B}, or (cc) CN;
wherein the heteroaryl in (34), (35), (36), or (37) is pyridyl, pyrimidinyl, pyrrolyl, thienyl, furanyl, pyrazolyl, imidazolyl, oxazolyl, or thiazolyl, and the heteroaryl is optionally substituted with 1 or 2 substitutents each of which is independently (a) Cl, (b) Br, (c) F, (d) OH, (e) CH₃, (f) OCH₃, (g) CH₂F, (h) CF₃, (i) OCH₂F, (j) OCF₃, (k) N(R^{A})R^{B}, (l) CH₂—N(R^{A})R^{B}, (m) CH₂CH₂—N(R^{A})R^{B}, (n) CO₂R^{A}, (o)CH₂CO₂R^{A}, or (p) CH₂CH₂—CO₂R^{A};
wherein the heterocyclyl in (38), (39), (40) or (41) is piperidinyl, piperazinyl, morpholinyl, pyrrolidinyl, or thiomorpholinyl, wherein the heterocyclyl is optionally substituted with oxo, and is also optionally substituted with (1) CO₂R^{A}, (2) CH₂—CO₂R^{A} (3) C(O)(R^{A}), (4) N(R^{A})R^{B}, or (5) (CH₂)₁₋₃—N(R^{A})R^{B};
the O-phenyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN;
the heteroaryl is pyridyl, pyrimidinyl, pyrrolyl, furanyl, thienyl, pyrazolyl, imidazolyl, or thiazolyl, and the heteroaryl is optionally substituted with 1 or 2 substitutents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, or (16) CH₂CH₂—CO₂R^{A};
R^{7B} is the R⁷ associated with R⁵ and is H or C₁-C₄ alkyl;
R^{8B} is the R⁸ associated with R⁵ and is H, C₁-C₄ alkyl, cyclopentyl, cyclohexyl, phenyl, CH₂-phenyl, CH₂CH₂-phenyl, or CH(CH₃)-phenyl; wherein
the C₁-C₄ alkyl is optionally substituted with 2 substituents one of which is phenyl and the other of which is OH, (CH₂)₁₋₂—N(R^{A})R^{B}, or heterocyclyl;
wherein the heterocyclyl is piperidinyl, piperazinyl, morpholinyl, pyrrolidinyl, or thiomorpholinyl, wherein the heterocyclyl is optionally substituted with oxo, and is also optionally substituted with (a) CO₂R^{A}, (b) CH₂—CO₂R^{A} (c) C(O)(R^{A}), (d) N(R^{A})R^{B}, (e) (CH₂)₁₋₃—N(R^{A})R^{B};
the phenyl which is or is part of the R^{8B} is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{A}, or (29) CN;
alternatively the R^{7B} and R^{8B} together with the N to which both are bonded form heterocycyl which is piperidinyl, piperazinyl, morpholinyl, pyrrolidinyl, or thiomorpholinyl, wherein the heterocyclyl is optionally substituted with oxo and is also optionally substituted with 1 or 2 substituents each of which is independently Cl, Br, F, OH, CH₃, OCH₃, CH₂F, CF₃, OCH₂F, OCF₃, C(O)R^{A}, CO₂R^{A}, CH₂C(O)R^{A}, CH₂CO₂R^{A}, phenyl, CH₂-phenyl, CH₂CH₂-phenyl, CH₂CH₂CH₂-phenyl, or CH(CH₃)-phenyl;
wherein phenyl which is or is part of a substituent on the heterocyclyl is optionally substituted with 1 or 2 substituents each of which is independently (1) Cl, (2) Br, (3) F, (4) OH, (5) CH₃, (6) OCH₃, (7) CH₂F, (8) CF₃, (9) OCH₂F, (10) OCF₃, (11) N(R^{A})R^{B}, (12) CH₂—N(R^{A})R^{B}, (13) CH₂CH₂—N(R^{A})R^{B}, (14) CO₂R^{A}, (15) CH₂—CO₂R^{A}, (16) CH₂CH₂—CO₂R^{A}, (17) NHSO₂CH₃, (18) CH₂NHSO₂CH₃, (19) C(O)N(R^{A})R^{B}, (20) CH₂C(O)N(R^{A})R^{B}, (21) CH₂OH, (22) CH₂CH₂OH, (23) SO₂N(R^{A})R^{B}, (24) SO₂(C₁-C₄ alkyl), (25) C(O)R^{A}, (26) CH₂C(O)R^{A}, (27) N(R^{A})C(O)R^{B}, (28) N(R^{A})CH₂C(O)N(R^{A})R^{B}, or (29) CN; and
R⁶ is H.

8. The compound according to claim 7, or a pharmaceutically acceptable salt thereof, wherein:
XR² is (1) H, (2) C(O)O—CH₂CH₃, (3) phenyl optionally substituted with, Cl, OCH₃, or CF₃, (4) CH₂-phenyl, (5) pyridyl, (6) C(O)NH—CH₂-phenyl, (7) C(O)NH—CH₂-pyrrolidinyl, (8) C(O)NH—CH₂-piperidinyl, or (9) C(O)NH—CH₂CF₃;
R³ is OH, methyl, phenyl, HetZ, or N(H)R^{8C}, wherein:
the methyl is:
(1) substituted with phenyl which is substituted with another phenyl which is substituted by CH₂—N(R^{A})R^{B}, or
(2) substituted with phenyl which is substituted with (CH₂)₁₋₂-phenyl which is substituted by 1 or 2 substituents each of which is independently Cl, Br, or F;
the phenyl is substituted (i) with CH₂—N(R^{A})R^{B} or (ii) with another phenyl which is substituted by CH₂—N(R^{A})R^{B};
R^{8C} is:
(1) CH₂-phenyl in which the phenyl is substituted with OCH₃, CH₂NH₂, 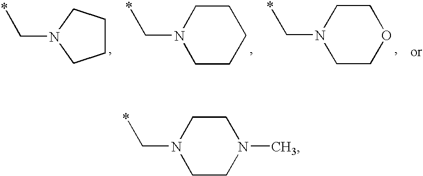
(2) CH(CH₃)-phenyl,
(3) CH₂-pyridyl in which the pyridyl is optionally substituted with 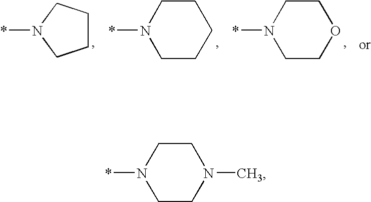
(4) methyl substituted with phenyl and with (CH₂)₁₋₂—N(R^{A})R^{B}, 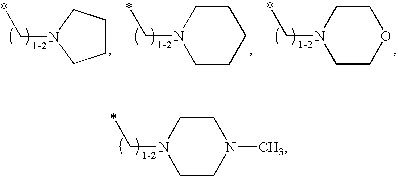
(5) phenyl substituted with phenyl which is optionally substituted with CH₂—N(R^{A})R^{B}, 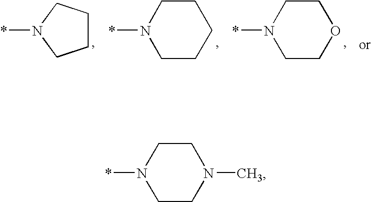
(6) substituted heterocyclyl selected from the group consisting of: 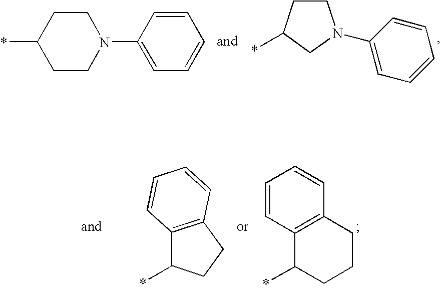
(6)
HetZ is: 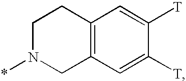
(1) wherein one T is phenyl, pyridyl, or C(O)OCH₃, and the other T is H, 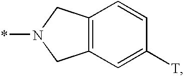
(2) wherein T is phenyl which is optionally substituted with CH₂—N(R^{A})R^{B}, or 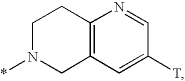
(3) wherein T is phenyl which is optionally substituted with CH₂—N(R^{A})R^{B};
R⁴ is H, C(O)OCH₃, C(O)OCH₂CH₃, or phenyl which is optionally substituted with Cl, Br, F, OH, CH₃, OCH₃, CF₃, OCF₃, or CH₂—N(R^{A})R^{B};
R⁵ is H, F, C(O)OCH₃, C(O)OCH₂CH₃, CH₂-phenyl, or phenyl which is optionally substituted with Cl, Br, F, OH, CH₃, OCH₃, CF₃, or OCF₃;
each R^{A} is independently H, CH₃, or CH₂CH₃; and
each R^{B} is independently H, CH₃, or CH₂CH₃.

9. The compound of claim 1, or a pharmaceutically acceptable salt thereof, which is a compound selected from the group consisting of Compounds 1-14, 16-59, and 61-268.

10. The compound according to claim 1, or a pharmaceutically acceptable salt thereof, wherein:
R¹ is O;
X is a bond or C(O);
R² is:
(1) H,
(2) halo,
(3) C₁-C₄ alkyl,
(4) O—C₁-C₄ alkyl,
(5) C₃-C₆ cycloalkyl,
(6) phenyl,
(7) C₁-C₄ alkylene-phenyl,
(8) NR^{7A}R^{8A}, or
(9) HetA
wherein phenyl is optionally substituted with a total of from 1 to 3 substituents where:
(i) from zero to 3 of the substituents are selected from the group consisting of halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), and C(O)N(C₁-C₄ alkyl)₂, and
(ii) from zero to 1 of the substituents is phenyl, C₁-C₄ alkylene-phenyl, O—C₁-C₄ alkylene-phenyl, C₁-C₄ alkylene-HetJ, or O—C₁-C₄ alkylene-HetJ;
wherein HetA and HetJ are each independently a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), or C(O)N(C₁-C₄ alkyl)₂;
and with the proviso (A) that XR² is not C(O)-halo;
R^{7A} is H or C₁-C₄ alkyl;
R^{8A} is:
(1) H,
(2) C₁-C₄ alkyl,
(3) C₁-C₄ fluoroalkyl,
(4) C₃-C₆ cycloalkyl,
(5) phenyl,
(6) C₁-C₄ alkylene-phenyl,
(7) HetB,
(8) C₁-C₄ alkylene-HetB,
(9) HetC, or
(10) C₁-C₄ alkylene-HetC;
wherein phenyl is optionally substituted with a total of from 1 to 3 substituents where:
(i) from zero to 3 of the substituents are selected from the group consisting of halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), and C(O)N(C₁-C₄ alkyl)₂, and
(ii) from zero to 1 of the substituents is phenyl, C₁-C₄ alkylene-phenyl, O—C₁-C₄ alkylene-phenyl, C₁-C₄ alkylene-HetJ, or O—C₁-C₄ alkylene-HetJ, where HetJ is as defined above;
wherein HetB is a 5- to 7-membered saturated heterocyclic ring containing from 1 to 3 heteroatoms selected from 1 to 3 N atoms, zero to 10 atom, and zero to 1 S atom optionally in the form S(O) or S(O)₂, wherein the saturated heterocyclic ring is attached to the rest of the molecule via a ring carbon atom, and wherein the saturated heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl; and
wherein HetC is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), C(O)N(C₁-C₄ alkyl)₂, phenyl, C₁-C₄ alkylene-phenyl or O—C₁-C₄ alkylene-phenyl;
alternatively, when X is C(O), R^{7A} and R^{8A} together with the N atom to which they are attached form a saturated heterocyclic ring selected from the group consisting of pyrrolidinyl, piperidinyl, piperazinyl, morpholinyl, thiomorpholinyl in which the S atom is optionally in the form S(O) or S(O)₂, and azepanyl, wherein the heterocyclic ring is optionally substituted with from 1 to 3 substituents each of which is independently oxo, C₁-C₄ alkyl, SO₂(C₁-C₄ alkyl), CO₂—C₁-C₄ alkyl, or C(O)—C₁-C₄ alkyl;
R³ is OH, NH₂, N(H)C(O)—C₁-C₄ alkyl, N(H)C(O)-phenyl, N(H)C(O)—C₁-C₄ alkylene-phenyl, N(H)-phenyl, or phenyl;
alternatively, R³ and XR² are taken together with the carbon atoms to which each is attached to provide: 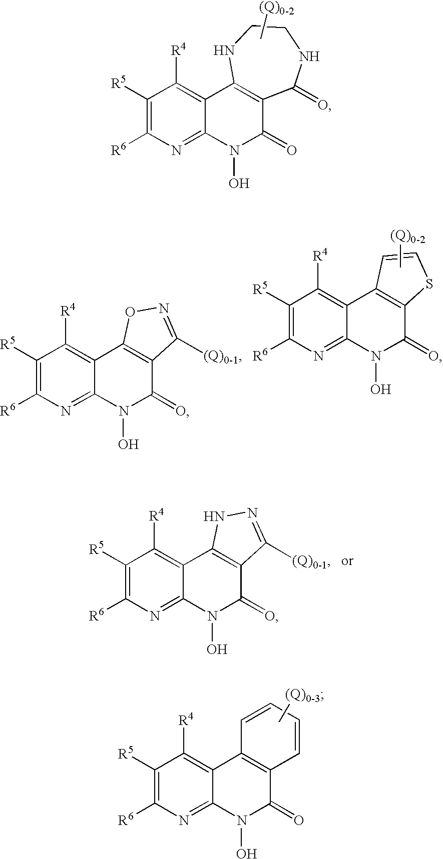
each Q is independently H, C₁-C₄ alkyl, halo, phenyl, or C₁-C₄ alkylene-phenyl;
R⁴ is H, CO₂—C₁-C₄ alkyl, or phenyl, wherein the phenyl is optionally substituted with from 1 to 3 substituents each of which is independently halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), or C(O)N(C₁-C₄ alkyl)₂;
R⁵ is:
(1) H,
(2) halo,
(3) C₁-C₄ alkyl,
(4) C₁-C₄ haloalkyl,
(5) C(O)O—C₁-C₄ alkyl,
(6) phenyl,
(7) C₁-C₄ alkylene-phenyl,
(8) C₁-C₄ alkenylene-phenyl,
(9) O-phenyl,
(10) SO₂N(H)-phenyl,
(11) SO₂N(C₁-C₄ alkyl)-phenyl,
(12) SO₂N(H)—C₁-C₄ alkylene-phenyl,
(13) SO₂N(C₁-C₄ alkyl)-C₁-C₄ alkylene-phenyl,
(14) naphthyl,
(15) C₁-C₄ alkylene-naphthyl,
(16) O-naphthyl,
(17) HetD,
(18) C₁-C₄ alkylene-N(H)—C₁-C₄ alkylene-phenyl,
(19) C(O)N(H)—C₁-C₄ alkylene-phenyl,
(20) C(O)N(C₁-C₄ alkyl)-C₁-C₄ alkylene-phenyl, or
(21) C(O)NR^{7B}R^{8B};
wherein:
phenyl or naphthyl is optionally substituted with from 1 to 3 substituents each of which is independently halo, OH, CN, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, N(H)SO₂—C₁-C₄ alkyl, C(O)NH₂, C(O)NH(C₁-C₄ alkyl), C(O)N(C₁-C₄ alkyl)₂, phenyl, C₁-C₄ alkylene-phenyl, O—C₁-C₄ alkylene-phenyl, HetK, C₁-C₄ alkylene-HetK, HetL, or C₁-C₄ alkylene-HetL; wherein
HetK is a 5- to 7-membered saturated heterocyclic ring containing from 1 to 3 heteroatoms selected from N, O and S optionally in the form S(O) or S(O)₂, wherein the saturated heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl;
HetL is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), or C(O)N(C₁-C₄ alkyl)₂;
HetD is a 5- or 6-membered heteroaromatic ring containing from 1 to 3 heteroatoms selected from N, O and S, wherein the heteroaromatic ring is optionally substituted with from 1 to 3 substituents each of which is independently halo, C₁-C₄ alkyl, O—C₁-C₄ alkyl, C₁-C₄ fluoroalkyl, O—C₁-C₄ fluoroalkyl, CN, SO₂(C₁-C₄ alkyl), CO₂—C₁-C₄ alkyl, C(O)—C₁-C₄ alkyl, NH₂, NH(C₁-C₄ alkyl), N(C₁-C₄ alkyl)₂, C(O)NH₂, C(O)NH(C₁-C₄ alkyl), C(O)N(C₁-C₄ alkyl)₂, phenyl, C₁-C₄ alkylene-phenyl or O—C₁-C₄ alkylene-phenyl;
R⁶ is H or C₁-C₄ alkyl;
R^{7B} is H or C₁-C₄ alkyl;
R^{8B} is H or C₁-C₄ alkyl; and
alternatively, R^{7B} and R^{8B} together with the N atom to which they are attached form a saturated heterocyclic ring selected from the group consisting of pyrrolidinyl, piperidinyl, piperazinyl, morpholinyl, thiomorpholinyl in which the S atom is optionally in the form S(O) or S(O)₂, and azepanyl, wherein the heterocyclic ring is optionally substituted with from 1 to 3 substituents each of which is independently oxo, C₁-C₄ alkyl, SO₂(C₁-C₄ alkyl), CO₂—C₁-C₄ alkyl, C(O)—C₁-C₄ alkyl, or C₁-C₄ alkylene-phenyl.

11. The compound according to claim 10, or a pharmaceutically acceptable salt thereof, wherein
R¹ is O;
XR² is:
(1) H,
(2) Cl, Br, or F,
(3) C₁-C₄ alkyl,
(4) C₃-C₆ cycloalkyl,
(5) C(O)OCH₃,
(6) C(O)OCH₂CH₃,
(6) phenyl,
(7) (CH₂)₁₋₂-phenyl,
(8) C(O)NR^{7A}R^{8A}, or
(9) HetA,
wherein phenyl is optionally substituted with from 1 or 2 substituents each of which is independently selected from the group consisting of Cl, Br, F, OH, CN, CH₃, OCH₃, CF₃, OCF₃, CN, SO₂CH₃, CO₂CH₃, C(O)CH₃, NH₂, NH(CH₃), N(CH₃)₂, N(H)SO₂CH₃, C(O)NH₂, C(O)NH(CH₃), and C(O)N(CH₃)₂, and
HetA is a heteroaromatic ring selected from the group consisting of pyridinyl, pyrimidinyl, and pyrazinyl, wherein the heteroaromatic ring is optionally substituted with 1 or 2 substituents each of which is independently Cl, Br, F, CH₃, OCH₃, CF₃, OCF₃, CN, SO₂CH₃, CO₂CH₃, C(O)CH₃, NH₂, NH(CH₃), N(CH₃)₂, C(O)NH₂, C(O)NH(CH₃), C(O)N(CH₃)₂, phenyl, CH₂-phenyl or OCH₂-phenyl;
R^{7A} is H or CH₃;
R^{8A} is:
(1) H,
(2) CH₃,
(3) CH₂CF₃,
(4) cyclopropyl,
(5) phenyl,
(6) CH₂-phenyl,
(6) CH(CH₃)-phenyl,
(7) HetB,
(8) CH₂—HetB,
(9) HetC, or
(10) CH₂—HetC;
wherein:
phenyl is optionally substituted with a total of 1 or 2 substituents where:
(i) from zero to 2 of the substituents are selected from the group consisting of Cl, Br, F, OH, CN, CH₃, OCH₃, CF₃, OCF₃, CN, SO₂CH₃, CO₂CH₃, C(O)CH₃, NH₂, NH(CH₃), N(CH₃)₂, N(H)SO₂CH₃, C(O)NH₂, C(O)NH(CH₃), and C(O)N(CH₃)₂, and
(ii) from zero to 1 of the substituents is phenyl, CH₂-phenyl, OCH₂-phenyl, CH₂-pyridinyl, or OCH₂-pyridinyl;
HetB is a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the saturated heterocyclic ring is attached to the rest of the molecule via a ring carbon atom, and wherein the saturated heterocyclic ring is optionally substituted with 1 or 2 substituents each of which is independently oxo, CH₃, SO₂CH₃, CO₂CH₃, C(O)CH₃, or CH₂-phenyl; and
HetC is a heteroaromatic ring selected from the group consisting of pyridinyl, pyrimidinyl, and pyrazinyl, wherein the heteroaromatic ring is optionally substituted with 1 or 2 substituents each of which is independently Cl, Br, F, CH₃, OCH₃, CF₃, OCF₃, CN, SO₂CH₃, CO₂CH₃, C(O)CH₃, NH₂, NH(CH₃), N(CH₃)₂, C(O)NH₂, C(O)NH(CH₃), C(O)N(CH₃)₂, phenyl, CH₂-phenyl or OCH₂-phenyl;
alternatively, R^{7A} and R^{8A} together with the N atom to which they are attached form a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the heterocyclic ring is optionally substituted with oxo, CH₃, SO₂CH₃, CO₂CH₃, or C(O)CH₃;
R³ is OH, NH₂, N(H)C(O)CH₃, N(H)C(O)-phenyl, N(H)C(O)CH₂-phenyl, N(H)-phenyl, or phenyl;
alternatively, R³ and XR² are taken together with the carbon atoms to which each is attached to provide: 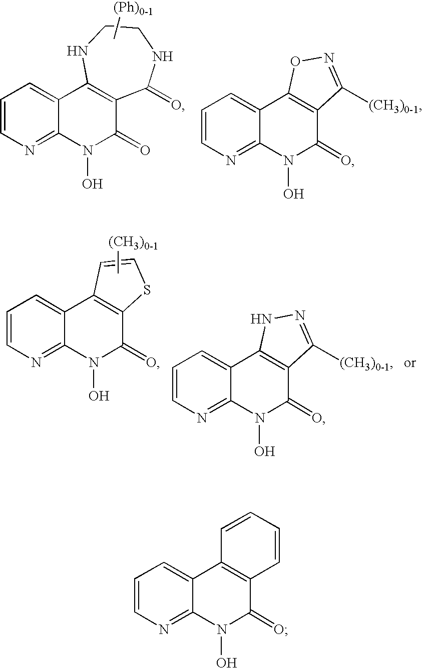
R⁴ is H, CO₂CH₃, CO₂CH₂CH₃, or phenyl;
R⁵ is:
(1) H,
(2) Cl, Br or F,
(3) C₁-C₄ alkyl,
(4) CH₂CF₃,
(5) CH₂CH(CH₃)Br,
(6) C(O)OCH₃,
(7) C(O)OCH₂CH₃,
(8) phenyl,
(9) CH₂-phenyl,
(10) CH(CH₃)-phenyl,
(11) CH═CH-phenyl,
(12) O-phenyl,
(13) SO₂N(H)-phenyl,
(14) SO₂N(CH₃)-phenyl,
(15) SO₂N(H)CH₂-phenyl,
(16) SO₂N(CH₃)CH₂-phenyl,
(17) naphthyl,
(18) CH₂-naphthyl,
(19) O-naphthyl,
(20) HetD,
(21) CH₂N(H)CH₂-phenyl,
(22) CH(CH₃)N(H)CH₂-phenyl,
(23) C(O)N(H)(CH₂)₁₋₂-phenyl,
(24) C(O)N(CH₃)(CH₂)₁₋₂-phenyl, or
(25) C(O)NR^{7B}R^{8B};
wherein:
phenyl is optionally substituted with a total of 1 or 2 substituents where:
(i) from zero to 2 of the substituents are selected from the group consisting of Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), and C(O)N(CH₂CH₃)₂, and
(ii) from zero to 1 of the substituents is phenyl, CH₂-phenyl, OCH₂-phenyl, HetK, CH₂—HetK, HetL, or CH₂—HetL; wherein
HetK is a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the saturated heterocyclic ring is attached to the rest of the molecule via a ring carbon atom, and wherein the saturated heterocyclic ring is optionally substituted with 1 or 2 substituents each of which is independently oxo, CH₃, CH₂CH₃, SO₂CH₃, SO₂CH₂CH₃, CO₂CH₃, CO₂CH₂CH₃, C(O)CH₃, C(O)CH₂CH₃, or CH₂-phenyl; and
HetL is a heteroaromatic ring selected from the group consisting of thienyl, pyrrolyl, pyrazolyl, imidazolyl, pyridinyl, pyrimidinyl, and pyrazinyl, wherein the heteroaromatic ring is optionally substituted with 1 or 2 substituents each of which is independently Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), C(O)N(CH₂CH₃)₂, phenyl, CH₂-phenyl or OCH₂-phenyl;
HetD is a heteroaromatic ring selected from the group consisting of thienyl, pyrrolyl, pyrazolyl, imidazolyl, pyridinyl, pyrimidinyl, and pyrazinyl, wherein the heteroaromatic ring is optionally substituted with 1 or 2 substituents each of which is independently Cl, Br, F, OH, CN, CH₃, CH₂CH₃, OCH₃, OCH₂CH₃, CF₃, OCF₃, CN, SO₂CH₃, CO₂CH₃, CO₂CH₂CH₃, C(O)CH₃, C(O)CH₂CH₃, NH₂, NH(CH₃), N(CH₃)₂, N(H)SO₂CH₃, NH(CH₂CH₃), N(CH₂CH₃)₂, N(H)SO₂CH₂CH₃, C(O)NH₂, C(O)NH(CH₃), C(O)N(CH₃)₂, C(O)NH(CH₂CH₃), C(O)N(CH₂CH₃)₂, phenyl, CH₂-phenyl or OCH₂-phenyl;
R^{7B} is H, CH₃, or CH₂CH₃;
R^{8B} is H, CH₃, or CH₂CH₃; and
alternatively, R^{7B} and R^{8B} together with the N atom to which they are attached form a saturated heterocyclic ring selected from the group consisting of pyrrolidinyl, piperidinyl, piperazinyl, morpholinyl, and thiomorpholinyl in which the S atom is optionally in the form S(O) or S(O)₂, wherein the heterocyclic ring is optionally substituted with oxo, CH₃, SO₂CH₃, CO₂CH₃, C(O)CH₃, or (CH₂)₁₋₂-phenyl; and
R⁶ is H.

12. A pharmaceutical composition comprising an effective amount of the compound according to claim 1 or a pharmaceutically acceptable salt thereof and a pharmaceutically acceptable carrier.

13. A method of inhibiting HIV integrase or HIV RHase H or both in a subject in need thereof which comprises administering to the subject an effective amount of the compound according to claim 1 or a pharmaceutically acceptable salt thereof.

14. A method for treating infection by HIV or for, treating or delaying the onset of AIDS in a subject in need thereof which comprises administering to the subject in need thereof an effective amount of the compound according to claim 1 or a pharmaceutically acceptable salt thereof.

15. The method of claim 14, further comprising administering to the subject a second HIV antiviral agent other than a compound of Formula I selected from the group consisting of HIV protease inhibitors, HIV integrase inhibitors, non-nucleoside HIV reverse transcriptase inhibitors, and nucleoside HIV reverse transcriptase inhibitors.

16. (canceled)

17. (canceled)

18. (canceled)

19. A pharmaceutical combination which is (i) a compound according to claim 1 or a pharmaceutically acceptable salt thereof, and (ii) a second HIV antiviral agent other than a compound of Formula I selected from the group consisting of HIV protease inhibitors, HIV integrase inhibitors, non-nucleoside HIV reverse transcriptase inhibitors, and nucleoside HIV reverse transcriptase inhibitors.
